SEQUENCE LISTING (22 sequences)

SEQ ID NO: 1 - protein, 450 aa, Bacillus sp.
  source 1..450: note = BK245
MTTKRNFATSRFERFSYNIYFLGQNSIFFIVSSFLAVYYTNVLGISAATVGSILLLARIWDALVDPMLATTIERLKLKGGKFKPWVNLAAVTVPLFTFLCFGFSNQLLEASGGVRIAYAAITYLVWGTLYAAADAPAFALSTVMTPNPEERTVLLSNNQLTGIIGILISVAAFPAVLKATNNNYMLSVAIFAIFSCLVMNMTRFAKERVVSNKREPNIKEIISSLMNNKYLKLIVLVNILASGANFANTLAPYVASDIYHDGAKVSLILGITILPVIIAAPIMPLLVRKFGKIKLYAVALSATVVFSVLSYFVAYDNFNLFLLISLIKAFLIAPQVIIYPMFFSDAVEYDFFKNGTRFEAVTFATQTFSNKIIGAISGGLGMWIIGLAGYEAAKNGHAVMQSTHTLNALWATFNLGSAVGSLLALVIFVKWYDFSDAKVKEMAIANEVKM

SEQ ID NO: 2 - protein, 471 aa, Bacillus sp.
  source 1..471: note = AFS096315
MGSGNMGKNSVVNERIIIEKNAKPKNAFVTKKSERFMYSSYFFGQNIIFFIVSSFLAVYYTSVLGISATTVGSILLLARIWDAFVDPTLATIIERLNLKSGKFKPWVNLASITVPIMTFLCFGFSHQLLAASEGVRIGYAAVTYLVWGTLYAAADAPAFAFSTVITPNPEERTVLLANNQITGIVGILVAVAAFPSVLKATDNNYMISVLIFSIIAFVVMSMTRFVKERVKSNKKEPAIKEILHSLFNNKYLKTIVIVNIIANGVNFTNTLAPFVATDIYKDATKVSLILGIGILPVVLVAPFVPKLVNKYGKIKLMSFALAANAILSIVTYFVAYENFGLFLGVSVLKAMLIAPQLVIYPMFFADSVEYDCYKNGTRFEAVIFATQTFSSKIIGAISGGLGMWIIGIAGYKAATNGHAVTQTATTLKSLWATFNLGGAVGCLIALFIFLKFYDLTEEQVKEMALANQSKN

SEQ ID NO: 3 - protein, 451 aa, Synthetic construct
MRNQKILLLQKKSERFMYSSYFFGQNIIFFIVSSFLAVYYTSVLGISATTVGSILLLARIWDAFVDPTLATIIERLNLKSGKFKPWVNLASITVPIMTFLCFGFSHQLLAASEGVRIGYAAVTYLVWGTLYAAADAPAFAFSTVITPNPEERTVLLANNQITGIVGILVAVAAFPSVLKATDNNYMISVLIFSIIAFVVMSMTRFVKERVKSNKKEPAIKEILHSLFNNKYLKTIVIVNIIANGVNFTNTLAPFVATDIYKDATKVSLILGIGILPVVLVAPFVPKLVNKYGKIKLMSFALAANAILSIVTYFVAYENFGLFLGVSVLKAMLIAPQLVIYPMFFADSVEYDCYKNGTRFEAVIFATQTFSSKIIGAISGGLGMWIIGIAGYKAATNGHAVTQTATTLKSLWATFNLGGAVGCLIALFIFLKFYDLTEEQVKEMALANQSKN

SEQ ID NO: 4 - protein, 451 aa, Synthetic construct
MLNRRMPLLPKKSERFMYSSYFFGQNIIFFIVSSFLAVYYTSVLGISATTVGSILLLARIWDAFVDPTLATIIERLNLKSGKFKPWVNLASITVPIMTFLCFGFSHQLLAASEGVRIGYAAVTYLVWGTLYAAADAPAFAFSTVITPNPEERTVLLANNQITGIVGILVAVAAFPSVLKATDNNYMISVLIFSIIAFVVMSMTRFVKERVKSNKKEPAIKEILHSLFNNKYLKTIVIVNIIANGVNFTNTLAPFVATDIYKDATKVSLILGIGILPVVLVAPFVPKLVNKYGKIKLMSFALAANAILSIVTYFVAYENFGLFLGVSVLKAMLIAPQLVIYPMFFADSVEYDCYKNGTRFEAVIFATQTFSSKIIGAISGGLGMWIIGIAGYKAATNGHAVTQTATTLKSLWATFNLGGAVGCLIALFIFLKFYDLTEEQVKEMALANQSKN

SEQ ID NO: 5 - protein, 559 aa, Acholeplasmataceae bacterium
MELTQTNGKLSKRTKWSYSLGGIGRDMTYALVSTFLMTYVQYGVGPTVAQYSVIGIILIVGRFWDAINDPIMGAIIENTKSRWGKFKPWILIGAVLTGIVIVLMFNWVRWVPWAKDGWNYVIFFGITYFLWEIAFTMNDISYWSMLPSLSKNEKERNSITTLAVVFAGVGAFAANAGINFITVGNAIKGYGMVAIIIALFLIGCQVLTVFGVKQPVQTTGEINEVTETVEAKKKEEKVTFKKMLLVIKNNDQLLWIILAMLFYNIGSGILVALGYNFIYMELGYDGFLAMVFVATFGVTTIGVQVFYSELVKKFGRKRLLFYSLIMIAFGYLFLLAAGNVPFLPLNIITVCLFGIFAFGGQAIFYMILTVNIANTVEYNEYRTNERNEAVIFSLRPLMVKFASALQQGITILVLTVTGIYALSQNVNFLEHQKALFDKMDVQQQEEYKQNIADRVVIFEGEDYKDLDPKVKETYYDMLQEISFNMNEKEGKEEMYINDAADIYFKDIASKPMRFSLRMTITLVPTILIALSYLVLNKKYIIDEKMYQMMVREIEARNIK

SEQ ID NO: 6 - protein, 541 aa, Tenericutes bacterium
MKQATNDYLKRNKWAYSLGGIGRDMTYTLVATFFMTYIQYAGLGLSVAQFSVIGILLIVGRIWDAINDPMMGAIIENTKSRYGKFKPWILIGAVLTAIVIVIMFNVRPLGNKGWNFVIFFGIIFLAWEITFTMNDISYWSMLPSLSTDPKHRDKIATLAVVFAGVGAFAAHAMITFLTVGNAVAGYSFISIIIALFLVGCQLLTVLGVKEPEVKDTIPQENVSLKKMIRVIAKNDQLLWLILAMLLYNLGSNILVALGYNFFFMELGYDPFKVTIFIVTFGVSNLSIQALYPKIVSKMGRKKLLGLSLLILAFGYLLLFLMGFLAILPVSLVTICLFGIFVFTGQALFYMVLTVNIANTIEYNQYKTGERNEGIIFSLRPFMAKMASALEQGFITLVLVTSGIYLLSQNVSELEKQKNIYDNLEVAEQLEYKANILLKDNIFVGEEYKKYSPEEIEQFYEMLKDVEFVEEKGKNVMLITDTADANFRDLATTKMRLLLRIAITVVPTLLITTAYIILKKKFIIDEAMYEKMVAEIKAKQAI

SEQ ID NO: 7 - protein, 452 aa, Clostridium disporicum
MEVLEKVNRNESDVSMKTLISFGMGSLGNNIICAMIGTYLTIYLTDSFGISAAAVGTLFLVARIIDAITDPIMGMIVDNTKSKMGKSRPYLLFVPIFMGIATIMCFSSPNLSDTGKIVWVYISYILWGICFTAMDTPYWSLSANITQSAQGRTKIITSARTIAFGGSLVVSVFTLPLVAKFGNWTIVAIVYSITAAICTWVTVWGVREINTNRKPQKQGIKQLKELFKTNRPLRIVLISMLMLEITYALRNGFSIYYIKYNFNAELYIPLVTGISITTGMLGGICTPSITKILGKRKTALFGIAINAIGFLSIFLLKYSNLKMMLVINAFMGLAEGAANISLASMVADCVEYGEWKTGKRSEGMIFSSNIFKTKLASAIGGALCGYVLAFVGYEANVAQTVNTLNGIHIMYSIIPGIIAVCSIISLRRYNLTEKEYEAILEDLNKDEPLETA

SEQ ID NO: 8 - protein, 451 aa, Paenibacillus yonginensis
MKPTVHRISSREKVGYATGDLAFNLIYQTVSSYLLFFYTDVFGISSAAAGLMFLIVRIFDAVVDPLIGTVVDRTNSKYGRFRPYLLYGAIPFAIMSVLCFTTPGFSDTGKLVYAYITYILLSVTYTTINVPYGALTSVITQDNKEVVSLTSVRVFFSNIGGMIVTYGVPLLVGVFAAANMKTSSSWQLTMTIMGVLGCLLLFYCFFNTKERVKVPDHAAEKINLKDAFEQFRVNRPLVLICLFFIINFGVNSIINSVGVYYITYNVDRPDLVKWYGLLGVLPALVCMPLMPFLYKWMGKRTLLFTTLTFKMVGLLALWVIPPSMVPLVLTARIISAIGTVTAGGLCWALVPETIDYGEYKTGKRMGGLIYAIIGFFYKFGMALGGIVPGLILDRFGYVANQAQTLTALHGILLTTTIIPAVFIIIELIAIYFYNLDEKEHKRVVAELEARQ

SEQ ID NO: 9 - protein, 509 aa, Oscillospiraceae bacterium
MANNAPQGRALKTYTKKESAMYYVGLAGQNVFYNIIGTALTYYLQFTILIPAFTVSVIMAIARVWDAVNDPMMGTIVDKTRTKWGKCRPYLLFVPFPIMIITILCFTNFGFYDPAASATSMHNILIVAYAAFTYILWGMTYTVGDIPLWGITALMTEKDDDRNKLLSFARIWASVGAGITLLTINSVALKLGSTFAVNGRTPAQGERLGFITAAVIFSVVGFILYQMAGIFVRERVPASKETRTMKQNFALMWQNKPFRQILLSGVLGSPKSTIALAAMSLVSYYYASKDPLLAMLYILLLGGALFIGQFLSMGLAPMLVKKYEKKSLYNWSNLIGAIPYGGLFLVYMIAPNHDLTTVACLIICAVLFFGSGFSMGITNALQSLMIADAVDYEEYCSGIRPDGVFFSGQTFIAKITAAIATVINGIAYSVVGFSDAKVQEVNDYIAAGGIPRLNPEYQPFMTVLFFIVSVPPIIGCILSVLPTIKYALSDKEHKRILDELNERRHAAEN

SEQ ID NO: 10 - protein, 479 aa, Erwinia iniecta
MSVSLKTKISYGFGAFGKDFAIGIIYMYLMYYYTDVVGVSVSMVASLFLVARILDALLDPVMGWIVERTRTRWGKFKPWILIGTLVNSAVLISVFCAHNFSGGALIAYIWVTYILWGFTYTIMDVPFWSLVPCITVDKREREALIPWPRFFASSAGYLTGVIGLPLVNLLGQGHQGEGFMRFTFILVFFFILSTLVTLINVKEKYSSSHPGATQVQHGLKSVLMMIVRNKPLFALLIMALSWNMAHNVIQSFAIYYFKYVVGREELFPWYMMYAGIANLATIALFPMLAKRFSRRVLWISASVLPVLSCLLLLYVGLYAPQSAALIALSGIMANSGGAFFWVLLVIMVADTVDYGDYALGMRIESIAFAVQTMVVKAGAAFAGLAIGVLLGAVGYQAGVAQTASTVMGMQGIMIVLPMLFFALTLWVYLRHYTLDGALLQQVQNHLKRKYDEPVDAAARAAAVATDFKPAAGEPTTVKS

SEQ ID NO: 11 - protein, 466 aa, Bacillus sp.
  source 1..466: note = AFS002410
MGKNSIVNERVIIENNANPKSLFATSRLERFMYNIYFFGQNIIFFIVSSFLAVYYTNVLGISATTVGSILLLARIWDAFVDPTLATVIERLNLKSGKFKPWVNLAAITVPVMTFLCFGFGNQLLHASEGARIAYAAITYLIWGTLYAAADAPAFAFSTVITPNPEERTVLLANNQITGIIGILVAVAAFPSVLKATHNNYMITVLIFSIIAFIVMSFTRFVKERVKSNKKEPAIKEILHSLLNNKYLKTIVIVNIIANGVNFTNTLAPFVATDIYKDATKVSLILGVGILPVVLVAPFVPRLIRKFGKIKLMSVALGANALLSFITFFVAYENFGLFLGISVLKAIFIAPQLVIYPMFFADSVEYDCYQNGTRFEAVIFATQTFSSKIIGAISGGLGMWIIGVAGYKAATGGHAITQTAGTLKSLWATFNLGGAVGCLIALIIFLKFYDLSEDRVKEMAVANQTKN

SEQ ID NO: 12 - protein, 450 aa, Paenibacillus sp.
  source 1..450: note = OV219
MTKQYAATRFERTSYDLFFSGQNIVYFILTSFLSVYYTGTLGIPATAVAGILLAARIWDAFVDPMLAAVVERSNFKSGKFRPWVLLAAITVPFFTILCFGFDGFLLDQSLSVRIAYAAITYFLWGTFYAAADAPAYALSTVMTPYPEERNLLLTYNKITGLVGGLAGMVAFPLVLSATDDNWFLTVLVFSLIAFGCMMMIRFAKERIKHVPDNPPSMSVIFKTILSNKYLVIIVIVSLIANGLNFMQTLTPFLASDIYHDSNATATLLGVSVLPMILVAPFAPVLIRKFGKINLVVISFAATAVFSVLLYFFAKESLTMFLVLSFLKGLFSAPFLVVYSLFFADCIEYDAHQKGKRFEAVTFAAQTFMAKVSAAISGGVGMWIIGIAGYKAAVEGETVTQSDTALNALWASVHWGPVVGSVIALVLFLKYYDLTDEKVKKLVESNLEKGV

SEQ ID NO: 13 - protein, 464 aa, Vibrio kanaloae
MYKYKLPVLEKVGFGAGDMAVNVVISSMMLIITFFYTDIFGIKPEDLAMLFIVVRLIDAVTDPLMGMITDKFTSRWGRYRQYLLFLAIPFGVSVYLAFSTPDGDYNTKLVYAYATYILITVMFTAVTIPYISLISVMTDDPKERLSANGYRLFFAKIAAFLVTIIVPQLSTAWGQDNIQLGYQYSMGLMGLMGTLLFLFCFATTKERIEHVVDKKSFKDQVKLLMRNDQWLVLCAVCVTGTIGYVIRGSVAAYYAKYYLSGDAGTISAFLATGVGAAILAMVASTWITKKYCKIKLFRYSQVSVLLLSLMLYVFVGREDLALAFVLYFLVSFVVDLHAPVFWSAIAEAVDYGAYKTGQRVSGLAFGGISFSQKLGMGIGGAVVGWLLTYFNYVPNEAQSDYALTGIALMLTVIPGFFHFLMGSLMSKYKVTDKYYQSMTAANIQGGKKNLSEVQPSKPQTSFEN

SEQ ID NO: 14 - DNA, 80 bp, Synthetic construct
  source 1..80: mol_type = other DNA
gtcaataaaggcatataaaaggaggttaataacatgaaagttaaagtaaaacatcttaatcatgctgcggagggtctcta

SEQ ID NO: 15 - DNA, 129 bp, Synthetic construct
  source 1..129: mol_type = other DNA
ccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 16 - protein, 506 aa, Synthetic construct
MHRKEERSKTYTKKESAMYYVGLAGQNVFYNIIGTALTYYLQFTILIPAFTVSVIMAIARVWDAVNDPMMGTIVDKTRTKWGKCRPYLLFVPFPIMIITILCFTNFGFYDPAASATSMHNILIVAYAAFTYILWGMTYTVGDIPLWGITALMTEKDDDRNKLLSFARIWASVGAGITLLTINSVALKLGSTFAVNGRTPAQGERLGFITAAVIFSVVGFILYQMAGIFVRERVPASKETRTMKQNFALMWQNKPFRQILLSGVLGSPKSTIALAAMSLVSYYYASKDPLLAMLYILLLGGALFIGQFLSMGLAPMLVKKYEKKSLYNWSNLIGAIPYGGLFLVYMIAPNHDLTTVACLIICAVLFFGSGFSMGITNALQSLMIADAVDYEEYCSGIRPDGVFFSGQTFIAKITAAIATVINGIAYSVVGFSDAKVQEVNDYIAAGGIPRLNPEYQPFMTVLFFIVSVPPIIGCILSVLPTIKYALSDKEHKRILDELNERRHAAEN

SEQ ID NO: 17-18 - skipped (2 entries)

SEQ ID NO: 19 - protein, 453 aa, Paenibacillus pectinilyticus
MKEQTTTYVATRFERISYDMFFTGQNIIYMVLSTFLGVYYTGNLGIPATAVAAILLVARIWDAFMDPLLATVIEKSNFKIGKFRPWVLMAAFTVPLFMLLCFSFNDFLIGQSLVVRIIYASVTYFIWGTLYAAADAPAYALSTAMTPVPEERNLLLSYNKLTGLIGVIIGMVLFPTILNATDNNYFISVLVMSVIAFLTMIMIRFTKERVKQEGRKQPAVSEIFKSIAGNKYLVLIVLIGIIANGTNFAMTLTPFVALDIYGDSTKVTAMLGLGIIPMLLIAPLSPLLIRKFGKKNLAVFSFAATAIFSILIYFFAKDNYMLYLLLSLIRGLLSAPFIIVSALFFTDCIEFDDYTNGKRFEAVTFAAQTFMSKLVGAISGGVGMWIIGVAGYKAAVAGETVTQTPSALNALWAAMNIGPAVGSVIAIIIMLKFYDLSEDKVQMMIESNLQKTK

SEQ ID NO: 20 - protein, 441 aa, Paenibacillus sp.
  source 1..441: note = FSL A5-0031
MHQVSAKEKWSYGVGAMGQNLVYGLMTFLLMVFYTDVLGISAVFVGTLFLLVRIWDALLDPVIAVIVDRIHTRWGKFRPFILAGGILVSILTVLCLYAPDFSPAMKITYVIITYALWNTAYALFDVPFWSLAPAMTSDPIERTKVIAIAKMLGIIGGVVAGGASLPIINAIGNGNASKGYFWTAVIFAALCILFTIILFRNTKEHIYEVKPEHESLKASLNVVLKNKPLLILTLAGLFTGTSLILKQTVTLYYVRYNLGNEGLFTAFSLSSMLFMLLAIGIMPRISAKFGKKKTFIAGGIICITANVLFFFGQSDQILFLFAMNAISMFGVGFSIVLAASMQADTIEYAQWKTGKRSESIVTAVGTFNGKIGTAIAGAVAGYGLTLFDYSPNVTQSDTALTGINLMMSIIPAMGIVLSIVIISFYDLSEKKYAEIVADLKA

SEQ ID NO: 21 - protein, 462 aa, Candidatus Fimenecus excrementigallinarum
MKETTLKNPNQTRVSERIAYGVFFLGQGFIYSMISRYLMIYLTDYVHISTAIVTVLLTASKFWDAVNDTLFGVVVDKVRFKSGNRFRPWLNLSTLILPVGTILLFAVPGGAAQGVKATWAVAAYLIWDLLYTMCDVPIFSLVTTMTNNIRERGNIYTIAGIGGAIATALTSLFLVQIFDRVGFLYTAITVSALGLLFMRPIVHLAKERHRPTEKEREQNATLRDMWVYVKGNKYLRYFIVYRLISGSTFISVLTYFTKHCLNDIRFETTIMLVAIPFSAVLYLCAPLLMKRFNKISIYRVCMVLVILAYGTLYFVGYESKWLMVALLVCALDAAIIPSILMSAIPADCVEYGCYKTGIRKEGITFAMQSFTNKFCSSVATALAGVLLMLIGYDSESGAMMNEAQQAGLFAGMCWFPIVGQLIGLPLLFKYKLRDRDVQIMADVNEGKLPREEAERLFAGRYD

SEQ ID NO: 22 - protein, 476 aa, Salmonella typhimurium
  source 1..476: note = strain LT2
MSISLTTKLSYGFGAFGKDFAIGIVYMYLMYYYTDVVGLSVGLVGTLFLVARIWDAINDPIMGWIVNATRSRWGKFKPWILIGTLTNSLVLFLLFSAHLFEGTAQVVFVCVTYILWGMTYTIMDIPFWSLVPTITLDKREREQLVPFPRFFASLAGFVTAGITLPFVSYVGGADRGFGFQMFTLVLIAFFIASTIVTLRNVHEVYSSDNGVTAGRPHLTLKTIVGLIYKNDQLSCLLGMALAYNIASNIINGFAIYYFTYVIGDADLFPYYLSYAGAANLLTLIVFPRLVKMLSRRILWAGASVMPVLSCAGLFAMALADIHNAALIVAAGIFLNIGTALFWVLQVIMVADTVDYGEFKLNIRCESIAYSVQTMVVKGGSAFAAFFIALVLGLIGYTPNVAQSAQTLQGMQFIMIVLPVLFFMMTLVLYFRYYRLNGDMLRKIQIHLLDKYRKTPPFVEQPDSPAISVVATSDVKA

(19) United States
(12) Patent Application Publication
(10) Pub. No.: US 2026/0139285 A1
(43) Pub. Date: May 21, 2026

(54) SACCHARIDE IMPORTERS FOR LACTO-N-TRIOSE

(71) Applicant: INBIOSE N.V., Zwijnaarde (BE)

(72) Inventors: Sofie Aesaert, Zwijnaarde (BE); Joeri Beauprez, Zwijnaarde (BE); Kristof Vandewalle, Zwijnaarde (BE); Annelies Vercauteren, Zwijnaarde (BE); Tom Verhaeghe, Zwijnaarde (BE)

(21) Appl. No.: 19/119,350

(22) PCT Filed: Oct. 25, 2023
(86) PCT No.: PCT/EP2023/079833

§ 371 Date: Apr. 8, 2025

(30) Foreign Application Priority Data
Oct. 25, 2022 (EP) 22203562.8
Oct. 25, 2022 (LU) LU502955
May 3, 2023 (EP) 23171352.0

(51) Int. Cl. C12P 19/04; C07K 14/00; C12N 9/10; C12N 15/52

(52) U.S. Cl. CPC C12P 19/04; C07K 14/00; C12N 9/1048; C12N 9/1051; C12N 15/52

(57) ABSTRACT

This disclosure is in the technical field of synthetic biology, metabolic engineering and cell cultivation. This disclosure provides a cell for the production of one or more bioproduct(s) wherein the cell is capable to produce one or more precursor(s) used in the production of the one or more bioproduct(s) and wherein the cell is genetically engineered to express a saccharide importer that internalizes at least one of the one or more precursor(s). This disclosure furthermore provides a cell that is genetically engineered to express or to overexpress a saccharide importer that has uptake activity for lacto-N-triose (LN3, GlcNAc-beta1,3-Gal-beta1,4-Glc). The invention also provides the use of the cells in a cultivation or incubation. The invention also describes methods for the production of one or more bioproduct(s) using any one of the saccharide importers as well as the purification of the bioproduct(s). The invention furthermore relates to saccharide importers having uptake activity for LN3 and the use of any one of the saccharide importers for the production of one or more bioproduct(s).

### CROSS-REFERENCE TO RELATED APPLICATIONS

This application is a national phase entry under 35 U.S.C. § 371 of International Patent Application PCT/EP2023/079833, filed Oct. 25, 2023, designating the United States of America and published as International Patent Publication WO 2024/089131 A1 on May 2, 2024, which claims the benefit under Article 8 of the Patent Cooperation Treaty of European Patent Application Serial No. 22203562.8, filed Oct. 25, 2022; European Patent Application Serial No. 23171352.0, filed May 3, 2023; and Luxembourg Patent Application Serial No. LU502955, filed Oct. 25, 2022.

### STATEMENT REGARDING ELECTRONIC FILING OF A SEQUENCE LISTING

Pursuant to 37 C.F.R. § 1.831 through 1.835, a Sequence Listing XML file entitled “065-PCT Sequence List” 28 KB in size, generated Oct. 25, 2023, has been submitted via EFS-Web is provided in lieu of a paper copy. This Sequence Listing is hereby incorporated by reference into the specification for its disclosures.

### TECHNICAL FIELD

This disclosure is in the technical field of synthetic biology, metabolic engineering and cell cultivation. This disclosure provides a cell for the production of one or more bioproduct(s) wherein the cell is capable to produce one or more precursor(s) used in the production of the one or more bioproduct(s) and wherein the cell is genetically engineered to express a saccharide importer that internalizes at least one of the one or more precursor(s). furthermore provided is a cell that is genetically engineered to express or to overexpress a saccharide importer that has uptake activity for lacto-N-triose (LN3, GlcNAc-beta1,3-Gal-beta1,4-Glc). Also provided is the use of the cells in a cultivation or incubation. Also described are methods for the production of one or more bioproduct(s) using any one of the saccharide importers as well as the purification of the bioproduct(s). The disclosure furthermore relates to saccharide importers having uptake activity for LN3 and the use of any one of the saccharide importers for the production of one or more bioproduct(s).

### BACKGROUND

Saccharides are widespread in nature and function in many vital phenomena such as differentiation, development and biological recognition processes related to the development and progress of fertilization, embryogenesis, inflammation, immunological processes, metastasis, and host pathogen adhesion. An important group of saccharides can be found in milk. Mammalian milk saccharides, like mammalian milk oligosaccharides (MMOs) including human milk saccharides like human milk oligosaccharides (HMOs), modulate key developmental and immunological processes in early life. HMOs, e.g., are milk bioproducts known to improve infant immediate and long-term health and development by shaping the infant's gut microbiome, affecting the infant's immune system, protecting the infant from intestinal and immunological disorders, and by being involved in proper brain development and cognition. Additionally, HMOs have been reported to have a role in boosting adults health (Azad et al. (2018), J. Nutr. 148, 1733-1742; Bode (2015), Early Hum. Dev. 1-4; Etzold and Bode (2014), Curr. Opin. Virol. 7, 101-107; Kellman et al. (2022), Nat. Commun. 13, 2455; Pérez-Escalante et al. (2022), Crit. Rev. Food Sci. Nutr. 62, 181-214; Reily et al. (2019), Nat. Rev. Nephrol. 15, 346-366; Varki (2017), Glycobiology 27, 3-49; Walsh et al. (2020), J. Funct. Foods 72, 104074).

Due to their positive impact on animal and human health, there is large scientific and commercial interest in saccharides, particularly milk saccharides like MMOs and HMOs; yet their availability is limited. Today, many saccharides including oligosaccharides are synthesized chemically, enzymatically by in vitro glycosylation reactions, by chemoenzymatic synthesis, by fermentative approaches and/or by chemical, physical and/or biological degradation of polysaccharides. In particular, fermentative approaches for the production of oligosaccharides have been successful. Yet, the synthesis of a saccharide or a mixture of different saccharides is often hampered by the production of non-desired compounds like e.g., by-products and intermediates, reducing the purity of the desired saccharide(s) and/or reducing the yield of the synthesis.

### BRIEF SUMMARY

Provided are tools and methods by means of which one or more bioproduct(s) can be produced, preferably in an efficient, time and cost-effective way and which yields high amounts of the desired bioproduct(s).

Also provided is a cell for production of one or more bioproduct(s) wherein the cell is capable to produce, preferably produces, more preferably is genetically engineered to produce, one or more precursor(s) used in the production of the one or more bioproduct(s) and wherein the cell is genetically engineered to express, preferably to overexpress, a saccharide importer that internalizes at least one of the one or more precursor(s). This disclosure furthermore provides saccharide importers having uptake activity for lacto-N-triose (LN3, GlcNAc-beta1,3-Gal-beta1,4-Glc), each of which can be used in a method for the production of one or more bioproduct(s). Furthermore, any one of the saccharide importers can be used in a cell for production of one or more bioproduct(s). Also provided are methods and cells for the production of one or more bioproduct(s) as well as methods for the separation and purification of the one or more bioproduct(s).

### Definitions

The words used in this disclosure to describe the disclosure and its various embodiments are to be understood not only in the sense of their commonly defined meanings, but to include by special definition in this disclosure structure, material or acts beyond the scope of the commonly defined meanings. Thus, if an element can be understood in the context of this disclosure as including more than one meaning, then its use in a claim must be understood as being generic to all possible meanings supported by this disclosure and by the word itself.

The various aspects and embodiments disclosed herein are to be understood not only in the order and context specifically described in this disclosure, but to include any order and any combination thereof. Each embodiment as identified herein may be combined together unless otherwise indicated. All publications, patents, and patent applications mentioned in this disclosure are herein incorporated by reference to the same extent as if each individual publication, patent, or patent application was specifically and individually indicated to be incorporated by reference in its entirety.

Whenever the context requires, unless specifically stated otherwise, all words used in the singular number shall be deemed to include the plural and vice versa. Unless defined otherwise, all technical and scientific terms used herein generally have the same meaning as commonly understood by one of ordinary skill in the art to which this disclosure belongs. Generally, the nomenclature used herein and the laboratory procedures in cell culture, molecular genetics, organic chemistry and nucleic acid chemistry and hybridization described herein are those well-known and commonly employed in the art. Standard techniques are used for nucleic acid and peptide synthesis. Generally, enzymatic reactions and purification steps are performed according to the manufacturer's specifications.

In this disclosure, there have been disclosed embodiments of the disclosure, and although specific terms are employed, the terms are used in a descriptive sense only and not for purposes of limitation, the scope of the disclosure being set forth in the following claims. It must be understood that the illustrated embodiments have been set forth only for the purposes of example and that it should not be taken as limiting the disclosure. It will be apparent to those skilled in the art that alterations, other embodiments, improvements, details and uses can be made consistent with the letter and spirit of the disclosure herein and within the scope of this disclosure, which is limited only by the claims, construed in accordance with the patent law, including the doctrine of equivalents. In the claims that follow, reference characters used to designate claim steps are provided for convenience of description only, and are not intended to imply any particular order for performing the steps, unless specifically stated otherwise.

Throughout this disclosure, unless explicitly stated otherwise, the features “synthesize,” “synthesized” and “synthesis” are interchangeably used with the features “produce,” “produced” and “production,” respectively. Throughout this disclosure, unless explicitly stated otherwise, the expressions “capable of . . . <verb>” and “capable to . . . <verb>” are preferably replaced with the active voice of the verb and vice versa. For example, the expression “capable of expressing” is preferably replaced with “expresses” and vice versa, i.e., “expresses” is preferably replaced with “capable of expressing.” In this disclosure and in its claims, the verb “to comprise,” “to have” and “to contain” and their conjugations are used in their non-limiting sense to mean that items following the word are included, but items not specifically mentioned are not excluded. Throughout this disclosure, the verb “to comprise” may be replaced by “to consist” or “to consist essentially of” and vice versa. In addition, the verb “to consist” may be replaced by “to consist essentially of” meaning that a composition as defined herein may comprise additional component(s) than the ones specifically identified, the additional component(s) not altering the unique characteristic of the disclosure. Throughout this disclosure and in its claims, unless specifically stated otherwise, the verbs “to comprise,” “to have” and “to contain,” and their conjugations, may be preferably replaced by “to consist of” (and its conjugations) or “to consist essentially of” (and its conjugations) and vice versa. In addition, reference to an element by the indefinite article “a” or “an” does not exclude the possibility that more than one of the elements is present, unless the context clearly requires that there be one and only one of the elements. The indefinite article “a” or “an” thus usually means “at least one.” Throughout this disclosure, unless explicitly stated otherwise, the articles “a” and “an” are preferably replaced by “at least two,” more preferably by “at least three,” even more preferably by “at least four,” even more preferably by “at least five,” even more preferably by “at least six,” most preferably by “at least two.” The word “about” or “approximately” when used in association with a numerical value (e.g., “about 10”) or with a range (e.g., “about x to approximately y”) preferably means that the value or range is interpreted as being as accurate as the method used to measure it. If no error margins are specified, the expression “about” or “approximately” when used in association with a numerical value is interpreted as having the same round-off as the given value. Throughout this disclosure and its claims, unless otherwise stated, the expression “from x to y,” wherein x and y represent numerical values, refers to a range of numerical values wherein x is the lower value of the range and y is the upper value of the range. Herein, x and y are also included in the range.

According to this disclosure, the term “polynucleotide(s)” generally refers to any polyribonucleotide or polydeoxyribonucleotide, which may be unmodified RNA or DNA or modified RNA or DNA. “Polynucleotide(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. In addition, “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term “polynucleotide(s)” also includes DNAs or RNAs as described above that contain one or more modified bases. Thus, DNAs or RNAs with backbones modified for stability or for other reasons are “polynucleotide(s)” according to this disclosure. Moreover, DNAs or RNAs comprising unusual bases, such as inosine, or modified bases, such as tritylated bases, are to be understood to be covered by the term “polynucleotides.” It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. The term “polynucleotide(s)” also embraces short polynucleotides often referred to as oligonucleotide(s).

“Polypeptide(s)” refers to any peptide or protein comprising two or more amino acids joined to each other by peptide bonds or modified peptide bonds. “Polypeptide(s)” refers to both short chains, commonly referred to as peptides, oligopeptides and oligomers and to longer chains generally referred to as proteins. Polypeptides may contain amino acids other than the 20 gene encoded amino acids. “Polypeptide(s)” include those modified either by natural processes, such as processing and other post-translational modifications, but also by chemical modification techniques. Such modifications are well described in basic texts and in more detailed monographs, as well as in a voluminous research literature, and they are well known to the skilled person. The same type of modification may be present in the same or varying degree at several sites in a given polypeptide. Furthermore, a given polypeptide may contain many types of modifications. Modifications can occur anywhere in a polypeptide, including the peptide backbone, the amino acid sidechains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulphide bond formation, demethylation, formation of covalent cross-links, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, lipid attachment, sulfation, gamma-carboxylation of glutamic acid residues, hydroxylation and ADP-ribosylation, selenoylation, transfer-RNA mediated addition of amino acids to proteins, such as arginylation, and ubiquitination. Polypeptides may be branched or cyclic, with or without branching. Cyclic, branched and branched circular polypeptides may result from post-translational natural processes and may be made by entirely synthetic methods, as well.

The term “polynucleotide encoding a polypeptide” as used herein encompasses polynucleotides that include a sequence encoding a polypeptide. The term also encompasses polynucleotides that include a single continuous region or discontinuous regions encoding the polypeptide (for example, interrupted by integrated phage or an insertion sequence or editing) together with additional regions that also may contain coding and/or non-coding sequences.

“Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated,” as the term is employed herein. Similarly, a “synthetic” sequence, as the term is used herein, means any sequence that has been generated synthetically and not directly isolated from a natural source. “Synthesized,” as the term is used herein, means any synthetically generated sequence and not directly isolated from a natural source.

“Recombinant” means genetically engineered DNA prepared by transplanting or splicing genes from one species into the cells of a host organism of a different species. Such DNA becomes part of the host's genetic makeup and is replicated.

The terms “recombinant” or “transgenic” or “metabolically engineered” or “genetically engineered” as used herein with reference to a cell or host cell are used interchangeably and indicates that the cell replicates a heterologous nucleic acid, or expresses a peptide or protein encoded by a heterologous nucleic acid (i.e., a sequence “foreign to the cell” or a sequence “foreign to the location or environment in the cell”). Such cells are described to be transformed with at least one heterologous or exogenous gene or are described to be transformed by the introduction of at least one heterologous or exogenous gene. Recombinant or genetically engineered cells can contain genes that are not found within the native (non-recombinant) form of the cell. Recombinant cells can also contain genes found in the native form of the cell wherein the genes are modified and re-introduced into the cell by artificial means. The terms also encompass cells that contain a nucleic acid endogenous to the cell that has been modified or its expression or activity has been modified without removing the nucleic acid from the cell; such modifications include those obtained by gene replacement, replacement of a promoter; site-specific mutation; CrispR; riboswitch; recombineering; ssDNA mutagenesis; transposon mutagenesis and related techniques as known to a person skilled in the art. Accordingly, a “recombinant polypeptide” is one which has been produced by a recombinant cell. The terms also encompass cells that have been modified by removing a nucleic acid endogenous to the cell by means of common well-known technologies for a skilled person (like, e.g., knocking-out genes).

A “heterologous sequence” or a “heterologous nucleic acid,” as used herein, is one that originates from a source foreign to the particular cell (e.g., from a different species), or, if from the same source, is modified from its original form or place in the genome. Thus, a heterologous nucleic acid operably linked to a promoter is from a source different from that from which the promoter was derived, or, if from the same source, is modified from its original form or place in the genome. The heterologous sequence may be stably introduced, e.g., by transfection, transformation, conjugation or transduction, into the genome of the host microorganism cell, wherein techniques may be applied which will depend on the cell and the sequence that is to be introduced. Various techniques are known to a person skilled in the art and are, e.g., disclosed in Sambrook et al., Molecular Cloning: A Laboratory Manual, 2nd Ed., Cold Spring Harbor Laboratory Press, Cold Spring Harbor, N.Y. (1989). The term “mutant” or “engineered” cell or microorganism as used within the context of this disclosure refers to a cell or microorganism which is genetically engineered.

The term “endogenous” within the context of the present disclosure refers to any polynucleotide, polypeptide or protein sequence that is a natural part of a cell and is occurring at its natural location in the cell chromosome. The term “exogenous” refers to any polynucleotide, polypeptide or protein sequence which originates from outside the cell under study and not a natural part of the cell or which is not occurring at its natural location in the cell chromosome or plasmid.

The term “heterologous” when used in reference to a polynucleotide, gene, nucleic acid, polypeptide, or enzyme refers to a polynucleotide, gene, nucleic acid, polypeptide, or enzyme that is from a source or derived from a source other than the host organism species. In contrast a “homologous” polynucleotide, gene, nucleic acid, polypeptide, or enzyme is used herein to denote a polynucleotide, gene, nucleic acid, polypeptide, or enzyme that is derived from the host organism species. When referring to a gene regulatory sequence or to an auxiliary nucleic acid sequence used for maintaining or manipulating a gene sequence (e.g., a promoter, a 5′ untranslated region, 3′ untranslated region, poly A addition sequence, intron sequence, splice site, ribosome binding site, internal ribosome entry sequence, genome homology region, recombination site, etc.), “heterologous” means that the regulatory sequence or auxiliary sequence is not naturally associated with the gene with which the regulatory or auxiliary nucleic acid sequence is juxtaposed in a construct, genome, chromosome, or episome. Thus, a promoter operably linked to a gene to which it is not operably linked to in its natural state (i.e., in the genome of a non-genetically engineered organism) is referred to herein as a “heterologous promoter,” even though the promoter may be derived from the same species (or, in some cases, the same organism) as the gene to which it is linked.

The term “modified expression” of a gene relates to a change in expression compared to the wild-type expression of the gene in any phase of the production process of the desired bioproduct(s). The modified expression is either a lower or higher expression compared to the wild-type, wherein the term “higher expression” is also defined as “overexpression” of the gene in the case of an endogenous gene or “expression” in the case of a heterologous gene that is not present in the wild-type strain. Lower expression is obtained by means of common well-known technologies for a skilled person (such as the usage of siRNA, CrispR, CrispRi, riboswitch, recombineering, homologous recombination, ssDNA mutagenesis, RNAi, miRNA, asRNA, mutating genes, knocking-out genes, transposon mutagenesis, etc.) which are used to change the genes in such a way that they are “less-able” (i.e., statistically significantly ‘less-able’ compared to a functional wild-type gene) or completely unable (such as knocked-out genes) to produce functional final products. The term “riboswitch” as used herein is defined to be part of the messenger RNA that folds into intricate structures that block expression by interfering with translation. Binding of an effector molecule induces conformational change(s) permitting regulated expression post-transcriptionally. Next to changing the gene of interest in such a way that lower expression is obtained as described above, lower expression can also be obtained by changing the transcription unit, the promoter, an untranslated region, the ribosome binding site, the Shine Dalgarno sequence or the transcription terminator. Lower expression or reduced expression can, for instance, be obtained by mutating one or more base pairs in the promoter sequence or changing the promoter sequence fully to a constitutive promoter with a lower expression strength compared to the wild-type or an inducible promoter which result in regulated expression or a repressible promoter which results in regulated expression. Overexpression or expression is obtained by means of common well-known technologies for a skilled person (such as the usage of artificial transcription factors, de novo design of a promoter sequence, ribosome engineering, introduction or re-introduction of an expression module at euchromatin, usage of high-copy-number plasmids), wherein the gene is part of an “expression cassette” that relates to any sequence in which a promoter sequence, untranslated region sequence (containing either a ribosome binding sequence, Shine Dalgarno or Kozak sequence), a coding sequence (for instance, a saccharide importer gene sequence) and optionally a transcription terminator is present, and leading to the expression of a functional active protein. The expression is either constitutive or conditional or regulated or tuneable.

The term “constitutive expression” is defined as expression that is not regulated by transcription factors other than the subunits of RNA polymerase (e.g., the bacterial sigma factors like s70, s54, or related s-factors and the yeast mitochondrial RNA polymerase specificity factor MTF1 that co-associate with the RNA polymerase core enzyme) under certain growth conditions. Non-limiting examples of such transcription factors are CRP, LacI, ArcA, Cra, IclR in E. coli, or Aft2p, Crzlp, Skn7 in Saccharomyces cerevisiae, or DeoR, GntR, Fur in B. subtilis. These transcription factors bind on a specific sequence and may block or enhance expression in certain growth conditions. The RNA polymerase is the catalytic machinery for the synthesis of RNA from a DNA template. RNA polymerase binds a specific DNA sequence to initiate transcription, for instance, via a sigma factor in prokaryotic hosts or via MTF1 in yeasts. Constitutive expression offers a constant level of expression with no need for induction or repression.

The term “regulated expression” is defined as expression that is regulated by transcription factors other than the subunits of RNA polymerase (e.g., bacterial sigma factors) under certain growth conditions. Examples of such transcription factors are described above. Commonly expression regulation is obtained by means of an inducer, such as but not limited to IPTG, arabinose, rhamnose, fucose, allo-lactose or pH shifts, or temperature shifts or carbon depletion or substrates or the produced bioproduct(s).

The term “control sequences” refers to sequences recognized by the cells transcriptional and translational systems, allowing transcription and translation of a polynucleotide sequence to a polypeptide. Such DNA sequences are thus necessary for the expression of an operably linked coding sequence in a particular host cell, cell or organism. Such control sequences can be, but are not limited to, promoter sequences, ribosome binding sequences, Shine Dalgarno sequences, Kozak sequences, transcription terminator sequences. The control sequences that are suitable for prokaryotes, for example, include a promoter, optionally an operator sequence, and a ribosome binding site. Eukaryotic cells are known to utilize promoters, polyadenylation signals, and enhancers. DNA for a presequence or secretory leader may be operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it affects the transcription of the sequence; or a ribosome binding site is operably linked to a coding sequence if it is positioned so as to facilitate translation. The control sequences can furthermore be controlled with external chemicals, such as, but not limited to, IPTG, arabinose, lactose, allo-lactose, rhamnose or fucose via an inducible promoter or via a genetic circuit that either induces or represses the transcription or translation of the polynucleotide to a polypeptide.

Generally, “operably linked” means that the DNA sequences being linked are contiguous, and, in the case of a secretory leader, contiguous and in reading phase. However, enhancers do not have to be contiguous.

The term “wildtype” refers to the commonly known genetic or phenotypical situation as it occurs in nature.

The term “modified expression of a protein” as used herein refers to i) higher expression or overexpression of an endogenous protein, ii) expression of a heterologous protein, iii) expression and/or overexpression of a variant protein that has a higher activity compared to the wild-type (i.e., native in the expression host) protein, iv) reduced expression of an endogenous protein or v) expression and/or overexpression of a variant protein that has a reduced activity compared to the wild-type (i.e., native in the expression host) protein. Preferably, the term “modified expression of a protein” as used herein refers to i) higher expression or overexpression of an endogenous protein, ii) expression of a heterologous protein or iii) expression and/or overexpression of a variant protein that has a higher activity compared to the wild-type (i.e., native in the expression host) protein.

The term “modified activity” of a protein relates to a non-native activity of the protein in any phase of the production process of the desired bioproduct(s). The term “non-native,” as used herein with reference to the activity of a protein indicates that the protein has been modified to have an abolished, impaired, reduced, delayed, higher, accelerated or improved activity compared to the native activity of the protein. A modified activity of a protein is obtained by modified expression of the protein or is obtained by expression of a modified, i.e., mutant form of the protein. A mutant form of the protein can be obtained by expression of a mutant form of the gene encoding the protein, e.g., comprising a deletion, an insertion and/or a mutation of one or more nucleotides compared to the native gene sequence. A mutant form of a gene can be obtained by techniques well-known to a person skilled in the art, such as but not limited to site-specific mutation; CrispR; riboswitch; recombineering; ssDNA mutagenesis; transposon mutagenesis.

The term “non-native,” as used herein with reference to a cell producing one or more bioproduct(s), indicates that the one or more bioproduct(s) is/are i) not naturally produced or ii) when naturally produced not in the same amounts by the cell; and that the cell has been genetically engineered to be able to produce the one or more bioproduct(s) or to have a higher production of the one or more bioproduct(s).

As used herein, the term “mammary cell(s)” generally refers to mammalian mammary epithelial cell(s), mammalian mammary-epithelial luminal cell(s), or mammalian epithelial alveolar cell(s), or any combination thereof. As used herein, the term “mammary-like cell(s)” generally refers to mammalian cell(s) having a phenotype/genotype similar (or substantially similar) to natural mammalian mammary cell(s) but is/are derived from mammalian non-mammary cell source(s). Such mammalian mammary-like cell(s) may be engineered to remove at least one undesired genetic component and/or to include at least one predetermined genetic construct that is typical of a mammalian mammary cell. Non-limiting examples of mammalian mammary-like cell(s) may include mammalian mammary epithelial-like cell(s), mammalian mammary epithelial luminal-like cell(s), mammalian non-mammary cell(s) that exhibits one or more characteristics of a cell of a mammalian mammary cell lineage, or any combination thereof. Further non-limiting examples of mammalian mammary-like cell(s) may include mammalian cell(s) having a phenotype similar (or substantially similar) to natural mammalian mammary cell(s), or more particularly a phenotype similar (or substantially similar) to natural mammalian mammary epithelial cell(s). A mammalian cell with a phenotype or that exhibits at least one characteristic similar to (or substantially similar to) a natural mammalian mammary cell or a mammalian mammary epithelial cell may comprise a mammalian cell (e.g., derived from a mammary cell lineage or a non-mammary cell lineage) that exhibits either naturally, or has been engineered to, be capable of expressing at least one milk component.

As used herein, the term “non-mammary cell(s)” may generally include any mammalian cell of non-mammary lineage. In the context of the disclosure, a non-mammary cell can be any mammalian cell capable of being engineered to express at least one milk component. Non-limiting examples of such non-mammary cell(s) include hepatocyte(s), blood cell(s), kidney cell(s), cord blood cell(s), epithelial cell(s), epidermal cell(s), myocyte(s), fibroblast(s), mesenchymal cell(s), or any combination thereof. In some instances, molecular biology and genome editing techniques can be engineered to eliminate, silence, or attenuate myriad genes simultaneously.

“Fragment,” with respect to a polynucleotide, refers to a clone or any part of a polynucleotide molecule, particularly a part of a polynucleotide that retains a usable, functional characteristic of the full-length polynucleotide molecule. Useful fragments include oligonucleotides and polynucleotides that may be used in hybridization or amplification technologies or in the regulation of replication, transcription or translation. A “polynucleotide fragment” refers to any subsequence of a polynucleotide SEQ ID NO, typically, comprising or consisting of at least about 9, 10, 11, 12 consecutive nucleotides from the polynucleotide SEQ ID NO, for example, at least about 30 nucleotides or at least about 50 nucleotides of any of the polynucleotide sequences provided herein. Exemplary fragments can additionally or alternatively include fragments that comprise, consist essentially of, or consist of a region that encodes a conserved family domain of a polypeptide. Exemplary fragments can additionally or alternatively include fragments that comprise a conserved domain of a polypeptide. As such, a fragment of a polynucleotide SEQ ID NO preferably means a nucleotide sequence which comprises or consists of the polynucleotide SEQ ID NO wherein no more than about 200, 150, 100, 50 or 25 consecutive nucleotides are missing, preferably no more than about 50 consecutive nucleotides are missing, and which retains a usable, functional characteristic (e.g., activity) of the full-length polynucleotide molecule which can be assessed by the skilled person through routine experimentation. Alternatively, a fragment of a polynucleotide SEQ ID NO preferably means a nucleotide sequence which comprises or consists of an amount of consecutive nucleotides from the polynucleotide SEQ ID NO and wherein the amount of consecutive nucleotides is at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 100%, preferably at least 80%, more preferably at least 85%, even more preferably at least 87%, even more preferably at least 90%, even more preferably at least 95%, most preferably at least 97%, of the full-length of the polynucleotide SEQ ID NO and retains a usable, functional characteristic (e.g., activity) of the full-length polynucleotide molecule which can be routinely assessed by the skilled person. As such, a fragment of a polynucleotide SEQ ID NO preferably means a nucleotide sequence which comprises or consists of the polynucleotide SEQ ID NO, wherein an amount of consecutive nucleotides is missing and wherein the amount is no more than 50%, 40%, 30% of the full-length of the polynucleotide SEQ ID NO, preferably no more than 20%, 15%, 10%, 9%, 8%, 7%, 6%, 5%, 4.5%, 4%, 3.5%, 3%, 2.5%, 2%, 1.5%, 1%, 0.5%, more preferably no more than 15%, even more preferably no more than 10%, even more preferably no more than 5%, most preferably no more than 2.5%, of the full-length of the polynucleotide SEQ ID NO and wherein the fragment retains a usable, functional characteristic (e.g., activity) of the full-length polynucleotide molecule which can be routinely assessed by the skilled person.

“Fragment,” with respect to a polypeptide, refers to a subsequence of the polypeptide which performs at least one biological function of the intact polypeptide in substantially the same manner, or to a similar extent, as does the intact polypeptide. A “subsequence of the polypeptide” or “a stretch of amino acid residues” as described herein refers to a sequence of contiguous amino acid residues derived from the polypeptide. For example, a polypeptide fragment can comprise a recognizable structural motif or functional domain such as a DNA-binding site or domain that binds to a DNA promoter region, an activation domain, or a domain for protein-protein interactions, and may initiate transcription. Fragments can vary in size from as few as 3 amino acid residues to the full length of the intact polypeptide, for example, at least about 10 amino acid residues in length, for example, at least about 20 amino acid residues in length, for example, at least about 30 amino acid residues in length, for example, at least about 100 amino acid residues in length, for example, at least about 150 amino acid residues in length, for example, at least about 200 amino acid residues in length. As such, a fragment of a polypeptide SEQ ID NO (or UniProt ID) preferably means a polypeptide sequence which comprises or consists of the polypeptide SEQ ID NO (or UniProt ID) wherein no more than about 200, 150, 125, 100, 80, 60, 50, 40, 30, 20 or 15 consecutive amino acid residues are missing, preferably no more than about 100 consecutive amino acid residues are missing, more preferably no more than about 50 consecutive amino acid residues are missing, even more preferably no more than about 40 consecutive amino acid residues are missing, and performs at least one biological function of the intact polypeptide in substantially the same manner, preferably to a similar or greater extent, as does the intact polypeptide which can be routinely assessed by the skilled person. Alternatively, a fragment of a polypeptide SEQ ID NO (or UniProt ID) preferably means a polypeptide sequence which comprises or consists of an amount of consecutive amino acid residues from the polypeptide SEQ ID NO (or UniProt ID) and wherein the amount of consecutive amino acid residues is at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 100%, preferably at least 80%, more preferably at least 85%, even more preferably at least 87%, even more preferably at least 90%, even more preferably at least 95%, most preferably at least 97% of the full-length of the polypeptide SEQ ID NO (or UniProt ID) and which performs at least one biological function of the intact polypeptide in substantially the same manner, preferably to a similar or greater extent, as does the intact polypeptide which can be routinely assessed by the skilled person. As such, a fragment of a polypeptide SEQ ID NO (or UniProt ID) preferably means a polypeptide sequence which comprises or consists of the polypeptide SEQ ID NO (or UniProt ID), wherein an amount of consecutive amino acid residues is missing and wherein the amount is no more than 50%, 40%, 30% of the full-length of the polypeptide SEQ ID NO (or UniProt ID), preferably no more than 20%, 15%, 10%, 9%, 8%, 7%, 6%, 5%, 4.5%, 4%, 3.5%, 3%, 2.5%, 2%, 1.5%, 1%, 0.5%, more preferably no more than 15%, even more preferably no more than 10%, even more preferably no more than 5%, most preferably no more than 2.5%, of the full-length of the polypeptide SEQ ID NO (or UniProt ID) and which performs at least one biological function of the intact polypeptide in substantially the same manner, preferably to a similar or greater extent, as does the intact polypeptide which can be routinely assessed by the skilled person.

Throughout this disclosure, the sequence of a polypeptide can be represented by a SEQ ID NO or alternatively by an UniProt ID. Therefore, the terms “polypeptide SEQ ID NO” and “polypeptide UniProt ID” can be interchangeably used, unless explicitly stated otherwise.

Homologs can be identified by analysis of nucleotide and polypeptide sequence alignments. For example, performing a query on a database of nucleotide or polypeptide sequences can identify homologs of the nucleotides or polypeptides of interest. Sequence analysis can involve BLAST, Reciprocal BLAST, or PSI-BLAST analysis of non-redundant databases using the amino acid sequence of a reference polypeptide sequence. The amino acid sequence is, in some instances, deduced from the nucleotide sequence. Typically, those polypeptides in the database that have greater than 40% sequence identity to a polypeptide of interest are candidates for further evaluation for suitability as a homologous polypeptide. Amino acid sequence similarity allows for conservative amino acid substitutions, such as substitution of one hydrophobic residue for another or substitution of one polar residue for another or substitution of one acidic amino acid for another or substitution of one basic amino acid for another etc. Preferably, by conservative substitutions is intended combinations such as glycine by alanine and vice versa; valine, isoleucine and leucine by methionine and vice versa; aspartate by glutamate and vice versa; asparagine by glutamine and vice versa; serine by threonine and vice versa; lysine by arginine and vice versa; cysteine by methionine and vice versa; and phenylalanine and tyrosine by tryptophan and vice versa. If desired, manual inspection of such candidates can be carried out in order to narrow the number of candidates to be further evaluated.

A domain can be characterized, for example, by a Pfam (El-Gebali et al., Nucleic Acids Res. 47 (2019) D427-D432), an IPR (InterPro domain) (ebi.ac.uk/interpro) (Mitchell et al., Nucleic Acids Res. 47 (2019) D351-D360), a protein fingerprint domain (PRINTS) (Attwood et al., Nucleic Acids Res. 31 (2003) 400-402), a SUBFAM domain (Gough et al., J. Mol. Biol. 313 (2001) 903-919), a TIGRFAM domain (Selengut et al., Nucleic Acids Res. 35 (2007) D260-D264), a Conserved Domain Database (CDD) designation (ncbi.nlm.nih.gov/cdd) (Lu et al., Nucleic Acids Res. 48 (2020) D265-D268), a PTHR domain (pantherdb.org) (Mi et al., Nucleic Acids. Res. 41 (2013) D377-D386; Thomas et al., Genome Research 13 (2003) 2129-2141) or a PATRIC identifier or PATRIC DB global family domain (patricbrc.org/) (Davis et al., Nucleic Acids Res. 48(D1) (2020) D606-D612). Protein or polypeptide sequence information and functional information can be provided by a comprehensive resource for protein sequence and annotation data like e.g., the Universal Protein Resource (UniProt) (uniprot.org) (Nucleic Acids Res. 2021, 49(D1), D480-D489). UniProt comprises the expertly and richly curated protein database called the UniProt Knowledgebase (UniProtKB), together with the UniProt Reference Clusters (UniRef) and the UniProt Archive (UniParc). The UniProt identifiers (UniProt ID) are unique for each protein present in the database. Throughout this disclosure, the sequence of a polypeptide is represented by a SEQ ID NO or an UniProt ID. Unless stated otherwise, the UniProt IDs of the proteins described correspond to their sequence version 01 as present in the UniProt Database (uniprot.org) version release 2021_03 and consulted on 9 Jun. 2021. InterPro provides functional analysis of proteins by classifying them into families and predicting domains and important sites. To classify proteins in this way, InterPro uses predictive models, known as signatures, provided by several different databases (referred to as member databases) that make up the InterPro consortium. Protein signatures from these member databases are combined into a single searchable resource, capitalizing on their individual strengths to produce a powerful integrated database and diagnostic tool.

The terms “IPR001927,” “PDOC00680,” “NA_GALACTOSIDE_SYMP,” “PS 00872” are used interchangeably and refer to the sodium: galactoside symporter family signature (Pourcher et al. 1991, Biochem. Biophys. Res. Commun. 178, 1176-1181; Reizer et al. 1994, Biochim. Biophys. Acta 1197, 133-166).

It should be understood for those skilled in the art that for the databases used herein, comprising InterPro 90.0 (released 4, August 2022), PFAM 32.0 as released on September 2018, PANTHER 18.0 as released on 17 Sep. 2023, the Conserved Domain Database CDD 3.20 as released on September 2022, the content of each database is fixed at each release and is not to be changed. When the content of a specific database is changed, this specific database receives a new release version with a new release date. All release versions for each database with their corresponding release dates and specific content as annotated at these specific release dates are available and known to those skilled in the art.

The terms “identical” or “percent identity” or “% identity” in the context of two or more nucleic acid or polypeptide sequences, refer to two or more sequences or subsequences that are the same or have a specified percentage of nucleotides or amino acid residues that are the same, when compared and aligned for maximum correspondence, as measured using sequence comparison algorithms or by visual inspection. For sequence comparison, one sequence acts as a reference sequence, to which test sequences are compared. When using a sequence comparison algorithm, test and reference sequences are inputted into a computer, subsequence coordinates are designated, if necessary, and sequence algorithm program parameters are designated. The sequence comparison algorithm then calculates the % sequence identity for the test sequence(s) relative to the reference sequence, based on the designated program parameters. The percentage of sequence identity can be, preferably is, determined by alignment of the two sequences and identification of the number of positions with identical residues divided by the number of residues in the shorter of the sequences×100. Percent identity may be calculated globally over the full-length sequence of a given SEQ ID NO, i.e., the reference sequence, resulting in a global % identity score. Alternatively, % identity may be calculated over a partial sequence of the reference sequence, resulting in a local percent identity score. A partial sequence preferably means at least about 25%, 30%, 35%, 40%, 45%, 50%, 55%, 60%, 65%, 70%, 75%, 80%, 85%, 87.5%, 90%, 91%, 92%, 93%, 94% or 95% of the full-length reference sequence. In another preferred embodiment, a partial sequence of a reference polypeptide sequence means a stretch of at least 150 amino acid residues up to the total number of amino acid residues of a reference polypeptide sequence. In another more preferred embodiment, a partial sequence of a reference polypeptide sequence means a stretch of at least 200 amino acid residues up to the total number of amino acid residues of a reference polypeptide sequence. Using the full-length of the reference sequence in a local sequence alignment results in a global percent identity score between the test and the reference sequence.

Percent identity can be determined using different algorithms like, for example, BLAST and PSI-BLAST (Altschul et al., 1990, e 215:3, 403-410; Altschul et al., 1997, Nucleic Acids Res 25:17, 3389-402), the Clustal Omega method (Sievers et al., 2011, Mol. Syst. Biol. 7:539), the MatGAT method (Campanella et al., 2003, BMC Bioinformatics, 4:29) or EMBOSS Needle.

As used herein, a polypeptide comprising or consisting of an amino acid sequence having 25% or more sequence identity over a stretch of at least 50 amino acid residues of a reference polypeptide sequence is to be understood as that the amino acid sequence has 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5%, 99.6%, 99.7%, 99.8%, 99.9%, 100% sequence identity over a stretch of at least 50 amino acid residues of the reference polypeptide sequence.

As used herein, a polypeptide comprising or consisting of an amino acid sequence having 25% or more sequence identity over a stretch of at least 100 amino acid residues of a reference polypeptide sequence is to be understood as that the amino acid sequence has 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5%, 99.6%, 99.7%, 99.8%, 99.9%, 100% sequence identity over a stretch of at least 100 amino acid residues of the reference polypeptide sequence.

As used herein, a polypeptide comprising or consisting of an amino acid sequence having 25% or more sequence identity over a stretch of at least 150 amino acid residues of a reference polypeptide sequence is to be understood as that the amino acid sequence has 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5%, 99.6%, 99.7%, 99.8%, 99.9%, 100% sequence identity over a stretch of at least 150 amino acid residues of the reference polypeptide sequence.

As used herein, a polypeptide comprising or consisting of an amino acid sequence having 25% or more sequence identity over a stretch of at least 200 amino acid residues of a reference polypeptide sequence is to be understood as that the amino acid sequence has 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5%, 99.6%, 99.7%, 99.8%, 99.9%, 100% sequence identity over a stretch of at least 200 amino acid residues of the reference polypeptide sequence.

As used herein, a polypeptide comprising or consisting of an amino acid sequence having 25% or more sequence identity over a stretch of at least 250 amino acid residues of a reference polypeptide sequence is to be understood as that the amino acid sequence has 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5%, 99.6%, 99.7%, 99.8%, 99.9%, 100% sequence identity over a stretch of at least 250 amino acid residues of the reference polypeptide sequence.

As used herein, a polypeptide comprising or consisting of an amino acid sequence having 25% or more sequence identity to the full-length sequence of a reference polypeptide sequence is to be understood as that the amino acid sequence has 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5%, 99.6%, 99.7%, 99.8%, 99.9%, 100% sequence identity to the full-length of the amino acid sequence of the reference polypeptide sequence.

Throughout this disclosure, unless explicitly specified otherwise, a polypeptide comprising, consisting of or having an amino acid sequence having 25% or more sequence identity to the full-length amino acid sequence of a reference polypeptide, usually indicated with a SEQ ID NO or UniProt ID, preferably has 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%, more preferably has at least 50%, even more preferably has at least 55%, even more preferably has at least 60%, even more preferably has at least 65%, even more preferably has at least 70%, even more preferably has at least 75%, even more preferably has at least 80%, even more preferably has at least 85%, most preferably has at least 90%, sequence identity to the full length reference sequence. Additionally, unless explicitly specified otherwise, a polynucleotide sequence comprising, consisting of or having a nucleotide sequence having 25%, 30%, 35%, 40%, 45%, 50%, 55%, 60%, 65%, 70%, 75%, 80%, 85%, 90%, 91%, 92%, 93%, 94%, 95%, 96%, 97%, 98% or 99%, more preferably has at least 50%, even more preferably has at least 55%, even more preferably has at least 60%, even more preferably has at least 65%, even more preferably has at least 70%, even more preferably has at least 75%, even more preferably has at least 80%, even more preferably has at least 85%, most preferably has at least 90% sequence identity to the full-length reference sequence.

For the purposes of this disclosure, percent identity is determined using MatGAT2.01 (Campanella et al., 2003, BMC Bioinformatics 4:29). The following default parameters for protein are employed: (1) Gap cost Existence: 12 and Extension: 2; (2) The Matrix employed was BLOSUM50.

The term “saccharide importer” as used herein refers to a polypeptide capable to internalize or to take up a saccharide from outside the cell to the inside of the cell. The saccharide can be a monosaccharide, an activated monosaccharide, a phosphorylated monosaccharide, a disaccharide, an oligosaccharide or a polysaccharide as defined herein.

The term “major facilitator superfamily (MFS) of transporters” as used herein refers to the largest known superfamily of secondary active transporters found ubiquitously in all living organisms. MFS transporters are responsible for transporting a broad spectrum of substrates, either down their concentration gradient or uphill using the energy stored in the electrochemical gradients. MFS transporters have a typical topology, mostly built from 12 transmembrane (TM) domains organized from two 6-TM bundles connected by a long and flexible intracellular loop; MFS transporters having more than 12 TM domains have also been identified (Chang et al. 2004, Mol. Membr. Biol. 21 (3), 171-181; Drew et al. 2021, Chem. Rev. 121 (9), 5289-5335; Pao et al., 1998, Microbiol. Mol. Biol. Rev. 62, 1-34; Reddy et al. 2012, FEBS J. 279, 2022-2035; Wang et al. 2020, Biochim. Biophys. Acta Biomembr. 1862 (9), 183277).

The terms “transmembrane domain” or “TM domain” as used herein are used interchangeably and refer to a membrane-spanning protein domain. The domain covers both alpha-helical transmembrane regions and the membrane spanning regions of beta-barrel transmembrane proteins. Alpha-helical transmembrane domains generally have an alpha helix topological conformation, wherein the amino acid residues are often hydrophobic. The presence of a transmembrane domain in a given polypeptide sequence can be determined by e.g., X-ray diffraction or can be predicted based on hydrophobicity scales, like e.g., via deep learning protein language model-based algorithms such as e.g., DeepTMHMM (Hallgren et al., 2022, bioRxiv 2022.04.08.487609), DMCTOP (Yang et al., 2022, IEEE/ACM Trans. Comput. Biol. Bioinform. 19, 295-304), Pureseq™ (Wang et al., 2019, bioRxiv, 627307), BetAware-Deep (Madeo et al., 2021, J. Mol. Biol. 433, 166729).

The term “a non-TM helix” as used herein refers to a helical polypeptide sequence which is not present in a TM domain as defined herein.

The term “glycosyltransferase” as used herein refers to an enzyme capable to catalyze the transfer of a sugar moiety of a donor to a specific acceptor, forming glycosidic bonds. The donor can be a precursor as defined herein. A classification of glycosyltransferases using nucleotide diphospho-sugar, nucleotide monophospho-sugar and sugar phosphates and related proteins into distinct sequence-based families has been described (Campbell et al., Biochem. J. 326, 929-939 (1997)) and is available on the CAZy (CArbohydrate-Active EnZymes) website (cazy.org).

As used herein the glycosyltransferase can be selected from the list comprising but not limited to: fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases and fucosaminyltransferases.

The term “monosaccharide” as used herein refers to a sugar that is not decomposable into simpler sugars by hydrolysis, is classed either an aldose or ketose, and contains one or more hydroxyl groups per molecule. Monosaccharides are saccharides containing only one simple sugar. Examples of monosaccharides comprise hexose, D-glucopyranose, D-galactofuranose, D-galactopyranose, L-galactopyranose, D-mannopyranose, D-allopyranose, L-altropyranose, D-gulopyranose, L-idopyranose, D-talopyranose, D-ribofuranose, D-ribopyranose, D-arabinofuranose, D-arabinopyranose, L-arabinofuranose, L-arabinopyranose, D-xylopyranose, D-lyxopyranose, D-erythrofuranose, D-threofuranose, heptose, L-glycero-D-manno-heptopyranose (LDmanHep), D-glycero-D-manno-heptopyranose (DDmanHep), 6-deoxy-L-altropyranose, 6-deoxy-D-gulopyranose, 6-deoxy-D-talopyranose, 6-deoxy-D-galactopyranose, 6-deoxy-L-galactopyranose, 6-deoxy-D-mannopyranose, 6-deoxy-L-mannopyranose, 6-deoxy-D-glucopyranose, 2-deoxy-D-arabino-hexose, 2-aeoxy-D-erythro-pentose, 2,6-dideoxy-D-arabino-hexopyranose, 3,6-dideoxy-D-arabino-hexopyranose, 3,6-dideoxy-L-arabino-hexopyranose, 3,6-dideoxy-D-xylo-hexopyranose, 3,6-dideoxy-D-ribo-hexopyranose, 2,6-dideoxy-D-ribo-hexopyranose, 3,6-dideoxy-L-xylo-hexopyranose, 2-amino-2-deoxy-D-glucopyranose, 2-amino-2-deoxy-D-galactopyranose, 2-amino-2-deoxy-D-mannopyranose, 2-amino-2-deoxy-D-allopyranose, 2-amino-2-deoxy-L-altropyranose, 2-amino-2-deoxy-D-gulopyranose, 2-amino-2-deoxy-L-idopyranose, 2-amino-2-deoxy-D-talopyranose, 2-acetamido-2-deoxy-D-glucopyranose, 2-acetamido-2-deoxy-D-galactopyranose, 2-acetamido-2-deoxy-D-mannopyranose, 2-acetamido-2-deoxy-D-allopyranose, 2-acetamido-2-deoxy-L-altropyranose, 2-acetamido-2-deoxy-D-gulopyranose, 2-acetamido-2-deoxy-L-idopyranose, 2-acetamido-2-deoxy-D-talopyranose, 2-acetamido-2,6-dideoxy-D-galactopyranose, 2-acetamido-2,6-dideoxy-L-galactopyranose, 2-acetamido-2,6-dideoxy-L-mannopyranose, 2-acetamido-2,6-dideoxy-D-glucopyranose, 2-acetamido-2,6-dideoxy-L-altropyranose, 2-acetamido-2,6-dideoxy-D-talopyranose, D-glucopyranuronic acid, D-Galactopyranuronic acid, D-Mannopyranuronic acid, D-allopyranuronic acid, L-altropyranuronic acid, D-gulopyranuronic acid, L-gulopyranuronic acid, L-idopyranuronic acid, D-talopyranuronic acid, sialic acid, 5-amino-3,5-dideoxy-D-glycero-D-galacto-non-2-ulosonic acid, 5-acetamido-3,5-dideoxy-D-glycero-D-galacto-non-2-ulosonic acid, 5-glycolylamido-3,5-dideoxy-D-glycero-D-galacto-non-2-ulosonic acid, erythritol, arabinitol, xylitol, ribitol, glucitol, galactitol, mannitol, D-ribo-hex-2-ulopyranose, D-arabino-hex-2-ulofuranose (D-fructofuranose), D-arabino-hex-2-ulopyranose, L-xylo-hex-2-ulopyranose, D-lyxo-hex-2-ulopyranose, D-threo-pent-2-ulopyranose, D-altro-hept-2-ulopyranose, 3-C-(hydroxymethyl)-D-erythofuranose, 2,4,6-trideoxy-2,4-diamino-D-glucopyranose, 6-deoxy-3-O-methyl-D-glucose, 3-O-methyl-D-rhamnose, 2,6-dideoxy-3-methyl-D-ribo-hexose, 2-amino-3-O-[(R)-1-carboxyethyl]-2-deoxy-D-glucopyranose, 2-acetamido-3-O-[(R)-carboxyethyl]-2-deoxy-D-glucopyranose, 2-glycolylamido-3-O-[(R)-1-carboxyethyl]-2-deoxy-D-glucopyranose, 3-deoxy-D-lyxo-hept-2-ulopyranosaric acid, 3-deoxy-D-manno-oct-2-ulopyranosonic acid, 3-deoxy-D-glycero-D-galacto-non-2-ulopyranosonic acid, 5,7-diamino-3,5,7,9-tetradeoxy-L-glycero-L-manno-non-2-ulopyranosonic acid, 5,7-diamino-3,5,7,9-tetradeoxy-L-glycero-L-altro-non-2-ulopyranosonic acid, 5,7-diamino-3,5,7,9-tetradeoxy-D-glycero-D-galacto-non-2-ulopyranosonic acid, 5,7-diamino-3,5,7,9-tetradeoxy-D-glycero-D-talo-non-2-ulopyranosonic acid, 2-acetamido-2,6-dideoxy--L-arabino-4-hexulose, 2-acetamido-2,6-dideoxy--L-lyxo-4-hexulose, N-acetyl-L-rhamnosamine, N-acetyl-D-fucosamine, N-acetyl-L-pneumosamine, N-acetylmuramic acid, N-acetyl-L-quinovosamine, glucose (Glc), galactose (Gal), N-acetylglucosamine (GlcNAc), glucosamine (Glcn), mannose (Man), xylose (Xyl), N-acetylmannosamine (ManNAc), N-glycolylneuraminic acid, N-acetylgalactosamine (GalNAc), galactosamine (Galn), fucose (Fuc), rhamnose (Rha), glucuronic acid, gluconic acid, fructose (Fru) and polyols. With the term polyol is meant an alcohol containing multiple hydroxyl groups. For example, glycerol, sorbitol, or mannitol.

The term “phosphorylated monosaccharide” as used herein refers to one of the above listed monosaccharides which is phosphorylated. Examples of phosphorylated monosaccharides include but are not limited to glucose-1-phosphate, glucose-6-phosphate, glucose-1,6-bisphosphate, galactose-1-phosphate, fructose-6-phosphate, fructose-1,6-bisphosphate, fructose-1-phosphate, glucosamine-1-phosphate, glucosamine-6-phosphate, N-acetylglucosamine-1-phosphate, mannose-1-phosphate, mannose-6-phosphate or fucose-1-phosphate. Some, but not all, of these phosphorylated monosaccharides are precursors or intermediates for the production of activated monosaccharide.

The terms “activated monosaccharide,” “nucleotide-activated sugar,” “nucleotide-sugar,” “activated sugar,” “nucleoside” or “nucleotide donor” are used herein interchangeably and refer to activated forms of monosaccharides. Examples of activated monosaccharides include but are not limited to UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), UDP-glucuronate, UDP-galacturonate, UDP-2-acetamido-2,6-dideoxy--L-arabino-4-hexulose, UDP-2-acetamido-2,6-dideoxy--L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), GDP-L-quinovose, CMP-sialic acid (CMP-Neu5Ac or CMP-N-acetylneuraminic acid), GDP-fucose (GDP-Fuc), GDP-rhamnose and UDP-xylose. Nucleotide-sugars act as glycosyl donors in glycosylation reactions. Glycosylation reactions are reactions that are catalyzed by glycosyltransferases.

The term “disaccharide” as used herein refers to a saccharide polymer containing two simple sugars, i.e., monosaccharides. Such disaccharides contain monosaccharides preferably selected from the list of monosaccharides as used herein above. Examples of disaccharides comprise lactose (Gal-b1,4-Glc), lacto-N-biose (Gal-b1,3-GlcNAc), N-acetyllactosamine (Gal-b1,4-GlcNAc), LacDiNAc (GalNAc-b1,4-GlcNAc), N-acetylgalactosaminylglucose (GalNAc-b1,4-Glc), Neu5Ac-a2,3-Gal, Neu5Ac-a2,6-Gal, fucopyranosyl-(I-4)-N-glycolylneuraminic acid (Fuc-(1-4)-Neu5Gc), sucrose (Glc-a1,2-Fru), maltose (Glc-a1,4-Glc) and melibiose (Gal-a1,6-Glc).

“Oligosaccharide” as the term is used herein and as generally understood in the state of the art, refers to a saccharide polymer containing a small number, typically three to twenty, preferably three to ten, of simple sugars, i.e., monosaccharides. Preferably, the oligosaccharide as described herein contains monosaccharides selected from the list as used herein above. The oligosaccharide as used in this disclosure can be a linear structure or can include branches. The linkage (e.g., glycosidic linkage, galactosidic linkage, glucosidic linkage, etc.) between two sugar units can be expressed, for example, as 1,4, 1->4, or (1-4), used interchangeably herein. For example, the terms “Gal-b1,4-Glc,” “Gal-β1,4-Glc,” “b-Gal-(1->4)-Glc,” “β-Gal-(1->4)-Glc,” “Galbeta 1-4-Glc,” “Gal-b (1-4)-Glc” and “Gal-β(1-4)-Glc” have the same meaning, i.e., a beta-glycosidic bond links carbon-1 of galactose (Gal) with the carbon-4 of glucose (Glc). Each monosaccharide can be in the cyclic form (e.g., pyranose or furanose form). Linkages between the individual monosaccharide units may include alpha 1->2, alpha 1->3, alpha 1->4, alpha 1->6, alpha 2->1, alpha 2->3, alpha 2->4, alpha 2->6, beta 1->2, beta 1->3, beta 1->4, beta 1->6, beta 2->1, beta 2->3, beta 2->4, and beta 2->6. An oligosaccharide can contain both alpha- and beta-glycosidic bonds or can contain only alpha-glycosidic or only beta-glycosidic bonds. The term “polysaccharide” refers to a compound consisting of a large number, typically more than twenty, of monosaccharides linked glycosidically.

Examples of oligosaccharides include but are not limited to a milk oligosaccharide, a mammalian milk oligosaccharide or MMO, a human milk oligosaccharide or HMO, a neutral (non-charged) oligosaccharide, a negatively charged oligosaccharide, a fucosylated oligosaccharide, a sialylated oligosaccharide, an N-acetylglucosamine containing oligosaccharide, an N-acetyllactosamine containing oligosaccharide, a lacto-N-biose containing oligosaccharide, a lactose containing oligosaccharide, a non-fucosylated neutral (non-charged) oligosaccharide, an N-acetyllactosamine containing fucosylated oligosaccharide, an N-acetyllactosamine non-fucosylated oligosaccharide, a lacto-N-biose containing fucosylated oligosaccharide, a lacto-N-biose containing non-fucosylated oligosaccharide, an N-acetyllactosamine containing negatively charged oligosaccharide, a lacto-N-biose containing negatively charged oligosaccharide, O-antigen, ECA, the glycan chain present in lipopolysaccharides (LPS), the oligosaccharide repeats present in capsular polysaccharides, peptidoglycan (PG), an amino-sugar, a Lewis-type antigen oligosaccharide, an antigen of the human ABO blood group system, an animal oligosaccharide, preferably selected from the group consisting of N-glycans and O-glycans, a plant oligosaccharide, preferably selected from the group consisting of N-glycans and O-glycans.

As used herein, a ‘sialylated oligosaccharide’ is to be understood as a negatively charged sialic acid containing oligosaccharide, i.e., an oligosaccharide having a sialic acid residue. It has an acidic nature. Some examples are 3-SL (3′-sialyllactose or 3′SL or Neu5Ac-a2,3-Gal-b1,4-Glc), 3′-sialyllactosamine, 6-SL (6′sialyllactose, 6′-sialyllactose or 6′SL or Neu5Ac-a2,6-Gal-b1,4-Glc), 8-SL (8′sialyllactose, 8′-sialyllactose or 8′SL or Neu5Ac-a2,8-Gal-β1,4-Glc), 3,6-disialyllactose (Neu5Ac-a2,3-(Neu5Ac-a2,6)-Gal-b1,4-Glc), 6,6′-disialyllactose (Neu5Ac-a2,6-Gal-b1,4-(Neu5Ac-a2,6)-Glc), 8,3-disialyllactose (Neu5Ac-a2,8-Neu5Ac-a2,3-Gal-b1,4-Glc), 6′-sialyllactosamine, oligosaccharides comprising 6′sialyllactose (also known as 6′sialyllactose, 6′SL and 6′-SL), SGG hexasaccharide (Neu5Acα-2,3Galβ-1,3GalNacβ-1,3Galα-1,4Galβ-1,4Gal), sialylated tetrasaccharide (Neu5Acα-2,3Galβ-1,4GlcNacβ-14GlcNAc), sialylated lacto-N-triose, sialylated lacto-N-tetraose, sialyllacto-N-neotetraose, LSTa, LSTb, LSTc, LSTd, monosialyllacto-N-hexaose, disialyllacto-N-hexaose I, monosialyllacto-N-neohexaose I, monosialyllacto-N-neohexaose II, disialyllacto-N-neohexaose, disialyllacto-N-tetraose, disialyllacto-N-hexaose II, sialyllacto-N-tetraose a, disialyllacto-N-hexaose I, sialyllacto-N-tetraose b, 3′-sialyl-3-fucosyllactose, fucodisialyllacto-N-hexaose, disialomonofucosyllacto-N-neohexaose, monofucosylmonosialyllacto-N-octaose (sialyl Lea), sialyllacto-N-fucohexaose II, disialyllacto-N-fucopentaose II, monofucosyldisialyllacto-N-tetraose and oligosaccharides bearing one or several sialic acid residue(s), including but not limited to: oligosaccharide moieties of the gangliosides selected from GM3 (3′sialyllactose, Neu5Acα-2,3Galβ-4Glc) and oligosaccharides comprising the GM3 motif, GD3 Neu5Acα-2,8Neu5Aca-2,3Galβ-1,4Glc GT3(Neu5Acα-2,8Neu5Acα-2,8Neu5Acα-2,3Galβ-1,4Glc); GM2 GalNAcβ-1,4(Neu5Acα-2,3)Galβ-1,4Glc, GM1 Galβ-1,3GalNAcβ-1,4(Neu5Acα-2,3)Galβ-1,4Glc, GDla Neu5Acα-2,3 Galβ-1,3GalNAcβ-1,4(Neu5Acα-2,3)Galβ-1,4Glc, GTla Neu5Acα-2,8Neu5Acα-2,3Galβ-1,3 GalNAcβ-1,4(Neu5Acα-2,3)Galβ-1,4Glc, GD2 GalNAcp-1,4(Neu5Acα-2,8Neu5Acα2,3) Galβ-1,4Glc, GT2 GalNAcβ-1,4(Neu5Acα-2,8Neu5Acα-2,8Neu5Acα2,3)Galβ-1,4Glc, GD1b, Galβ-1,3GalNAcp-1,4(Neu5Acα-2,8Neu5Acα2,3)Galβ-1,4Glc, GT1b Neu5Acα-2,3Galβ-1,3GalNAcβ-1,4(Neu5Acα-2,8Neu5Acα2,3)Galβ-1,4Glc, GQ1b Neu5Acα-2,8Neu5Acα-2,3Galβ-1,3GalNAc β-1,4(Neu5Acα-2,8Neu5Acα2,3)Galβ-1,4Glc, GTlc Galβ-1,3GalNAcβ-1,4(Neu5Acα-2,8Neu5Acα-2,8Neu5Acα2,3)Galβ-1,4Glc, GQlc Neu5Acα-2,3Galβ-1,3GalNAc β-1,4(Neu5Acα-2,8Neu5Acα-2,8Neu5Acα2,3)Galβ-1,4Glc, GP1c Neu5Acα-2,8Neu5Acα-2,3Galβ-1,3GalNAc β-1,4(Neu5Acα-2,8Neu5Acα-2,8Neu5Acα2,3)Galβ-1,4Glc, GDla Neu5Acα-2,3Galβ-1,3(Neu5Acα-2,6)GalNAcβ-1,4Galβ-1,4Glc, Fucosyl-GM1 Fuca-1,2Galβ-1,3GalNAcβ-1,4(Neu5Acα-2,3)Galβ-1,4Glc; all of which may be extended to the production of the corresponding gangliosides by reacting the above oligosaccharide moieties with ceramide or synthetizing the above oligosaccharides on a ceramide.

The terms “LNT II,” “LNT-II,” “LN3,” “lacto-N-triose II,” “lacto-N-triose II,” “lacto-N-triose,” “lacto-N-triose” or “GlcNAcβ1-3Galβ1-4Glc” as used in this disclosure, are used interchangeably.

The terms “LN3-derived oligosaccharide,” “LN3 derived oligosaccharide” or “oligosaccharide derived from LN3” as used herein are used interchangeably and refer to an oligosaccharide with a degree of polymerization of at least 4 that comprises an LN3 core that has been extended by at least one additional monosaccharide subunit as described herein. The oligosaccharide can be a linear or a branched oligosaccharide. The oligosaccharide can contain alpha-glycosidic and/or beta-glycosidic linkages. Examples comprise but are not limited to LNT, LNnT, LSTa, LSTb, LSTc, LSTd, DSLNnT, DSLNT, LNFP-I, LNFP-II, LNFP-III, LNFP-V, LNFP-VI, lacto-N-neofucopentaose I, lacto-N-difucohexaose I (LDFH I), lacto-N-difucohexaose II (LDFH II), Monofucosyllacto-N-hexaose III (MFLNH III), Difucosyllacto-N-hexaose (DFLNHa), difucosyl-lacto-N-neohexaose, 3′-sialyl-3-fucosyllactose, disialomonofucosyllacto-N-neohexaose, monofucosylmonosialyllacto-N-octaose (sialyl Lea), sialyllacto-N-fucohexaose II, disialyllacto-N-fucopentaose II, monofucosyldisialyllacto-N-tetraose, GalNAc-LNFP-I, LNnDFH II, LNDFH I, LNDFH II, LNH, para-LNH, LNnH, para-LNnH, F-LNH I, F-LNH-II, DF-LNH I, DF-LNH II, DFLNH c, DF-LNnH, DF-para-LNH, DF-para-LNnH, TF-LNH, F-LST a, F-LST b, F-LST c, FS-LNH, FS-LNnH I and FDS-LNH II.

The terms “LNT,” “lacto-N-tetraose,” “lacto-N-tetraose” or “Galβ1-3GlcNAcβ1-3Galβ1-4Glc” as used in this disclosure, are used interchangeably.

The terms “LNnT,” “lacto-N-neotetraose,” “lacto-N-neotetraose,” “neo-LNT” or “Galβ1-4GlcNAcβ1-3Galβ1-4Glc” as used in this disclosure, are used interchangeably.

The terms “LSTa,” “LS-Tetrasaccharide a,” “Sialyl-lacto-N-tetraose a,” “sialyllacto-N-tetraose a” or “Neu5Ac-a2,3-Gal-b1,3-GlcNAc-b1,3-Gal-b1,4-Glc” as used in this disclosure, are used interchangeably.

The terms “LSTb,” “LS-Tetrasaccharide b,” “Sialyl-lacto-N-tetraose b,” “sialyllacto-N-tetraose b” or “Gal-b1,3-(Neu5Ac-a2,6)-GlcNAc-b1,3-Gal-b1,4-Glc” as used in this disclosure, are used interchangeably.

The terms “LSTc,” “LS-Tetrasaccharide c,” “Sialyl-lacto-N-tetraose c,” “sialyllacto-N-tetraose c,” “sialyllacto-N-neotetraose c” or “Neu5Ac-a2,6-Gal-b1,4-GlcNAc-b1,3-Gal-b1,4-Glc” as used in this disclosure, are used interchangeably.

The terms “LSTd,” “LS-Tetrasaccharide d,” “Sialyl-lacto-N-tetraose d,” “sialyllacto-N-tetraose d,” “sialyllacto-N-neotetraose d” or “Neu5Ac-a2,3-Gal-b1,4-GlcNAc-b1,3-Gal-b1,4-Glc” as used in this disclosure, are used interchangeably.

The terms “DSLNnT” and “Disialyllacto-N-neotetraose” are used interchangeably and refer to Neu5Ac-a2,6-Gal-b1,4-GlcNAc-b1,3-[Neu5Ac-a2,6]-Gal-b1,4-Glc.

The terms “DSLNT,” “DS-LNT” and “Disialyllacto-N-tetraose” are used interchangeably and refer to Neu5Ac-a2,3-Gal-b1,3-[Neu5Ac-a2,6]-GlcNAc-b1,3-Gal-b1,4-Glc.

“Charged oligosaccharides” are oligosaccharide structures that contain one or more negatively charged monosaccharide subunits including N-acetylneuraminic acid (Neu5Ac), commonly known as sialic acid, N-glycolylneuraminic acid (Neu5Gc), glucuronate and galacturonate. Charged oligosaccharides are also referred to as acidic oligosaccharides. Sialic acid belongs to the family of derivatives of neuraminic acid (5-amino-3,5-dideoxy-D-glycero-D-galacto-non-2-ulosonic acid). Neu5Gc is a derivative of sialic acid, which is formed by hydroxylation of the N-acetyl group at C5 of Neu5Ac. In contrast, neutral oligosaccharides are non-sialylated oligosaccharides, and thus do not contain an acidic monosaccharide subunit. Neutral oligosaccharides comprise non-charged fucosylated oligosaccharides that contain one or more fucose subunits in their glycan structure as well as non-charged non-fucosylated oligosaccharides that lack any fucose subunit. Other examples of charged oligosaccharides are sulphated chitosans and deacetylated chitosans.

The terms ‘neutral oligosaccharide’ and ‘non-charged oligosaccharide’ as used herein are used interchangeably and refer, as generally understood in the state of the art, to an oligosaccharide that has no negative charge originating from a carboxylic acid group. Examples of such neutral oligosaccharide are 2′-fucosyllactose (2′FL), 3-fucosyllactose (3FL), 2′, 3-difucosyllactose (diFL), lacto-N-triose II (LN3), lacto-N-tetraose (LNT), lacto-N-neotetraose (LNnT), lacto-N-fucopentaose I, lacto-N-neofucopentaose I, lacto-N-fucopentaose II, lacto-N-fucopentaose III, lacto-N-fucopentaose V, lacto-N-fucopentaose VI, lacto-N-neofucopentaose V, lacto-N-difucohexaose I, lacto-N-difucohexaose II, 6′-galactosyllactose, 3′-galactosyllactose, lacto-N-hexaose, lacto-N-neohexaose, para-lacto-N-hexaose, para-lacto-N-neohexaose, difucosyl-lacto-N-hexaose and difucosyl-lacto-N-neohexaose.

A ‘fucosylated oligosaccharide’ as used herein and as generally understood in the state of the art is an oligosaccharide that is carrying a fucose-residue. Such fucosylated oligosaccharide is a saccharide structure comprising at least three monosaccharide subunits linked to each other via glycosidic bonds, wherein at least one of the monosaccharide subunit is a fucose. A fucosylated oligosaccharide can contain more than one fucose residue, e.g., two, three or more. A fucosylated oligosaccharide can be a neutral oligosaccharide or a charged oligosaccharide e.g., also comprising sialic acid structures. Fucose can be linked to other monosaccharide subunits comprising glucose, galactose, GlcNAc via alpha-glycosidic bonds comprising alpha-1,2 alpha-1,3, alpha-1,4, alpha-1,6 linkages. Examples comprise 2′-fucosyllactose (2′FL), 3-fucosyllactose (3FL), 4-fucosyllactose (4FL), 6-fucosyllactose (6FL), difucosyllactose (diFL), Lacto-N-fucopentaose I (LNFP I), Lacto-N-fucopentaose II (LNFP II), Lacto-N-fucopentaose III (LNFP III), lacto-N-fucopentaose V (LNFP V), lacto-N-fucopentaose VI (LNFP VI), lacto-N-neofucopentaose I, lacto-N-difucohexaose I (LDFH I), lacto-N-difucohexaose II (LDFH II), Monofucosyllacto-N-hexaose III (MFLNH III), Difucosyllacto-N-hexaose (DFLNHa), difucosyl-lacto-N-neohexaose, 3′-sialyl-3-fucosyllactose, disialomonofucosyllacto-N-neohexaose, monofucosylmonosialyllacto-N-octaose (sialyl Lea), sialyllacto-N-fucohexaose II, disialyllacto-N-fucopentaose II, monofucosyldisialyllacto-N-tetraose.

The terms “LNFP-I,” “lacto-N-fucopentaose I,” “LNFP I,” “LNFPI,” “LNF I OH type I determinant,” “LNF I,” “LNF1,” “LNF 1” and “Blood group H antigen pentaose type 1” are used interchangeably and refer to Fuc-a1,2-Gal-b1,3-GlcNAc-b1,3-Gal-b1,4-Glc. The terms “GalNAc-LNFP-I” and “blood group A antigen hexaose type I” are used interchangeably and refer to GalNAc-a1,3-(Fuc-a1,2)-Gal-b1,3-GlcNAc-b1,3-Gal-b1,4-Glc.

The terms “LNFP-II” and “lacto-N-fucopentaose II” are used interchangeably and refer to Gal-b1,3-[Fuc-a1,4]-GlcNAc-b1,3-Gal-b1,4-Glc. The terms “LNFP-III,” “LNFP III,” “LNFP III” and “lacto-N-fucopentaose III” are used interchangeably and refer to Gal-b1,4-(Fuc-a1,3)-GlcNAc-b1,3-Gal-b1,4-Glc. The terms “LNFP-V,” “LNFP V,” “LNFPV” and “lacto-N-fucopentaose V” are used interchangeably and refer to Gal-b1,3-GlcNAc-b1,3-Gal-b1,4-(Fuc-a1,3)-Glc. The terms “LNFP-VI,” “LNFP VI,” “LNnFP V” and “lacto-N-neofucopentaose V” are used interchangeably and refer to Gal-b1,4-GlcNAc-b1,3-Gal-b1,4-(Fuc-a1,3)-Glc. The terms “LNnFP I” and “Lacto-N-neofucopentaose I” are used interchangeably and refer to Fuc-a1,2-Gal-b1,4-GlcNAc-b1,3-Gal-b1,4-Glc. The terms “LNDFH I,” “Lacto-N-difucohexaose I,” “LNDFH-I,” “LDFH I,” “Le^{b}-lactose” and “Lewis-b hexasaccharide” are used interchangeably and refer to Fuc-a1,2-Gal-b1,3-[Fuc-a1,4]-GlcNAc-b1,3-Gal-b1,4-Glc. The terms “LNDFH II,” “Lacto-N-difucohexaose II,” “LNDFH-II,” “Lewis a-Lewis x” and “LDFH II” are used interchangeably and refer to Gal-b1,3-[Fuc-a1,4]-GlcNAc-b1,3-Gal-b1,4-(Fuc-a1,3)-Glc. The terms “LNnDFH II,” “Lacto-N-neodifucohexaose II,” “LNDFH III,” “Lewis x hexaose” and “LeX hexaose” are used interchangeably and refer to Gal-b1,4-(Fuc-a1,3)-GlcNAc-b1,3-Gal-b1,4-(Fuc-a1,3)-Glc. The terms “alpha-tetrasaccharide” and “A-tetrasaccharide” are used interchangeably and refer to GalNAc-a1,3-(Fuc-a1,2)-Gal-b1,4-Glc. The terms “LNH” and “lacto-N-hexaose” are used interchangeably and refer to Gal-b1,3-GlcNAc-b1,3-(Gal-b1,4-GlcNAc-b1,6)-Gal-b1,4-Glc. The terms “para-LNH,” “pLNH” and “para-lacto-N-hexaose” are used interchangeably and refer to Gal-b1,3-GlcNAc-b1,3-Gal-b1,4-GlcNAc-b1,3-Gal-b1,4-Glc. The terms “LNnH” and “lacto-N-neohexaose” are used interchangeably and refer to Gal-b1,4-GlcNAc-b1,3-[Gal-b1,4-GlcNAc-b1,6]-Gal-b1,4-Glc. The terms “para-LNnH,” “pLNnH” and “para-lacto-N-neohexaose” are used interchangeably and refer to Gal-b1,4-GlcNAc-b1,3-Gal-b1,4-GlcNAc-b1,3-Gal-b1,4-Glc.

The terms “F-LNH I,” “FLNH I” and “fucosyllacto-N-hexaose I” are used interchangeably and refer to Fuc-a1,2-Gal-b1,3-GlcNAc-b1,3-[Gal-b1,4-GlcNAc-b1,6]-Gal-b1,4-Glc. The terms “F-LNH-II,” “FLNH II” and “fucosyllacto-N-hexaose II” are used interchangeably and refer to Gal-b1,3-GlcNAc-b1,3-[Gal-b1,4-[Fuc-a1,3]-GlcNAc-b1,6]-Gal-b1,4-Glc. The terms “DF-LNH I,” “difucosyllacto-N-hexaose I,” “DF-LNH a,” “DFLNH a,” “difucosyllacto-N-hexaose a” and “2,3-Difucosyllacto-N-hexaose” are used interchangeably and refer to Fuc-a1,2-Gal-b1,3-GlcNAc-b1,3-[Gal-b1,4-[Fuc-a1,3]-GlcNAc-b1,6]-Gal-b1,4-Glc. The terms “DF-LNH II,” “DF-LNH b,” “DFLNH b” and “difucosyllacto-N-hexaose II” are used interchangeably and refer to Gal-b1,3-[Fuc-a1,4]-GlcNAc-b1,3-[Gal-b1,4-[Fuc-a1,3]-GlcNAc-b1,6]-Gal-b1,4-Glc. The terms “DFLNH c,” “DF-LNH c” and “difucosyllacto-N-hexaose c” are used interchangeably and refer to Fuc-a1,2-Gal-b1,3-[Fuc-a1,4]-GlcNAc-b1,3-[Gal-b1,4-GlcNAc-b1,6]-Gal-b1,4-Glc. The terms “DF-LNnH” and “difucosyllacto-N-neohexaose” are used interchangeably and refer to Gal-b1,4-[Fuc-a1,3]-GlcNAc-b1,3-[Gal-b1,4-[Fuc-a1,3]-GlcNAc-b1,6]-Gal-b1,4-Glc. The terms “DF-para-LNH,” “DF-p-LNH,” “DF-pLNH” and “difucosyl-para-lacto-N-hexaose” are used interchangeably and refer to Gal-b1,3-[Fuc-a1,4]-GlcNAc-b1,3-Gal-b1,4-[Fuc-a1,3]-GlcNAc-b1,3-Gal-b1,4-Glc. The terms “DF-para-LNnH,” “DF-p-LNnH” and “difucosyl-para-lacto-N-neohexaose” are used interchangeably and refer to Gal-b1,4-[Fuc-a1,3]-GlcNAc-b1,3-Gal-b1,4-[Fuc-a1,3]-GlcNAc-b1,3-Gal-b1,4-Glc. The terms “TF-LNH” and “trifucosyllacto-N-hexaose” are used interchangeably and refer to Fuc-a1,2-Gal-b1,3-[Fuc-a1,4]-GlcNAc-b1,3-[Gal-b1,4-[Fuc-a1,3]-GlcNAc-b1,6]-Gal-b1,4-Glc.

The terms “F-LST a,” “F-LSTa,” “S-LNF II” and “fucosyl-sialyllacto-N-tetraose a” are used interchangeably and refer to Neu5Ac-a2,3-Gal-b1,3-[Fuc-a1,4]-GlcNAc-b1,3-Gal-b1,4-Glc. The terms “F-LST b,” “F-LSTb,” “S-LNF I” and “fucosyl-sialyllacto-N-tetraose b” are used interchangeably and refer to Fuc-a1,2-Gal-b1,3-(Neu5Ac-a2,6)-GlcNAc-b1,3-Gal-b1,4-Glc. The terms “F-LST c,” “F-LSTc” and “fucosyl-sialyllacto-N-neotetraose” are used interchangeably and refer to Neu5Ac-a2,6-Gal-b1,4-GlcNAc-b1,3-Gal-b1,4-[Fuc-a1,3]-Glc.

The terms “FS-LNH” and “fucosyl-sialyllacto-N-hexaose” are used interchangeably and refer to Fuc-a1,2-Gal-b1,3-GlcNAc-b1,3-(Neu5Ac-a2,6-Gal-b1,4-GlcNAc-b1,6)-Gal-b1,4-Glc.

The terms “FS-LNnH I” and “fucosyl-sialyllacto-N-neohexaose I” are used interchangeably and refer to Neu5Ac-a2,6-Gal-b1,4-GlcNAc-b1,3-[Gal-b1,4-[Fuc-a1,3]-GlcNAc-b1,6]-Gal-b1,4-Glc.

The terms “FDS-LNH II” and “fucosyldisialyllacto-N-hexaose II” are used interchangeably and refer to Neu5Ac-a2,3-Gal-b1,3-[Neu5Ac-a2,6]-GlcNAc-b1,3-[Gal-b1,4-[Fuc-a1,3]-GlcNAc-b1,6]-Gal-b1,4-Glc.

Mammalian milk oligosaccharides comprise oligosaccharides present in milk found in any phase during lactation including colostrum milk from humans and mammals including but not limited to cows (Bos Taurus), sheep (Ovis aries), goats (Capra aegagrus hircus), bactrian camels (Camelus bactrianus), horses (Equus ferus caballus), pigs (Sus scropha), dogs (Canis lupus familiaris), ezo brown bears (Ursus arctos yesoensis), polar bear (Ursus maritimus), Japanese black bears (Ursus thibetanus japonicus), striped skunks (Mephitis mephitis), hooded seals (Cystophora cristata), Asian elephants (Elephas maximus), African elephant (Loxodonta africana), giant anteater (Myrmecophaga tridactyla), common bottlenose dolphins (Tursiops truncates), northern minke whales (Balaenoptera acutorostrata), tammar wallabies (Macropus eugenii), red kangaroos (Macropus rufus), common brushtail possum (Trichosurus vulpecula), koalas (Phascolarctos cinereus), eastern quolls (Dasyurus viverrinus), platypus (Ornithorhynchus anatinus). As used herein, “mammalian milk oligosaccharide” or MMO refers to oligosaccharides such as but not limited to 3-fucosyllactose, 2′-fucosyllactose, 6-fucosyllactose, 2′,3-difucosyllactose, 2′,2-difucosyllactose, 3,4-difucosyllactose, 6′-sialyllactose, 3′-sialyllactose, 3,6-disialyllactose, 6,6′-disialyllactose, 8,3-disialyllactose, 3,6-disialyllacto-N-tetraose, lactodifucotetraose, lacto-N-tetraose, lacto-N-neotetraose, lacto-N-fucopentaose II, lacto-N-fucopentaose I, lacto-N-fucopentaose III, lacto-N-fucopentaose V, lacto-N-fucopentaose VI, sialyllacto-N-tetraose c, sialyllacto-N-tetraose b, sialyllacto-N-tetraose a, lacto-N-difucohexaose I, lacto-N-difucohexaose II, lacto-N-hexaose, lacto-N-neohexaose, para-lacto-N-hexaose, monofucosylmonosialyllacto-N-tetraose c, monofucosyl para-lacto-N-hexaose, monofucosyllacto-N-hexaose III, isomeric fucosylated lacto-N-hexaose III, isomeric fucosylated lacto-N-hexaose I, sialyllacto-N-hexaose, sialyllacto-N-neohexaose II, difucosyl-para-lacto-N-hexaose, difucosyllacto-N-hexaose, difucosyllacto-N-hexaose a, difucosyllacto-N-hexaose c, galactosylated chitosan, fucosylated oligosaccharides, neutral oligosaccharide and/or sialylated oligosaccharides.

The terms “human milk oligosaccharide” or “HMO” refer to oligosaccharides found in human breast milk, including preterm human milk, colostrum and term human milk. HMOs comprise fucosylated oligosaccharides, non-fucosylated neutral oligosaccharides and sialylated oligosaccharides (see e.g., Chen X., Chapter Four: Human Milk Oligosaccharides (HMOS): Structure, Function, and Enzyme-Catalyzed Synthesis in Adv. Carbohydr. Chem. Biochem. 72, 113 (2015)). Examples of HMOs comprise 3-fucosyllactose, 2′-fucosyllactose, 2′,3-difucosyllactose, 6′-sialyllactose, 3′-sialyllactose, LN3, lacto-N-tetraose, lacto-N-neotetraose, lacto-N-fucopentaose II, lacto-N-fucopentaose I, lacto-N-fucopentaose III, lacto-N-fucopentaose V, lacto-N-fucopentaose VI, sialyllacto-N-tetraose c, sialyllacto-N-tetraose b, sialyllacto-N-tetraose a, difucosyllacto-N-tetraose, lacto-N-hexaose, lacto-N-difucohexaose I, lacto-N-difucohexaose II, disialyllacto-N-tetraose, fucosyllacto-N-hexaose, difucosyllacto-N-hexaose, fucodisialyllacto-N-hexaose, disialyllacto-N-hexaose.

The term “cultivation” refers to the culture medium wherein the cell is cultivated, or fermented, the cell itself, and one or more bioproduct(s) that is/are produced by the cell in whole broth, i.e., inside (intracellularly) as well as outside (extracellularly) of the cell.

The term “incubation” refers to a mixture wherein one or more bioproduct(s) is/are produced. The mixture can comprise one or more enzyme(s), one or more precursor(s) and one or more acceptor(s) as defined herein present in a buffered solution and incubated for a certain time at a certain temperature enabling production of one or more bioproduct(s), catalyzed by the one or more enzyme(s) using the one or more precursor(s) and the one or more acceptor(s) in the mixture. The mixture can also comprise i) the cell obtained after cultivation or incubation, optionally the cell is subjected to cell lysis, ii) a buffered solution or the cultivation or incubation medium wherein the cell was cultivated or fermented, and iii) one or more bioproduct(s) that is/are produced by the cell in whole broth, i.e., inside (intracellularly) as well as outside (extracellularly) of the cell. The incubation can also be the cultivation as defined herein.

The terms “reactor” and “incubator” refer to the recipient filled with the cultivation or incubation. Examples of reactors and incubators comprise but are not limited to microfluidic devices, well plates, tubes, shake flasks, fermenters, bioreactors, process vessels, cell culture incubators, CO2 incubators. The reactor and incubator can each vary from lab-scale dimensions to large-scale industrial dimensions.

As used herein, the term “cell productivity index (CPI)” refers to the mass of the bioproduct produced by the cells divided by the mass of the cells produced in the culture.

The term “purified” refers to material that is substantially or essentially free from components that interfere with the activity of the biological molecule. For cells, saccharides, nucleic acids, and polypeptides, the term “purified” refers to material that is substantially or essentially free from components that normally accompany the material as found in its native state. Typically, purified saccharides, oligosaccharides, proteins or nucleic acids of the disclosure are at least about 50%, 55%, 60%, 65%, 70%, 75%, 80% or 85% pure, usually at least about 90%, 91%, 92%, 93%, 94%, 95%, 96%, 97%, 98%, or 99.0% pure as measured by band intensity on a silver-stained gel or other method for determining purity. Purity or homogeneity can be indicated by a number of means well known in the art, such as polyacrylamide gel electrophoresis of a protein or nucleic acid sample, followed by visualization upon staining. For certain purposes high resolution will be needed and HPLC or a similar means for purification utilized. For di- and oligosaccharides, purity can be determined using methods such as but not limited to thin layer chromatography, gas chromatography, NMR, HPLC, capillary electrophoresis or mass spectroscopy. Further herein, the terms “contaminants” and “impurities” preferably mean particulates, cells, cell components, metabolites, cell debris, proteins, peptides, amino acids, nucleic acids, glycolipids and/or endotoxins which can be present in an aqueous medium like e.g., a cultivation or an incubation.

The term “clarifying” as used herein refers to the act of treating an aqueous medium like e.g., a cultivation or an incubation, to remove suspended particulates and contaminants from the production process, like e.g., cells, cell components, insoluble metabolites and debris, that could interfere with the eventual purification of the one or more bioproduct(s). Such treatment can be carried out in a conventional manner by centrifugation, flocculation, flocculation with optional ultrasonic treatment, gravity filtration, microfiltration, foam separation or vacuum filtration (e.g., through a ceramic filter which can include a Celite™ filter aid).

The term “precursor” as used herein refers to substances which are taken up or synthetized by the cell for the specific production of one or more bioproduct(s) according to this disclosure. In this sense a precursor can be an acceptor as defined herein, but can also be another substance, metabolite, which is first modified within the cell as part of the biochemical synthesis route(s) of one or more bioproduct(s). The term “precursor” as used herein is also to be understood as a chemical compound that participates in a chemical or enzymatic reaction to produce another compound like e.g., an intermediate or an acceptor as defined herein, as part in the metabolic pathway of one or more bioproduct(s). The term “precursor” as used herein is also to be understood as a donor that is used by a glycosyltransferase to modify an acceptor as defined herein with a sugar moiety in a glycosidic bond, as part in the metabolic pathway of one or more bioproduct(s). Examples of such precursors comprise the acceptors as defined herein, and/or dihydroxyacetone, glucosamine, N-acetylglucosamine, N-acetylmannosamine, galactosamine, N-acetylgalactosamine, galactosyllactose, phosphorylated sugars or sugar phosphates like e.g., but not limited to glucose-1-phosphate, galactose-1-phosphate, glucose-6-phosphate, fructose-6-phosphate, fructose-1,6-bisphosphate, mannose-6-phosphate, mannose-1-phosphate, glycerol-3-phosphate, glyceraldehyde-3-phosphate, dihydroxyacetone-phosphate, glucosamine-6-phosphate, N-acetylglucosamine-6-phosphate, N-acetylmannosamine-6-phosphate, N-acetylglucosamine-1-phosphate, N-acetylneuraminic acid-9-phosphate and nucleotide-activated sugars like nucleotide diphospho-sugars and nucleotide monophospho-sugars as defined herein like e.g., UDP-glucose, UDP-galactose, UDP-N-acetylglucosamine, CMP-sialic acid, GDP-mannose, GDP-4-dehydro-6-deoxy-α-D-mannose, GDP-fucose.

Optionally, the cell is transformed to comprise and to express at least one nucleic acid sequence encoding a protein selected from the group consisting of lactose transporter, N-acetylneuraminic acid transporter, fucose transporter, glucose transporter, galactose transporter, transporter for a nucleotide-activated sugar wherein the transporter internalizes a to the medium added precursor for the synthesis of one or more bioproduct(s) of this disclosure.

The term “acceptor” as used herein refers to a mono-, di- or oligosaccharide, which can be modified by a glycosyltransferase. Examples of such acceptors comprise glucose, galactose, fructose, glycerol, sialic acid, fucose, mannose, maltose, sucrose, lactose, lacto-N-triose, lacto-N-tetraose (LNT), LNnT, lacto-N-pentaose (LNP), lacto-N-neopentaose, para lacto-N-pentaose, para lacto-N-neopentaose, lacto-N-novopentaose I, lacto-N-hexaose (LNH), lacto-N-neohexaose (LNnH), para lacto-N-neohexaose (pLNnH), para lacto-N-hexaose (pLNH), lacto-N-heptaose, lacto-N-neoheptaose, para lacto-N-neoheptaose, para lacto-N-heptaose, lacto-N-octaose (LNO), lacto-N-neooctaose, iso lacto-N-octaose, para lacto-N-octaose, iso lacto-N-neooctaose, novo lacto-N-neooctaose, para lacto-N-neooctaose, iso lacto-N-nonaose, novo lacto-N-nonaose, lacto-N-nonaose, lacto-N-decaose, iso lacto-N-decaose, novo lacto-N-decaose, lacto-N-neodecaose, and oligosaccharide containing 1 or more N-acetyllactosamine units and/or 1 or more lacto-N-biose units or an intermediate into oligosaccharide, fucosylated and sialylated versions thereof, ceramide, N-acylated sphingoid, glucosylceramide, lactosylceramide, sphingosine, phytosphingosine, sphingosine synthons, peptide backbones with beta-GlcNAc-Asn residues, glycoproteins with terminal GlcNAc and Gal residues, immunoglobulins.

### DETAILED DESCRIPTION

According to a first aspect, provided is a cell for the production of one or more bioproduct(s), wherein the cell is capable to produce, preferably produces, one or more precursor(s) used in the production of at least one of the one or more bioproduct(s) and wherein the cell is genetically engineered to express, preferably to overexpress, a polynucleotide sequence that encodes a saccharide importer that internalizes at least one of the one or more precursor(s) into the cell.

In the scope of the disclosure, the one or more bioproduct(s) as described herein is/are chosen from the list comprising saccharide; monosaccharide; activated monosaccharide; phosphorylated monosaccharide; disaccharide; oligosaccharide; polysaccharide; milk saccharide; mammalian milk saccharide; MMO; human milk saccharide; HMO; neutral (non-charged) saccharide; negatively charged saccharide; fucosylated saccharide; sialylated saccharide; neutral (non-charged) oligosaccharide; negatively charged oligosaccharide; fucosylated oligosaccharide; sialylated oligosaccharide; N-acetylglucosamine containing oligosaccharide; N-acetyllactosamine containing oligosaccharide; lacto-N-biose containing oligosaccharide; lactose containing oligosaccharide; non-fucosylated neutral (non-charged) oligosaccharide; O-antigen; ECA; the oligosaccharide repeats present in capsular polysaccharides; peptidoglycan; amino-sugar; Lewis-type antigen oligosaccharide; antigen of the human ABO blood group system; animal oligosaccharide, preferably selected from the group consisting of N-glycans and O-glycans; plant oligosaccharide, preferably selected from the group consisting of N-glycans and O-glycans; LN3; lacto-N-tetraose (LNT); LNnT; oligosaccharide derived from LN3; 3′sialyllactose (3′SL); 6′sialyllactose (6′SL); sialyllacto-N-tetraose a (LSTa); sialyllacto-N-tetraose b (LSTb); sialyllacto-N-tetraose c (LSTc); sialyllacto-N-tetraose d (LSTd); lacto-N-fucopentaose I; lacto-N-neofucopentaose; lacto-N-fucopentaose II; lacto-N-fucopentaose III; lacto-N-fucopentaose V; lacto-N-neofucopentaose V; lacto-N-difucohexaose I; lacto-N-neodifucohexaose; lacto-N-difucohexaose II; monofucosyllacto-n-hexaose III; difucosyllacto-N-hexaose a; 6′-galactosyllactose; 3′-galactosyllactose; lacto-N-hexaose; lacto-N-neohexaose; 2′-fucosyllactose (2′FL); 3-fucosyllactose (3-FL); difucosyllactose (DiFL); chitosan; chitosan comprising oligosaccharide; heparosan; glycosaminoglycan oligosaccharide; heparin; heparan sulphate; chondroitin sulphate; dermatan sulphate; hyaluronan; hyaluronic acid and keratan sulphate. In a preferred embodiment of this disclosure, the one or more bioproduct(s) is/are LN3 and/or one or more LN3-derived oligosaccharide(s) as defined herein.

In a preferred embodiment of the disclosure, the one or more precursor(s) as described herein is/are chosen from the list comprising saccharide; monosaccharide; activated monosaccharide; phosphorylated monosaccharide; disaccharide; oligosaccharide; polysaccharide; milk saccharide; mammalian milk saccharide; MMO; human milk saccharide; HMO; neutral (non-charged) saccharide; negatively charged saccharide; fucosylated saccharide; sialylated saccharide; neutral (non-charged) oligosaccharide; negatively charged oligosaccharide; fucosylated oligosaccharide; sialylated oligosaccharide; N-acetylglucosamine containing oligosaccharide; N-acetyllactosamine containing oligosaccharide; lacto-N-biose containing oligosaccharide; lactose containing oligosaccharide; non-fucosylated neutral (non-charged) oligosaccharide; O-antigen; ECA; the oligosaccharide repeats present in capsular polysaccharides; peptidoglycan; amino-sugar; Lewis-type antigen oligosaccharide; antigen of the human ABO blood group system; animal oligosaccharide, preferably selected from the group consisting of N-glycans and O-glycans; plant oligosaccharide, preferably selected from the group consisting of N-glycans and O-glycans; LN3; lacto-N-tetraose (LNT); LNnT; oligosaccharide derived from LN3; 3′sialyllactose (3′SL); 6′sialyllactose (6′SL); sialyllacto-N-tetraose a (LSTa); sialyllacto-N-tetraose b (LSTb); sialyllacto-N-tetraose c (LSTc); sialyllacto-N-tetraose d (LSTd); lacto-N-fucopentaose I; lacto-N-neofucopentaose; lacto-N-fucopentaose II; lacto-N-fucopentaose III; lacto-N-fucopentaose V;

lacto-N-neofucopentaose V; lacto-N-difucohexaose I; lacto-N-neodifucohexaose; lacto-N-difucohexaose II; monofucosyllacto-n-hexaose III; difucosyllacto-N-hexaose a; 6′-galactosyllactose; 3′-galactosyllactose; lacto-N-hexaose; lacto-N-neohexaose; 2′-fucosyllactose (2′FL); 3-fucosyllactose (3-FL); difucosyllactose (DiFL); chitosan; chitosan comprising oligosaccharide; heparosan; glycosaminoglycan oligosaccharide; heparin; heparan sulphate; chondroitin sulphate; dermatan sulphate; hyaluronan; hyaluronic acid and keratan sulphate. In a more preferred embodiment of this disclosure, the one or more precursor(s) is/are LN3 and/or one or more LN3-derived oligosaccharide(s) as defined herein. In an even more preferred embodiment, one of the precursors is LN3.

In a preferred embodiment of the cell and/or method of this disclosure, the cell produces one bioproduct. In another preferred embodiment of the cell and/or method of this disclosure, the cell produces more than one bioproduct. In another preferred embodiment of the cell and/or method of this disclosure, the cell produces a mixture comprising at least one bioproduct as described herein. In a more preferred embodiment, the cell produces LN3. In another and/or additional preferred embodiment, the cell produces an LN3-derived oligosaccharide. In another and/or additional preferred embodiment, the cell produces LNT and/or LNnT. In another and/or additional preferred embodiment, the cell produces a mixture of LN3-derived oligosaccharides.

In another preferred embodiment of the cell and/or method of this disclosure, the cell produces one bioproduct and one precursor that is used in the production of the bioproduct, wherein the precursor is present outside the cell by any one or more of passive transport out of the cell, active transport out of the cell, secretion, excretion and/or cell lysis during and/or at the end of the cultivation and/or incubation of the cell, and the cell is genetically engineered to (over) express a polynucleotide sequence that encodes a saccharide importer that internalizes the precursor inside the cell to be used for the production of the bioproduct.

In another preferred embodiment of the cell and/or method of this disclosure, the cell produces one bioproduct and two precursors that are used in the production of the bioproduct, wherein one of the precursors is present outside the cell by any one or more of passive transport out of the cell, active transport out of the cell, secretion, excretion and/or cell lysis during and/or at the end of the cultivation and/or incubation of the cell and wherein the other one of the two precursors is present inside the cell, and the cell is genetically engineered to (over) express a polynucleotide sequence that encodes a saccharide importer that internalizes the extracellular precursor inside the cell to be used for the production of the bioproduct.

In another preferred embodiment of the cell and/or method of this disclosure, the cell produces one bioproduct and two precursors that are used in the production of the bioproduct, wherein both of the precursors are present outside the cell by any one or more of passive transport out of the cell, active transport out of the cell, secretion, excretion and/or cell lysis during and/or at the end of the cultivation and/or incubation of the cell, and the cell is genetically engineered to (over) express a polynucleotide sequence that encodes a saccharide importer that internalizes one of the extracellular precursors inside the cell to be used for the production of the bioproduct. In a more preferred embodiment, the cell produces one bioproduct and two precursors that are used in the production of the bioproduct, wherein both of the precursors are present outside the cell by any one or more of passive transport out of the cell, active transport out of the cell, secretion, excretion and/or cell lysis during and/or at the end of the cultivation and/or incubation of the cell, and the cell is genetically engineered to (over) express a polynucleotide sequence that encodes a saccharide importer that internalizes both of the extracellular precursors inside the cell to be used for the production of the bioproduct. In another more preferred embodiment, the cell produces one bioproduct and two precursors that are used in the production of the bioproduct, wherein both of the precursors are present outside the cell by any one or more of passive transport out of the cell, active transport out of the cell, secretion, excretion and/or cell lysis during and/or at the end of the cultivation and/or incubation of the cell, and the cell is genetically engineered to (over) express two polynucleotide sequences that each encode a different saccharide importer, wherein a first saccharide importer internalizes one of the extracellular precursors and a second saccharide importer internalizes the other one of the extracellular precursors inside the cell to be used for the production of the bioproduct.

In another preferred embodiment of the cell and/or method of this disclosure, the cell produces one bioproduct and three or more precursors that are used in the production of the bioproduct, wherein one of the precursors is present outside the cell by any one or more of passive transport out of the cell, active transport out of the cell, secretion, excretion and/or cell lysis during and/or at the end of the cultivation and/or incubation of the cell and the other precursors are present inside the cell, and the cell is genetically engineered to (over) express a polynucleotide sequence that encodes a saccharide importer that internalizes the extracellular precursor inside the cell to be used for the production of the bioproduct.

In another preferred embodiment of the cell and/or method of this disclosure, the cell produces one bioproduct and three or more precursors that are used in the production of the bioproduct, wherein two of the precursors are present outside the cell by any one or more of passive transport out of the cell, active transport out of the cell, secretion, excretion and/or cell lysis during and/or at the end of the cultivation and/or incubation of the cell and the other precursor(s) is/are present inside the cell, and the cell is genetically engineered to (over) express a polynucleotide sequence that encodes a saccharide importer that internalizes one of the extracellular precursors inside the cell to be used for the production of the bioproduct. In a more preferred embodiment, the cell produces one bioproduct and three or more precursors that are used in the production of the bioproduct, wherein two of the precursors are present outside the cell by any one or more of passive transport out of the cell, active transport out of the cell, secretion, excretion and/or cell lysis during and/or at the end of the cultivation and/or incubation of the cell and the other precursor(s) is/are present inside the cell, and the cell is genetically engineered to (over) express a polynucleotide sequence that encodes a saccharide importer that internalizes both of the extracellular precursors inside the cell to be used for the production of the bioproduct. In another more preferred embodiment, the cell produces one bioproduct and three or more precursors that are used in the production of the bioproduct, wherein two of the precursors are present outside the cell by any one or more of passive transport out of the cell, active transport out of the cell, secretion, excretion and/or cell lysis during and/or at the end of the cultivation and/or incubation of the cell and the other precursor(s) is/are present inside the cell, and the cell is genetically engineered to (over) express two polynucleotide sequences that each encode a different saccharide importer, wherein a first saccharide importer internalizes one of the extracellular precursors and a second saccharide importer internalizes the other one of the extracellular precursors inside the cell to be used for the production of the bioproduct.

In another preferred embodiment of the cell and/or method of this disclosure, the cell produces one bioproduct and three or more precursors that are used in the production of the bioproduct, wherein at least three of the precursors are present outside the cell by any one or more of passive transport out of the cell, active transport out of the cell, secretion, excretion and/or cell lysis during and/or at the end of the cultivation and/or incubation of the cell and the other precursor(s), if present, is/are present inside the cell, and the cell is genetically engineered to (over) express a polynucleotide sequence that encodes a saccharide importer that internalizes one of the extracellular precursors inside the cell to be used for the production of the bioproduct. In a more preferred embodiment, the cell produces one bioproduct and three or more precursors that are used in the production of the bioproduct, wherein at least three of the precursors are present outside the cell by any one or more of passive transport out of the cell, active transport out of the cell, secretion, excretion and/or cell lysis during and/or at the end of the cultivation and/or incubation of the cell and the other precursor(s), if present, is/are present inside the cell, and the cell is genetically engineered to (over) express a polynucleotide sequence that encodes a saccharide importer that internalizes two of the extracellular precursors inside the cell to be used for the production of the bioproduct. In an even more preferred embodiment, the cell produces one bioproduct and three or more precursors that are used in the production of the bioproduct, wherein at least three of the precursors are present outside the cell by any one or more of passive transport out of the cell, active transport out of the cell, secretion, excretion and/or cell lysis during and/or at the end of the cultivation and/or incubation of the cell and the other precursor(s), if present, is/are present inside the cell, and the cell is genetically engineered to (over) express a polynucleotide sequence that encodes a saccharide importer that internalizes three or more, if present, of the extracellular precursors inside the cell to be used for the production of the bioproduct. In another more preferred embodiment, the cell produces one bioproduct and three or more precursors that are used in the production of the bioproduct, wherein at least three of the precursors are present outside the cell by any one or more of passive transport out of the cell, active transport out of the cell, secretion, excretion and/or cell lysis during and/or at the end of the cultivation and/or incubation of the cell and the other precursor(s), if present, is/are present inside the cell, and the cell is genetically engineered to (over) express two or more polynucleotide sequences that encodes two or more different saccharide importers that internalizes three or more, if present, of the extracellular precursors inside the cell to be used for the production of the bioproduct.

In another preferred embodiment of the cell and/or method of this disclosure, the cell produces two bioproducts and one precursor that is used in the production of one or both of the bioproducts, wherein the precursor is present outside the cell by any one or more of passive transport out of the cell, active transport out of the cell, secretion, excretion and/or cell lysis during and/or at the end of the cultivation and/or incubation of the cell, and the cell is genetically engineered to (over) express a polynucleotide sequence that encodes a saccharide importer that internalizes the precursor inside the cell to be used for the production of one or both of the bioproducts.

In another preferred embodiment of the cell and/or method of this disclosure, the cell produces two bioproducts and two precursors that each are used in the production of one or both of the bioproducts, wherein one of the precursors is present outside the cell by any one or more of passive transport out of the cell, active transport out of the cell, secretion, excretion and/or cell lysis during and/or at the end of the cultivation and/or incubation of the cell and wherein the other one of the two precursors is present inside the cell, and the cell is genetically engineered to (over) express a polynucleotide sequence that encodes a saccharide importer that internalizes the extracellular precursor inside the cell to be used for the production of one or both of the bioproducts.

In another preferred embodiment of the cell and/or method of this disclosure, the cell produces two bioproducts and two precursors that each are used in the production of one or both of the bioproducts, wherein both of the precursors are present outside the cell by any one or more of passive transport out of the cell, active transport out of the cell, secretion, excretion and/or cell lysis during and/or at the end of the cultivation and/or incubation of the cell, and the cell is genetically engineered to (over) express a polynucleotide sequence that encodes a saccharide importer that internalizes one of the extracellular precursors inside the cell to be used for the production of one or both of the bioproducts. In a more preferred embodiment, the cell produces two bioproducts and two precursors that each are used in the production of one or both of the bioproducts, wherein both of the precursors are present outside the cell by any one or more of passive transport out of the cell, active transport out of the cell, secretion, excretion and/or cell lysis during and/or at the end of the cultivation and/or incubation of the cell, and the cell is genetically engineered to (over) express a polynucleotide sequence that encodes a saccharide importer that internalizes both of the extracellular precursors inside the cell to be used for the production of one or both of the bioproducts. In another more preferred embodiment, the cell produces two bioproducts and two precursors that each are used in the production of one or both of the bioproducts, wherein both of the precursors are present outside the cell by any one or more of passive transport out of the cell, active transport out of the cell, secretion, excretion and/or cell lysis during and/or at the end of the cultivation and/or incubation of the cell, and the cell is genetically engineered to (over) express two polynucleotide sequences that each encode a different saccharide importer, wherein a first saccharide importer internalizes one of the extracellular precursors and a second saccharide importer internalizes the other one of the extracellular precursors inside the cell to be used for the production of the bioproducts.

In another preferred embodiment of the cell and/or method of this disclosure, the cell produces two bioproducts and three or more precursors that each are used in the production of one or both of the bioproducts, wherein one of the precursors is present outside the cell by any one or more of passive transport out of the cell, active transport out of the cell, secretion, excretion and/or cell lysis during and/or at the end of the cultivation and/or incubation of the cell and the other precursors are present inside the cell, and the cell is genetically engineered to (over) express a polynucleotide sequence that encodes a saccharide importer that internalizes the extracellular precursor inside the cell to be used for the production of one or both of the bioproducts.

In another preferred embodiment of the cell and/or method of this disclosure, the cell produces two bioproducts and three or more precursors that each are used in the production of one or both of the bioproducts, wherein two of the precursors are present outside the cell by any one or more of passive transport out of the cell, active transport out of the cell, secretion, excretion and/or cell lysis during and/or at the end of the cultivation and/or incubation of the cell and the other precursor(s) is/are present inside the cell, and the cell is genetically engineered to (over) express a polynucleotide sequence that encodes a saccharide importer that internalizes one of the extracellular precursors inside the cell to be used for the production of one or both of the bioproducts. In a more preferred embodiment, the cell produces two bioproducts and three or more precursors that each are used in the production of one or both of the bioproducts, wherein two of the precursors are present outside the cell by any one or more of passive transport out of the cell, active transport out of the cell, secretion, excretion and/or cell lysis during and/or at the end of the cultivation and/or incubation of the cell and the other precursor(s) is/are present inside the cell, and the cell is genetically engineered to (over) express a polynucleotide sequence that encodes a saccharide importer that internalizes both of the extracellular precursors inside the cell to be used for the production of one or both of the bioproducts. In another more preferred embodiment, the cell produces two bioproducts and three or more precursors that each are used in the production of one or both of the bioproducts, wherein two of the precursors are present outside the cell by any one or more of passive transport out of the cell, active transport out of the cell, secretion, excretion and/or cell lysis during and/or at the end of the cultivation and/or incubation of the cell and the other precursor(s) is/are present inside the cell, and the cell is genetically engineered to (over) express two polynucleotide sequences that each encode a different saccharide importer, wherein a first saccharide importer internalizes one of the extracellular precursors and a second saccharide importer internalizes the other one of the extracellular precursors inside the cell to be used for the production of the bioproducts.

In another preferred embodiment of the cell and/or method of this disclosure, the cell produces two bioproducts and three or more precursors that each are used in the production of one or both of the bioproducts, wherein at least three of the precursors are present outside the cell by any one or more of passive transport out of the cell, active transport out of the cell, secretion, excretion and/or cell lysis during and/or at the end of the cultivation and/or incubation of the cell and the other precursor(s), if present, is/are present inside the cell, and the cell is genetically engineered to (over) express a polynucleotide sequence that encodes a saccharide importer that internalizes one of the extracellular precursors inside the cell to be used for the production of one or both of the bioproducts. In a more preferred embodiment, the cell produces two bioproducts and three or more precursors that each are used in the production of one or both of the bioproducts, wherein at least three of the precursors are present outside the cell by any one or more of passive transport out of the cell, active transport out of the cell, secretion, excretion and/or cell lysis during and/or at the end of the cultivation and/or incubation of the cell and the other precursor(s), if present, is/are present inside the cell, and the cell is genetically engineered to (over) express a polynucleotide sequence that encodes a saccharide importer that internalizes two of the extracellular precursors inside the cell to be used for the production of one or both of the bioproducts. In an even more preferred embodiment, the cell produces two bioproducts and three or more precursors that each are used in the production of one or both of the bioproducts, wherein at least three of the precursors are present outside the cell by any one or more of passive transport out of the cell, active transport out of the cell, secretion, excretion and/or cell lysis during and/or at the end of the cultivation and/or incubation of the cell and the other precursor(s), if present, is/are present inside the cell, and the cell is genetically engineered to (over) express a polynucleotide sequence that encodes a saccharide importer that internalizes three or more, if present, of the extracellular precursors inside the cell to be used for the production of one or both of the bioproducts. In another more preferred embodiment, the cell produces two bioproducts and three or more precursors that each are used in the production of one or both of the bioproducts, wherein at least three of the precursors are present outside the cell by any one or more of passive transport out of the cell, active transport out of the cell, secretion, excretion and/or cell lysis during and/or at the end of the cultivation and/or incubation of the cell and the other precursor(s), if present, is/are present inside the cell, and the cell is genetically engineered to (over) express two or more polynucleotide sequences that encodes two or more different saccharide importers that internalizes three or more, if present, of the extracellular precursors inside the cell to be used for the production of one or both of the bioproducts.

In another preferred embodiment of the cell and/or method of this disclosure, the cell produces three or more bioproducts and one precursor that is used in the production of one, two, three, more than three if present, or all of the bioproducts, wherein the precursor is present outside the cell by any one or more of passive transport out of the cell, active transport out of the cell, secretion, excretion and/or cell lysis during and/or at the end of the cultivation and/or incubation of the cell, and the cell is genetically engineered to (over) express a polynucleotide sequence that encodes a saccharide importer that internalizes the precursor inside the cell to be used for the production of the one, two, three, more than three if present, or all of the bioproducts.

In another preferred embodiment of the cell and/or method of this disclosure, the cell produces three or more bioproducts and two precursors that are used in the production of one, two, three, more than three if present, or all of the bioproducts, wherein one of the precursors is present outside the cell by any one or more of passive transport out of the cell, active transport out of the cell, secretion, excretion and/or cell lysis during and/or at the end of the cultivation and/or incubation of the cell and wherein the other one of the two precursors is present inside the cell, and the cell is genetically engineered to (over) express a polynucleotide sequence that encodes a saccharide importer that internalizes the extracellular precursor inside the cell to be used for the production of one, two, three, more than three if present, or all of the bioproducts.

In another preferred embodiment of the cell and/or method of this disclosure, the cell produces three or more bioproducts and two precursors that are used in the production of one, two, three, more than three if present, or all of the bioproducts, wherein both of the precursors are present outside the cell by any one or more of passive transport out of the cell, active transport out of the cell, secretion, excretion and/or cell lysis during and/or at the end of the cultivation and/or incubation of the cell, and the cell is genetically engineered to (over) express a polynucleotide sequence that encodes a saccharide importer that internalizes one of the extracellular precursors inside the cell to be used for the production of one, two, three, more than three if present, or all of the bioproducts. In a more preferred embodiment, the cell produces three or more bioproducts and two precursors that are used in the production of one, two, three, more than three if present, or all of the bioproducts, wherein both of the precursors are present outside the cell by any one or more of passive transport out of the cell, active transport out of the cell, secretion, excretion and/or cell lysis during and/or at the end of the cultivation and/or incubation of the cell, and the cell is genetically engineered to (over) express a polynucleotide sequence that encodes a saccharide importer that internalizes both of the extracellular precursors inside the cell to be used for the production of one, two, three, more than three if present, or all of the bioproducts. In another more preferred embodiment, the cell produces three or more bioproducts and two precursors that are used in the production of one, two, three, more than three if present, or all of the bioproducts, wherein both of the precursors are present outside the cell by any one or more of passive transport out of the cell, active transport out of the cell, secretion, excretion and/or cell lysis during and/or at the end of the cultivation and/or incubation of the cell, and the cell is genetically engineered to (over) express two polynucleotide sequences that each encode a different saccharide importer, wherein a first saccharide importer internalizes one of the extracellular precursors and a second saccharide importer internalizes the other one of the extracellular precursors inside the cell to be used for the production of one, two, three, more than three if present, or all of the bioproducts.

In another preferred embodiment of the cell and/or method of this disclosure, the cell produces three or more bioproducts and three or more precursors that are used in the production of one, two, three, more than three if present, or all of the bioproducts, wherein one of the precursors is present outside the cell by any one or more of passive transport out of the cell, active transport out of the cell, secretion, excretion and/or cell lysis during and/or at the end of the cultivation and/or incubation of the cell and the other precursors are present inside the cell, and the cell is genetically engineered to (over) express a polynucleotide sequence that encodes a saccharide importer that internalizes the extracellular precursor inside the cell to be used for the production of one, two, three, more than three if present, or all of the bioproducts.

In another preferred embodiment of the cell and/or method of this disclosure, the cell produces three or more bioproducts and three or more precursors that are used in the production of one, two, three, more than three if present, or all of the bioproducts, wherein two of the precursors are present outside the cell by any one or more of passive transport out of the cell, active transport out of the cell, secretion, excretion and/or cell lysis during and/or at the end of the cultivation and/or incubation of the cell and the other precursor(s) is/are present inside the cell, and the cell is genetically engineered to (over) express a polynucleotide sequence that encodes a saccharide importer that internalizes one of the extracellular precursors inside the cell to be used for the production of one, two, three, more than three if present, or all of the bioproducts. In a more preferred embodiment, the cell produces three or more bioproducts and three or more precursors that are used in the production of one, two, three, more than three if present, or all of the bioproducts, wherein two of the precursors are present outside the cell by any one or more of passive transport out of the cell, active transport out of the cell, secretion, excretion and/or cell lysis during and/or at the end of the cultivation and/or incubation of the cell and the other precursor(s) is/are present inside the cell, and the cell is genetically engineered to (over) express a polynucleotide sequence that encodes a saccharide importer that internalizes both of the extracellular precursors inside the cell to be used for the production of one, two, three, more than three if present, or all of the bioproducts. In another more preferred embodiment, the cell produces three or more bioproducts and three or more precursors that are used in the production of one, two, three, more than three if present, or all of the bioproducts, wherein two of the precursors are present outside the cell by any one or more of passive transport out of the cell, active transport out of the cell, secretion, excretion and/or cell lysis during and/or at the end of the cultivation and/or incubation of the cell and the other precursor(s) is/are present inside the cell, and the cell is genetically engineered to (over) express two polynucleotide sequences that each encode a different saccharide importer, wherein a first saccharide importer internalizes one of the extracellular precursors and a second saccharide importer internalizes the other one of the extracellular precursors inside the cell to be used for the production of one, two, three, more than three if present, or all of the bioproducts.

In another preferred embodiment of the cell and/or method of this disclosure, the cell produces three or more bioproducts and three or more precursors that are used in the production of one, two, three, more than three if present, or all of the bioproducts, wherein at least three of the precursors are present outside the cell by any one or more of passive transport out of the cell, active transport out of the cell, secretion, excretion and/or cell lysis during and/or at the end of the cultivation and/or incubation of the cell and the other precursor(s), if present, is/are present inside the cell, and the cell is genetically engineered to (over) express a polynucleotide sequence that encodes a saccharide importer that internalizes one of the extracellular precursors inside the cell to be used for the production of one, two, three, more than three if present, or all of the bioproducts. In a more preferred embodiment, the cell produces three or more bioproducts and three or more precursors that are used in the production of one, two, three, more than three if present, or all of the bioproducts, wherein at least three of the precursors are present outside the cell by any one or more of passive transport out of the cell, active transport out of the cell, secretion, excretion and/or cell lysis during and/or at the end of the cultivation and/or incubation of the cell and the other precursor(s), if present, is/are present inside the cell, and the cell is genetically engineered to (over) express a polynucleotide sequence that encodes a saccharide importer that internalizes two of the extracellular precursors inside the cell to be used for the production of one, two, three, more than three if present, or all of the bioproducts. In an even more preferred embodiment, the cell produces three or more bioproducts and three or more precursors that are used in the production of one, two, three, more than three if present, or all of the bioproducts, wherein at least three of the precursors are present outside the cell by any one or more of passive transport out of the cell, active transport out of the cell, secretion, excretion and/or cell lysis during and/or at the end of the cultivation and/or incubation of the cell and the other precursor(s), if present, is/are present inside the cell, and the cell is genetically engineered to (over) express a polynucleotide sequence that encodes a saccharide importer that internalizes three or more, if present, of the extracellular precursors inside the cell to be used for the production of one, two, three, more than three if present, or all of the bioproducts. In another more preferred embodiment, the cell produces three or more bioproducts and three or more precursors that are used in the production of one, two, three, more than three if present, or all of the bioproducts, wherein at least three of the precursors are present outside the cell by any one or more of passive transport out of the cell, active transport out of the cell, secretion, excretion and/or cell lysis during and/or at the end of the cultivation and/or incubation of the cell and the other precursor(s), if present, is/are present inside the cell, and the cell is genetically engineered to (over) express two or more polynucleotide sequences that encodes two or more different saccharide importers that internalizes three or more, if present, of the extracellular precursors inside the cell to be used for the production of one, two, three, more than three if present, or all of the bioproducts.

Preferably, the cell is genetically engineered to produce at least one of the one or more precursor(s) that is/are used in the production of at least one of the one or more bioproduct(s). More preferably, the cell is genetically engineered to produce all of the one or more precursor(s) that is/are used in the production of at least one of the one or more bioproduct(s).

In a specific embodiment of this disclosure, the cell is genetically engineered to express, preferably to overexpress, a polynucleotide sequence that encodes a saccharide importer that internalizes at least one of the one or more precursor(s) into the cell. In a preferred embodiment of the cell and/or method of this disclosure, the saccharide importer as described herein internalizes at least two of the one or more precursor(s) into the cell. In an even more preferred embodiment, the saccharide importer as described herein internalizes all of the one or more precursor(s) into the cell.

In another preferred embodiment of the cell and/or method of this disclosure, the saccharide importer has uptake activity for lacto-N-triose (LN3, GlcNAc-beta1,3-Gal-beta1,4-Glc). The LN3 can be synthesized chemically, enzymatically by in vitro glycosylation reactions, by chemoenzymatic synthesis, by fermentative approaches and/or by chemical, physical and/or biological degradation of polysaccharides as well-known by a person skilled in the art. Preferably, the LN3 is produced by a cell. More preferably, the LN3 is produced by a cell of this disclosure.

In another and/or additional preferred embodiment of the cell and/or method of this disclosure, one of the one or more precursor(s) that are produced by the cell is LN3.

In another and/or additional preferred embodiment of the cell and/or method of this disclosure, one of the one or more precursor(s) that are produced by the cell is LN3 and the saccharide importer encoded by a polynucleotide sequence in the cell that is genetically engineered to express, preferably to overexpress, the polynucleotide sequence, has uptake activity for the LN3 being produced by the cell.

According to a second aspect, this disclosure provides a cell for the production of one or more bioproduct(s) as described herein, wherein the cell is genetically engineered to express, preferably to overexpress, a polynucleotide sequence that encodes a saccharide importer that has uptake activity for lacto-N-triose (LN3, GlcNAc-beta1,3-Gal-beta1,4-Glc).

In a preferred embodiment of the cell and/or method of this disclosure, the saccharide importer as described herein originates from the major facilitator superfamily (MFS) of transporters.

In another and/or additional preferred embodiment of the cell and/or method of this disclosure, the saccharide importer as described herein comprises a polypeptide sequence comprising an IPR domain selected from the list comprising IPR001927, IPR002178, IPR016152, IPR018043, IPR020846, IPR036259 and IPR039672 as defined by InterPro 90.0 as released on 4th August 2022. In a more preferred embodiment of the cell and/or method of this disclosure, the saccharide importer as described herein comprises a polypeptide sequence comprising an IPR001927 domain as defined by InterPro 90.0 as released on 4 Aug. 2022.

In another and/or additional preferred embodiment of the cell and/or method of this disclosure, the saccharide importer as described herein comprises a polypeptide sequence comprising PF13347 domain and/or PF00359 domain as defined by PFAM 32.0 as released on September 2018.

In another and/or additional preferred embodiment of the cell and/or method of this disclosure, the saccharide importer as described herein comprises a polypeptide sequence comprising a PANTHER domain selected from the list comprising PTHR11328, PTHR11328:SF24, PTHR11328:SF36 and PTHR11328:SF39 as defined by PANTHER 18.0 as released on 17 Sep. 2023.

In another and/or additional preferred embodiment of the cell and/or method of this disclosure, the saccharide importer as described herein comprises a polypeptide sequence comprising cd17332 domain and/or cd00211 domain as defined by the Conserved Domain Database CDD 3.20 as released on September 2022.

In another and/or additional preferred embodiment of the cell and/or method of this disclosure, the saccharide importer as described herein comprises a polypeptide sequence that is at least 25%, at least 30%, at least 35%, at least 40%, at least 45%, at least 50%, at least 55%, at least 60%, at least 65%, at least 70%, at least 75%, at least 80% identical over a stretch of at least 50 amino acid residues, at least 100 amino acid residues, at least 150 amino acid residues, at least 200 amino acid residues, at least 250 amino acid residues to any one of the polypeptide sequences as represented by SEQ ID NOs: 04, 12, 02, 01, 03, 09, 16, 19, 20 or 21. In a more preferred embodiment, the saccharide importer of this disclosure comprises a polypeptide sequence that is at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8% or at least 99.9% identical to any one of the polypeptide sequences as represented by SEQ ID NOs: 04, 12, 02, 01, 03, 09, 16, 19, 20 or 21 over a stretch of at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290 or at least 300 amino acid residues.

In another and/or additional preferred embodiment of the cell and/or method of this disclosure, the saccharide importer as described herein comprises a polypeptide sequence that is at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8% or at least 99.9% identical to any one of the full-length polypeptide sequences as represented by SEQ ID NOs: 04, 12, 02, 01, 03, 09, 16, 19, 20 or 21 and having saccharide importer activity as described herein.

In another and/or additional preferred embodiment of the cell and/or method of this disclosure, the saccharide importer as described herein comprises a polypeptide sequence as represented by any one of SEQ ID NOs: 04, 12, 02, 01, 03, 09, 16, 19, 20 or 21.

In another and/or additional preferred embodiment of the cell and/or method of this disclosure, the saccharide importer as described here has uptake activity for LN3 and comprises a polypeptide sequence that has a deletion, an insertion and/or a mutation of one or more amino acid residue(s) i) N-terminally from the first transmembrane (TM) domain of any one of SEQ ID NOs: 12, 02, 01, 09, 19, 20 or 21 and/or ii) C-terminally from the last TM domain of any one of SEQ ID NOs: 12, 02, 01, 09, 19, 20 or 21. In a more preferred embodiment, the saccharide importer has uptake activity for LN3 and comprises a polypeptide sequence that has a deletion, an insertion and/or a mutation of one or more amino acid residue(s) N-terminally from the first transmembrane (TM) domain of any one of SEQ ID NOs: 12, 02, 01, 09, 19, 20 or 21 wherein the deletion, insertion and/or mutation of one or more amino acid residue(s) is in or N-terminally of a non-TM helix.

In another and/or additional more preferred embodiment, the saccharide importer of this disclosure has uptake activity for LN3 and comprises a polypeptide sequence that has a deletion, an insertion and/or a mutation of one or more amino acid residue(s) C-terminally from the last TM domain of any one of SEQ ID NOs: 12, 02, 01, 09, 19, 20 or 21, wherein the deletion, insertion and/or mutation of one or more amino acid residue(s) is in or C-terminally of a non-TM helix.

Preferably, a variant saccharide importer having a deletion, an insertion and/or a mutation of one or more amino acid residue(s) i) N-terminally from the first transmembrane (TM) domain of any one of SEQ ID NOs: 12, 02, 01, 09, 19, 20 or 21 and/or ii) C-terminally from the last TM domain of any one of SEQ ID NOs: 12, 02, 01, 09, 19, 20 or 21 has a higher, faster and/or more efficient, i.e., less energy-consuming, uptake ratio for the saccharide compared to the native saccharide importer lacking the deletion, insertion and/or mutation.

In another preferred embodiment of the cell and/or method of this disclosure, a cell for production of one or more bioproduct(s) as described herein and expressing a variant saccharide importer having a deletion, an insertion and/or a mutation of one or more amino acid residue(s) i) N-terminally from the first transmembrane (TM) domain of any one of SEQ ID NOs: 12, 02, 01, 09, 19, 20 or 21 and/or ii) C-terminally from the last TM domain of any one of SEQ ID NOs: 12, 02, 01, 09, 19, 20 or 21 produces higher titers of the bioproduct(s), shows a higher purity of the bioproduct(s), has a higher production rate, has a higher cell performance index, has a higher specific productivity, and/or has a higher growth speed compared to a cell with the same genetic make-up but expressing any one of the native SEQ ID NOs: 12, 02, 01, 09, 19, 20 or 21.

In a more preferred embodiment of this disclosure, the saccharide importer as described herein comprises, consists, or consists essentially of the polypeptide sequence with SEQ ID NO: 03 and has uptake activity for LN3; SEQ ID NO:03 is a mutant polypeptide of the saccharide importer with SEQ ID NO:02 wherein the SEQ ID NO:03 has a deletion of 39 amino acid residues N-terminally from the first TM domain of SEQ ID NO:02 and an insertion of 19 amino acid residues N-terminally from the first TM domain of SEQ ID NO:02. The first TM domain of SEQ ID NO:02 starts at Ser at position 40. Both polypeptides with SEQ ID NOs: 02 and 03 have uptake activity for LN3. In an even more preferred embodiment, the saccharide importer as described herein is the polypeptide with SEQ ID NO:03 and has uptake activity for LN3.

In another more preferred embodiment of this disclosure, the saccharide importer as described herein comprises, consists, or consists essentially of the polypeptide sequence with SEQ ID NO:04 and has uptake activity for LN3; SEQ ID NO:04 is a mutant polypeptide of the saccharide importer with SEQ ID NO:02 wherein the SEQ ID NO:04 has a deletion of 39 amino acid residues N-terminally from the first TM domain of SEQ ID NO:02 and an insertion of 19 amino acid residues N-terminally from the first TM domain of SEQ ID NO:02. The first TM domain of SEQ ID NO:02 starts at Ser at position 40. Both polypeptides with SEQ ID NOs: 02 and 04 have uptake activity for LN3. In an even more preferred embodiment, the saccharide importer as described herein is the polypeptide with SEQ ID NO:04 and has uptake activity for LN3.

In another more preferred embodiment of this disclosure, the saccharide importer has uptake activity for LN3 and comprises a polypeptide sequence with any one of SEQ ID NO: 16, comprising a mutation of one or more amino acid residue(s) N-terminally from the first transmembrane (TM) domain of any one of SEQ ID NO:09. SEQ ID NO:16 is a mutant polypeptide of the saccharide importer with SEQ ID NO:09 wherein the SEQ ID NO: 16 has a deletion of 23 amino acid residues N-terminally from the first TM domain of SEQ ID NO:09 and an insertion of 20 amino acid residues N-terminally from the first TM domain of SEQ ID NO:09. The first TM domain of SEQ ID NO:09 starts at Val at position 24. In an even more preferred embodiment, the saccharide importer as described herein is the polypeptide with SEQ ID NO:16 and has uptake activity for LN3.

A polypeptide sequence that has a deletion, an insertion and/or a mutation of one or more amino acid residue(s) compared to a reference polypeptide sequence can be obtained by expression of a mutant form of the polynucleotide sequence encoding the reference polypeptide sequence. The mutant form of a polynucleotide sequence may contain a deletion of one or more nucleotides, an insertion of one or more nucleotides, a frameshift of the coding sequence of one or more nucleotides, and/or one or more single nucleotide polymorphisms (SNPs) compared to the native polynucleotide sequence. A mutant form of a polynucleotide sequence can be obtained by techniques well-known to a person skilled in the art, such as but not limited to PCR cloning; TA cloning; ligation independent cloning (LIC); seamless ligation cloning extract (SLICE); mating-assisted genetically integrated cloning (MAGIC); sequence and ligation independent cloning (SLIC); In-Fusion; Gibson assembly; polymerase incomplete primer extension (PIPE); circular polymerase extension cloning (CPEC); site-specific mutation; CrispR; riboswitch; recombineering; ssDNA mutagenesis; transposon mutagenesis.

In another and/or additional preferred embodiment of this disclosure, the saccharide importer as described herein originates from the major facilitator superfamily (MFS) of transporters and comprises a polypeptide sequence comprising an IPR001927 domain as defined by InterPro 90.0 as released on 4 Aug. 2022, and comprises a polypeptide sequence comprising 12 transmembrane (TM) domains with a conserved domain [AGSV][HNQ][ACDEGNQSTV]XX[FWY]XXXXX (no L) as represented by SEQ ID NO:17, wherein X can be any amino acid residue, present in the first TM domain, wherein the second amino acid residue of SEQ ID NO: 17 is aligned to Lys18 of the polypeptide with SEQ ID NO:22.

In a more preferred embodiment, the saccharide importer as described herein originates from the major facilitator superfamily (MFS) of transporters and comprises a polypeptide sequence comprising an IPR001927 domain as defined by InterPro 90.0 as released on 4 Aug. 2022, and comprises a polypeptide sequence comprising 12 transmembrane (TM) domains with a conserved domain [AGS]Q[ACGNQSTV]XX[FWY] as represented by SEQ ID NO: 18, wherein X can be any amino acid residue, present in the first TM domain, wherein the second amino acid residue of SEQ ID NO: 18 is aligned to Lys18 of the polypeptide with SEQ ID NO: 22.

In another and/or additional preferred embodiment of this disclosure, the saccharide importer as described herein further comprises uptake activity for one or more other saccharide(s) different from LN3, wherein the one or more other saccharide(s) different from LN3 are chosen from the list comprising monosaccharide, disaccharide, oligosaccharide and polysaccharide.

In another and/or additional preferred embodiment of this disclosure, the saccharide importer as described has uptake activity for LN3 but not for LNT (lacto-N-tetraose, Galβ1-3GlcNAcβ1-3Galβ1-4Glc).

In another and/or additional preferred embodiment of this disclosure, the saccharide importer as described has uptake activity for LN3 but not for LNnT (lacto-N-neotetraose, Galβ1-4GlcNAcβ1-3Galβ1-4Glc). In a more preferred embodiment of this disclosure, the saccharide importer as described has uptake activity for LN3 but not for LNT and not for LNnT.

In the scope of this disclosure, the cell is genetically engineered to express, preferably to overexpress, a polynucleotide sequence that encodes a saccharide importer as described herein.

In a preferred embodiment of the cell and/or method, the cell is modified with one or more expression modules. The expression modules are also known as transcriptional units and comprise polynucleotides for expression of recombinant genes including coding gene sequences and appropriate transcriptional and/or translational control signals that are operably linked to the coding genes. The control signals comprise promoter sequences, untranslated regions, ribosome binding sites, terminator sequences. The expression modules can contain elements for expression of one single recombinant gene but can also contain elements for expression of more recombinant genes or can be organized in an operon structure for integrated expression of two or more recombinant genes. The polynucleotides may be produced by recombinant DNA technology using techniques well-known in the art. Methods which are well known to those skilled in the art to construct expression modules include, for example, in vitro recombinant DNA techniques, synthetic techniques, and in vivo genetic recombination. See, for example, the techniques described in Sambrook et al. (2001) Molecular Cloning: a laboratory manual, 3rd Edition, Cold Spring Harbor Laboratory Press, CSH, New York or to Current Protocols in Molecular Biology, John Wiley and Sons, N.Y. (1989 and yearly updates).

The expression of each of the expression modules can be constitutive or is created by a natural or chemical inducer. As used herein, constitutive expression should be understood as expression of a gene that is transcribed continuously in an organism. Expression that is created by a natural inducer should be understood as a facultative or regulatory expression of a gene that is only expressed upon a certain natural condition of the host (e.g., organism being in labor, or during lactation), as a response to an environmental change (e.g., including but not limited to hormone, heat, cold, pH shifts, light, oxidative or osmotic stress/signaling), or dependent on the position of the developmental stage or the cell cycle of the host cell including but not limited to apoptosis and autophagy. Expression that is created by a chemical inducer should be understood as a facultative or regulatory expression of a gene that is only expressed upon sensing of external chemicals (e.g., IPTG, arabinose, lactose, allo-lactose, rhamnose or fucose) via an inducible promoter or via a genetic circuit that either induces or represses the transcription or translation of the polynucleotide to a polypeptide.

The expression modules can be integrated in the genome of the cell or can be presented to the cell on a vector. The vector can be present in the form of a plasmid, cosmid, phage, liposome, or virus, which is to be stably transformed/transfected into the genetically engineered cell. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses, and vectors derived from combinations thereof, such as those derived from plasmid and bacteriophage genetic elements, such as cosmids and phagemids. These vectors may contain selection markers such as but not limited to antibiotic markers, auxotrophic markers, toxin-antitoxin markers, RNA sense/antisense markers. The expression system constructs may contain control regions that regulate as well as engender expression. Generally, any system or vector suitable to maintain, propagate or express polynucleotides and/or to express a polypeptide in a host may be used for expression in this regard. The appropriate DNA sequence may be inserted into the expression system by any of a variety of well-known and routine techniques, such as, for example, those set forth in Sambrook et al., see above. For recombinant production, cells can be genetically engineered to incorporate expression systems or portions thereof or polynucleotides of the disclosure. Introduction of a polynucleotide into the cell can be effected by methods described in many standard laboratory manuals, such as Davis et al., Basic Methods in Molecular Biology, (1986), and Sambrook et al., 1989, supra.

As used herein an expression module comprises polynucleotides for expression of at least one recombinant gene. The recombinant gene is involved in the expression of a polypeptide acting in the synthesis of one or more bioproduct(s) of this disclosure; or the recombinant gene is linked to other pathways in the cell that are not involved in the synthesis of the one or more bioproduct(s). The recombinant genes encode endogenous proteins with a modified expression or activity, preferably the endogenous proteins are overexpressed; or the recombinant genes encode heterologous proteins that are heterogeneously introduced and expressed in the modified cell, preferably overexpressed. The endogenous proteins can have a modified expression in the cell which also expresses a heterologous protein.

In another and/or additional preferred embodiment of the cell and/or method of the disclosure, the expression of each of the expression modules present in the genetically engineered cell is constitutive or tuneable as described herein.

In another and/or additional preferred embodiment of the cell and/or method, the polynucleotide sequence encoding the saccharide importer is operably linked to control sequences recognized by the cell. In another and/or additional preferred embodiment of the cell and/or method, the polynucleotide sequence encoding the saccharide importer is foreign to the cell. In a further preferred embodiment, the polynucleotide sequence is integrated in the genome of the cell and/or is presented to the cell on a vector.

In another and/or additional preferred embodiment of the cell and/or method of this disclosure, the cell is capable to produce, preferably produces, the one or more bioproduct(s) from one or more precursor(s) as described herein. In a more preferred embodiment, the cell is capable to produce, preferably produces, at least one of the one or more precursor(s) that is/are used for the production of the one or more bioproduct(s). In an even more preferred embodiment, the cell is capable to produce, preferably produces, all of the one or more precursor(s).

In a further preferred embodiment, the cell is genetically engineered for the production of at least one of the one or more precursor(s) that is/are used for the production of the one or more bioproduct(s). In a further more preferred embodiment, the cell is genetically engineered for the production of all of the one or more precursor(s).

In another and/or additional further preferred embodiment, at least one of the one or more precursor(s) that is/are produced by the cell of this disclosure and that is/are used by the cell for the production of the one or more bioproduct(s) as described herein is internalized in the cell via a saccharide importer as described herein.

In another and/or additional preferred embodiment of the cell and/or method of this disclosure, the cell is further genetically engineered for the production of one or more bioproduct(s) as described herein.

In another and/or additional preferred embodiment of the cell and/or method of this disclosure, the cell comprises one or more pathway(s) for monosaccharide synthesis. The pathways for monosaccharide synthesis comprise enzymes like e.g., carboxylases, decarboxylases, isomerases, epimerases, reductases, enolases, phosphorylases, carboxykinases, kinases, phosphatases, aldolases, hydrolases, dehydrogenases, enzymes involved in the synthesis of one or more nucleoside triphosphate(s) like UTP, GTP, ATP and CTP, enzymes involved in the synthesis of any one or more nucleoside mono- or diphosphates like e.g., UMP and UDP, respectively, and enzymes involved in the synthesis of phosphoenolpyruvate (PEP). More preferably, the cell is genetically engineered for production of one or more monosaccharides.

In another and/or additional preferred embodiment of the cell and/or method of this disclosure, the cell comprises one or more pathway(s) for phosphorylated monosaccharide synthesis. The pathways for phosphorylated monosaccharide synthesis comprise enzymes involved in the synthesis of one or more monosaccharide(s), one or more nucleoside mono-, di- and/or triphosphate(s) and enzymes involved in the synthesis of phosphoenolpyruvate (PEP) like e.g., but not limited to PEP synthase, carboxylases, decarboxylases, isomerases, epimerases, reductases, enolases, phosphorylases, carboxykinases, kinases, phosphatases, aldolases, hydrolases and dehydrogenases. More preferably, the cell is genetically engineered for production of one or more phosphorylated monosaccharides.

In another and/or additional preferred embodiment of the cell and/or method of this disclosure, the cell is capable to produce, preferably produces, one or more nucleotide-activated sugars preferably chosen from the list comprising UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), GDP-fucose, (GDP-Fuc), UDP-glucuronate, UDP-galacturonate, UDP-2-acetamido-2,6-dideoxy--L-arabino-4-hexulose, UDP-2-acetamido-2,6-dideoxy--L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid (CMP-Neu5Ac), CMP-Neu4Ac, CMP-Neu5Ac9N₃, CMP-Neu4,5Ac₂, CMP-Neu5,7Ac₂, CMP-Neu5,9Ac₂, CMP-Neu5,7 (8,9)Ac₂, CMP-N-glycolylneuraminic acid (CMP-Neu5Gc), GDP-rhamnose and UDP-xylose. Preferably, the cell comprises one or more pathways for the synthesis of one or more of the nucleotide-activated sugars. The pathways for nucleotide-activated sugar synthesis comprise enzymes like e.g., PEP synthase, carboxylases, decarboxylases, isomerases, epimerases, reductases, enolases, phosphorylases, carboxykinases, kinases, phosphatases, aldolases, hydrolases, dehydrogenases, mannose-6-phosphate isomerase, phosphomannomutase, mannose-1-phosphate guanylyltransferase, GDP-mannose 4,6-dehydratase, GDP-L-fucose synthase, L-fucokinase/GDP-fucose pyrophosphorylase, L-glutamine-D-fructose-6-phosphate aminotransferase, glucosamine-6-phosphate deaminase, phosphoglucosamine mutase, N-acetylglucosamine-6-phosphate deacetylase, N-acylglucosamine 2-epimerase, UDP-N-acetylglucosamine 2-epimerase, N-acetylglucosamine-6P 2-epimerase, glucosamine 6-phosphate N-acetyltransferase, N-acetylglucosamine-6-phosphate phosphatase, N-acetylmannosamine-6-phosphate 2-epimerase, N-acetylmannosamine-6-phosphate phosphatase, N-acetylmannosamine kinase, phosphoacetylglucosamine mutase, N-acetylglucosamine-1-phosphate uridylyltransferase, glucosamine-1-phosphate acetyltransferase, sialic acid synthase, N-acetylneuraminate lyase, N-acylneuraminate-9-phosphate synthase, N-acylneuraminate-9-phosphatase, CMP-sialic acid synthase, galactose-1-epimerase, galactokinase, glucokinase, galactose-1-phosphate uridylyltransferase, UDP-glucose 4-epimerase, glucose-1-phosphate uridylyltransferase and/or phosphoglucomutase. In a more preferred embodiment of the cell and/or method of this disclosure, the cell uses at least one of the produced nucleotide-activated sugar(s) for the production of one or more bioproduct(s) of this disclosure. In an even more preferred embodiment of the cell and/or method of this disclosure, the cell is genetically engineered for production of one or more of the nucleotide-activated sugar(s).

The cell used herein is optionally genetically engineered to express the de novo synthesis of UDP-GlcNAc. UDP-GlcNAc can be provided by an enzyme expressed in the cell or by the metabolism of the cell. Such cell producing an UDP-GlcNAc can express enzymes converting, e.g., GlcNAc, which is to be added to the cell, to UDP-GlcNAc. These enzymes may be any one or more of the list comprising an N-acetyl-D-glucosamine kinase, an N-acetylglucosamine-6-phosphate deacetylase, a phosphoglucosamine mutase, and an N-acetylglucosamine-1-phosphate uridylyltransferase/glucosamine-1-phosphate acetyltransferase from several species including Homo sapiens, Escherichia coli. Preferably, the cell is modified to produce UDP-GlcNAc. More preferably, the cell is modified for enhanced UDP-GlcNAc production. The modification can be any one or more chosen from the group comprising knock-out of an N-acetylglucosamine-6-phosphate deacetylase, over-expression of an L-glutamine-D-fructose-6-phosphate aminotransferase, over-expression of a phosphoglucosamine mutase, and over-expression of an N-acetylglucosamine-1-phosphate uridylyltransferase/glucosamine-1-phosphate acetyltransferase.

Additionally, or alternatively, the cell used herein is optionally genetically engineered to express the de novo synthesis of CMP-Neu5Ac. CMP-Neu5Ac can be provided by an enzyme expressed in the cell or by the metabolism of the cell. Such cell producing CMP-Neu5Ac can express an enzyme converting, e.g., sialic acid to CMP-Neu5Ac. This enzyme may be a CMP-sialic acid synthetase, like the N-acylneuraminate cytidylyltransferase from several species including Homo sapiens, Neisseria meningitidis, and Pasteurella multocida. Preferably, the cell is modified to produce CMP-Neu5Ac. More preferably, the cell is modified for enhanced CMP-Neu5Ac production. The modification can be any one or more chosen from the group comprising knock-out of an N-acetylglucosamine-6-phosphate deacetylase, knock-out of a glucosamine-6-phosphate deaminase, over-expression of a CMP-sialic acid synthetase, and over-expression of an N-acetyl-D-glucosamine-2-epimerase encoding gene.

Additionally, or alternatively, the cell used herein is optionally genetically engineered to express the de novo synthesis of GDP-fucose. GDP-fucose can be provided by an enzyme expressed in the cell or by the metabolism of the cell. Such cell producing GDP-fucose can express an enzyme converting, e.g., fucose, which is to be added to the cell, to GDP-fucose. This enzyme may be, e.g., a bifunctional fucose kinase/fucose-1-phosphate guanylyltransferase, like Fkp from Bacteroides fragilis, or the combination of one separate fucose kinase together with one separate fucose-1-phosphate guanylyltransferase like they are known from several species including Homo sapiens, Sus scrofa and Rattus norvegicus. Preferably, the cell is modified to produce GDP-fucose. More preferably, the cell is modified for enhanced GDP-fucose production. The modification can be any one or more chosen from the group comprising knock-out of an UDP-glucose: undecaprenyl-phosphate glucose-1-phosphate transferase encoding gene, over-expression of a GDP-L-fucose synthase encoding gene, over-expression of a GDP-mannose 4,6-dehydratase encoding gene, over-expression of a mannose-1-phosphate guanylyltransferase encoding gene, over-expression of a phosphomannomutase encoding gene and over-expression of a mannose-6-phosphate isomerase encoding gene.

Additionally, or alternatively, the cell used herein is optionally genetically engineered to express the de novo synthesis of UDP-Gal. UDP-Gal can be provided by an enzyme expressed in the cell or by the metabolism of the cell. Such cell producing UDP-Gal can express an enzyme converting, e.g., UDP-glucose, to UDP-Gal. This enzyme may be, e.g., the UDP-glucose-4-epimerase GalE like as known from several species including Homo sapiens, Escherichia coli, and Rattus norvegicus. Preferably, the cell is modified to produce UDP-Gal. More preferably, the cell is modified for enhanced UDP-Gal production. The modification can be any one or more chosen from the group comprising knock-out of a bifunctional 5′-nucleotidase/UDP-sugar hydrolase encoding gene, knock-out of a galactose-1-phosphate uridylyltransferase encoding gene and over-expression of a UDP-glucose-4-epimerase encoding gene.

Additionally, or alternatively, the cell used herein is optionally genetically engineered to express the de novo synthesis of UDP-GalNAc. UDP-GalNAc can be synthesized from UDP-GlcNAc by the action of a single-step reaction using a UDP-N-acetylglucosamine 4-epimerase like e.g., wbgU from Plesiomonas shigelloides, gne from Yersinia enterocolitica or wbpP from Pseudomonas aeruginosa serotype O6. Preferably, the cell is modified to produce UDP-GalNAc. More preferably, the cell is modified for enhanced UDP-GalNAc production.

Additionally, or alternatively, the cell used herein is optionally genetically engineered to express the de novo synthesis of UDP-ManNAc. UDP-ManNAc can be synthesized directly from UDP-GlcNAc via an epimerization reaction performed by a UDP-GlcNAc 2-epimerase (like e.g., cap5P from Staphylococcus aureus, RffE from E. coli, Cps19fK from S. pneumoniae, and RfbC from S. enterica). Preferably, the cell is modified to produce UDP-ManNAc. More preferably, the cell is modified for enhanced UDP-ManNAc production.

According to another preferred embodiment of the cell and/or method of the disclosure, the cell possesses, preferably expresses, more preferably overexpresses, one or more glycosyltransferase(s) chosen from the list comprising fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases and fucosaminyltransferases.

In a preferred embodiment of the cell and/or method of the disclosure, the fucosyltransferase is chosen from the list comprising alpha-1,2-fucosyltransferase, alpha-1,3-fucosyltransferase, alpha-1,3/4-fucosyltransferase, alpha-1,4-fucosyltransferase and alpha-1,6-fucosyltransferase.

In an alternative and/or additional embodiment of the method and/or cell of the disclosure, the sialyltransferase is chosen from the list comprising alpha-2,3-sialyltransferase, alpha-2,6-sialyltransferase, and alpha-2,8-sialyltransferase.

In an alternative and/or additional embodiment of the cell and/or method of the disclosure, the galactosyltransferase is chosen from the list comprising beta-1,3-galactosyltransferase, N-acetylglucosamine beta-1,3-galactosyltransferase, beta-1,4-galactosyltransferase, N-acetylglucosamine beta-1,4-galactosyltransferase, alpha-1,3-galactosyltransferase and alpha-1,4-galactosyltransferase.

In an alternative and/or additional embodiment of the cell and/or method of the disclosure, the glucosyltransferase is chosen from the list comprising alpha-glucosyltransferase, beta-1,2-glucosyltransferase, beta-1,3-glucosyltransferase and beta-1,4-glucosyltransferase.

In an alternative and/or additional embodiment of the cell and/or method of the disclosure, the mannosyltransferase is chosen from the list comprising alpha-1,2-mannosyltransferase, alpha-1,3-mannosyltransferase and alpha-1,6-mannosyltransferase.

In an alternative and/or additional embodiment of the cell and/or method of the disclosure, the N-acetylglucosaminyltransferase is chosen from the list comprising galactoside beta-1,3-N-acetylglucosaminyltransferase and beta-1,6-N-acetylglucosaminyltransferase.

In an alternative and/or additional embodiment of the cell and/or method of the disclosure, the N-acetylgalactosaminyltransferase is chosen from the list comprising alpha-1,3-N-acetylgalactosaminyltransferase.

In a further embodiment of the cell and/or method of the disclosure, the cell is modified in the expression or activity of at least one of the glycosyltransferases. In a preferred embodiment, the glycosyltransferase is an endogenous protein of the cell with a modified expression or activity, preferably the endogenous glycosyltransferase is overexpressed; alternatively the glycosyltransferase is a heterologous protein that is heterogeneously introduced and expressed in the cell, preferably overexpressed. The endogenous glycosyltransferase can have a modified expression in the cell which also expresses a heterologous glycosyltransferase.

In another and/or additional preferred embodiment of the cell and/or method of this disclosure, the cell comprises at least one pathway chosen from the list comprising fucosylation pathway, sialylation pathway, galactosylation pathway, N-acetylglucosaminylation pathway, N-acetylgalactosaminylation pathway, mannosylation pathway and N-acetylmannosaminylation pathway as described herein. In a more preferred embodiment, the cell is genetically engineered to comprise at least one of the pathway(s). In an even more preferred embodiment, the cell comprises at least one of the pathway(s) wherein at least one of the pathway(s) has/have been genetically engineered.

In a more preferred embodiment, the cell comprises a sialyation pathway. A sialylation pathway is a biochemical pathway consisting of at least one of the enzymes and their respective genes chosen from the list comprising an L-glutamine-D-fructose-6-phosphate aminotransferase, a phosphoglucosamine mutase, an N-acetylglucosamine-6-P deacetylase, an N-acylglucosamine 2-epimerase, a UDP-N-acetylglucosamine 2-epimerase, an N-acetylmannosamine-6-phosphate 2-epimerase, a UDP-GlcNAc 2-epimerase/kinase, a glucosamine 6-phosphate N-acetyltransferase, an N-acetylglucosamine-6-phosphate phosphatase, a phosphoacetylglucosamine mutase, an N-acetylglucosamine 1-phosphate uridylyltransferase, a glucosamine-1-phosphate acetyltransferase, an Neu5Ac synthase, an N-acetylneuraminate lyase, an N-acylneuraminate-9-phosphate synthase, an N-acylneuraminate-9-phosphatase, a sialic acid transporter, a cytidine monophosphate (CMP) kinase and a CMP-sialic acid synthase, combined with a sialyltransferase leading to any one or more of a 2,3; a 2,6 and/or a 2,8 sialylated oligosaccharides.

In an even more preferred embodiment, the cell is genetically engineered to comprise a sialylation pathway. In another even more preferred embodiment, the cell has been genetically engineered to comprise a sialylation pathway wherein any one or more of the genes chosen from the list comprising L-glutamine-D-fructose-6-phosphate aminotransferase, phosphoglucosamine mutase, N-acetylglucosamine-6-P deacetylase, N-acylglucosamine 2-epimerase, UDP-N-acetylglucosamine 2-epimerase, N-acetylmannosamine-6-phosphate 2-epimerase, UDP-GlcNAc 2-epimerase/kinase, glucosamine 6-phosphate N-acetyltransferase, N-acetylglucosamine-6-phosphate phosphatase, phosphoacetylglucosamine mutase, N-acetylglucosamine 1-phosphate uridylyltransferase, glucosamine-1-phosphate acetyltransferase, Neu5Ac synthase, N-acetylneuraminate lyase, N-acylneuraminate-9-phosphate synthase, N-acylneuraminate-9-phosphatase, sialic acid transporter, CMP kinase, CMP-sialic acid synthase and sialyltransferase has/have a modified and/or enhanced expression.

In another and/or additional preferred embodiment, the cell comprises a fucosylation pathway. A fucosylation pathway is a biochemical pathway consisting of at least one of the enzymes and their respective genes chosen from the list comprising mannose-6-phosphate isomerase, phosphomannomutase, mannose-1-phosphate guanylyltransferase, GDP-mannose 4,6-dehydratase, GDP-L-fucose synthase, fucose permease, fucose kinase, fucose-1-phosphate guanylyltransferase combined with a fucosyltransferase leading to a 1,2; a 1,3; a 1,4 and/or a 1,6 fucosylated oligosaccharides.

In a more preferred additional and/or alternative embodiment, the cell is genetically engineered to comprise a fucosylation pathway. In another even more preferred additional and/or alternative embodiment, the cell has been genetically engineered to comprise a fucosylation pathway wherein any one or more of the genes chosen from the list comprising mannose-6-phosphate isomerase, phosphomannomutase, mannose-1-phosphate guanylyltransferase, GDP-mannose 4,6-dehydratase, GDP-L-fucose synthase, fucose permease, fucose kinase, fucose-1-phosphate guanylyltransferase and fucosyltransferase has/have a modified and/or enhanced expression.

In another and/or additional preferred embodiment, the cell comprises a galactosylation pathway. A galactosylation pathway is a biochemical pathway consisting of at least one of the enzymes and their respective genes chosen from the list comprising galactose-1-epimerase, galactokinase, glucokinase, galactose-1-phosphate uridylyltransferase, UDP-glucose 4-epimerase, glucose-1-phosphate uridylyltransferase, phosphoglucomutase combined with a galactosyltransferase leading to a galactosylated compound comprising a mono-, di-, or oligosaccharide having an alpha or beta bound galactose on any one or more of the 2, 3, 4 and 6 hydroxyl group of the mono-, di-, or oligosaccharide.

In a more preferred additional and/or alternative embodiment, the cell is genetically engineered to comprise a galactosylation pathway. In another even more preferred additional and/or alternative embodiment, the cell has been genetically engineered to comprise a galactosylation pathway wherein any one or more of the genes chosen from the list comprising galactose-1-epimerase, galactokinase, glucokinase, galactose-1-phosphate uridylyltransferase, UDP-glucose 4-epimerase, glucose-1-phosphate uridylyltransferase, phosphoglucomutase and galactosyltransferase has/have a modified and/or enhanced expression.

In another and/or additional preferred embodiment, the cell comprises an ‘N-acetylglucosaminylation’ pathway. An N-acetylglucosaminylation pathway is a biochemical pathway consisting of at least one of the enzymes and their respective genes chosen from the list comprising L-glutamine-D-fructose-6-phosphate aminotransferase, N-acetylglucosamine-6-phosphate deacetylase, phosphoglucosamine mutase, N-acetylglucosamine-1-phosphate uridylyltransferase, glucosamine-1-phosphate acetyltransferase combined a with glycosyltransferase leading to a GlcNAc-modified compound comprising a mono-, di-, or oligosaccharide having an alpha or beta bound N-acetylglucosamine (GlcNAc) on any one or more of the 3, 4 and 6 hydroxyl group of the mono-, di- or oligosaccharide.

In a more preferred additional and/or alternative embodiment, the cell is genetically engineered to comprise an N-acetylglucosaminylation pathway. In another even more preferred additional and/or alternative embodiment, the cell has been genetically engineered to comprise an N-acetylglucosaminylation pathway wherein any one or more of the genes chosen from the list comprising L-glutamine-D-fructose-6-phosphate aminotransferase, N-acetylglucosamine-6-phosphate deacetylase, phosphoglucosamine mutase, N-acetylglucosamine-1-phosphate uridylyltransferase, glucosamine-1-phosphate acetyltransferase and a glycosyltransferase transferring GlcNAc has/have a modified and/or enhanced expression.

In another and/or additional preferred embodiment, the cell comprises an ‘N-acetylgalactosaminylation’ pathway. An N-acetylgalactosaminylation pathway is a biochemical pathway consisting of at least one of the enzymes and their respective genes chosen from the list comprising L-glutamine-D-fructose-6-phosphate aminotransferase, phosphoglucosamine mutase, N-acetylglucosamine 1-phosphate uridylyltransferase, glucosamine-1-phosphate acetyltransferase, UD) P-N-acetylglucosamine 4-epimerase, UDP-glucose 4-epimerase, N-acetylgalactosamine kinase and or UDP-N-acetylgalactosamine pyrophosphorylase combined with a glycosyltransferase leading to a GalNAc-modified compound comprising a mono-, di- or oligosaccharide having an alpha or beta bound N-acetylgalactosamine on the mono-, di- or oligosaccharide.

In a more preferred additional and/or alternative embodiment, the cell is genetically engineered to comprise an N-acetylgalactosaminylation pathway. In another even more preferred additional and/or alternative embodiment, the cell has been genetically engineered to comprise an N-acetylgalactosaminylation pathway wherein any one or more of the genes chosen from the list comprising L-glutamine-D-fructose-6-phosphate aminotransferase, phosphoglucosamine mutase, N-acetylglucosamine 1-phosphate uridylyltransferase, glucosamine-1-phosphate acetyltransferase, (ID) P-N-acetylglucosamine 4-epimerase, UDP-glucose 4-epimerase, N-acetylgalactosamine kinase and or UDP-N-acetylgalactosamine pyrophosphorylase and a glycosyltransferase transferring GalNAc has/have a modified and/or enhanced expression.

In another and/or additional preferred embodiment, the cell comprises a ‘mannosylation’ pathway. A mannosylation pathway is a biochemical pathway consisting of at least one of the enzymes and their respective genes chosen from the list comprising mannose-6-phosphate isomerase, phosphomannomutase and/or mannose-1-phosphate guanylyltransferase combined with a mannosyltransferase leading to a mannosylated compound comprising a mono-, di- or oligosaccharide having an alpha or beta bound mannose on the mono-, di- or oligosaccharide.

In a more preferred additional and/or alternative embodiment, the cell is genetically engineered to comprise a mannosylation pathway. In another even more preferred additional and/or alternative embodiment, the cell has been genetically engineered to comprise a mannosylation pathway wherein any one or more of the genes chosen from the list comprising mannose-6-phosphate isomerase, phosphomannomutase and/or mannose-1-phosphate guanylyltransferase and mannosyltransferase has/have a modified and/or enhanced expression.

In another and/or additional preferred embodiment, the cell comprises an ‘N-acetylmannosaminylation’ pathway. An N-acetylmannosaminylation pathway is a biochemical pathway consisting of at least one of the enzymes and their respective genes chosen from the list comprising L-glutamine-D-fructose-6-phosphate aminotransferase, glucosamine-6-phosphate deaminase, phosphoglucosamine mutase, N-acetylglucosamine-6-phosphate deacetylase, glucosamine 6-phosphate N-acetyltransferase, N-acetylglucosamine-1-phosphate uridyltransferase, glucosamine-1-phosphate acetyltransferase, glucosamine-1-phosphate acetyltransferase, UDP-GlcNAc 2-epimerase and/or ManNAc kinase combined with a glycosyltransferase leading to a ManNAc-modified compound comprising a mono-, di- or oligosaccharide having an alpha or beta bound N-acetylmannosamine on the mono-, di- or oligosaccharide.

In a more preferred additional and/or alternative embodiment, the cell is genetically engineered to comprise an N-acetylmannosaminylation pathway. In another even more preferred additional and/or alternative embodiment, the cell has been genetically engineered to comprise an N-acetylmannosaminylation pathway wherein any one or more of the genes chosen from the list comprising L-glutamine-D-fructose-6-phosphate aminotransferase, glucosamine-6-phosphate deaminase, phosphoglucosamine mutase, N-acetylglucosamine-6-phosphate deacetylase, glucosamine 6-phosphate N-acetyltransferase, N-acetylglucosamine-1-phosphate uridyltransferase, glucosamine-1-phosphate acetyltransferase, glucosamine-1-phosphate acetyltransferase, UDP-GlcNAc 2-epimerase and/or ManNAc kinase and a glycosyltransferase transferring ManNAc has/have a modified and/or enhanced expression.

In another and/or additional preferred embodiment, the cell is genetically engineered for an enhanced production of one or more bioproduct(s), an enhanced uptake of one or more precursor(s) and/or acceptor(s) that is/are used in the synthesis of one or more bioproduct(s), a better efflux of one or more bioproduct(s), a decreased production of by-products like e.g., acids, an increased availability of co-factors like e.g., ATP, NADP, NADPH, and/or better metabolic flux through any one of the sialylation, fucosylation, galactosylation, N-N-acetylgalactosaminylation, mannosylation, and/or N-acetylglucosaminylation, acetylmannosaminylation pathway present in the cell.

According to another preferred embodiment of the cell and/or method of the disclosure, the cell comprises a catabolic pathway for selected mono-, di- or oligosaccharides which is at least partially inactivated, the mono-, di-, or oligosaccharides being involved in and/or required for the synthesis of one or more bioproduct(s).

In another preferred embodiment of this disclosure, the cell is a bacterium, fungus, yeast, a plant cell, an animal cell, or a protozoan cell. The latter bacterium preferably belongs to the phylum of the Proteobacteria or the phylum of the Firmicutes or the phylum of the Cyanobacteria or the phylum Deinococcus-Thermus or the phylum of Actinobacteria. The latter bacterium belonging to the phylum Proteobacteria belongs preferably to the family Enterobacteriaceae, preferably to the species Escherichia coli. The latter bacterium preferably relates to any strain belonging to the species Escherichia coli such as but not limited to Escherichia coli B, Escherichia coli C, Escherichia coli W, Escherichia coli K12, Escherichia coli Nissle. More specifically, the latter term relates to cultivated Escherichia coli strains-designated as E. coli K12 strains-which are well-adapted to the laboratory environment, and, unlike wild type strains, have lost their ability to thrive in the intestine. Well-known examples of the E. coli K12 strains are K12 Wild type, W3110, MG1655, M182, MC1000, MC1060, MC1061, MC4100, JM101, NZN111 and AA200. Hence, this disclosure specifically relates to a mutated and/or transformed Escherichia coli cell or strain as indicated above wherein the E. coli strain is a K12 strain. More preferably, the Escherichia coli K12 strain is E. coli MG1655. The latter bacterium belonging to the phylum Firmicutes belongs preferably to the Bacilli, preferably Lactobacilliales, with members such as Lactobacillus lactis, Leuconostoc mesenteroides, or Bacillales with members such as from the genus Bacillus, such as Bacillus subtilis or, B. amyloliquefaciens. The latter Bacterium belonging to the phylum Actinobacteria, preferably belonging to the family of the Corynebacteriaceae, with members Corynebacterium glutamicum or C. afermentans, or belonging to the family of the Streptomycetaceae with members Streptomyces griseus or S. fradiae. The latter bacterium belonging to the phylum Proteobacteria, preferably belonging to the family of the Vibrionaceae, with member Vibrio natriegens. The latter yeast preferably belongs to the phylum of the Ascomycota or the phylum of the Basidiomycota or the phylum of the Deuteromycota or the phylum of the Zygomycetes. The latter yeast belongs preferably to the genus Saccharomyces (with members like e.g., Saccharomyces cerevisiae, S. bayanus, S. boulardii), Zygosaccharomyces, Pichia (with members like e.g., Pichia pastoris, P. anomala, P. kluyveri), Komagataella, Hansenula, Kluyveromyces (with members like e.g., Kluyveromyces lactis, K. marxianus, K. thermotolerans), Debaromyces, Candida, Schizosaccharomyces, Schwanniomyces, Torulaspora, Yarrowia (like e.g., Yarrowia lipolytica) or Starmerella (like e.g., Starmerella bombicola). The latter yeast is preferably selected from Pichia pastoris, Yarrowia lipolitica, Saccharomyces cerevisiae, Kluyveromyces lactis, Hansemila polymorpha, Kluyveromyces marxianus, Pichia methanolica, Pichia stipites, Candida boidinii, Schizosaccharomyces pombe, Schwanniomyces occidentalis, Torulaspora delbrueckii, Zygosaccharomyces rouxii, and Zygosaccharomyces bailii. The latter fungus belongs preferably to the genus Rhizopus, Dictyostelium, Penicillium, Mucor or Aspergillus. Plant cells include cells of flowering and non-flowering plants, as well as algal cells, for example, Chlamydomonas, Chlorella, etc. Preferably, the plant is a tobacco, alfalfa, rice, tomato, cotton, rapeseed, soy, maize, or corn plant. The latter animal cell is preferably derived from non-human mammals (e.g., cattle, buffalo, pig, sheep, mouse, rat, primate (e.g., chimpanzee, orangutan, gorilla, monkey (e.g., Old World, New World), lemur), dog, cat, rabbit, horse, cow, goat, ox, deer, musk deer, bovid, whale, dolphin, hippopotamus, elephant, rhinoceros, giraffe, zebra, lion, cheetah, tiger, panda, red panda, otter), birds (e.g., chicken, duck, ostrich, turkey, pheasant), fish (e.g., swordfish, salmon, tuna, sea bass, trout, catfish), invertebrates (e.g., lobster, crab, shrimp, clams, oyster, mussel, sea urchin), reptiles (e.g., snake, alligator, turtle), amphibians (e.g., frogs) or insects (e.g., fly, nematode) or is a genetically modified cell line derived from human cells excluding embryonic stem cells. Both human and non-human mammalian cells are preferably chosen from the list comprising an epithelial cell like e.g., a mammary epithelial cell, an embryonic kidney cell (e.g., HEK293 or HEK 293T cell), a fibroblast cell, a COS cell, a Chinese hamster ovary (CHO) cell, a murine myeloma cell like e.g., an N20, SP2/0 or YB2/0 cell, an NIH-3T3 cell, a non-mammary adult stem cell or derivatives thereof such as described in WO21067641, preferably mesenchymal stem cell or derivates thereof as described in WO21067641, a lactocyte derived from mammalian induced pluripotent stem cells, preferably human induced pluripotent stem cells, a lactocyte as part of mammary-like gland organoids, a post-parturition mammary epithelium cell, a polarized mammary cell, preferably a polarized mammary cell selected from the group comprising live primary mammary epithelial cells, live mammary myoepithelial cells, live mammary progenitor cells, live immortalized mammary epithelial cells, live immortalized mammary myoepithelial cells, live immortalized mammary progenitor cells, a non-mammary adult stem cell or derivatives thereof as well-known to the person skilled in the art from e.g., WO2021/219634, WO 2022/054053, WO 2021/141762, WO 2021/142241, WO 2021/067641 and WO2021/242866. The latter insect cell is preferably derived from Spodoptera frugiperda like e.g., Sf9 or Sf21 cells, Bombyx mori, Mamestra brassicae, Trichoplusia ni like e.g., BTI-TN-5B1-4 cells or Drosophila melanogaster like e.g., Drosophila S2 cells. The latter protozoan cell preferably is a Leishmania tarentolae cell.

More preferably, the cell is selected from the group consisting of prokaryotic cells and eukaryotic cells, preferably from the group consisting of yeast cells, bacterial cells, archaebacterial cells, algae cells, and fungal cells as described herein.

In another and/or additional preferred embodiment, the cell is an E. coli or yeast with a lactose permease positive phenotype, preferably wherein the lactose permease is coded by the gene LacY or LAC12, respectively.

According to another aspect, this disclosure provides a method for the production of one or more bioproduct(s) as described herein, wherein the method comprises the steps of cultivating and/or incubating a cell, preferably a single cell, as described herein, under conditions permissive to express a saccharide importer as described herein and to produce one or more bioproduct(s), and preferably of separating the one or more bioproduct(s) from the cultivation or incubation. Preferably, the one or more bioproduct(s) is/are purified from the cultivation or incubation as described herein.

In the scope of this disclosure, permissive conditions are understood to be conditions relating to physical or chemical parameters including but not limited to temperature, pH, pressure, osmotic pressure and product/precursor/acceptor concentration.

In a particular embodiment, the permissive conditions may include a temperature-range of about 30+/−20 degrees centigrade, a pH-range of 2-10, preferably a pH range of 3-7.

In a preferred embodiment of the method and/or cell of this disclosure, the one or more bioproduct(s) as described herein is/are produced by a cell as described herein that is cultured in a cell cultivation or incubated in a cell incubation. Within the context of this disclosure, the cell cultivation and/or incubation comprises in vitro and/or ex vivo cultivation and/or incubation of cells.

In a preferred embodiment of the method of this disclosure, the cultivation medium contains at least one carbon source selected from the group consisting of glucose, fructose, sucrose, and glycerol.

In another preferred embodiment of the method of this disclosure, the cultivation or incubation medium contains at least one compound selected from the group consisting of lactose, galactose, glucose, UDP-GlcNAc, GlcNAc, UDP-Gal, UDP-Glc and LN3. In a preferred embodiment, the cultivation or incubation medium contains LN3. In a more preferred embodiment, the cultivation or incubation medium contains LN3 that is produced by a cell. In an even more preferred embodiment, the cultivation or incubation medium contains LN3 that is produced by a cell of this disclosure that is cultivated or incubated in the cultivation or incubation medium for the production of one or more bioproduct(s) as described herein.

Preferably, the one or more bioproduct(s) produced is/are recovered from the cultivation or incubation medium and/or the cell or separated from the cultivation or incubation as explained herein. More preferably, all bioproducts produced are recovered from the cultivation or incubation medium and/or the cell or separated from the cultivation or incubation as explained herein.

According to an embodiment of the method of the disclosure, the conditions permissive to produce the one or more bioproduct(s) comprise the use of a cultivation or incubation medium comprising one or more precursor(s) that is/are used for the production of one or more bioproduct(s) as described herein.

Preferably, the cultivation or incubation medium contains at least one precursor, wherein the precursor is selected from the group comprising a monosaccharide like e.g., galactose, glucose, fucose, sialic acid, GlcNAc, GalNAc; a nucleotide-activated sugar like e.g., CMP-sialic acid, UDP-Gal, UDP-GlcNAc, GDP-fucose; a disaccharide like e.g., lactose, melibiose, lacto-N-biose and N-acetyllactosamine; and an oligosaccharide like e.g., lacto-N-triose (LN3), lacto-N-tetraose (LNT) and LNnT. In a more preferred embodiment of the method of the disclosure, the precursor is chosen from the list comprising galactose, glucose, UDP-GlcNAc, GlcNAc, UDP-Gal, UDP-Glc and LN3. In an even more preferred embodiment of the method of the disclosure, the precursor is LN3.

According to another and/or additional preferred embodiment of the method of this disclosure, the one or more precursor(s) that is/are present in the cultivation or incubation medium is/are produced by a cell of this disclosure that is cultivated or incubated in the cultivation or incubation medium for the production of one or more bioproduct(s) as described herein. More preferably, all precursors that are present in the cultivation or incubation medium are produced by a cell of this disclosure that is cultivated or incubated in the cultivation or incubation medium.

According to another and/or additional preferred embodiment of the method of this disclosure, the one or more precursor(s) that is/are present in the cultivation or incubation medium is/are taken up by a cell of this disclosure, that is cultivated or incubated in the cultivation or incubation medium via a saccharide importer expressed in the cell as described herein. More preferably, all precursors that are present in the cultivation or incubation medium are taken up by a cell of this disclosure that is cultivated or incubated in the cultivation or incubation medium via a saccharide importer expressed in the cell as described herein.

According to a more preferred embodiment, the one or more precursor(s) that is/are present in the cultivation or incubation medium is/are produced and is/are taken up by a cell of this disclosure that is cultivated or incubated in the cultivation or incubation medium for the production of one or more bioproduct(s) wherein the one or more precursor(s) are taken up by the cell via a saccharide importer as described herein. In an even more preferred embodiment, all precursors that are present in the cultivation or incubation medium are produced and are taken up by a cell of this disclosure that is cultivated or incubated in the cultivation or incubation medium for the production of one or more bioproduct(s) wherein all the precursors are taken up by the cell via a saccharide importer as described herein.

In another more preferred embodiment, one of the one or more precursor(s) that is/are present in the cultivation or incubation medium is LN3, wherein the LN3 is produced by a cell of this disclosure that is cultivated or incubated in the cultivation or incubation medium. In another more preferred embodiment, one of the one or more precursor(s) that is/are present in the cultivation or incubation medium is LN3, wherein the LN3 is taken up by a cell of this disclosure that is cultivated or incubated in the cultivation or incubation medium via a saccharide importer as described herein. In another even more preferred embodiment, one of the one or more precursor(s) that is/are present in the cultivation or incubation medium is LN3, wherein the LN3 is produced by and taken up by a cell of this disclosure that is cultivated or incubated in the cultivation or incubation medium via a saccharide importer as described herein.

According to another and/or additional preferred embodiment of the method of this disclosure, the conditions permissive to produce the one or more bioproduct(s) comprise the use of a cultivation or incubation medium comprising one or more acceptor(s) as defined herein for the production of one or more bioproduct(s) as described herein.

According to an alternative and/or additional embodiment of the method of the invention, the conditions permissive to produce the one or more bioproduct(s) comprise adding to the cultivation or incubation medium at least one precursor and/or acceptor feed for the production of the one or more bioproduct(s).

According to an alternative embodiment of the method of the disclosure, the conditions permissive to produce the one or more bioproduct(s) comprise the use of a cultivation or incubation medium wherein the cultivation or incubation medium lacks any precursor and/or acceptor for the production of the one or more bioproduct(s) and is combined with a further addition to the cultivation or incubation medium of at least one precursor and/or acceptor feed for the production of the one or more bioproduct(s).

According to an embodiment of the method of the disclosure, the cultivation or incubation is contained in a reactor or incubator, as defined herein. The volume of the reactor or incubator ranges from microliter (μL) scale to 10,000 m³ (cubic meter). In a preferred embodiment, the volume of the reactor or incubator ranges from 250 mL (milliliter) to 10,000 m³.

According to another and/or additional preferred embodiment of the method of this disclosure, the cell produces one or more bioproduct(s) with a higher yield and/or higher purity compared to a cell with the same genetic make-up but lacking expression of a saccharide importer as described herein. In a more preferred embodiment, the one or more bioproduct(s) is/are one or more LN3-derived oligosaccharide(s) as described herein. According to a more preferred embodiment, the cell produces one or more LN3-derived oligosaccharide(s) with a higher yield and/or higher purity compared to a cell with the same genetic make-up but lacking expression of a saccharide importer as described herein.

In a preferred embodiment of this disclosure, the method results in a production of at least 50 g/L, preferably at least 75 g/L, more preferably at least 90 g/L of the one or more bioproduct(s) in the final volume of the cultivation or incubation.

In another preferred embodiment, the method for the production of one or more bioproduct(s) as described herein comprises at least one of the following steps:
i) Use of a cultivation or incubation medium comprising at least one precursor and/or acceptor;
ii) Adding to the cultivation or incubation medium in a reactor or incubator at least one precursor and/or acceptor feed wherein the total reactor or incubator volume ranges from 250 mL to 10,000 m³, preferably in a continuous manner, and preferably so that the final volume of the cultivation or incubation medium is not more than three-fold, preferably not more than two-fold, more preferably less than two-fold of the volume of the cultivation or incubation medium before the addition of the precursor and/or acceptor feed;
iii) Adding to the cultivation or incubation medium in a reactor or incubator at least one precursor and/or acceptor feed wherein the total reactor or incubator volume ranges from 250 mL to 10,000 m³, preferably in a continuous manner, and preferably so that the final volume of the cultivation or incubation medium is not more than three-fold, preferably not more than two-fold, more preferably less than two-fold of the volume of the cultivation or incubation medium before the addition of the precursor and/or acceptor feed and wherein preferably, the pH of the precursor and/or acceptor feed is set between 2.0 and 10.0 and wherein preferably, the temperature of the precursor and/or acceptor feed is kept between 20° C. and 80° C.;
iv) Adding at least one precursor and/or acceptor feed in a continuous manner to the cultivation or incubation medium over the course of 1 day, 2 days, 3 days, 4 days, 5 days by means of a precursor and/or acceptor feeding solution;
v) Adding at least one precursor and/or acceptor feed in a continuous manner to the cultivation or incubation medium over the course of 1 day, 2 days, 3 days, 4 days, 5 days by means of a precursor and/or acceptor feeding solution and wherein preferably, the pH of the precursor and/or acceptor feeding solution is set between 2.0 and 10.0 and wherein preferably, the temperature of the precursor and/or acceptor feeding solution is kept between 20° C. and 80° C.;
the method resulting in one or more bioproduct(s) with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the cultivation or incubation. In a more preferred embodiment of the method of the disclosure, the precursor is chosen from the list comprising galactose, glucose, UDP-GlcNAc, GlcNAc, UDP-Gal, UDP-Glc and LN3. In another more preferred embodiment of the method of the disclosure, the acceptor is lactose.

In another and/or additional preferred embodiment, the method for the production of one or more bioproduct(s) as described herein comprises at least one of the following steps:
i) Use of a cultivation or incubation medium comprising at least one precursor and/or acceptor;
ii) Adding to the cultivation or incubation medium in a reactor or incubator at least one precursor and/or acceptor in one pulse or in a discontinuous (pulsed) manner wherein the total reactor or incubator volume ranges from 250 mL to 10,000 m³, preferably so that the final volume of the cultivation or incubation medium is not more than three-fold, preferably not more than two-fold, more preferably less than two-fold of the volume of the cultivation or incubation medium before the addition of the precursor and/or acceptor feed pulse(s);
iii) Adding to the cultivation or incubation medium in a reactor or incubator at least one precursor and/or acceptor feed in one pulse or in a discontinuous (pulsed) manner wherein the total reactor or incubator volume ranges from 250 mL to 10,000 m³, preferably so that the final volume of the cultivation or incubation medium is not more than three-fold, preferably not more than two-fold, more preferably less than two-fold of the volume of the cultivation or incubation medium before the addition of the precursor and/or acceptor feed and wherein preferably, the pH of the precursor and/or acceptor feed pulse(s) is set between 2.0 and 10.0 and wherein preferably, the temperature of the precursor and/or acceptor feed pulse(s) is kept between 20° C. and 80° C.;
iv) Adding at least one precursor and/or acceptor feed in a discontinuous (pulsed) manner to the cultivation or incubation medium over the course of 5 minutes, 10 minutes, 30 minutes, 1 hour, 2 hours, 4 hours, 10 hours, 12 hours, 1 day, 2 days, 3 days, 4 days, 5 days by means of a precursor and/or acceptor feeding solution;
v) Adding at least one precursor and/or acceptor feed in a discontinuous (pulsed) manner to the cultivation or incubation medium over the course of 5 minutes, 10 minutes, 30 minutes, 1 hour, 2 hours, 4 hours, 10 hours, 12 hours, 1 day, 2 days, 3 days, 4 days, 5 days by means of a precursor and/or acceptor feeding solution and wherein preferably, the pH of the precursor and/or acceptor feeding solution is set between 2.0 and 10.0 and wherein preferably, the temperature of the precursor and/or acceptor feeding solution is kept between 20° C. and 80° C.;
the method resulting in one or more bioproduct(s) with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the cultivation or incubation. In a more preferred embodiment of the method of the disclosure, the precursor is chosen from the list comprising galactose, glucose, UDP-GlcNAc, GlcNAc, UDP-Gal, UDP-Glc and LN3. In another more preferred embodiment of the method of the disclosure, the acceptor is lactose.

In another and/or additional preferred embodiment, the method for the production of one or more bioproduct(s) as described herein comprises at least one of the following steps:
i) Use of a cultivation or incubation medium comprising at least 50, more preferably at least 75, more preferably at least 100, more preferably at least 120, more preferably at least 150 grams of precursor per liter of initial reactor or incubator volume wherein the reactor or incubator volume ranges from 250 mL to 10,000 m³;
ii) Adding to the cultivation or incubation medium in a reactor or incubator at least one precursor feed comprising at least 50, more preferably at least 75, more preferably at least 100, more preferably at least 120, more preferably at least 150 grams of precursor per liter of initial reactor or incubator volume wherein the total reactor or incubator volume ranges from 250 mL to 10,000 m³, preferably in a continuous manner, and preferably so that the final volume of the cultivation or incubation medium is not more than three-fold, preferably not more than two-fold, more preferably less than two-fold of the volume of the cultivation or incubation medium before the addition of the precursor feed;
iii) Adding to the cultivation or incubation medium in a reactor or incubator at least one precursor feed comprising at least 50, more preferably at least 75, more preferably at least 100, more preferably at least 120, more preferably at least 150 grams of precursor per liter of initial reactor or incubator volume wherein the total reactor or incubator volume ranges from 250 mL to 10,000 m³, preferably in a continuous manner, and preferably so that the final volume of the cultivation or incubation medium is not more than three-fold, preferably not more than two-fold, more preferably less than two-fold of the volume of the cultivation or incubation medium before the addition of the precursor feed and wherein preferably, the pH of the precursor feed is set between 2.0 and 10.0 and wherein preferably, the temperature of the precursor feed is kept between 20° C. and 80° C.;
iv) Adding at least one precursor feed in a continuous manner to the cultivation or incubation medium over the course of 1 day, 2 days, 3 days, 4 days, 5 days by means of a precursor feeding solution;
v) Adding at least one precursor feed in a continuous manner to the cultivation or incubation medium over the course of 1 day, 2 days, 3 days, 4 days, 5 days by means of a precursor feeding solution and wherein the concentration of the precursor feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably, the pH of the precursor feeding solution is set between 2.0 and 10.0 and wherein preferably, the temperature of the precursor feeding solution is kept between 20° C. and 80° C.;
the method resulting in one or more bioproduct(s) with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the cultivation or incubation. In a more preferred embodiment of the method of the disclosure, the precursor is chosen from the list comprising galactose, glucose, UDP-GlcNAc, GlcNAc, UDP-Gal, UDP-Glc and LN3.

In another and/or additional preferred embodiment, the method for the production of one or more bioproduct(s) as described herein comprises at least one of the following steps:
i) Use of a cultivation or incubation medium comprising at least 50, more preferably at least 75, more preferably at least 100, more preferably at least 120, more preferably at least 150 grams of acceptor per liter of initial reactor or incubator volume wherein the reactor or incubator volume ranges from 250 mL to 10,000 m³;
ii) Adding to the cultivation or incubation medium in a reactor or incubator at least one acceptor feed comprising at least 50, more preferably at least 75, more preferably at least 100, more preferably at least 120, more preferably at least 150 grams of acceptor per liter of initial reactor or incubator volume wherein the total reactor or incubator volume ranges from 250 mL to 10,000 m³, preferably in a continuous manner, and preferably so that the final volume of the cultivation or incubation medium is not more than three-fold, preferably not more than two-fold, more preferably less than two-fold of the volume of the cultivation or incubation medium before the addition of the acceptor feed;
iii) Adding to the cultivation or incubation medium in a reactor or incubator at least one acceptor feed comprising at least 50, more preferably at least 75, more preferably at least 100, more preferably at least 120, more preferably at least 150 grams of acceptor per liter of initial reactor or incubator volume wherein the total reactor or incubator volume ranges from 250 mL to 10,000 m³, preferably in a continuous manner, and preferably so that the final volume of the cultivation or incubation medium is not more than three-fold, preferably not more than two-fold, more preferably less than two-fold of the volume of the cultivation or incubation medium before the addition of the acceptor feed and wherein preferably, the pH of the acceptor feed is set between 2.0 and 10.0 and wherein preferably, the temperature of the acceptor feed is kept between 20° C. and 80° C.;
iv) Adding at least one acceptor feed in a continuous manner to the cultivation or incubation medium over the course of 1 day, 2 days, 3 days, 4 days, 5 days by means of an acceptor feeding solution;
v) Adding at least one acceptor feed in a continuous manner to the cultivation or incubation medium over the course of 1 day, 2 days, 3 days, 4 days, 5 days by means of an acceptor feeding solution and wherein the concentration of the acceptor feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably, the pH of the acceptor feeding solution is set between 2.0 and 10.0 and wherein preferably, the temperature of the acceptor feeding solution is kept between 20° C. and 80° C.;
the method resulting in one or more bioproduct(s) with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the cultivation or incubation. In a more preferred embodiment of the method of the disclosure, the acceptor is lactose.

In another and/or additional preferred embodiment, the method for the production of one or more bioproduct(s) as described herein comprises at least one of the following steps:
i) Use of a cultivation or incubation medium comprising at least 50, more preferably at least 75, more preferably at least 100, more preferably at least 120, more preferably at least 150 grams of precursor per liter of initial reactor or incubator volume wherein the reactor or incubator volume ranges from 250 mL to 10,000 m³;
ii) Adding to the cultivation or incubation medium in a reactor or incubator at least one precursor feed comprising at least 50, more preferably at least 75, more preferably at least 100, more preferably at least 120, more preferably at least 150 grams of precursor per liter of initial reactor or incubator volume wherein the total reactor or incubator volume ranges from 250 mL to 10,000 m³ in one pulse or in a discontinuous (pulsed), preferably so that the final volume of the cultivation or incubation medium is not more than three-fold, preferably not more than two-fold, more preferably less than two-fold of the volume of the cultivation or incubation medium before the addition of the precursor feed pulse(s);
iii) Adding to the cultivation or incubation medium in a reactor or incubator at least one precursor feed comprising at least 50, more preferably at least 75, more preferably at least 100, more preferably at least 120, more preferably at least 150 grams of precursor per liter of initial reactor or incubator volume wherein the total reactor or incubator volume ranges from 250 mL to 10,000 m³ in one pulse or in a discontinuous (pulsed) manner, preferably so that the final volume of the cultivation or incubation medium is not more than three-fold, preferably not more than two-fold, more preferably less than two-fold of the volume of the cultivation or incubation medium before the addition of the precursor feed and wherein preferably, the pH of the precursor feed pulse(s) is set between 2.0 and 10.0 and wherein preferably, the temperature of the precursor feed pulse(s) is kept between 20° C. and 80° C.;
iv) Adding at least one precursor feed in a discontinuous (pulsed) manner to the cultivation or incubation medium over the course of 5 minutes, 10 minutes, 30 minutes, 1 hour, 2 hours, 4 hours, 10 hours, 12 hours, 1 day, 2 days, 3 days, 4 days, 5 days by means of a precursor feeding solution;
v) Adding at least one precursor feed in a discontinuous (pulsed) manner to the cultivation or incubation medium over the course of 5 minutes, 10 minutes, 30 minutes, 1 hour, 2 hours, 4 hours, 10 hours, 12 hours, 1 day, 2 days, 3 days, 4 days, 5 days by means of a precursor feeding solution and wherein the concentration of the precursor feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably, the pH of the precursor feeding solution is set between 2.0 and 10.0 and wherein preferably, the temperature of the precursor feeding solution is kept between 20° C. and 80° C.;
the method resulting in one or more bioproduct(s) with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the cultivation or incubation. In a more preferred embodiment of the method of the disclosure, the precursor is chosen from the list comprising galactose, glucose, UDP-GlcNAc, GlcNAc, UDP-Gal, UDP-Glc and LN3.

In another and/or additional preferred embodiment, the method for the production of one or more bioproduct(s) as described herein comprises at least one of the following steps:
i) Use of a cultivation or incubation medium comprising at least 50, more preferably at least 75, more preferably at least 100, more preferably at least 120, more preferably at least 150 grams of acceptor per liter of initial reactor or incubator volume wherein the reactor or incubator volume ranges from 250 mL to 10,000 m³;
ii) Adding to the cultivation or incubation medium in a reactor or incubator at least one acceptor feed comprising at least 50, more preferably at least 75, more preferably at least 100, more preferably at least 120, more preferably at least 150 grams of acceptor per liter of initial reactor or incubator volume wherein the total reactor or incubator volume ranges from 250 mL to 10,000 m³ in one pulse or in a discontinuous (pulsed), preferably so that the final volume of the cultivation or incubation medium is not more than three-fold, preferably not more than two-fold, more preferably less than two-fold of the volume of the cultivation or incubation medium before the addition of the acceptor feed pulse(s);
iii) Adding to the cultivation or incubation medium in a reactor or incubator at least one acceptor feed comprising at least 50, more preferably at least 75, more preferably at least 100, more preferably at least 120, more preferably at least 150 grams of acceptor per liter of initial reactor or incubator volume wherein the total reactor or incubator volume ranges from 250 mL to 10,000 m³ in one pulse or in a discontinuous (pulsed) manner, preferably so that the final volume of the cultivation or incubation medium is not more than three-fold, preferably not more than two-fold, more preferably less than two-fold of the volume of the cultivation or incubation medium before the addition of the acceptor feed and wherein preferably, the pH of the acceptor feed pulse(s) is set between 2.0 and 10.0 and wherein preferably, the temperature of the acceptor feed pulse(s) is kept between 20° C. and 80° C.;
iv) Adding at least one acceptor feed in a discontinuous (pulsed) manner to the cultivation or incubation medium over the course of 5 minutes, 10 minutes, 30 minutes, 1 hour, 2 hours, 4 hours, 10 hours, 12 hours, 1 day, 2 days, 3 days, 4 days, 5 days by means of an acceptor feeding solution;
v) Adding at least one acceptor feed in a discontinuous (pulsed) manner to the cultivation or incubation medium over the course of 5 minutes, 10 minutes, 30 minutes, 1 hour, 2 hours, 4 hours, 10 hours, 12 hours, 1 day, 2 days, 3 days, 4 days, 5 days by means of an acceptor feeding solution and wherein the concentration of the acceptor feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably, the pH of the acceptor feeding solution is set between 2.0 and 10.0 and wherein preferably, the temperature of the acceptor feeding solution is kept between 20° C. and 80° C.;
the method resulting in one or more bioproduct(s) with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the cultivation or incubation. In a more preferred embodiment of the method of the disclosure, the acceptor is lactose.

In a more preferred embodiment, the method for the production of one or more bioproduct(s) as described herein comprises at least one of the following steps:
i) Use of a cultivation or incubation medium comprising at least 50, more preferably at least 75, more preferably at least 100, more preferably at least 120, more preferably at least 150 grams of lactose per liter of initial reactor or incubator volume wherein the reactor or incubator volume ranges from 250 mL to 10,000 m³;
ii) Adding to the cultivation or incubation medium in a reactor or incubator a lactose feed comprising at least 50, more preferably at least 75, more preferably at least 100, more preferably at least 120, more preferably at least 150 gram of lactose per liter of initial reactor or incubator volume wherein the total reactor or incubator volume ranges from 250 mL to 10,000 m³, preferably in a continuous manner, and preferably so that the final volume of the cultivation or incubation medium is not more than three-fold, preferably not more than two-fold, more preferably less than two-fold of the volume of the cultivation or incubation medium before the addition of the lactose feed;
iii) Adding to the cultivation or incubation medium in a reactor or incubator a lactose feed comprising at least 50, more preferably at least 75, more preferably at least 100, more preferably at least 120, more preferably at least 150 grams of lactose per liter of initial reactor or incubator volume wherein the total reactor or incubator volume ranges from 250 mL to 10,000 m³, preferably in a continuous manner, and preferably so that the final volume of the cultivation or incubation medium is not more than three-fold, preferably not more than two-fold, more preferably less than two-fold of the volume of the cultivation or incubation medium before the addition of the lactose feed and wherein preferably, the pH of the lactose feed is set between 2.0 and 10.0, preferably between 3.0 and 7.0, and wherein preferably, the temperature of the lactose feed is kept between 20° C. and 80° C.;
iv) Adding a lactose feed in a continuous manner to the cultivation or incubation medium over the course of 1 day, 2 days, 3 days, 4 days, 5 days by means of a feeding solution;
v) Adding a lactose feed in a continuous manner to the cultivation or incubation medium over the course of 1 day, 2 days, 3 days, 4 days, 5 days by means of a feeding solution and wherein the concentration of the lactose feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably, the pH of the lactose feed is set between 2.0 and 10.0, preferably between 3.0 and 7.0 and wherein preferably, the temperature of the lactose feed is kept between 20° C. and 80° C.;
the method resulting in one or more bioproduct(s) with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the cultivation or incubation.

Preferably, the lactose feed is accomplished by adding lactose from the beginning of the cultivation or incubation in a concentration of at least 5 mM, preferably in a concentration of 30, 40, 50, 60, 70, 80, 90, 100, 150 mM, more preferably in a concentration >300 mM.

In another embodiment of the methods, the lactose feed is accomplished by adding lactose to the cultivation or incubation medium in a concentration, such that throughout the production phase of the cultivation or incubation a lactose concentration of at least 5 mM, preferably 10 mM or 30 mM is obtained.

In a further embodiment of the methods described herein the cells are cultivated or incubated for at least about 60, 80, 100, or about 120 hours or in a continuous manner.

In a preferred embodiment, a carbon source is provided, preferably sucrose, in the cultivation medium for 3 or more days, preferably up to 7 days; and/or provided, in the cultivation medium, at least 100, advantageously at least 105, more advantageously at least 110, even more advantageously at least 120 grams of sucrose per liter of initial cultivation volume in a continuous manner, so that the final volume of the cultivation medium is not more than three-fold, advantageously not more than two-fold, more advantageously less than two-fold of the volume of the cultivation medium before the cultivation.

Preferably, when performing the method as described herein, a first phase of exponential cell growth is provided by adding a carbon source, preferably glucose or sucrose, to the cultivation medium before the lactose is added to the cultivation medium in a second phase.

In an alternative preferable embodiment, in the method as described herein, the lactose is added already in the first phase of exponential growth together with the carbon-based substrate.

Another aspect provides for a cell to be stably cultured in a medium, wherein the medium can be any type of growth medium comprising minimal medium, complex medium or growth medium enriched in certain compounds like, for example, but not limited to, vitamins, trace elements, amino acids.

The microorganism or cell as used herein is capable to grow on a monosaccharide, disaccharide, oligosaccharide, polysaccharide, polyol, glycerol, a complex medium or a mixture thereof as the main carbon source. With the term main is meant the most important carbon source for the microorganism or cell for the production of one of more bioproduct(s) of interest, biomass formation, carbon dioxide and/or by-products formation (such as acids and/or alcohols, such as acetate, lactate, and/or ethanol), i.e., 20, 30, 40, 50, 60, 70, 75, 80, 85, 90, 95, 98, 99% of all the required carbon is derived from the above-indicated carbon source. In one embodiment of the disclosure, the carbon source is the sole carbon source for the organism, i.e., 100% of all the required carbon is derived from the above-indicated carbon source. Common main carbon sources comprise but are not limited to glucose, glycerol, fructose, sucrose, maltose, lactose, arabinose, malto-oligosaccharides, maltotriose, sorbitol, xylose, rhamnose, galactose, mannose, methanol, ethanol, trehalose, starch, cellulose, hemi-cellulose, molasses, corn-steep liquor, high-fructose syrup, acetate, citrate, lactate and pyruvate. As used herein, a precursor as defined herein cannot be used as a carbon source for the production of one or more bioproduct(s) of this disclosure.

The methods as described herein preferably comprises a step of separating one or more bioproduct(s) of this disclosure from the cultivation or incubation, otherwise the recovering one or more bioproduct(s) from the cultivation or incubation medium and/or the cell.

The terms “separating from the cultivation or incubation” means harvesting, collecting, or retrieving the one or more bioproduct(s) from the cell and/or the medium of its cultivation or incubation.

The one or more bioproduct(s) can be separated in a conventional manner from the aqueous culture medium, in which the cell was cultivated or incubated. In case the one or more bioproduct(s) is/are still present in the cells producing the one or more bioproduct(s), conventional manners to free or to extract the one or more bioproduct(s) out of the cells can be used, such as cell destruction using high pH, heat shock, sonication, French press, homogenization, enzymatic hydrolysis, chemical hydrolysis, solvent hydrolysis, detergent, hydrolysis, etc. The cultivation or incubation medium and/or cell extract together and separately can then be further used for separating the one or more bioproduct(s).

This preferably involves clarifying the one or more bioproduct(s) to remove suspended particulates and contaminants, particularly cells, cell components, insoluble metabolites and debris produced by culturing or incubating the genetically engineered cell. In this step, the one or more bioproduct(s) can be clarified in a conventional manner. Preferably, the one or more bioproduct(s) is clarified by centrifugation, flocculation, decantation and/or filtration. Another step of separating the one or more bioproduct(s) preferably involves removing substantially all the eventually remaining proteins, peptides, amino acids, RNA and DNA, and any endotoxins and glycolipids that could interfere with the subsequent separation step, from the one or more bioproduct(s), preferably after it/these has/have been clarified. In this step, remaining proteins and related impurities can be removed from the one or more bioproduct(s) in a conventional manner. Preferably, remaining proteins, salts, by-products, color, endotoxins and other related impurities are removed from the one or more bioproduct(s) by ultrafiltration, nanofiltration, two-phase partitioning, reverse osmosis, microfiltration, activated charcoal or carbon treatment, treatment with non-ionic surfactants, enzymatic digestion, tangential flow high-performance filtration, tangential flow ultrafiltration, electrophoresis (e.g., using slab-polyacrylamide or sodium dodecyl sulphate-polyacrylamide gel electrophoresis (PAGE)), affinity chromatography (using affinity ligands including e.g., DEAE-Sepharose, poly-L-lysine and polymyxin-B, endotoxin-selective adsorber matrices), ion exchange chromatography (such as but not limited to cation exchange, anion exchange, mixed bed ion exchange, inside-out ligand attachment), hydrophobic interaction chromatography and/or gel filtration (i.e., size exclusion chromatography), particularly by chromatography, more particularly by ion exchange chromatography or hydrophobic interaction chromatography or ligand exchange chromatography or electrodialysis. With the exception of size exclusion chromatography, remaining proteins and related impurities are retained by a chromatography medium or a selected membrane.

In a further preferred embodiment, the methods as described herein also provide for a further purification of one or more bioproduct(s) of this disclosure. A further purification of the one or more bioproduct(s) may be accomplished, for example, by use of (activated) charcoal or carbon, nanofiltration, ultrafiltration, electrophoresis, enzymatic treatment or ion exchange, temperature adjustment, pH adjustment or pH adjustment with an alkaline or acidic solution to remove any remaining DNA, protein, LPS, endotoxins, or other impurity. Alcohols, such as ethanol, and aqueous alcohol mixtures can also be used. Another purification step is accomplished by crystallization, evaporation or precipitation of the one or more bioproduct(s). Another purification step is to dry, e.g., spray dry, lyophilize, spray freeze dry, freeze spray dry, band dry, belt dry, vacuum band dry, vacuum belt dry, drum dry, roller dry, vacuum drum dry or vacuum roller dry the produced bioproduct(s).

In an exemplary embodiment, the separation and purification of one or more bioproduct(s) is made in a process, comprising the following steps in any order:
a) contacting the cultivation or incubation or a clarified version thereof with a nanofiltration membrane with a molecular weight cut-off (MWCO) of 600-3500 Da ensuring the retention of the produced bioproduct(s) and allowing at least a part of the proteins, salts, by-products, color and other related impurities to pass,
b) conducting a diafiltration process on the retentate from step a), using the membrane, with an aqueous solution of an inorganic electrolyte, followed by optional diafiltration with pure water to remove excess of the electrolyte,
c) and collecting the retentate enriched in the one or more bioproduct(s) in the form of a salt from the cation of the electrolyte.

In an alternative exemplary embodiment, the separation and purification of the one or more bioproduct(s) is made in a process, comprising the following steps in any order: subjecting the cultivation or incubation or a clarified version thereof to two membrane filtration steps using different membranes, wherein
one membrane has a molecular weight cut-off of between about 300 to about 500 Dalton, and
the other membrane as a molecular weight cut-off of between about 600 to about 800 Dalton.

In an alternative exemplary embodiment, the separation and purification of the one or more bioproduct(s) is made in a process, comprising treating the cultivation or incubation or a clarified version thereof with a strong cation exchange resin in H+-form in a step and with a weak anion exchange resin in free base form in another step, wherein the steps can be performed in any order.

In an alternative exemplary embodiment, the separation and purification of the one or more bioproduct(s) is made in the following way. The cultivation or incubation comprising the produced bioproduct(s), biomass, medium components and contaminants is applied to the following purification steps:
i) separation of biomass from the cultivation or incubation,
ii) cationic ion exchanger treatment for the removal of positively charged material,
iii) anionic ion exchanger treatment for the removal of negatively charged material,
iv) nanofiltration step and/or electrodialysis step,
wherein a purified solution comprising the produced bioproduct(s) at a purity of greater than or equal to 80% is provided. Optionally the purified solution is dried by any one or more drying steps chosen from the list comprising spray drying, lyophilization, spray freeze drying, freeze spray drying, band drying, belt drying, vacuum band drying, vacuum belt drying, drum drying, roller drying, vacuum drum drying and vacuum roller drying.

In an alternative exemplary embodiment, the separation and purification of the one or more bioproduct(s) is made in a process, comprising the following steps in any order: enzymatic treatment of the cultivation or incubation; removal of the biomass from the cultivation or incubation; ultrafiltration; nanofiltration; and a column chromatography step. Preferably, such column chromatography is a single column or a multiple column. Further preferably, the column chromatography step is simulated moving bed chromatography. Such simulated moving bed chromatography preferably comprises i) at least 4 columns, wherein at least one column comprises a weak or strong cation exchange resin; and/or ii) four zones I, II, III and IV with different flow rates; and/or iii) an eluent comprising water; and/or iv) an operating temperature of 15 degrees to 60 degrees centigrade.

In a specific embodiment, this disclosure provides the produced bioproduct(s) which is/are dried to powder by any one or more drying steps chosen from the list comprising spray drying, lyophilization, spray freeze drying, freeze spray drying, band drying, belt drying, vacuum band drying, vacuum belt drying, drum drying, roller drying, vacuum drum drying and vacuum roller drying, wherein the dried powder contains <15%-wt. of water, preferably <10%-wt. of water, more preferably <7%-wt. of water, most preferably <5%-wt. of water.

A further aspect of this disclosure provides for an isolated nucleic acid molecule encoding a saccharide importer having uptake activity for lacto-N-triose as described herein.

Another aspect of this disclosure provides for a vector comprising an isolated nucleic acid molecule a saccharide importer having uptake activity for lacto-N-triose as described herein.

This disclosure further provides a saccharide importer that has uptake activity for LN3 and that comprises a polypeptide sequence that has a deletion, an insertion and/or a mutation of one or more amino acid residue(s) i) N-terminally from the first transmembrane (TM) domain of any one of SEQ ID NOs: 12, 02, 01, 09, 19, 20 or 21 and/or ii) C-terminally from the last TM domain of any one of SEQ ID NOs: 12, 02, 01, 09, 19, 20 or 21. In a preferred embodiment, the saccharide importer of this disclosure has uptake activity for LN3 and comprises a polypeptide sequence that has a deletion, an insertion and/or a mutation of one or more amino acid residue(s) N-terminally from the first transmembrane (TM) domain of any one of SEQ ID NOs: 12, 02, 01, 09, 19, 20 or 21, wherein the deletion, insertion and/or mutation of one or more amino acid residue(s) is in or N-terminally of a non-TM helix.

In another and/or additional preferred embodiment, the saccharide importer of this disclosure has uptake activity for LN3 and comprises a polypeptide sequence that has a deletion, an insertion and/or a mutation of one or more amino acid residue(s) C-terminally from the last TM domain of any one of SEQ ID NOs: 12, 02, 01, 09, 19, 20 or 21, wherein the deletion, insertion and/or mutation of one or more amino acid residue(s) is in or C-terminally of a non-TM helix.

In another and/or additional preferred embodiment, the variant saccharide importer having a deletion, an insertion and/or a mutation of one or more amino acid residue(s) i) N-terminally from the first transmembrane (TM) domain of any one of SEQ ID NOs: 12, 02, 01, 09, 19, 20 or 21 and/or ii) C-terminally from the last TM domain of any one of SEQ ID NOs: 12, 02, 01, 09, 19, 20 or 21 has a higher, faster and/or more efficient, i.e., less energy-consuming, uptake ratio for the saccharide compared to the native saccharide importer lacking the deletion, insertion and/or mutation.

Preferably, the saccharide importer comprises, consists, or consists essentially of the polypeptide sequence with SEQ ID NO:03 and has uptake activity for LN3; SEQ ID NO:03 is a mutant polypeptide of the saccharide importer with SEQ ID NO:02 wherein the SEQ ID NO:03 has a deletion of 39 amino acid residues N-terminally from the first TM domain of SEQ ID NO:02 and an insertion of 19 amino acid residues N-terminally from the first TM domain of SEQ ID NO: 02. The first TM domain of SEQ ID NO:02 starts at Ser at position 40. Both polypeptides with SEQ ID NOs: 02 and 03 have uptake activity for LN3. In an even more preferred embodiment, the saccharide importer as described herein is the polypeptide with SEQ ID NO:03 and has uptake activity for LN3.

Alternatively, the saccharide importer as described herein comprises, consists, or consists essentially of the polypeptide sequence with SEQ ID NO:04 and has uptake activity for LN3; SEQ ID NO:04 is a mutant polypeptide of the saccharide importer with SEQ ID NO:02 wherein the SEQ ID NO:04 has a deletion of 39 amino acid residues N-terminally from the first TM domain of SEQ ID NO:02 and an insertion of 19 amino acid residues N-terminally from the first TM domain of SEQ ID NO:02. The first TM domain of SEQ ID NO:02 starts at Ser at position 40. Both polypeptides with SEQ ID NOs: 02 and 04 have uptake activity for LN3. In an even more preferred embodiment, the saccharide importer as described herein is the polypeptide with SEQ ID NO:04 and has uptake activity for LN3.

Alternatively, the saccharide importer as described herein comprises, consists, or consists essentially of the polypeptide sequence with SEQ ID NO:16 and has uptake activity for LN3; SEQ ID NO:16 is a mutant polypeptide of the saccharide importer with SEQ ID NO:09 wherein the SEQ ID NO: 16 has a deletion of 23 amino acid residues N-terminally from the first TM domain of SEQ ID NO:09 and an insertion of 20 amino acid residues N-terminally from the first TM domain of SEQ ID NO:09. The first TM domain of SEQ ID NO:09 starts at Val at position 24. In an even more preferred embodiment, the saccharide importer as described herein is the polypeptide with SEQ ID NO: 16 and has uptake activity for LN3.

Another aspect of this disclosure provides a saccharide importer having uptake activity for LN3 as described herein for use in the production of one or more bioproduct(s) chosen from the list comprising saccharide; monosaccharide; activated monosaccharide; phosphorylated monosaccharide; disaccharide; oligosaccharide; polysaccharide; milk saccharide; mammalian milk saccharide; MMO; human milk saccharide; HMO; neutral (non-charged) saccharide; negatively charged saccharide; fucosylated saccharide; sialylated saccharide; neutral (non-charged) oligosaccharide; negatively charged oligosaccharide; fucosylated oligosaccharide; sialylated oligosaccharide; N-acetylglucosamine containing oligosaccharide; N-acetyllactosamine containing oligosaccharide; lacto-N-biose containing oligosaccharide; lactose containing oligosaccharide; non-fucosylated neutral (non-charged) oligosaccharide; O-antigen; ECA; the oligosaccharide repeats present in capsular polysaccharides; peptidoglycan; amino-sugar; Lewis-type antigen oligosaccharide; antigen of the human ABO blood group system; animal oligosaccharide, preferably selected from the group consisting of N-glycans and O-glycans; plant oligosaccharide, preferably selected from the group consisting of N-glycans and O-glycans; LN3; lacto-N-tetraose (LNT); LNnT; oligosaccharide derived from LN3; 3′sialyllactose (3′SL); 6′sialyllactose (6′SL); sialyllacto-N-tetraose a (LSTa); sialyllacto-N-tetraose b (LSTb); sialyllacto-N-tetraose c (LSTc); sialyllacto-N-tetraose d (LSTd); lacto-N-fucopentaose I; lacto-N-neofucopentaose; lacto-N-fucopentaose II; lacto-N-fucopentaose III; lacto-N-fucopentaose V; lacto-N-neofucopentaose V; lacto-N-difucohexaose I; lacto-N-neodifucohexaose; lacto-N-difucohexaose II; monofucosyllacto-n-hexaose III; difucosyllacto-N-hexaose a; 6′-galactosyllactose; 3′-galactosyllactose; lacto-N-hexaose; lacto-N-neohexaose; 2′-fucosyllactose (2′FL); 3-fucosyllactose (3-FL); difucosyllactose (DiFL); chitosan; chitosan comprising oligosaccharide; heparosan; glycosaminoglycan oligosaccharide; heparin; heparan sulphate; chondroitin sulphate; dermatan sulphate; hyaluronan; hyaluronic acid and keratan sulphate. In a preferred embodiment, the one or more bioproduct(s) is/are one or more LN3 derived oligosaccharide(s). In another preferred embodiment, the saccharide importer having uptake activity for LN3 for use in the production of one or more bioproduct(s), further comprises uptake activity for one or more other saccharide(s) different from LN3, wherein the one or more other saccharide(s) different from LN3 are chosen from the list comprising monosaccharide, disaccharide, oligosaccharide and polysaccharide.

Other further aspects of this disclosure provide the use of i) a cell as described herein, ii) a method as described herein, iii) an isolated nucleic acid molecule as described herein, iv) a vector as described herein or v) a saccharide importer having uptake activity for LN3 as described herein for the production of one or more bioproduct(s) chosen from the list comprising saccharide; monosaccharide; activated monosaccharide; phosphorylated monosaccharide; disaccharide; oligosaccharide; polysaccharide; milk saccharide; mammalian milk saccharide; MMO; human milk saccharide; HMO; neutral (non-charged) saccharide; negatively charged saccharide; fucosylated saccharide; sialylated saccharide; neutral (non-charged) oligosaccharide; negatively charged oligosaccharide; fucosylated oligosaccharide; sialylated oligosaccharide; N-acetylglucosamine containing oligosaccharide; N-acetyllactosamine containing oligosaccharide; lacto-N-biose containing oligosaccharide; lactose containing oligosaccharide; non-fucosylated neutral (non-charged) oligosaccharide; O-antigen; ECA; the oligosaccharide repeats present in capsular polysaccharides; peptidoglycan; amino-sugar; Lewis-type antigen oligosaccharide; antigen of the human ABO blood group system; animal oligosaccharide, preferably selected from the group consisting of N-glycans and O-glycans; plant oligosaccharide, preferably selected from the group consisting of N-glycans and O-glycans; LN3; lacto-N-tetraose (LNT); LNnT; oligosaccharide derived from LN3; 3′sialyllactose (3′SL); 6′sialyllactose (6′SL); sialyllacto-N-tetraose a (LSTa); sialyllacto-N-tetraose b (LSTb); sialyllacto-N-tetraose c (LSTc); sialyllacto-N-tetraose d (LSTd); lacto-N-fucopentaose I; lacto-N-neofucopentaose; lacto-N-fucopentaose II; lacto-N-fucopentaose III; lacto-N-fucopentaose V; lacto-N-neofucopentaose V; lacto-N-difucohexaose I; lacto-N-neodifucohexaose; lacto-N-difucohexaose II; monofucosyllacto-n-hexaose III; difucosyllacto-N-hexaose a; 6′-galactosyllactose; 3′-galactosyllactose; lacto-N-hexaose; lacto-N-neohexaose; 2′-fucosyllactose (2′FL); 3-fucosyllactose (3-FL); difucosyllactose (DiFL); chitosan; chitosan comprising oligosaccharide; heparosan; glycosaminoglycan oligosaccharide; heparin; heparan sulphate; chondroitin sulphate; dermatan sulphate; hyaluronan; hyaluronic acid and keratan sulphate. In a preferred embodiment, the one or more bioproduct(s) is/are one or more LN3 derived oligosaccharide(s).

Furthermore, the disclosure also relates to the one or more bioproduct(s) obtained by the methods according to the disclosure. The one or more bioproduct(s) may be used for the manufacture of a preparation, as food additive, prebiotic, symbiotic, for the supplementation of baby food, adult food, infant animal feed, adult animal feed, or as either therapeutically or pharmaceutically active compound or in cosmetic applications. In a preferred embodiment, the preparation comprises at least one bioproduct that is obtainable, preferably obtained, by the methods as described herein. In another preferred embodiment, a preparation is provided that further comprises at least one probiotic microorganism. In another preferred embodiment of this disclosure, the preparation is a nutritional composition. In a more preferred embodiment, the preparation is a medicinal formulation, a dietary supplement, a dairy drink or an infant formula.

With the novel methods, the one or more bioproduct(s) can easily and effectively be provided, without the need for complicated, time and cost consuming synthetic processes.

For identification of the bioproduct(s) of this disclosure produced as described herein, the monosaccharide or the monomeric building blocks (e.g., the monosaccharide or glycan unit composition), the anomeric configuration of side chains, the presence and location of substituent groups, degree of polymerization/molecular weight and the linkage pattern can be identified by standard methods known in the art, such as, e.g., methylation analysis, reductive cleavage, hydrolysis, GC-MS (gas chromatography-mass spectrometry), MALDI-MS (Matrix-assisted laser desorption/ionization-mass spectrometry), ESI-MS (Electrospray ionization-mass spectrometry), HPLC (High-Performance Liquid chromatography with ultraviolet or refractive index detection), HPAEC-PAD (High-Performance Anion-Exchange chromatography with Pulsed Amperometric Detection), CE (capillary electrophoresis), IR (infrared)/Raman spectroscopy, and NMR (Nuclear magnetic resonance) spectroscopy techniques. The crystal structure can be solved using, e.g., solid-state NMR, FT-IR (Fourier transform infrared spectroscopy), and WAXS (wide-angle X-ray scattering). The degree of polymerization (DP), the DP distribution, and polydispersity can be determined by, e.g., viscosimetry and SEC (SEC-HPLC, high performance size-exclusion chromatography). To identify the monomeric components of the bioproduct(s) methods such as e.g., acid-catalyzed hydrolysis, HPLC (high performance liquid chromatography) or GLC (gas-liquid chromatography) (after conversion to alditol acetates) may be used. To determine the glycosidic linkages, the bioproduct(s) is/are methylated with methyl iodide and strong base in DMSO, hydrolysis is performed, a reduction to partially methylated alditols is achieved, an acetylation to methylated alditol acetates is performed, and the analysis is carried out by GLC/MS (gas-liquid chromatography coupled with mass spectrometry). To determine the glycan sequence, a partial depolymerization is carried out using an acid or enzymes to determine the structures. To identify the anomeric configuration, the bioproduct(s) is/are subjected to enzymatic analysis, e.g., it is contacted with an enzyme that is specific for a particular type of linkage, e.g., beta-galactosidase, or alpha-glucosidase, etc., and NMR may be used to analyze the products.

The separated and preferably also purified bioproduct(s) as described herein is/are incorporated into a food (e.g., human food or feed), dietary supplement, pharmaceutical ingredient, cosmetic ingredient or medicine. In some embodiments, the bioproduct(s) is/are mixed with one or more ingredients suitable for food, feed, dietary supplement, pharmaceutical ingredient, cosmetic ingredient or medicine.

In some embodiments, the dietary supplement comprises at least one prebiotic ingredient and/or at least one probiotic ingredient.

A “prebiotic” is a substance that promotes growth of microorganisms beneficial to the host, particularly microorganisms in the gastrointestinal tract. In some embodiments, a dietary supplement provides multiple prebiotics, including the bioproduct(s) being (a) prebiotic molecule(s) produced and/or purified by a process disclosed in this disclosure, to promote growth of one or more beneficial microorganisms. Examples of prebiotic ingredients for dietary supplements include other prebiotic molecules (such as HMOs) and plant polysaccharides (such as inulin, pectin, b-glucan and xylooligosaccharide). A “probiotic” product typically contains live microorganisms that replace or add to gastrointestinal microflora, to the benefit of the recipient. Examples of such microorganisms include Lactobacillus species (for example, L. acidophilus and L. bulgaricus), Bifidobacterium species (for example, B. animalis, B. longum and B. infantis (e.g., Bi-26)), and Saccharomyces boulardii. In some embodiments, one or more bioproduct(s) produced and/or purified by a process of this disclosure is/are orally administered in combination with such microorganism.

Examples of further ingredients for dietary supplements include oligosaccharides (such as 2′-fucosyllactose, 3-fucosyllactose, 6′-sialyllactose), disaccharides (such as lactose), monosaccharides (such as glucose, galactose, L-fucose, sialic acid, glucosamine and N-acetylglucosamine), thickeners (such as gum arabic), acidity regulators (such as trisodium citrate), water, skimmed milk, and flavorings.

In some embodiments, the bioproduct(s) is/are incorporated into a human baby food (e.g., infant formula). Infant formula is generally a manufactured food for feeding to infants as a complete or partial substitute for human breast milk. In some embodiments, infant formula is sold as a powder and prepared for bottle- or cup-feeding to an infant by mixing with water. The composition of infant formula is typically designed to be roughly mimic human breast milk. In some embodiments, one or more bioproduct(s) produced and/or purified by a process in this disclosure is/are included in infant formula to provide nutritional benefits similar to those provided by the oligosaccharides in human breast milk. In some embodiments, one or more bioproduct(s) is/are mixed with one or more ingredients of the infant formula. Examples of infant formula ingredients include non-fat milk, carbohydrate sources (e.g., lactose), protein sources (e.g., whey protein concentrate and casein), fat sources (e.g., vegetable oils-such as palm, high oleic safflower oil, rapeseed, coconut and/or sunflower oil; and fish oils), vitamins (such as vitamins A, Bb, Bi2, C and D), minerals (such as potassium citrate, calcium citrate, magnesium chloride, sodium chloride, sodium citrate and calcium phosphate) and possibly human milk oligosaccharides (HMOs). Such HMOs may include, for example, 2′FL, 3-FL, DiFL, 3′SL, 6′SL, lacto-N-triose II, LNT, LNnT, lacto-N-fucopentaose I, lacto-N-neofucopentaose, lacto-N-fucopentaose II, lacto-N-fucopentaose III, lacto-N-fucopentaose V, lacto-N-neofucopentaose V, lacto-N-difucohexaose I, lacto-N-difucohexaose II, 6′-galactosyllactose, 3′-galactosyllactose, lacto-N-hexaose and lacto-N-neohexaose.

In some embodiments, the one or more infant formula ingredients comprise non-fat milk, a carbohydrate source, a protein source, a fat source, and/or a vitamin and mineral.

In some embodiments, the one or more infant formula ingredients comprise lactose, whey protein concentrate and/or high oleic safflower oil.

In some embodiments, the concentration of the bioproduct(s) in the infant formula is/are approximately the same concentration as the concentration of the bioproduct(s) generally present in human breast milk.

In some embodiments, the bioproduct(s) is/are incorporated into a feed preparation, wherein the feed is chosen from the list comprising pet food, animal milk replacer, veterinary product, veterinary feed supplement, nutrition supplement, post weaning feed, or creep feed.

As will be shown in the examples herein, the saccharide importers having uptake activity for LN3 have proven to be useful in cell-based production of one or more bioproduct(s), preferably LN3 derived oligosaccharides, more preferably LNT and/or LNnT. The methods and the cell of the disclosure preferably provide at least one of the following further surprising advantages when using the saccharide importers as described herein:
Higher titers of the bioproduct(s) (g/L),
Higher purity of the bioproduct(s),
Higher production rate r (g bioproduct/L/h),
Higher cell performance index CPI (g bioproduct/g X),
Higher specific productivity Qp (g bioproduct/g X/h),
Higher yield on the carbon source used Y (g bioproduct/g carbon source used),
Higher yield on sucrose Ys (g bioproduct/g sucrose),
Higher uptake/conversion rate of the carbon source used Q (g carbon source/gX/h),
Higher sucrose uptake/conversion rate Qs (g sucrose/gX/h),
Higher lactose conversion/consumption rate rs (g lactose/h),
Higher secretion of the bioproduct(s), and/or
Higher growth speed of the production host,
when compared to a method or a cell using an identical setup or genetic background but lacking the use of the saccharide importers having uptake activity for LN3 as described herein.

Unless defined otherwise, all technical and scientific terms used herein generally have the same meaning as commonly understood by one of ordinary skill in the art to which this disclosure belongs. Generally, the nomenclature used herein and the laboratory procedures in cell culture, molecular genetics, organic chemistry and nucleic acid chemistry and hybridization described above and below are those well-known and commonly employed in the art. Standard techniques are used for nucleic acid and peptide synthesis. Generally, purification steps are performed according to the manufacturer's specifications.

Further advantages follow from the specific embodiments and the examples. It goes without saying that the abovementioned features and the features which are still to be explained below can be used not only in the respectively specified combinations, but also in other combinations or on their own, without departing from the scope of this disclosure.

Moreover, this disclosure relates to the following specific embodiments:

1. A cell for the production of one or more bioproduct(s), wherein the cell:
is capable to produce, preferably produces, one or more precursor(s) used in the production of at least one of the one or more bioproduct(s), preferably the cell is genetically engineered to produce at least one of the one or more precursor(s) used in the production of at least one of the one or more bioproduct(s), more preferably the cell is genetically engineered to produce all of the one or more precursor(s) used in the production of at least one of the one or more bioproduct(s), and
is genetically engineered to express, preferably to overexpress, a polynucleotide sequence that encodes a saccharide importer that internalizes at least one of the one or more precursor(s) into the cell.

2. Cell according to embodiment 1, wherein the saccharide importer has uptake activity for lacto-N-triose (LN3, GlcNAc-beta1,3-Gal-beta1,4-Glc).

3. Cell according to any one of embodiment 1 or 2, wherein one of the one or more precursor(s) is LN3.

4. Cell according to any one of embodiments 1 to 3, wherein one of the one or more precursor(s) is LN3 and wherein the saccharide importer has uptake activity for the LN3 being produced by the cell.

5. A cell for the production of one or more bioproduct(s), wherein the cell is genetically engineered to express, preferably to overexpress, a polynucleotide sequence that encodes a saccharide importer that has uptake activity for lacto-N-triose (LN3, GlcNAc-beta1,3-Gal-beta1,4-Glc).

6. Cell according to any one of previous embodiments, wherein the saccharide importer:
originates from the major facilitator superfamily (MFS) of transporters,
comprises a polypeptide sequence comprising an IPR001927 domain as defined by InterPro 90.0 as released on 4 Aug. 2022,
comprises a polypeptide sequence that is at least 25% identical over a stretch of at least 150 amino acid residues, preferably at least 200 amino acid residues, to any one of the polypeptide sequences as represented by SEQ ID NOs: 04, 12, 02, 01, 03, 05, 06, 07, 08, 09, 10, 11 or 13,
comprises a polypeptide sequence that is at least 25% identical to any one of the full-length polypeptide sequences as represented by SEQ ID NOs: 04, 12, 02, 01, 03, 05, 06, 07, 08, 09, 10, 11 or 13,
comprises a polypeptide sequence as represented by any one of SEQ ID NOs: 04, 12, 02, 01, 03, 05, 06, 07, 08, 09, 10, 11 or 13,
comprises a polypeptide sequence that has a deletion, insertion and/or a mutation of one or more amino acid residue(s) N-terminally from the first transmembrane (TM) domain of any one of SEQ ID NOs: 04, 12, 02, 01, 03, 05, 06, 07, 08, 09, 10, 11 or 13, preferably the deletion, insertion and/or mutation is in or N-terminally of a non-TM helix, and/or
comprises a polypeptide sequence that has a deletion, insertion and/or a mutation of one or more amino acid residue(s) C-terminally from the last transmembrane (TM) domain of any one of SEQ ID NOs: 04, 12, 02, 01, 03, 05, 06, 07, 08, 09, 10, 11 or 13, preferably the deletion, insertion and/or mutation is in or C-terminally of a non-TM helix.

7. Cell according to any one of embodiments 2 to 6, wherein the saccharide importer further comprises uptake activity for one or more other saccharide(s) different from LN3, wherein the one or more other saccharide(s) different from LN3 are chosen from the list comprising monosaccharide, disaccharide, oligosaccharide and polysaccharide.

8. Cell according to any one of previous embodiments, wherein the one or more bioproduct(s) is/are chosen from the list comprising saccharide; monosaccharide; activated monosaccharide; phosphorylated monosaccharide; disaccharide; oligosaccharide; polysaccharide; milk saccharide; mammalian milk saccharide; MMO; human milk saccharide; HMO; neutral (non-charged) saccharide; negatively charged saccharide; fucosylated saccharide; sialylated saccharide; neutral (non-charged) oligosaccharide; negatively charged oligosaccharide; fucosylated oligosaccharide; sialylated oligosaccharide; N-acetylglucosamine containing oligosaccharide; N-acetyllactosamine containing oligosaccharide; lacto-N-biose containing oligosaccharide; lactose containing oligosaccharide; non-fucosylated neutral (non-charged) oligosaccharide; O-antigen; ECA; the oligosaccharide repeats present in capsular polysaccharides; peptidoglycan; amino-sugar; Lewis-type antigen oligosaccharide; antigen of the human ABO blood group system; animal oligosaccharide, preferably selected from the group consisting of N-glycans and O-glycans; plant oligosaccharide, preferably selected from the group consisting of N-glycans and O-glycans; LN3; lacto-N-tetraose (LNT); LNnT; oligosaccharide derived from LN3; 3′sialyllactose (3′SL); 6′sialyllactose (6′SL); sialyllacto-N-tetraose a (LSTa); sialyllacto-N-tetraose b (LSTb); sialyllacto-N-tetraose c (LSTc); sialyllacto-N-tetraose d (LSTd); lacto-N-fucopentaose I; lacto-N-neofucopentaose; lacto-N-fucopentaose II; lacto-N-fucopentaose III; lacto-N-fucopentaose V; lacto-N-neofucopentaose V; lacto-N-difucohexaose I; lacto-N-neodifucohexaose; lacto-N-difucohexaose II; monofucosyllacto-n-hexaose III; difucosyllacto-N-hexaose a; 6′-galactosyllactose; 3′-galactosyllactose; lacto-N-hexaose; lacto-N-neohexaose; 2′-fucosyllactose (2′FL); 3-fucosyllactose (3-FL); difucosyllactose (DiFL); chitosan; chitosan comprising oligosaccharide; heparosan; glycosaminoglycan oligosaccharide; heparin; heparan sulphate; chondroitin sulphate; dermatan sulphate; hyaluronan; hyaluronic acid and keratan sulphate, preferably, the one or more bioproduct(s) is/are one or more LN3 derived oligosaccharide(s).

9. Cell according to any one of previous embodiments, wherein the polynucleotide sequence is operably linked to control sequences recognized by the cell and/or wherein the polynucleotide sequence is foreign to the cell, the polynucleotide sequence further i) being integrated in the genome of the cell and/or ii) presented to the cell on a vector.

10. Cell according to any one of previous embodiments, wherein the cell is further genetically engineered for the production of the one or more bioproduct(s).

11. Cell according to any one of embodiments 5 to 10, wherein the cell is capable to produce, preferably produces, the one or more bioproduct(s) from one or more precursor(s).

12. Cell according to embodiment 11, wherein the cell is capable to produce, preferably produces, at least one of the one or more precursor(s), preferably the cell is capable to produce, preferably produces, all of the one or more precursor(s).

13. Cell according to embodiment 12, wherein the cell is genetically engineered for the production of at least one of the one or more precursor(s), preferably the cell is genetically engineered for the production of all of the one or more precursor(s).

14. Cell according to any one of embodiments 11 to 13, wherein at least one of the one or more precursor(s) is internalized in the cell via the saccharide importer.

15. Cell according to any one of embodiments 1 to 4, 11 to 14, wherein the one or more precursor(s) is/are chosen from the list comprising saccharide; monosaccharide; activated monosaccharide; phosphorylated monosaccharide; disaccharide; oligosaccharide; polysaccharide; milk saccharide; mammalian milk saccharide; MMO; human milk saccharide; HMO; neutral (non-charged) saccharide; negatively charged saccharide; fucosylated saccharide; sialylated saccharide; neutral (non-charged) oligosaccharide; negatively charged oligosaccharide; fucosylated oligosaccharide; sialylated oligosaccharide; N-acetylglucosamine containing oligosaccharide; N-acetyllactosamine oligosaccharide; lacto-N-biose containing oligosaccharide; lactose containing oligosaccharide; non-fucosylated neutral (non-charged) oligosaccharide; O-antigen; ECA; the oligosaccharide repeats present in capsular polysaccharides; peptidoglycan; amino-sugar; Lewis-type antigen oligosaccharide; antigen of the human ABO blood group system; animal oligosaccharide, preferably selected from the group consisting of N-glycans and O-glycans; plant oligosaccharide, preferably selected from the group consisting of N-glycans and O-glycans; LN3; lacto-N-tetraose (LNT); LNnT; oligosaccharide derived from LN3; 3′sialyllactose (3′SL); 6′sialyllactose (6′SL); sialyllacto-N-tetraose a (LSTa); sialyllacto-N-tetraose b (LSTb); sialyllacto-N-tetraose c (LSTc); sialyllacto-N-tetraose d (LSTd); lacto-N-fucopentaose I; lacto-N-neofucopentaose; lacto-N-fucopentaose II; lacto-N-fucopentaose III; lacto-N-fucopentaose V; lacto-N-neofucopentaose V; lacto-N-difucohexaose I; lacto-N-neodifucohexaose; lacto-N-difucohexaose II; monofucosyllacto-n-hexaose III; difucosyllacto-N-hexaose a; 6′-galactosyllactose; 3′-galactosyllactose; lacto-N-hexaose; lacto-N-neohexaose; 2′-fucosyllactose (2′FL); 3-fucosyllactose (3-FL); difucosyllactose (DiFL); chitosan; chitosan comprising oligosaccharide; heparosan; glycosaminoglycan oligosaccharide; heparin; heparan sulphate; chondroitin sulphate; dermatan sulphate; hyaluronan; hyaluronic acid and keratan sulphate; preferably, the one or more bioproduct(s) is/are one or more LN3 derived oligosaccharide(s).

16. Cell according to any one of previous embodiments, wherein the cell possesses, preferably expresses, more preferably overexpresses, one or more glycosyltransferase(s) chosen from the list comprising fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases and fucosaminyltransferases,
preferably, the fucosyltransferase is chosen from the list comprising alpha-1,2-fucosyltransferase, alpha-1,3-fucosyltransferase, alpha-1,3/4-fucosyltransferase, alpha-1,4-fucosyltransferase and alpha-1,6-fucosyltransferase,
preferably, the sialyltransferase is chosen from the list comprising alpha-2,3-sialyltransferase, alpha-2,6-sialyltransferase and alpha-2,8-sialyltransferase,
preferably, the galactosyltransferase is chosen from the list comprising beta-1,3-galactosyltransferase, N-acetylglucosamine beta-1,3-galactosyltransferase, beta-1,4-galactosyltransferase, N-acetylglucosamine beta-1,4-galactosyltransferase, alpha-1,3-galactosyltransferase and alpha-1,4-galactosyltransferase,
preferably, the glucosyltransferase is chosen from the list comprising alpha-glucosyltransferase, beta-1,2-glucosyltransferase, beta-1,3-glucosyltransferase and beta-1,4-glucosyltransferase,
preferably, the mannosyltransferase is chosen from the list comprising alpha-1,2-mannosyltransferase, alpha-1,3-mannosyltransferase and alpha-1,6-mannosyltransferase,
preferably, the N-acetylglucosaminyltransferase is chosen from the list comprising galactoside beta-1,3-N-acetylglucosaminyltransferase and beta-1,6-N-acetylglucosaminyltransferase,
preferably, the N-acetylgalactosaminyltransferase is an alpha-1,3-N-acetylgalactosaminyltransferase.

17. Cell according to any one of previous embodiments, wherein the cell is capable to produce, preferably produces, one or more nucleotide-activated sugars preferably chosen from the list comprising UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), GDP-fucose, (GDP-Fuc), UDP-glucuronate, UDP-galacturonate, UDP-2-acetamido-2,6-dideoxy--L-arabino-4-hexulose, UDP-2-acetamido-2,6-dideoxy--L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid (CMP-Neu5Ac), CMP-Neu4Ac, CMP-Neu5Ac9N₃, CMP-Neu4,5Ac₂, CMP-Neu5,7Ac₂, CMP-Neu5,9Ac₂, CMP-Neu5,7(8,9)Ac₂, CMP-N-glycolylneuraminic acid (CMP-Neu5Gc), GDP-rhamnose and UDP-xylose, preferably the cell is genetically engineered for production of one or more of the nucleotide-activated sugar(s).

18. Cell according to any one of previous embodiments, wherein the cell comprises at least one pathway chosen from the list comprising fucosylation pathway, sialylation pathway, galactosylation pathway, N-acetylglucosaminylation pathway, N-acetylgalactosaminylation pathway, mannosylation pathway and N-acetylmannosaminylation pathway, preferably the cell is genetically engineered to comprise at least one of the pathway(s), more preferably the cell comprises at least one of the pathway(s) wherein at least one of the pathway(s) has/have been genetically engineered.

19. Cell according to any one of previous embodiments, wherein the cell comprises a catabolic pathway for selected mono-, di- or oligosaccharides which is at least partially inactivated, the mono-, di-, or oligosaccharides being involved in and/or required for the production of the one or more bioproduct(s).

20. Cell according to any one of previous embodiments, wherein the cell is a bacterium, fungus, yeast, a plant cell, an animal cell, or a protozoan cell,
preferably, the bacterium belongs to a phylum chosen from the group comprising Proteobacteria, Firmicutes, Cyanobacteria, Deinococcus-Thermus and Actinobacteria; more preferably, the bacterium belongs to a family chosen from the group comprising Enterobacteriaceae, Bacillaceae, Lactobacillus, Corynebacteriaceae and Vibrionaceae; even more preferably, the bacterium is chosen from the list comprising an Escherichia coli strain, a Bacillus subtilis strain, a Vibrio natriegens strain; even more preferably the Escherichia coli strain is a K-12 strain, most preferably the Escherichia coli K-12 strain is E. coli MG1655,
preferably, the fungus belongs to a genus chosen from the group comprising Rhizopus, Dictyostelium, Penicillium, Mucor or Aspergillus,
preferably, the yeast belongs to a genus chosen from the group comprising Saccharomyces, Zygosaccharomyces, Pichia, Komagataella, Hansemila, Yarrowia, Starmerella, Kluyveromyces, Debaromyces, Candida, Schizosaccharomyces, Schwanniomyces or Torulaspora; more preferably, the yeast is selected from the group consisting of: Saccharomyces cerevisiae, Hansemila polymorpha, Kluyveromyces lactis, Kluyveromyces marxianus, Pichia pastoris, Pichia methanolica, Pichia stipites, Candida boidinii, Schizosaccharomyces pombe, Schwanniomyces occidentalis, Torulaspora delbrueckii, Yarrowia lipolytica, Zygosaccharomyces rouxii, and Zygosaccharomyces bailii,
preferably, the plant cell is an algal cell or is derived from tobacco, alfalfa, rice, tomato, cotton, rapeseed, soy, maize, or corn plant,
preferably, the animal cell is derived from insects, amphibians, reptiles, invertebrates, fish, birds or mammalian cells excluding human embryonic stem cells, more preferably the mammalian cell is chosen from the list comprising an epithelial cell, an embryonic kidney cell, a fibroblast cell, a COS cell, a Chinese hamster ovary (CHO) cell, a murine myeloma cell, an NIH-3T3 cell, a lactocyte derived from mammalian induced pluripotent stem cells, more preferably the mammalian induced pluripotent stem cells are human induced pluripotent stem cells, a post-parturition mammary epithelium cell, a polarized mammary cell, more preferably the polarized mammary cell is selected from the group comprising live primary mammary epithelial cells, live mammary myoepithelial cells, live mammary progenitor cells, live immortalized mammary epithelial cells, live immortalized mammary myoepithelial cells, live immortalized mammary progenitor cells, a non-mammary adult stem cell or derivatives thereof, more preferably the insect cell is derived from Spodoptera frugiperda, Bombyx mori, Mamestra brassicae, Trichoplusia ni or Drosophila melanogaster,
preferably, the protozoan cell is a Leishmania tarentolae cell.

21. Cell according to any one of previous embodiments, wherein the cell is selected from the group consisting of prokaryotic cells and eukaryotic cells, preferably from the group consisting of yeast cells, bacterial cells, archaebacterial cells, algae cells, and fungal cells.

22. Cell according to any one of previous embodiments, wherein the cell is an E. coli or yeast with a lactose permease positive phenotype, preferably wherein the lactose permease is coded by the gene LacY or LAC12, respectively.

23. Method for the production of one or more bioproduct(s), the method comprising the steps of:
a) cultivating and/or incubating a cell, preferably a single cell, according to any one of embodiments 1 to 22 under conditions permissive to express the saccharide importer and to produce the one or more bioproduct(s),
b) preferably, separating, preferably purifying, the one or more bioproduct(s) from the cultivation or incubation.

24. Method according to embodiment 23, wherein the one or more bioproduct(s) is/are chosen from the list comprising saccharide; monosaccharide; activated monosaccharide; phosphorylated d monosaccharide; disaccharide; oligosaccharide; polysaccharide; milk saccharide; mammalian milk saccharide; MMO; human milk saccharide; HMO; neutral (non-charged) saccharide; negatively charged saccharide; fucosylated saccharide; sialylated saccharide; neutral (non-charged) oligosaccharide; negatively charged oligosaccharide; fucosylated oligosaccharide; sialylated oligosaccharide; N-acetylglucosamine containing oligosaccharide; N-acetyllactosamine containing oligosaccharide; lacto-N-biose containing oligosaccharide; lactose containing oligosaccharide; non-fucosylated neutral (non-charged) oligosaccharide; O-antigen; ECA; the oligosaccharide repeats present in capsular polysaccharides; peptidoglycan; amino-sugar; Lewis-type antigen oligosaccharide; antigen of the human ABO blood group system; animal oligosaccharide, preferably selected from the group consisting of N-glycans and O-glycans; plant oligosaccharide, preferably selected from the group consisting of N-glycans and O-glycans; LN3; lacto-N-tetraose (LNT); LNnT; oligosaccharide derived from LN3; 3′sialyllactose (3′SL); 6′sialyllactose (6′SL); sialyllacto-N-tetraose a (LSTa); sialyllacto-N-tetraose b (LSTb); sialyllacto-N-tetraose c (LSTc); sialyllacto-N-tetraose d (LSTd); lacto-N-fucopentaose I; lacto-N-neofucopentaose; lacto-N-fucopentaose II; lacto-N-fucopentaose III; lacto-N-fucopentaose V; lacto-N-neofucopentaose V; lacto-N-difucohexaose I; lacto-N-neodifucohexaose; lacto-N-difucohexaose II; monofucosyllacto-n-hexaose III; difucosyllacto-N-hexaose a; 6′-galactosyllactose; 3′-galactosyllactose; lacto-N-hexaose; lacto-N-neohexaose; 2′-fucosyllactose (2′FL); 3-fucosyllactose (3-FL); difucosyllactose (DiFL); chitosan; chitosan comprising oligosaccharide; heparosan; glycosaminoglycan oligosaccharide; heparin; heparan sulphate; chondroitin sulphate; dermatan sulphate; hyaluronan; hyaluronic acid and keratan sulphate; preferably, the one or more bioproduct(s) is/are one or more LN3 derived oligosaccharide(s).

25. Method according to any one of embodiment 23 or 24, wherein the cell produces the one or more bioproduct(s) with a higher yield and/or higher purity compared to a cell with the same genetic make-up but lacking expression of the saccharide importer.

26. Method according to any one of embodiments 23 to 25, wherein the one or more bioproduct(s) is/are one or more LN3-derived oligosaccharide(s) and wherein the cell produces the one or more LN3-derived oligosaccharide(s) with a higher yield and/or higher purity compared to a cell with the same genetic make-up but lacking expression of the saccharide importer.

27. Method according to any one of embodiments 23 to 26, wherein the cultivation medium contains at least one carbon source selected from the group consisting of glucose, fructose, sucrose, and glycerol.

28. Method according to any one of embodiments 23 to 27, wherein the cultivation or incubation medium contains at least one compound selected from the group consisting of lactose, galactose, glucose, UDP-GlcNAc, GlcNAc, UDP-Gal, UDP-Glc and LN3.

29. Method according to any one of embodiments 23 to 28, wherein the cultivation or incubation medium comprises one or more precursor(s) that is/are used for production of the one or more bioproduct(s).

30. Method according to embodiment 29, wherein the one or more precursor(s) in the cultivation or incubation medium is/are produced by the cell and/or wherein the one or more precursor(s) is/are taken up by the cell via the saccharide importer.

31. Method according to any one of embodiments 23 to 30, wherein the one or more bioproduct(s), preferably all of the bioproduct(s), is/are recovered from the cultivation or incubation medium and/or the cell.

32. Method according to any one of embodiments 23 to 31, wherein the method results in a production of at least 50 g/L, preferably at least 75 g/L, more preferably at least 90 g/L of the one or more bioproduct(s) in the final volume of the cultivation or incubation.

33. Use of a cell according to any one of embodiments 1 to 22 for production of one or more bioproduct(s), preferably the one or more bioproduct(s) is/are one or more LN3 derived oligosaccharide(s).

34. Use of a method according to any one of embodiments 23 to 32 for production of one or more bioproduct(s), preferably the one or more bioproduct(s) is/are one or more LN3 derived oligosaccharide(s).

35. A saccharide importer having uptake activity for lacto-N-triose (LN3, GlcNAc-beta1,3-Gal-beta1,4-Glc) and comprising a polypeptide sequence that has a deletion, insertion and/or a mutation of one or more amino acid residue(s):
N-terminally from the first transmembrane (TM) domain of any one of SEQ ID NOs: 04, 12, 02, 01, 03, 05, 06, 07, 08, 09, 10, 11 or 13, preferably the deletion, insertion and/or mutation is in or N-terminally of a non-TM helix and/or
C-terminally from the last TM domain of any one of SEQ ID NOs: 04, 12, 02, 01, 03, 05, 06, 07, 08, 09, 10, 11 or 13, preferably the deletion, insertion and/or mutation is in or C-terminally of a non-TM helix.

36. A saccharide importer according to embodiment 35, wherein the saccharide importer further comprises uptake activity for one or more other saccharide(s) different from LN3, wherein the one or more other saccharide(s) different from LN3 are chosen from the list comprising monosaccharide, disaccharide, oligosaccharide and polysaccharide.

37. A saccharide importer having uptake activity for lacto-N-triose (LN3, GlcNAc-beta1,3-Gal-beta1,4-Glc) for use in the production of one or more bioproduct(s), wherein the saccharide importer:
originates from the major facilitator superfamily (MFS) of transporters,
comprises a polypeptide sequence comprising an IPR001927 domain as defined by InterPro 90.0 as released on 4 Aug. 2022,
comprises a polypeptide sequence that is at least 25% identical over a stretch of at least 150 amino acid residues, preferably at least 200 amino acid residues, to any one of the polypeptide sequences as represented by SEQ ID NOs: 04, 12, 02, 01, 03, 05, 06, 07, 08, 09, 10, 11 or 13,
comprises a polypeptide sequence that is at least 25% identical to any one of the full-length polypeptide sequences as represented by SEQ ID NOs: 04, 12, 02, 01, 03, 05, 06, 07, 08, 09, 10, 11 or 13,
comprises a polypeptide sequence as represented by any one of SEQ ID NOs: 04, 12, 02, 01, 03, 05, 06, 07, 08, 09, 10, 11 or 13,
comprises a polypeptide sequence that has a deletion, insertion and/or a mutation of one or more amino acid residue(s) N-terminally from the first transmembrane (TM) domain of any one of SEQ ID NOs: 04, 12, 02, 01, 03, 05, 06, 07, 08, 09, 10, 11 or 13, preferably the deletion, insertion and/or mutation is in or N-terminally of a non-TM helix, and/or
comprises a polypeptide sequence that has a deletion, insertion and/or a mutation of one or more amino acid residue(s) C-terminally from the last transmembrane (TM) domain of any one of SEQ ID NOs: 04, 12, 02, 01, 03, 05, 06, 07, 08, 09, 10, 11 or 13, preferably the deletion, insertion and/or mutation is in or C-terminally of a non-TM helix,
and wherein the bioproduct(s) is/are chosen from the list comprising saccharide; monosaccharide; activated monosaccharide; phosphorylated monosaccharide; disaccharide; oligosaccharide; polysaccharide; milk saccharide; mammalian milk saccharide; MMO; human milk saccharide; HMO; neutral (non-charged) saccharide; negatively charged saccharide; fucosylated saccharide; sialylated saccharide; neutral (non-charged) oligosaccharide; negatively charged oligosaccharide; fucosylated oligosaccharide; sialylated oligosaccharide; N-acetylglucosamine containing oligosaccharide; N-acetyllactosamine containing oligosaccharide; lacto-N-biose containing oligosaccharide; lactose containing oligosaccharide; non-fucosylated neutral (non-charged) oligosaccharide; O-antigen; ECA; the oligosaccharide repeats present in capsular polysaccharides; peptidoglycan; amino-sugar; Lewis-type antigen oligosaccharide; antigen of the human ABO blood group system; animal oligosaccharide, preferably selected from the group consisting of N-glycans and O-glycans; plant oligosaccharide, preferably selected from the group consisting of N-glycans and O-glycans; LN3; lacto-N-tetraose (LNT); LNnT; oligosaccharide derived from LN3; 3′sialyllactose (3′SL); 6′sialyllactose (6′SL); sialyllacto-N-tetraose a (LSTa); sialyllacto-N-tetraose b (LSTb); sialyllacto-N-tetraose c (LSTc); sialyllacto-N-tetraose d (LSTd); lacto-N-fucopentaose I; lacto-N-neofucopentaose; lacto-N-fucopentaose II; lacto-N-fucopentaose III; lacto-N-fucopentaose V; lacto-N-neofucopentaose V; lacto-N-difucohexaose I; lacto-N-neodifucohexaose; lacto-N-difucohexaose II; monofucosyllacto-n-hexaose III; difucosyllacto-N-hexaose a; 6′-galactosyllactose; 3′-galactosyllactose; lacto-N-hexaose; lacto-N-neohexaose; 2′-fucosyllactose (2′FL); 3-fucosyllactose (3-FL); difucosyllactose (DiFL); chitosan; chitosan comprising oligosaccharide; heparosan; glycosaminoglycan oligosaccharide; heparin; heparan sulphate; chondroitin sulphate; dermatan sulphate; hyaluronan; hyaluronic acid and keratan sulphate; preferably, the one or more bioproduct(s) is/are one or more LN3 derived oligosaccharide(s).

38. A saccharide importer having uptake activity for LN3 for use in the production of one or more bioproduct(s) according to embodiment 37, wherein the saccharide importer further comprises uptake activity for one or more other saccharide(s) different from LN3, wherein the one or more other saccharide(s) different from LN3 are chosen from the list comprising monosaccharide, disaccharide, oligosaccharide and polysaccharide.

39. An isolated nucleic acid molecule encoding a saccharide importer having uptake activity for lacto-N-triose (LN3, GlcNAc-beta1,3-Gal-beta1,4-Glc) wherein the saccharide importer:
originates from the major facilitator superfamily (MFS) of transporters,
comprises a polypeptide sequence comprising an IPR001927 domain as defined by InterPro 90.0 as released on 4 Aug. 2022,
comprises a polypeptide sequence that is at least 25% identical over a stretch of at least 150 amino acid residues, preferably at least 200 amino acid residues, to any one of the polypeptide sequences as represented by SEQ ID NOs: 04, 12, 02, 01, 03, 05, 06, 07, 08, 09, 10, 11 or 13,
comprises a polypeptide sequence that is at least 25% identical to any one of the full-length polypeptide sequences as represented by SEQ ID NOs: 04, 12, 02, 01, 03, 05, 06, 07, 08, 09, 10, 11 or 13,
comprises a polypeptide sequence as represented by any one of SEQ ID NOs: 04, 12, 02, 01, 03, 05, 06, 07, 08, 09, 10, 11 or 13,
comprises a polypeptide sequence that has a deletion, insertion and/or a mutation of one or more amino acid residue(s) N-terminally from the first transmembrane (TM) domain of any one of SEQ ID NOs: 04, 12, 02, 01, 03, 05, 06, 07, 08, 09, 10, 11 or 13, preferably the deletion, insertion and/or mutation is in or N-terminally of a non-TM helix, and/or
comprises a polypeptide sequence that has a deletion, insertion and/or a mutation of one or more amino acid residue(s) C-terminally from the last transmembrane (TM) domain of any one of SEQ ID NOs: 04, 12, 02, 01, 03, 05, 06, 07, 08, 09, 10, 11 or 13, preferably the deletion, insertion and/or mutation is in or C-terminally of a non-TM helix.

40. A vector comprising the isolated nucleic acid molecule of embodiment 39.

41. Use of an isolated nucleic acid molecule according to embodiment 39 for production of one or more bioproduct(s) chosen from the list comprising saccharide; monosaccharide; activated monosaccharide; phosphorylated monosaccharide; disaccharide; oligosaccharide; polysaccharide; milk saccharide; mammalian milk saccharide; MMO; human milk saccharide; HMO; neutral (non-charged) saccharide; negatively charged saccharide; fucosylated saccharide; sialylated saccharide; neutral (non-charged) oligosaccharide; negatively charged oligosaccharide; fucosylated oligosaccharide; sialylated oligosaccharide; N-acetylglucosamine containing oligosaccharide; N-acetyllactosamine containing oligosaccharide; lacto-N-biose containing oligosaccharide; lactose containing oligosaccharide; non-fucosylated neutral (non-charged) oligosaccharide; O-antigen; ECA; the oligosaccharide repeats present in capsular polysaccharides; peptidoglycan; amino-sugar; Lewis-type antigen oligosaccharide; antigen of the human ABO blood group system; animal oligosaccharide, preferably selected from the group consisting of N-glycans and O-glycans; plant oligosaccharide, preferably selected from the group consisting of N-glycans and O-glycans; LN3; lacto-N-tetraose (LNT); LNnT; oligosaccharide derived from LN3; 3′sialyllactose (3′SL); 6′sialyllactose (6′SL); sialyllacto-N-tetraose a (LSTa); sialyllacto-N-tetraose b (LSTb); sialyllacto-N-tetraose c (LSTc); sialyllacto-N-tetraose d (LSTd); lacto-N-fucopentaose I; lacto-N-neofucopentaose; lacto-N-fucopentaose II; lacto-N-fucopentaose III; lacto-N-fucopentaose V; lacto-N-neofucopentaose V; lacto-N-difucohexaose I; lacto-N-neodifucohexaose; lacto-N-difucohexaose II; monofucosyllacto-n-hexaose III; difucosyllacto-N-hexaose a; 6′-galactosyllactose; 3′-galactosyllactose; lacto-N-hexaose; lacto-N-neohexaose; 2′-fucosyllactose (2′FL); 3-fucosyllactose (3-FL); difucosyllactose (DiFL); chitosan; chitosan comprising oligosaccharide; heparosan; glycosaminoglycan oligosaccharide; heparin; heparan sulphate; chondroitin sulphate; dermatan sulphate; hyaluronan; hyaluronic acid and keratan sulphate; preferably, the one or more bioproduct(s) is/are one or more LN3 derived oligosaccharide(s).

42. Use of a vector according to embodiment 40 for production of one or more bioproduct(s) chosen from the list comprising saccharide, monosaccharide, activated monosaccharide, phosphorylated monosaccharide, disaccharide, oligosaccharide, polysaccharide, a milk saccharide, a mammalian milk saccharide, an MMO, a human milk saccharide, an HMO, a neutral (non-charged) saccharide, a negatively charged saccharide, a fucosylated saccharide, a sialylated saccharide, a neutral (non-charged) oligosaccharide, a negatively charged oligosaccharide, a fucosylated oligosaccharide, a sialylated oligosaccharide, N-acetylglucosamine containing oligosaccharide, N-acetyllactosamine containing oligosaccharide, lacto-N-biose containing oligosaccharide; lactose containing oligosaccharide, non-fucosylated neutral (non-charged) oligosaccharide; O-antigen, ECA, the oligosaccharide repeats present in capsular polysaccharides, peptidoglycan, an amino-sugar, a Lewis-type antigen oligosaccharide, an antigen of the human ABO blood group system, an animal oligosaccharide, preferably selected from the group consisting of N-glycans and O-glycans, a plant oligosaccharide, preferably selected from the group consisting of N-glycans and O-glycans; LN3; lacto-N-tetraose (LNT), LNnT; an oligosaccharide derived from LN3; 3′sialyllactose (3′SL); 6′sialyllactose (6′SL); sialyllacto-N-tetraose a (LSTa); sialyllacto-N-tetraose b (LSTb); sialyllacto-N-tetraose c (LSTc); sialyllacto-N-tetraose d (LSTd); lacto-N-fucopentaose I; lacto-N-neofucopentaose; lacto-N-fucopentaose II; lacto-N-fucopentaose III; lacto-N-fucopentaose V; lacto-N-neofucopentaose V; lacto-N-difucohexaose I; lacto-N-neodifucohexaose; lacto-N-difucohexaose II; monofucosyllacto-n-hexaose III; difucosyllacto-N-hexaose a; 6′-galactosyllactose; 3′-galactosyllactose; lacto-N-hexaose; lacto-N-neohexaose; 2′-fucosyllactose (2′FL); 3-fucosyllactose (3-FL); difucosyllactose (DiFL); chitosan; chitosan comprising oligosaccharide; heparosan; chondroitin sulphate; glycosaminoglycan oligosaccharide; heparin; heparan sulphate; chondroitin sulphate; dermatan sulphate; hyaluronan; hyaluronic acid; and keratan sulphate; preferably, the one or more bioproduct(s) is/are one or more LN3 derived oligosaccharide(s).

43. Use of a saccharide importer having uptake activity for lacto-N-triose (LN3, GlcNAc-beta1,3-Gal-beta1,4-Glc), wherein the saccharide importer:
originates from the major facilitator superfamily (MFS) of transporters,
comprises a polypeptide sequence comprising an IPR001927 domain as defined by InterPro 90.0 as released on 4 Aug. 2022,
comprises a polypeptide sequence that is at least 25% identical over a stretch of at least 150 amino acid residues, preferably at least 200 amino acid residues, to any one of the polypeptide sequences as represented by SEQ ID NOs: 04, 12, 02, 01, 03, 05, 06, 07, 08, 09, 10, 11 or 13,
comprises a polypeptide sequence that is at least 25% identical to any one of the full-length polypeptide sequences as represented by SEQ ID NOs: 04, 12, 02, 01, 03, 05, 06, 07, 08, 09, 10, 11 or 13,
comprises a polypeptide sequence as represented by any one of SEQ ID NOs: 04, 12, 02, 01, 03, 05, 06, 07, 08, 09, 10, 11 or 13,
comprises a polypeptide sequence that has a deletion, insertion and/or a mutation of one or more amino acid residue(s) N-terminally from the first transmembrane (TM) domain of any one of SEQ ID NOs: 04, 12, 02, 01, 03, 05, 06, 07, 08, 09, 10, 11 or 13, preferably the deletion, insertion and/or mutation is in or N-terminally of a non-TM helix, and/or
comprises a polypeptide sequence that has a deletion, insertion and/or a mutation of one or more amino acid residue(s) C-terminally from the last transmembrane (TM) domain of any one of SEQ ID NOs: 04, 12, 02, 01, 03, 05, 06, 07, 08, 09, 10, 11 or 13, preferably the deletion, insertion and/or mutation is in or C-terminally of a non-TM helix,
for production of one or more bioproduct(s) chosen from the list comprising saccharide; monosaccharide; activated monosaccharide; phosphorylated monosaccharide; disaccharide; oligosaccharide; polysaccharide; milk saccharide; mammalian milk saccharide; MMO; human milk saccharide; HMO; neutral (non-charged) saccharide; negatively charged saccharide; fucosylated saccharide; sialylated saccharide; neutral (non-charged) oligosaccharide; negatively charged oligosaccharide; fucosylated oligosaccharide; sialylated oligosaccharide; N-acetylglucosamine containing oligosaccharide; N-acetyllactosamine containing oligosaccharide; lacto-N-biose containing oligosaccharide; lactose containing oligosaccharide; non-fucosylated neutral (non-charged) oligosaccharide; O-antigen; ECA; the oligosaccharide repeats present in capsular polysaccharides; peptidoglycan; amino-sugar; Lewis-type antigen oligosaccharide; antigen of the human ABO blood group system; animal oligosaccharide, preferably selected from the group consisting of N-glycans and O-glycans; plant oligosaccharide, preferably selected from the group consisting of N-glycans and O-glycans; LN3; lacto-N-tetraose (LNT); LNnT; oligosaccharide derived from LN3; 3′sialyllactose (3′SL); 6′sialyllactose (6′SL); sialyllacto-N-tetraose a (LSTa); sialyllacto-N-tetraose b (LSTb); sialyllacto-N-tetraose c (LSTc); sialyllacto-N-tetraose d (LSTd); lacto-N-fucopentaose I; lacto-N-neofucopentaose; lacto-N-fucopentaose II; lacto-N-fucopentaose III; lacto-N-fucopentaose V; lacto-N-neofucopentaose V; lacto-N-difucohexaose I; lacto-N-neodifucohexaose; lacto-N-difucohexaose II; monofucosyllacto-n-hexaose III; difucosyllacto-N-hexaose a; 6′-galactosyllactose; 3′-galactosyllactose; lacto-N-hexaose; lacto-N-neohexaose; 2′-fucosyllactose (2′FL); 3-fucosyllactose (3-FL); difucosyllactose (DiFL); chitosan; chitosan comprising oligosaccharide; heparosan; glycosaminoglycan oligosaccharide; heparin; heparan sulphate; chondroitin sulphate; dermatan sulphate; hyaluronan; hyaluronic acid and keratan sulphate; preferably, the one or more bioproduct(s) is/are one or more LN3 derived oligosaccharide(s).

More specifically, this disclosure relates to the following preferred specific embodiments:

1. A cell for the production of one or more bioproduct(s), wherein the cell:
is capable to produce, preferably produces, one or more precursor(s) used in the production of at least one of the one or more bioproduct(s), preferably the cell is genetically engineered to produce at least one of the one or more precursor(s) used in the production of at least one of the one or more bioproduct(s), more preferably the cell is genetically engineered to produce all of the one or more precursor(s) used in the production of at least one of the one or more bioproduct(s), and
is genetically engineered to express, preferably to overexpress, a polynucleotide sequence that encodes a saccharide importer that internalizes at least one of the one or more precursor(s) into the cell.

2. Cell according to specific embodiment 1, wherein the saccharide importer has uptake activity for lacto-N-triose (LN3, GlcNAc-beta1,3-Gal-beta1,4-Glc).

3. Cell according to any one of specific embodiment 1 or 2, wherein:
one of the one or more precursor(s) is LN3, or
one of the one or more precursor(s) is LN3 and the saccharide importer has uptake activity for the LN3 being produced by the cell.

4. A cell for the production of one or more bioproduct(s), wherein the cell is genetically engineered to express, preferably to overexpress, a polynucleotide sequence that encodes a saccharide importer that has uptake activity for lacto-N-triose (LN3, GlcNAc-beta1,3-Gal-beta1,4-Glc).

5. Cell according to any one of previous specific embodiments, wherein the saccharide importer comprises a polypeptide sequence:
that originates from the major facilitator superfamily (MFS) of transporters,
comprising an IPR domain selected from the list comprising IPR001927, IPR002178, IPR016152, IPR018043, IPR020846, IPR036259 and IPR039672 as defined by InterPro 90.0 as released on 4 Aug. 2022,
comprising PF13347 domain and/or PF00359 domain as defined by PFAM 32.0 as released on September 2018,
comprising a PANTHER domain selected from the list comprising PTHR11328, PTHR11328:SF24, PTHR11328:SF36 and PTHR11328:SF39 as defined by PANTHER 18.0 as released on 17 Sep. 2023,
comprising cd17332 domain and/or cd00211 domain as defined by the Conserved Domain Database CDD 3.20 as released on September 2022,
that is at least 25%, at least 30%, at least 35%, at least 40%, at least 45%, at least 50%, at least 55%, at least 60%, at least 65%, at least 70%, at least 75%, at least 80% identical over a stretch of at least 50 amino acid residues, at least 100 amino acid residues, at least 150 amino acid residues, at least 200 amino acid residues, at least 250 amino acid residues to any one of the polypeptide sequences as represented by SEQ ID NOs: 04, 12, 02, 01, 03, 09, 16, 19, 20 or 21,
that is at least 25%, at least 30%, at least 35%, at least 40%, at least 45%, at least 50%, at least 55%, at least 60%, at least 65%, at least 70%, at least 75%, at least 80% identical to any one of the full-length polypeptide sequences as represented by SEQ ID NOs: 04, 12, 02, 01, 03, 09, 16, 19, 20 or 21,
as represented by any one of SEQ ID NOs: 04, 12, 02, 01, 03, 09, 16, 19, 20 or 21,
that has a deletion, insertion and/or a mutation of one or more amino acid residue(s) N-terminally from the first transmembrane (TM) domain of any one of SEQ ID NOs: 12, 02, 01, 09, 19, 20 or 21, preferably the deletion, insertion and/or mutation is in or N-terminally of a non-TM helix, and/or
that has a deletion, insertion and/or a mutation of one or more amino acid residue(s) C-terminally from the last transmembrane (TM) domain of any one of SEQ ID NOs: 12, 02, 01, 09, 19, 20 or 21, preferably the deletion, insertion and/or mutation is in or C-terminally of a non-TM helix.

6. Cell according to any one of previous specific embodiments, wherein the saccharide importer originates from the major facilitator superfamily (MFS) of transporters and comprises a polypeptide sequence comprising:
an IPR001927 domain as defined by InterPro 90.0 as released on 4 Aug. 2022, and
comprises a polypeptide sequence comprising 12 transmembrane (TM) domains with a conserved domain [AGSV][HNQ][ACDEGNQSTV]XX[FWY]XXXXX(no L) as represented by SEQ ID NO: 17, wherein X can be any amino acid residue, present in the first TM domain, preferably with a conserved domain [AGS]Q[ACGNQSTV]XX[FWY] as represented by SEQ ID NO:18, wherein X can be any amino acid residue, wherein the second amino acid residue of SEQ ID NO: 17, preferably the second amino acid residue of SEQ ID NO:18, is aligned to Lys18 of the polypeptide with SEQ ID NO:22.

7. Cell according to any one of specific embodiments 2 to 6, wherein the saccharide importer further comprises uptake activity for one or more other saccharide(s) different from LN3, wherein the one or more other saccharide(s) different from LN3 are chosen from the list comprising monosaccharide, disaccharide, oligosaccharide and polysaccharide.

8. Cell according to any one of specific embodiments 2 to 7, wherein the saccharide importer has uptake activity for LN3 but not for a) LNT (lacto-N-tetraose, Galβ1-3GlcNAcβ1-3Galβ1-4Glc) and/or b) LNnT (lacto-N-neotetraose, Galβ1-4GlcNAcβ1-3Galβ1-4Glc).

9. Cell according to any one of previous specific embodiments, wherein the one or more bioproduct(s) is/are chosen from the list comprising saccharide; monosaccharide; activated monosaccharide; phosphorylated monosaccharide; disaccharide; oligosaccharide; polysaccharide; milk saccharide; mammalian milk saccharide; MMO; human milk saccharide; HMO; neutral (non-charged) saccharide; negatively charged saccharide; fucosylated saccharide; sialylated saccharide; neutral (non-charged) oligosaccharide; negatively charged oligosaccharide; fucosylated oligosaccharide; sialylated oligosaccharide; N-acetylglucosamine containing oligosaccharide; N-acetyllactosamine containing oligosaccharide; lacto-N-biose containing oligosaccharide; lactose containing oligosaccharide; non-fucosylated neutral (non-charged) oligosaccharide; O-antigen; ECA; the oligosaccharide repeats present in capsular polysaccharides; peptidoglycan; amino-sugar; Lewis-type antigen oligosaccharide; antigen of the human ABO blood group system; animal oligosaccharide, preferably selected from the group consisting of N-glycans and O-glycans; plant oligosaccharide, preferably selected from the group consisting of N-glycans and O-glycans; LN3; lacto-N-tetraose (LNT); LNnT; oligosaccharide derived from LN3; 3′sialyllactose (3′SL); 6′sialyllactose (6′SL); sialyllacto-N-tetraose a (LSTa); sialyllacto-N-tetraose b (LSTb); sialyllacto-N-tetraose c (LSTc); sialyllacto-N-tetraose d (LSTd); lacto-N-fucopentaose I; lacto-N-neofucopentaose; lacto-N-fucopentaose II; lacto-N-fucopentaose III; lacto-N-fucopentaose V; lacto-N-neofucopentaose V; lacto-N-difucohexaose I; lacto-N-neodifucohexaose; lacto-N-difucohexaose II; monofucosyllacto-n-hexaose III; difucosyllacto-N-hexaose a; 6′-galactosyllactose; 3′-galactosyllactose; lacto-N-hexaose; lacto-N-neohexaose; 2′-fucosyllactose (2′FL); 3-fucosyllactose (3-FL); difucosyllactose (DiFL); chitosan; chitosan comprising oligosaccharide; heparosan; glycosaminoglycan oligosaccharide; heparin; heparan sulphate; chondroitin sulphate; dermatan sulphate; hyaluronan; hyaluronic acid and keratan sulphate, preferably, the one or more bioproduct(s) is/are one or more LN3 derived oligosaccharide(s).

10. Cell according to any one of previous specific embodiments, wherein the cell is further genetically engineered for the production of the one or more bioproduct(s).

11. Cell according to any one of specific embodiments 4 to 10, wherein the cell is capable to produce, preferably produces, the one or more bioproduct(s) from one or more precursor(s).

12. Cell according to specific embodiment 11, wherein the cell is capable to produce, preferably produces, at least one of the one or more precursor(s), preferably, the cell is genetically engineered for the production of at least one of the one or more precursor(s); more preferably the cell is capable to produce, preferably produces, all of the one or more precursor(s), even more preferably, the cell is genetically engineered for the production of all of the one or more precursor(s).

13. Cell according to any one of specific embodiment 11 or 12, wherein at least one of the one or more precursor(s) is internalized in the cell via the saccharide importer.

14. Cell according to any one of specific embodiments 1 to 3, 11 to 13, wherein the one or more precursor(s) is/are chosen from the list comprising saccharide; monosaccharide; activated monosaccharide; phosphorylated monosaccharide; disaccharide; oligosaccharide; polysaccharide; milk saccharide; mammalian milk saccharide; MMO; human milk saccharide; HMO; neutral (non-charged) saccharide; negatively charged saccharide; fucosylated saccharide; sialylated saccharide; neutral (non-charged) oligosaccharide; negatively charged oligosaccharide; fucosylated oligosaccharide; sialylated oligosaccharide; N-acetylglucosamine containing oligosaccharide; N-acetyllactosamine containing oligosaccharide; lacto-N-biose containing oligosaccharide; lactose containing oligosaccharide; non-fucosylated neutral (non-charged) oligosaccharide; O-antigen; ECA; the oligosaccharide repeats present in capsular polysaccharides; peptidoglycan; amino-sugar; Lewis-type antigen oligosaccharide; antigen of the human ABO blood group system; animal oligosaccharide, preferably selected from the group consisting of N-glycans and O-glycans; plant oligosaccharide, preferably selected from the group consisting of N-glycans and O-glycans; LN3; lacto-N-tetraose (LNT); LNnT; oligosaccharide derived from LN3; 3′sialyllactose (3′SL); 6′sialyllactose (6′SL); sialyllacto-N-tetraose a (LSTa); sialyllacto-N-tetraose b (LSTb); sialyllacto-N-tetraose c (LSTc); sialyllacto-N-tetraose d (LSTd); lacto-N-fucopentaose I; lacto-N-neofucopentaose; lacto-N-fucopentaose II; lacto-N-fucopentaose III; lacto-N-fucopentaose V; lacto-N-neofucopentaose V; lacto-N-difucohexaose I; lacto-N-neodifucohexaose; lacto-N-difucohexaose II; monofucosyllacto-n-hexaose III; difucosyllacto-N-hexaose a; 6′-galactosyllactose; 3′-galactosyllactose; lacto-N-hexaose; lacto-N-neohexaose; 2′-fucosyllactose (2′FL); 3-fucosyllactose (3-FL); difucosyllactose (DiFL); chitosan; chitosan comprising oligosaccharide; heparosan; glycosaminoglycan oligosaccharide; heparin; heparan sulphate; chondroitin sulphate; dermatan sulphate; hyaluronan; hyaluronic acid and keratan sulphate; preferably, the one or more bioproduct(s) is/are one or more LN3 derived oligosaccharide(s).

15. Cell according to any one of previous specific embodiments, wherein the cell:
i) possesses, preferably expresses, more preferably overexpresses, one or more glycosyltransferase(s) chosen from the list comprising fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases and fucosaminyltransferases,
preferably, the fucosyltransferase is chosen from the list comprising alpha-1,2-fucosyltransferase, alpha-1,3-fucosyltransferase, alpha-1,3/4-fucosyltransferase, alpha-1,4-fucosyltransferase and alpha-1,6-fucosyltransferase,
preferably, the sialyltransferase is chosen from the list comprising alpha-2,3-sialyltransferase, alpha-2,6-sialyltransferase and alpha-2,8-sialyltransferase,
preferably, the galactosyltransferase is chosen from the list comprising beta-1,3-galactosyltransferase, N-acetylglucosamine beta-1,3-galactosyltransferase, beta-1,4-galactosyltransferase, N-acetylglucosamine beta-1,4-galactosyltransferase, alpha-1,3-galactosyltransferase and alpha-1,4-galactosyltransferase,
preferably, the glucosyltransferase is chosen from the list comprising alpha-glucosyltransferase, beta-1,2-glucosyltransferase, beta-1,3-glucosyltransferase and beta-1,4-glucosyltransferase,
preferably, the mannosyltransferase is chosen from the list comprising alpha-1,2-mannosyltransferase, alpha-1,3-mannosyltransferase and alpha-1,6-mannosyltransferase,
preferably, the N-acetylglucosaminyltransferase is chosen from the list comprising galactoside beta-1,3-N-acetylglucosaminyltransferase and beta-1,6-N-acetylglucosaminyltransferase,
preferably, the N-acetylgalactosaminyltransferase is an alpha-1,3-N-acetylgalactosaminyltransferase,
ii) is capable to produce, preferably produces, one or more nucleotide-activated sugars preferably chosen from the list comprising UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), GDP-fucose, (GDP-Fuc), UDP-glucuronate, UDP-galacturonate, UDP-2-acetamido-2,6-dideoxy--L-arabino-4-hexulose, UDP-2-acetamido-2,6-dideoxy--L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid (CMP-Neu5Ac), CMP-Neu4Ac, CMP-Neu5Ac9N₃, CMP-Neu4,5Ac₂, CMP-Neu5,7Ac₂, CMP-Neu5,9Ac₂, CMP-Neu5,7(8,9) Ac₂, CMP-N-glycolylneuraminic acid (CMP-Neu5Gc), GDP-rhamnose and UDP-xylose, preferably the cell is genetically engineered for production of one or more of the nucleotide-activated sugar(s),
iii) comprises at least one pathway chosen from the list comprising fucosylation pathway, sialylation pathway, galactosylation pathway, N-acetylglucosaminylation pathway, N-acetylgalactosaminylation pathway, mannosylation pathway and N-acetylmannosaminylation pathway, preferably the cell is genetically engineered to comprise at least one of the pathway(s), more preferably the cell comprises at least one of the pathway(s) wherein at least one of the pathway(s) has/have been genetically engineered, and/or
iv) comprises a catabolic pathway for selected mono-, di- or oligosaccharides which is at least partially inactivated, the mono-, di-, or oligosaccharides being involved in and/or required for the production of the one or more bioproduct(s).

16. Cell according to any one of previous specific embodiments, wherein the cell is selected from the group consisting of prokaryotic cells and eukaryotic cells, preferably from the group consisting of yeast cells, bacterial cells, archaebacterial cells, algae cells, and fungal cells.

17. Cell according to any one of previous specific embodiments, wherein the cell is a bacterium, fungus, yeast, a plant cell, an animal cell, or a protozoan cell,
preferably, the bacterium belongs to a phylum chosen from the group comprising Proteobacteria, Firmicutes, Cyanobacteria, Deinococcus-Thermus and Actinobacteria; more preferably, the bacterium belongs to a family chosen from the group comprising Enterobacteriaceae, Bacillaceae, Lactobacillus, Corynebacteriaceae and Vibrionaceae; even more preferably, the bacterium is chosen from the list comprising an Escherichia coli strain, a Bacillus subtilis strain, a Vibrio natriegens strain; even more preferably the Escherichia coli strain is a K-12 strain, most preferably the Escherichia coli K-12 strain is E. coli MG1655,
preferably, the fungus belongs to a genus chosen from the group comprising Rhizopus, Dictyostelium, Penicillium, Mucor or Aspergillus,
preferably, the yeast belongs to a genus chosen from the group comprising Saccharomyces, Zygosaccharomyces, Pichia, Komagataella, Hansenula, Yarrowia, Starmerella, Kluyveromyces, Debaromyces, Candida, Schizosaccharomyces, Schwanniomyces or Torulaspora; more preferably, the yeast is selected from the group consisting of: Saccharomyces cerevisiae, Hansenula polymorpha, Kluyveromyces lactis, Kluyveromyces marxianus, Pichia pastoris, Pichia methanolica, Pichia stipites, Candida boidinii, Schizosaccharomyces pombe, Schwanniomyces occidentalis, Torulaspora delbrueckii, Yarrowia lipolytica, Zygosaccharomyces rouxii, and Zygosaccharomyces bailii,
preferably, the plant cell is an algal cell or is derived from tobacco, alfalfa, rice, tomato, cotton, rapeseed, soy, maize, or corn plant,
preferably, the animal cell is derived from insects, amphibians, reptiles, invertebrates, fish, birds or mammalian cells excluding human embryonic stem cells, more preferably the mammalian cell is chosen from the list comprising an epithelial cell, an embryonic kidney cell, a fibroblast cell, a COS cell, a Chinese hamster ovary (CHO) cell, a murine myeloma cell, an NIH-3T3 cell, a lactocyte derived from mammalian induced pluripotent stem cells, more preferably the mammalian induced pluripotent stem cells are human induced pluripotent stem cells, a post-parturition mammary epithelium cell, a polarized mammary cell, more preferably the polarized mammary cell is selected from the group comprising live primary mammary epithelial cells, live mammary myoepithelial cells, live mammary progenitor cells, live immortalized mammary epithelial cells, live immortalized mammary myoepithelial cells, live immortalized mammary progenitor cells, a non-mammary adult stem cell or derivatives thereof, more preferably the insect cell is derived from Spodoptera frugiperda, Bombyx mori, Mamestra brassicae, Trichoplusia ni or Drosophila melanogaster,
preferably, the protozoan cell is a Leishmania tarentolae cell.

18. Cell according to any one of previous specific embodiments, wherein the cell is an E. coli or yeast with a lactose permease positive phenotype, preferably wherein the lactose permease is coded by the gene LacY or LAC12, respectively.

19. Method for the production of one or more bioproduct(s), the method comprising the steps of:
a) cultivating and/or incubating a cell, preferably a single cell, according to any one of specific embodiments 1 to 18 under conditions permissive to express the saccharide importer and to produce the one or more bioproduct(s),
b) preferably, separating, preferably purifying, the one or more bioproduct(s) from the cultivation or incubation.

20. Method according to specific embodiment 19, wherein the one or more bioproduct(s) is/are chosen from the list comprising saccharide; monosaccharide; activated monosaccharide; phosphorylated monosaccharide; disaccharide; oligosaccharide; polysaccharide; milk saccharide; mammalian milk saccharide; MMO; human milk saccharide; HMO; neutral (non-charged) saccharide; negatively charged saccharide; fucosylated saccharide; sialylated saccharide; neutral (non-charged) oligosaccharide; negatively charged oligosaccharide; fucosylated oligosaccharide; sialylated oligosaccharide; N-acetylglucosamine containing oligosaccharide; N-acetyllactosamine containing oligosaccharide; lacto-N-biose containing oligosaccharide; lactose containing oligosaccharide; non-fucosylated neutral (non-charged) oligosaccharide; O-antigen; ECA; the oligosaccharide repeats present in capsular polysaccharides; peptidoglycan; amino-sugar; Lewis-type antigen oligosaccharide; antigen of the human ABO blood group system; animal oligosaccharide, preferably selected from the group consisting of N-glycans and O-glycans; plant oligosaccharide, preferably selected from the group consisting of N-glycans and O-glycans; LN3; lacto-N-tetraose (LNT); LNnT; oligosaccharide derived from LN3; 3′sialyllactose (3′SL); 6′sialyllactose (6′SL); sialyllacto-N-tetraose a (LSTa); sialyllacto-N-tetraose b (LSTb); sialyllacto-N-tetraose c (LSTc); sialyllacto-N-tetraose d (LSTd); lacto-N-fucopentaose I; lacto-N-neofucopentaose; lacto-N-fucopentaose II; lacto-N-fucopentaose III; lacto-N-fucopentaose V; lacto-N-neofucopentaose V; lacto-N-difucohexaose I; lacto-N-neodifucohexaose; lacto-N-difucohexaose II; monofucosyllacto-n-hexaose III; difucosyllacto-N-hexaose a; 6′-galactosyllactose; 3′-galactosyllactose; lacto-N-hexaose; lacto-N-neohexaose; 2′-fucosyllactose (2′FL); 3-fucosyllactose (3-FL); difucosyllactose (DiFL); chitosan; chitosan comprising oligosaccharide; heparosan; glycosaminoglycan oligosaccharide; heparin; heparan sulphate; chondroitin sulphate; dermatan sulphate; hyaluronan; hyaluronic acid and keratan sulphate; preferably, the one or more bioproduct(s) is/are one or more LN3 derived oligosaccharide(s).

21. Method according to any one of specific embodiment 19 or 20, wherein the cell produces the one or more bioproduct(s) with a higher yield and/or higher purity compared to a cell with the same genetic make-up but lacking expression of the saccharide importer.

22. Method according to any one of specific embodiments 19 to 21, wherein the one or more bioproduct(s) is/are one or more LN3-derived oligosaccharide(s) and wherein the cell produces the one or more LN3-derived oligosaccharide(s) with a higher yield and/or higher purity compared to a cell with the same genetic make-up but lacking expression of the saccharide importer.

23. Method according to any one of specific embodiments 19 to 22, wherein the:
cultivation medium contains at least one carbon source selected from the group consisting of glucose, fructose, sucrose and glycerol,
cultivation or incubation medium contains at least one compound selected from the group consisting of lactose, galactose, glucose, UDP-GlcNAc, GlcNAc, UDP-Gal, UDP-Glc and LN3, and/or
cultivation or incubation medium comprises one or more precursor(s) that is/are used for production of the one or more bioproduct(s).

24. Method according to specific embodiment 23, wherein the one or more precursor(s) in the cultivation or incubation medium is/are produced by the cell and/or wherein the one or more precursor(s) is/are taken up by the cell via the saccharide importer.

25. Method according to any one of specific embodiments 19 to 24, wherein the one or more bioproduct(s), preferably all of the bioproduct(s), is/are recovered from the cultivation or incubation medium and/or the cell.

26. Method according to any one of specific embodiments 19 to 25, wherein the method results in a production of at least 50 g/L, preferably at least 75 g/L, more preferably at least 90 g/L of the one or more bioproduct(s) in the final volume of the cultivation or incubation.

27. Use of a cell according to any one of specific embodiments 1 to 18 for production of one or more bioproduct(s), preferably the one or more bioproduct(s) is/are one or more LN3 derived oligosaccharide(s).

28. Use of a method according to any one of specific embodiments 19 to 26 for production of one or more bioproduct(s), preferably the one or more bioproduct(s) is/are one or more LN3 derived oligosaccharide(s).

29. A saccharide importer having uptake activity for lacto-N-triose (LN3, GlcNAc-beta1,3-Gal-beta1,4-Glc) and comprising a polypeptide sequence that has a deletion, insertion and/or a mutation of one or more amino acid residue(s):
N-terminally from the first transmembrane (TM) domain of any one of SEQ ID NOs: 12, 02, 01, 09, 19, 20 or 21, preferably the deletion, insertion and/or mutation is in or N-terminally of a non-TM helix and/or
C-terminally from the last TM domain of any one of SEQ ID NOs: 12, 02, 01, 09, 19, 20 or 21, preferably the deletion, insertion and/or mutation is in or C-terminally of a non-TM helix.

30. A saccharide importer according to specific embodiment 29, wherein the saccharide importer further comprises uptake activity for one or more other saccharide(s) different from LN3, wherein the one or more other saccharide(s) different from LN3 are chosen from the list comprising monosaccharide, disaccharide, oligosaccharide and polysaccharide.

31. A saccharide importer according to any one of specific embodiment 29 or 30, wherein the saccharide importer has uptake activity for LN3 but not for a) LNT and/or b) LNnT.

32. Use of a saccharide importer having uptake activity for lacto-N-triose (LN3, GlcNAc-beta1,3-Gal-beta1,4-Glc) for the production of one or more bioproduct(s), wherein the saccharide importer comprises a polypeptide sequence:
that originates from the major facilitator superfamily (MFS) of transporters,
comprising an IPR domain selected from the list comprising IPR001927, IPR002178, IPR016152, IPR018043, IPR020846, IPR036259 and IPR039672 as defined by InterPro 90.0 as released on 4 Aug. 2022,
comprising PF13347 domain and/or PF00359 domain as defined by PFAM 32.0 as released on September 2018,
comprising a PANTHER domain selected from the list comprising PTHR 11328, PTHR11328:SF24, PTHR11328:SF36 and PTHR11328:SF39 as defined by PANTHER 18.0 as released on 17 Sep. 2023,
comprising cd17332 domain and/or cd00211 domain as defined by the Conserved Domain Database CDD 3.20 as released on September 2022,
that is at least 25%, at least 30%, at least 35%, at least 40%, at least 45%, at least 50%, at least 55%, at least 60%, at least 65%, at least 70%, at least 75%, at least 80% identical over a stretch of at least 50 amino acid residues, at least 100 amino acid residues, at least 150 amino acid residues, at least 200 amino acid residues, at least 250 amino acid residues to any one of the polypeptide sequences as represented by SEQ ID NOs: 04, 12, 02, 01, 03, 09, 16, 19, 20 or 21,
that is at least 25%, at least 30%, at least 35%, at least 40%, at least 45%, at least 50%, at least 55%, at least 60%, at least 65%, at least 70%, at least 75%, at least 80% identical to any one of the full-length polypeptide sequences as represented by SEQ ID NOs: 04, 12, 02, 01, 03, 09, 16, 19, 20 or 21,
as represented by any one of SEQ ID NOs: 04, 12, 02, 01, 03, 09, 16, 19, 20 or 21,
that has a deletion, insertion and/or a mutation of one or more amino acid residue(s) N-terminally from the first transmembrane (TM) domain of any one of SEQ ID NOs: 12, 02, 01, 09, 19, 20 or 21, preferably the deletion, insertion and/or mutation is in or N-terminally of a non-TM helix, and/or
that has a deletion, insertion and/or a mutation of one or more amino acid residue(s) C-terminally from the last transmembrane (TM) domain of any one of SEQ ID NOs: 12, 02, 01, 09, 19, 20 or 21, preferably the deletion, insertion and/or mutation is in or C-terminally of a non-TM helix,
and wherein the bioproduct(s) is/are chosen from the list comprising saccharide; monosaccharide; activated monosaccharide; phosphorylated monosaccharide; disaccharide; oligosaccharide; polysaccharide; milk saccharide; mammalian milk saccharide; MMO; human milk saccharide; HMO; neutral (non-charged) saccharide; negatively charged saccharide; fucosylated saccharide; sialylated saccharide; neutral (non-charged) oligosaccharide; negatively charged oligosaccharide; fucosylated oligosaccharide; sialylated oligosaccharide; N-acetylglucosamine containing oligosaccharide; N-acetyllactosamine containing oligosaccharide; lacto-N-biose containing oligosaccharide; lactose containing oligosaccharide; non-fucosylated neutral (non-charged) oligosaccharide; O-antigen; ECA; the oligosaccharide repeats present in capsular polysaccharides; peptidoglycan; amino-sugar; Lewis-type antigen oligosaccharide; antigen of the human ABO blood group system; animal oligosaccharide, preferably selected from the group consisting of N-glycans and O-glycans; plant oligosaccharide, preferably selected from the group consisting of N-glycans and O-glycans; LN3; lacto-N-tetraose (LNT); LNnT; oligosaccharide derived from LN3; 3′sialyllactose (3′SL); 6′sialyllactose (6′SL); sialyllacto-N-tetraose a (LSTa); sialyllacto-N-tetraose b (LSTb); sialyllacto-N-tetraose c (LSTc); sialyllacto-N-tetraose d (LSTd); lacto-N-fucopentaose I; lacto-N-neofucopentaose; lacto-N-fucopentaose II; lacto-N-fucopentaose III; lacto-N-fucopentaose V; lacto-N-neofucopentaose V; lacto-N-difucohexaose I; lacto-N-neodifucohexaose; lacto-N-difucohexaose II; monofucosyllacto-n-hexaose III; difucosyllacto-N-hexaose a; 6′-galactosyllactose; 3′-galactosyllactose; lacto-N-hexaose; lacto-N-neohexaose; 2′-fucosyllactose (2′FL); 3-fucosyllactose (3-FL); difucosyllactose (DiFL); chitosan; chitosan comprising oligosaccharide; heparosan; glycosaminoglycan oligosaccharide; heparin; heparan sulphate; chondroitin sulphate; dermatan sulphate; hyaluronan; hyaluronic acid and keratan sulphate; preferably, the one or more bioproduct(s) is/are one or more LN3 derived oligosaccharide(s).

33. Use according to specific embodiment 32, wherein the saccharide importer originates from the major facilitator superfamily (MFS) of transporters and comprises a polypeptide sequence comprising:
an IPR001927 domain as defined by InterPro 90.0 as released on 4 Aug. 2022, and
comprises a polypeptide sequence comprising 12 transmembrane (TM) domains with a conserved domain [AGSV][HNQ][ACDEGNQSTV]XX[FWY]XXXXX(no L) as represented by SEQ ID NO:17, wherein X can be any amino acid residue, present in the first TM domain, preferably with a conserved domain [AGS]Q[ACGNQSTV]XX[FWY] as represented by SEQ ID NO:18, wherein X can be any amino acid residue, wherein the second amino acid residue of SEQ ID NO: 17, preferably the second amino acid residue of SEQ ID NO: 18, is aligned to Lys18 of the polypeptide with SEQ ID NO:22.

34. Use according to any one of specific embodiments 32 or 33, wherein the saccharide importer further comprises uptake activity for one or more other saccharide(s) different from LN3, wherein the one or more other saccharide(s) different from LN3 are chosen from the list comprising monosaccharide, disaccharide, oligosaccharide and polysaccharide.

35. Use according to any one of specific embodiments 32 to 34, wherein saccharide importer has uptake activity for LN3 but not for a) LNT and/or b) LNnT.

36. An isolated nucleic acid molecule encoding a saccharide importer having uptake activity for lacto-N-triose (LN3, GlcNAc-beta1,3-Gal-beta1,4-Glc) wherein the saccharide importer comprises a polypeptide sequence:
that originates from the major facilitator superfamily (MFS) of transporters,
comprising an IPR domain selected from the list comprising IPR001927, IPR002178, IPR016152, IPR018043, IPR020846, IPR036259 and IPR039672 as defined by InterPro 90.0 as released on 4 Aug. 2022,
comprising PF13347 domain and/or PF00359 domain as defined by PFAM 32.0 as released on September 2018,
comprising a PANTHER domain selected from the list comprising PTHR 11328, PTHR11328:SF24, PTHR11328:SF36 and PTHR11328:SF39 as defined by PANTHER 18.0 as released on 17 Sep. 2023,
comprising cd17332 domain and/or cd00211 domain as defined by the Conserved Domain Database CDD 3.20 as released on September 2022,
that is at least 25%, at least 30%, at least 35%, at least 40%, at least 45%, at least 50%, at least 55%, at least 60%, at least 65%, at least 70%, at least 75%, at least 80% identical over a stretch of at least 50 amino acid residues, at least 100 amino acid residues, at least 150 amino acid residues, at least 200 amino acid residues, at least 250 amino acid residues to any one of the polypeptide sequences as represented by SEQ ID NOs: 04, 12, 02, 01, 03, 09, 16, 19, 20 or 21,
that is at least 25%, at least 30%, at least 35%, at least 40%, at least 45%, at least 50%, at least 55%, at least 60%, at least 65%, at least 70%, at least 75%, at least 80% identical to any one of the full-length polypeptide sequences as represented by SEQ ID NOs: 04, 12, 02, 01, 03, 09, 16, 19, 20 or 21,
as represented by any one of SEQ ID NOs: 04, 12, 02, 01, 03, 09, 16, 19, 20 or 21,
that has a deletion, insertion and/or a mutation of one or more amino acid residue(s) N-terminally from the first transmembrane (TM) domain of any one of SEQ ID NOs: 12, 02, 01, 09, 19, 20 or 21, preferably the deletion, insertion and/or mutation is in or N-terminally of a non-TM helix, and/or
that has a deletion, insertion and/or a mutation of one or more amino acid residue(s) C-terminally from the last transmembrane (TM) domain of any one of SEQ ID NOs: 12, 02, 01, 09, 19, 20 or 21, preferably the deletion, insertion and/or mutation is in or C-terminally of a non-TM helix.

37. Isolated nucleic acid molecule according to specific embodiment 36, wherein the saccharide importer originates from the major facilitator superfamily (MFS) of transporters and comprises a polypeptide sequence comprising:
an IPR001927 domain as defined by InterPro 90.0 as released on 4 Aug. 2022, and
comprises a polypeptide sequence comprising 12 transmembrane (TM) domains with a conserved domain [AGSV][HNQ][ACDEGNQSTV]XX[FWY]XXXXX(no L) as represented by SEQ ID NO:17, wherein X can be any amino acid residue, present in the first TM domain, preferably with a conserved domain [AGS]Q[ACGNQSTV]XX[FWY] as represented by SEQ ID NO:18, wherein X can be any amino acid residue, wherein the second amino acid residue of SEQ ID NO:17, preferably the second amino acid residue of SEQ ID NO: 18, is aligned to Lys18 of the polypeptide with SEQ ID NO:22.

38. A vector comprising the isolated nucleic acid molecule of any one of specific embodiment 36 or 37.

39. Use of an isolated nucleic acid molecule according to any one of specific embodiment 36 or 37 for production of one or more bioproduct(s) chosen from the list comprising saccharide; monosaccharide; activated monosaccharide; phosphorylated monosaccharide; disaccharide; oligosaccharide; polysaccharide; milk saccharide; mammalian milk saccharide; MMO; human milk saccharide; HMO; neutral (non-charged) saccharide; negatively charged saccharide; fucosylated saccharide; sialylated saccharide; neutral (non-charged) oligosaccharide; negatively charged oligosaccharide; fucosylated oligosaccharide; sialylated oligosaccharide; N-acetylglucosamine containing oligosaccharide; N-acetyllactosamine containing oligosaccharide; lacto-N-biose containing oligosaccharide; lactose containing oligosaccharide; non-fucosylated neutral (non-charged) oligosaccharide; O-antigen; ECA; the oligosaccharide repeats present in capsular polysaccharides; peptidoglycan; amino-sugar; Lewis-type antigen oligosaccharide; antigen of the human ABO blood group system; animal oligosaccharide, preferably selected from the group consisting of N-glycans and O-glycans; plant oligosaccharide, preferably selected from the group consisting of N-glycans and O-glycans; LN3; lacto-N-tetraose (LNT); LNnT; oligosaccharide derived from LN3; 3′sialyllactose (3′SL); 6′sialyllactose (6′SL); sialyllacto-N-tetraose a (LSTa); sialyllacto-N-tetraose b (LSTb); sialyllacto-N-tetraose c (LSTc); sialyllacto-N-tetraose d (LSTd); lacto-N-fucopentaose I; lacto-N-neofucopentaose; lacto-N-fucopentaose II; lacto-N-fucopentaose III; lacto-N-fucopentaose V; lacto-N-neofucopentaose V; lacto-N-difucohexaose I; lacto-N-neodifucohexaose; lacto-N-difucohexaose II; monofucosyllacto-n-hexaose III; difucosyllacto-N-hexaose a; 6′-galactosyllactose; 3′-galactosyllactose; lacto-N-hexaose; lacto-N-neohexaose; 2′-fucosyllactose (2′FL); 3-fucosyllactose (3-FL); difucosyllactose (DiFL); chitosan; chitosan comprising oligosaccharide; heparosan; glycosaminoglycan oligosaccharide; heparin; heparan sulphate; chondroitin sulphate; dermatan sulphate; hyaluronan; hyaluronic acid and keratan sulphate; preferably, the one or more bioproduct(s) is/are one or more LN3 derived oligosaccharide(s).

40. Use of a vector according to specific embodiment 38 for production of one or more bioproduct(s) chosen from the list comprising saccharide, monosaccharide, activated monosaccharide, phosphorylated monosaccharide, disaccharide, oligosaccharide, polysaccharide, a milk saccharide, a mammalian milk saccharide, an MMO, a human milk saccharide, an HMO, a neutral (non-charged) saccharide, a negatively charged saccharide, a fucosylated saccharide, a sialylated saccharide, a neutral (non-charged) oligosaccharide, a negatively charged oligosaccharide, a fucosylated oligosaccharide, a sialylated oligosaccharide, N-acetylglucosamine containing oligosaccharide, N-acetyllactosamine containing oligosaccharide, lacto-N-biose containing oligosaccharide; lactose containing oligosaccharide, non-fucosylated neutral (non-charged) oligosaccharide; O-antigen, ECA, the oligosaccharide repeats present in capsular polysaccharides, peptidoglycan, an amino-sugar, a Lewis-type antigen oligosaccharide, an antigen of the human ABO blood group system, an animal oligosaccharide, preferably selected from the group consisting of N-glycans and O-glycans, a plant oligosaccharide, preferably selected from the group consisting of N-glycans and O-glycans; LN3; lacto-N-tetraose (LNT), LNnT; an oligosaccharide derived from LN3; 3′sialyllactose (3′SL); 6′sialyllactose (6′SL); sialyllacto-N-tetraose a (LSTa); sialyllacto-N-tetraose b (LSTb); sialyllacto-N-tetraose c (LSTc); sialyllacto-N-tetraose d (LSTd); lacto-N-fucopentaose I; lacto-N-neofucopentaose; lacto-N-fucopentaose II; lacto-N-fucopentaose III; lacto-N-fucopentaose V; lacto-N-neofucopentaose V; lacto-N-difucohexaose I; lacto-N-neodifucohexaose; lacto-N-difucohexaose II; monofucosyllacto-n-hexaose III; difucosyllacto-N-hexaose a; 6′-galactosyllactose; 3′-galactosyllactose; lacto-N-hexaose; lacto-N-neohexaose; 2′-fucosyllactose (2′FL); 3-fucosyllactose (3-FL); difucosyllactose (DiFL); chitosan; chitosan comprising oligosaccharide; heparosan; chondroitin sulphate; glycosaminoglycan oligosaccharide; heparin; heparan sulphate; chondroitin sulphate; dermatan sulphate; hyaluronan; hyaluronic acid; and keratan sulphate; preferably, the one or more bioproduct(s) is/are one or more LN3 derived oligosaccharide(s).

The disclosure will be described in more detail in the examples. The following examples will serve as further illustration and clarification of this disclosure and are not intended to be limiting.

### EXAMPLES

### Example 1. Calculation of Percentage Identity Between Nucleotide or Polypeptide Sequences

Methods for the alignment of sequences for comparison are well known in the art, such methods include GAP, BESTFIT, BLAST, FASTA and TFASTA. GAP uses the algorithm of Needleman and Wunsch (J. Mol. Biol. (1970) 48:443-453) to find the global (i.e., spanning the full-length sequences) alignment of two sequences that maximizes the number of matches and minimizes the number of gaps. The BLAST algorithm (Altschul et al., J. Mol. Biol. (1990) 215:403-10) calculates the global percentage sequence identity (i.e., over the full-length sequence) and performs a statistical analysis of the similarity between the two sequences. The software for performing BLAST analysis is publicly available through the National Centre for Biotechnology Information (NCBI). Homologs may readily be identified using, for example, the ClustalW multiple sequence alignment algorithm (version 1.83), with the default pairwise alignment parameters, and a scoring method in percentage. Global percentages of similarity and identity (i.e., spanning the full-length sequences) may also be determined using one of the methods available in the MatGAT software package (Campanella et al., BMC Bioinformatics (2003) 4:29). Minor manual editing may be performed to optimize alignment between conserved motifs, as would be apparent to a person skilled in the art. Furthermore, instead of using full-length sequences for the identification of homologs, specific domains may also be used, to determine the so-called local sequence identity. The sequence identity values may be determined over the entire nucleic acid or amino acid sequence (=local sequence identity search over the full-length sequence resulting in a global sequence identity score) or over selected domains or conserved motif(s) (=local sequence identity search over a partial sequence resulting in a local sequence identity score), using the programs mentioned above using the default parameters. For local alignments, the Smith-Waterman algorithm is particularly useful (Smith T F, Waterman M S (1981) J. Mol. Biol 147 (1); 195-7).

### Example 2. Materials and Methods Escherichia coli

A. Escherichia coli

### Media

The Luria Broth (LB) medium consisted of 1% tryptone peptone (Difco, Erembodegem, Belgium), 0.5% yeast extract (Difco) and 0.5% sodium chloride (VWR. Leuven, Belgium). The minimal medium used in the cultivation experiments in 96-well plates or in shake flasks contained 2.00 g/L NH₄Cl, 5.00 g/L (NH₄)₂SO₄, 2.993 g/L KH₂PO₄, 7.315 g/L K₂HPO₄, 8.372 g/L MOPS, 0.5 g/L NaCl, 0.5 g/L MgSO₄·7H₂O, 30 g/L sucrose or 30 g/L glycerol, 1 ml/L vitamin solution, 100 μl/L molybdate solution, and 1 mL/L selenium solution. As specified in the respective examples, 0.30 g/L sialic acid, 20 g/L lactose and/or 20 g/L LN3 were additionally added to the medium. The minimal medium was set to a pH of 7.0 with 1M KOH. Vitamin solution consisted of 3.6 g/L FeCl₂·4H₂O, 5.0 g/L CaCl₂·2H₂O, 1.3 g/L MnCl₂·2H₂O, 0.38 g/L CuCl₂·2H₂O, 0.5 g/L CoCl₂·6H₂O, 0.94 g/L ZnCl₂, 0.0311 g/L H₃BO₄, 0.4 g/L Na₂EDTA·2H₂O and 1.01 g/L thiamine.HCl. The molybdate solution contained 0.967 g/L NaMoO₄·2H₂O. The selenium solution contained 42 g/L Seo₂.

The minimal medium for fermentations contained 6.75 g/L NH₄Cl, 1.25 g/L (NH₄)₂SO₄, 2.93 g/L KH₂PO₄ and 7.31 g/L KH₂PO₄, 0.5 g/L NaCl, 0.5 g/L MgSO₄·7H₂O, 30 g/L sucrose or 30 g/L glycerol, 1 mL/L vitamin solution, 100 μL/L molybdate solution, and 1 mL/L selenium solution with the same composition as described above. As specified in the respective examples, 0.30 g/L sialic acid, 20 g/L lactose and/or 20 g/L LN3 were additionally added to the medium.

Complex medium was sterilized by autoclaving (121° C., 21 min) and minimal medium by filtration (0.22 μm Sartorius). When necessary, the medium was made selective by adding an antibiotic: e.g., chloramphenicol (20 mg/L), carbenicillin (100 mg/L), spectinomycin (40 mg/L) and/or kanamycin (50 mg/L).

### Plasmids

pKD46 (Red helper plasmid, Ampicillin resistance), pKD3 (contains an FRT-flanked chloramphenicol resistance (cat) gene), pKD4 (contains an FRT-flanked kanamycin resistance (kan) gene), and pCP20 (expresses FLP recombinase activity) plasmids were obtained from Prof. R. Cunin (Vrije Universiteit Brussel, Belgium in 2007). Plasmids were maintained in the host E. coli DH5alpha (F⁻, phi80dlacZΔM15, Δ(lacZYA-argF) U169, deoR, recAl, endAl, hsdR17 (rk⁻, mk⁺), phoA, supE44, lambda⁻, thi-1, gyrA96, relA1) bought from Invitrogen.

### Strains and Mutations

Escherichia coli K12 MG1655 [λ⁻, F⁻, rph-1] was obtained from the Coli Genetic Stock Center (US), CGSC Strain #: 7740, in March 2007. Gene disruptions, gene introductions and gene replacements were performed using the technique published by Datsenko and Wanner (PNAS 97 (2000), 6640-6645) as described in e.g., WO22034067. Gene mutations were created via a PCR-based method described by Sanchis et al. (Appl. Microbiol. Biotechnol. (2008) 81 (2), 387-397).

In an example to produce LN3, the mutant strain was derived from E. coli K12 MG1655 and modified with a knock-out of the E. coli lacZ and nagB genes and with a constitutive transcriptional unit delivered to the strain either via genomic knock-in or from an expression plasmid like e.g., a pSC101-derived plasmid, for a galactoside beta-1,3-N-acetylglucosaminyltransferase like e.g., lgtA with UniProt ID Q9JXQ6 from Neisseria meningitidis. In an example for production of LN3 derived oligosaccharides like lacto-N-tetraose (LNT, Gal-b1,3-GlcNAc-b1,3-Gal-b1,4-Glc), the mutant LN3 producing strain was further modified with a constitutive transcriptional unit delivered to the strain either via genomic knock-in or from an expression plasmid for an N-acetylglucosamine beta-1,3-galactosyltransferase like e.g., wbgO (Uniprot ID D3QY14) from E. coli 055:H7. In an example for production of LN3 derived oligosaccharides like lacto-N-neotetraose (LNnT, Gal-b1,4-GlcNAc-b1,3-Gal-b1,4-Glc), the mutant LN3 producing strain was further modified with a constitutive transcriptional unit delivered to the strain either via genomic knock-in or from an expression plasmid for an N-acetylglucosamine beta-1,4-galactosyltransferase like e.g., LgtB (Uniprot ID Q51116, sequence version 02, 1 Dec. 2000) from N. meningitidis.

Optionally, the LN3, LNT and/or LNnT production can further be optimized in the mutant E. coli strains with genomic knock-out of the E. coli LacY gene and with a genomic knock-in of one or more constitutive transcriptional units for a lactose permease like e.g., the E. coli LacY (UniProt ID P02920).

Additionally and/or alternatively, LN3, LNT and/or LNnT production can further be optimized in the mutant E. coli strains with expression of a saccharide importer having uptake activity for LN3 chosen from the list comprising SEQ ID NOs: 01, 02, 03, 04, 09, 12, 16, 19, 20 and 21, wherein the saccharide importer is expressed from one or more constitutive transcriptional units that is/are presented to the cell via (a) genomic knock-in(s) or via a plasmid transformed to the cell.

Additionally and/or alternatively, LN3, LNT and/or LNnT production can further be optimized in the mutant E. coli strains with genomic knock-outs of the E. coli genes comprising any one or more of galT, ushA, IdhA and agp.

The mutant LN3, LNT and/or LNnT producing strains can also be optionally modified for enhanced UDP-GlcNAc production with a genomic knock-in of a constitutive transcriptional unit for an L-glutamine-D-fructose-6-phosphate aminotransferase like e.g., the mutant glmS*54 from E. coli (differing from the wild-type E. coli glmS protein, having UniProt ID P17169 (sequence version 04, 23 Jan. 2007), by an A39T, an R250C and an G472S mutation as described by Deng et al. (Biochimie 2006, 88:419-429).

The mutant E. coli strains can also optionally be adapted with a genomic knock-in of a constitutive transcriptional unit for an UDP-glucose-4-epimerase like e.g., galE from E. coli (UniProt ID P09147), a phosphoglucosamine mutase like e.g., glmM from E. coli (UniProt ID P31120, sequence version 03, 23 Jan. 2007) and an N-acetylglucosamine-1-phosphate uridylyltransferase/glucosamine-1-phosphate acetyltransferase like e.g., glmU from E. coli (UniProt ID POACC7).

The mutant LN3, LNT and/or LNnT producing E. coli strains can also optionally be adapted for growth on sucrose via genomic knock-ins of constitutive transcriptional units containing a sucrose transporter like e.g., CscB from E. coli W (UniProt ID EOIXR1), a fructose kinase like e.g., Frk originating from Zymomonas mobilis (UniProt ID Q03417) and a sucrose phosphorylase like e.g., BaSP originating from Bifidobacterium adolescentis (UniProt ID A0ZZH6).

Alternatively, and/or additionally, production of LN3, LNT, LNnT and oligosaccharides derived thereof can further be optimized in the mutant E. coli strains with genomic knock-ins of constitutive transcriptional units comprising a membrane transporter protein like e.g., MdfA from Cronobacter muytjensii (UniProt ID A0A2T7ANQ9), MdfA from Citrobacter youngae (UniProt ID D4BC23) or MdfA from E. coli (UniProt ID POAEY8).

In an example for sialic acid and CMP-sialic acid production, the mutant strain was derived from E. coli K12 MG1655 as described e.g., in WO2018122225, WO2021123113, WO22034067, WO22034068 or WO22034070. To allow sialylated oligosaccharide production, the mutant E. coli strain producing CMP-sialic acid was further modified with one or more transcriptional unit(s) encoding one or more sialyltransferases. The strain could additionally be modified to comprise a transcriptional unit for a lactose permease like e.g., E. coli LacY (UniProt ID P02920). The mutant E. coli strain can also optionally be adapted for growth on sucrose via genomic knock-ins of constitutive transcriptional units containing a sucrose transporter like e.g., CscB from E. coli W (UniProt ID EOIXR1), a fructose kinase like e.g., Frk originating from Zymomonas mobilis (UniProt ID Q03417) and a sucrose phosphorylase like e.g., BaSP originating from Bifidobacterium adolescentis (UniProt ID AOZZH6).

In an example for GDP-fucose production, the mutant derived from E. coli K12 MG1655 as described e.g., in WO2020127417, WO2021013708, WO22034067, WO220234068 or WO22034069. To allow fucosylated oligosaccharide production, the mutant E. coli strain producing GDP-fucose was further modified with one or more transcriptional unit(s) encoding one or more fucosyltransferases. The mutant E. coli strain can also optionally be adapted for growth on sucrose via genomic knock-ins of constitutive transcriptional units containing a sucrose transporter like e.g., CscB from E. coli W (UniProt ID EOIXR1), a fructose kinase like e.g., Frk originating from Zymomonas mobilis (UniProt ID Q03417) and a sucrose phosphorylase like e.g., BaSP originating from Bifidobacterium adolescentis (UniProt ID AOZZH6).

In an example to produce one or more fucosylated non-charged oligosaccharide(s), an E. coli K12 M1655 strain is modified for production of GDP-fucose, LN3, LNT and/or LNnT as described herein and for expression of one or more compatible fucosyltransferase(s). Optionally, the strain can further be modified for expression of a saccharide importer having uptake activity for LN3 chosen from the list comprising SEQ ID NOs: 01, 02, 03, 04, 09, 12, 16, 19, 20 and 21.

In an example to produce one or more sialylated oligosaccharide(s) like e.g., LSTa and LSTb, an E. coli K12 MG1655 strain is modified for production of CMP-sialic acid, LN3 and LNT as described herein and for expression of one or more compatible sialyltransferase(s). Optionally, the strain can further be modified for expression of a saccharide importer having uptake activity for LN3 chosen from the list comprising SEQ ID NOs: 01, 02, 03, 04, 09, 12, 16, 19, 20 and 21.

In an example to produce one or more sialylated oligosaccharide(s) like e.g., LSTc and LSTd, an E. coli K12 MG1655 strain is modified for production of CMP-sialic acid, LN3 and LNnT as described herein and for expression of one or more compatible sialyltransferase(s). Optionally, the strain can further be modified for expression of a saccharide importer having uptake activity for LN3 chosen from the list comprising SEQ ID NOs: 01, 02, 03, 04, 09, 12, 16, 19, 20 and 21.

Preferably, but not necessarily, any one or more of the glycosyltransferases and/or the proteins involved in nucleotide-activated sugar synthesis were N- and/or C-terminally fused to a solubility enhancer tag like e.g., a SUMO-tag, an MBP-tag, His, FLAG, Strep-II, Halo-tag, NusA, thioredoxin, GST and/or the Fh8-tag to enhance their solubility (Costa et al., Front. Microbiol. 2014, doi.org/10.3389/fmicb.2014.00063; Fox et al., Protein Sci. 2001, 10 (3), 622-630; Jia and Jeaon, Open Biol. 2016, 6:160196).

Optionally, the modified E. coli strains were modified with a genomic knock-ins of a constitutive transcriptional unit encoding a chaperone protein like e.g., DnaK, DnaJ, GrpE or the GroEL/ES chaperonin system (Baneyx F., Palumbo J. L. (2003) Improving Heterologous Protein Folding via Molecular Chaperone and Foldase Co-Expression. In: Vaillancourt P.E. (eds) E. coli Gene Expression Protocols. Methods in Molecular Biology™, vol 205. Humana Press).

All constitutive promoters, UTRs and terminator sequences originated from the libraries described by Cambray et al. (Nucleic Acids Res. 2013, 41(9), 5139-5148), Dunn et al. (Nucleic Acids Res. 1980, 8, 2119-2132), Edens et al. (Nucleic Acids Res. 1975, 2, 1811-1820), Kim and Lee (FEBS Letters 1997, 407, 353-356) and Mutalik et al. (Nat. Methods 2013, No. 10, 354-360). Genes were ordered synthetically at Twist Bioscience (twistbioscience.com) or IDT (eu.idtdna.com) and the codon usage was adapted using the tools of the supplier. Proteins described in present disclosure are summarized in Tables 1 and 2. All strains were stored in cryovials at −80° C. (overnight LB culture mixed in a 1:1 ratio with 70% glycerol).

### Cultivation Conditions

A preculture of 96-well microtiter plate experiments was started from a cryovial, in 150 μL LB and was incubated overnight at 37° C. on an orbital shaker at 800 rpm. This culture was used as inoculum for a 96 well square microtiter plate, with 400 μL minimal medium by diluting 400×. These final 96-well culture plates were then incubated at 37° C. on an orbital shaker at 800 rpm for 72 h, or shorter, or longer. To measure sugar concentrations at the end of the cultivation experiment whole broth samples were taken from each well by boiling the culture broth for 1 hour at 60° C. before spinning down the cells (=average of intra- and extracellular sugar concentrations).

A preculture for the bioreactor was started from an 250 μl cryovial of a certain strain, inoculated in 250 mL or 500 mL minimal medium in a 1 L or 2.5 L shake flask and incubated for 24 h at 37° C. on an orbital shaker at 200 rpm. A 5 L bioreactor was then inoculated (250 mL inoculum in 2 L batch medium); the process was controlled by MFCS control software (Sartorius Stedim Biotech, Melsungen, Germany). Culturing condition were set to 37° C., and maximal stirring; pressure gas flow rates were dependent on the strain and bioreactor. The pH was controlled at 6.8 using 0.5 M H2S04 and 20% NH₄OH. The exhaust gas was cooled. 10% solution of silicone antifoaming agent was added when foaming raised during the fermentation.

### Optical Density

Cell density of the cultures was frequently monitored by measuring optical density at 600 nm (Implen Nanophotometer NP80, Westburg, Belgium or with a Spark 10M microplate reader, Tecan, Switzerland). The maximum growth speed (mumax) was calculated based on the observed optical densities at 600 nm using the R package grofit.

B. Saccharomyces cerevisiae

### Media

Strains were grown on Synthetic Defined yeast medium with Complete Supplement Mixture (SD CSM) or CSM drop-out (SD CSM-Ura, SD CSM-Trp, SD CSM-His) containing 6.7 g/L Yeast Nitrogen Base without amino acids (YNB w/o AA, Difco), 20 g/L agar (Difco) (solid cultures), 22 g/L glucose monohydrate, 20 g/L lactose and/or 20 g/L LN3 and 0.79 g/L CSM or 0.77 g/L CSM-Ura, 0.77 g/L CSM-Trp, or 0.77 g/L CSM-His (MP Biomedicals).

### Strains

S. cerevisiae BY4742 created by Brachmann et al. (Yeast (1998) 14:115-32) was used, available in the Euroscarf culture collection. All mutant strains were created by homologous recombination or plasmid transformation using the method of Gietz (Yeast 11:355-360, 1995).

In an example to produce UDP-galactose, a yeast expression plasmid derived from the pRS420-plasmid series (Christianson et al., 1992, Gene 110:119-122) containing the HIS3 selection marker was modified as described e.g., in WO2022157213 or WO22034067. In an example to produce LN3, the plasmid was further modified with transcriptional units encoding a lactose permease like e.g., LAC12 from K. lactis (UniProt ID P07921) and a galactoside beta-1,3-N-acetylglucosaminyltransferase like e.g., LgtA from N. meningitidis (UniProt ID Q9JXQ6). In another example, the S. cerevisiae strain engineered for LN3 production is further modified with a transcriptional unit for an N-acetylglucosamine beta-1,3-galactosyltransferase like e.g., WbgO (Uniprot ID D3QY14) from E. coli 055:H7 to produce LNT or with an N-acetylglucosamine beta-1,4-galactosyltransferase like e.g., LgtB (Uniprot ID Q51116, sequence version 02, 1 Dec. 2000) from N. meningitidis to produce LNnT.

In an example to produce sialic acid and CMP-sialic acid, a yeast expression plasmid derived from the pRS420-plasmid series (Christianson et al., 1992, Gene 110:119-122) containing the TRPI selection was modified as described in e.g., WO2021123113 or WO22034067. To allow sialylated oligosaccharide production, the mutant S. cerevisiae strain producing CMP-sialic acid was further modified with one or more transcriptional unit(s) encoding one or more sialyltransferases. In an example to produce GDP-fucose, the yeast expression plasmid p2a_2u_Fuc (Chan 2013, Plasmid 70, 2-17) comprising an ampicillin resistance gene and a bacterial origin of replication to allow for selection and maintenance in E. coli and the 2u yeast ori and the Ura3 selection marker for selection and maintenance in yeast was modified as described in e.g., WO2020127417 or WO22034067. To allow fucosylated oligosaccharide production, the mutant S. cerevisiae strain producing CMP-sialic acid was further modified with one or more transcriptional unit(s) encoding one or more fucosyltransferases.

Optionally, the mutant strains as described herein can further be modified for expression of a saccharide importer having uptake activity for LN3 chosen from the list comprising SEQ ID NOs: 01, 02, 03, 04, 09, 12, 16, 19, 20 and 21.

### Gene Expression Promoters

Genes were expressed using synthetic constitutive promoters, as described by e.g., Blazeck (Biotechnology and Bioengineering, Vol. 109, No. 11, 2012), Redden and Alper (Nat. Commun. 2015, 6, 7810), Liu et al. (Microb. Cell Fact. 2020, 19, 38), Xu et al. (Microb. Cell Fact. 2021, 20, 148) and Lee et al. (ACS Synth. Biol. 2015, 4(9), 975-986).

### Cultivations Conditions

In general, yeast strains were initially grown on SD CSM plates to obtain single colonies. These plates were grown for 2-3 days at 30° C. Starting from a single colony, a preculture was grown over night in 5 mL at 30° C., shaking at 200 rpm. Subsequent 125 mL shake flask experiments were inoculated with 2% of this preculture, in 25 mL media. These shake flasks were incubated at 30° C. with an orbital shaking of 200 rpm for 72 h, or shorter of longer. At the end of the cultivation experiment samples were taken to measure the supernatant concentration (extracellular sugar concentrations, after 5 min. spinning down the cells), or by boiling the culture broth for 60 min at 60° C. before spinning down the cells (=whole broth concentration, i.e., intra- and extracellular sugar concentrations).

C. Bacillus subtilis

### Media

Two media are used to cultivate B. subtilis: i.e., a complex medium like a rich Luria Broth (LB) and a minimal medium for shake flask cultures. The LB medium consisted of 1% tryptone peptone (Difco), 0.5% yeast extract (Difco) and 0.5% sodium chloride (VWR). Luria Broth agar (LBA) plates consisted of the LB media, with 12 g/L agar (Difco) added. The minimal medium contained 2.00 g/L (NH₄)₂SO₄, 7.5 g/L KH₂PO₄, 17.5 g/L K₂HPO₄, 1.25 g/L Na-citrate, 0.25 g/L MgSO₄·7H₂O, 0.05 g/L tryptophan, from 10 up to 30 g/L glucose (or another carbon source including but not limited to fructose, maltose, sucrose, glycerol and maltotriose), 10 mL/L trace element mix and 10 mL/L Fe-citrate solution. The medium was set to a pH of 7 with 1 M KOH. Depending on the experiment lactose and/or LN3 is added as a precursor. The trace element mix consisted of 0.735 g/L CaCl₂·2H_{c}O, 0.1 g/L MnCl₂·2H₂O, 0.033 g/L CuCl₂·2H₂O, 0.06 g/L CoCl₂·6H₂O, 0.17 g/L ZnCl₂, 0.0311 g/L H₃BO₄, 0.4 g/L Na₂EDTA.2H₂O and 0.06 g/L Na₂MoO₄. The Fe-citrate solution contained 0.135 g/L FeCl₃·6H₂O, 1 g/L Na-citrate (Hoch 1973 PMC1212887). Complex medium, e.g., LB, was sterilized by autoclaving (121° C., 21 min) and minimal medium by filtration (0.22 μm Sartorius). When necessary, the medium was made selective by adding an antibiotic.

### Strains, Plasmids and Mutations

B. subtilis 168 is used as available at the Bacillus Genetic Stock Center (Ohio, USA). Plasmids for gene deletion via Cre/lox are constructed as described by Yan et al. (Appl & Environm microbial, September 2008, p5556-5562). Gene disruption is done via homologous recombination with linear DNA and transformation via the electroporation as described by Xue et al. (J. Mircrob. Meth. 34 (1999) 183-191). The method of gene knockouts is described by Liu et al. (Metab. Engine. 24 (2014) 61-69). Integrative vectors as described by Popp et al. (Sci. Rep., 2017, 7, 15158) are used as expression vector and could be further used for genomic integrations if necessary. A suitable promoter for expression can be derived from the part repository (iGem): sequence id: BBa_K143012, BBa_K823000, BBa_K823002 or BBa_K823003. Cloning can be performed using Gibson Assembly, Golden Gate assembly, Cliva assembly, LCR or restriction ligation.

In an example for LN3 production, the mutant strain was derived from B. subtilis 168 and modified to comprise a transcriptional unit for a lactose permease like e.g., E. coli LacY (UniProt ID P02920) and a galactoside beta-1,3-N-acetylglucosaminyltransferase like e.g., LgtA from N. meningitidis (UniProt ID Q9JXQ6). In another example, the B. subtilis strain engineered for LN3 production is further modified with a transcriptional unit for an N-acetylglucosamine beta-1,3-galactosyltransferase like e.g., WbgO (Uniprot ID D3QY14) from E. coli 055:H7 to produce LNT or with an N-acetylglucosamine beta-1,4-galactosyltransferase like e.g., LgtB (Uniprot ID Q51116, sequence version 02, 1 Dec. 2000) from N. meningitidis to produce LNnT.

In an example for sialic acid and CMP-sialic acid production, the mutant strain was derived from B. subtilis 168 and modified as described e.g., in WO1822225, WO2021123113 or WO22034067. To allow sialylated oligosaccharide production, the mutant B. subtilis strain producing CMP-sialic acid was further modified with one or more transcriptional unit(s) encoding one or more sialyltransferases. The strain could additionally be modified to comprise a transcriptional unit for a lactose permease like e.g., E. coli LacY (UniProt ID P02920).

In an example for fucosylated oligosaccharide production, the mutant strain was derived from B. subtilis 168 and modified as described e.g., in WO2022157213 or WO22034069 to comprise one or more transcriptional unit(s) encoding one or more fucosyltransferases. Additionally, the mutant strain could be further modified with a transcriptional unit for a lactose permease like e.g., E. coli LacY (UniProt ID P02920) if necessary.

In an example to produce one or more fucosylated non-charged oligosaccharide(s), a B. subtilis strain is modified for production of GDP-fucose, LN3, LNT and/or LNnT as described herein and for expression of one or more compatible fucosyltransferase(s). In an example to produce one or more sialylated oligosaccharide(s) like e.g., LSTa and LSTb, a B. subtilis strain is modified for production of CMP-sialic acid, LN3 and LNT as described herein and for expression of one or more compatible sialyltransferase(s). In an example to produce one or more sialylated oligosaccharide(s) like e.g., LSTc and LSTd, a B. subtilis strain is modified for production of CMP-sialic acid, LN3 and LNnT as described herein and for expression of one or more compatible sialyltransferase(s).

Optionally, the mutant strains as described herein can further be modified for expression of a saccharide importer having uptake activity for LN3 chosen from the list comprising SEQ ID NOs: 01, 02, 03, 04, 09, 12, 16, 19, 20 and 21.

### Cultivation Conditions

A preculture was started from a cryovial or a single colony from an LBA plate, in 6 mL LB and was incubated overnight at 37° C. on an orbital shaker at 200 rpm. Subsequent 125 mL shake flask experiments were inoculated with 2% of this preculture, in 25 mL minimal medium. These shake flasks were incubated at 37° C. with an orbital shaking of 200 rpm for 72 h, or shorter of longer. At the end of the cultivation experiment samples were taken to measure the supernatant concentration (extracellular sugar concentrations, after 5 min. spinning down the cells), or by boiling the culture broth for 60 min at 60° C. before spinning down the cells (=whole broth concentration, i.e., intra- and extracellular sugar concentrations).

D. Corynebacterium glutamicum

### Media

Two different media are used, namely complex medium like e.g., a rich tryptone-yeast extract (TY) medium, and a minimal medium for shake flask (MMsf). The minimal medium uses a 1000×stock trace element mix. Trace element mix consisted of 10 g/L CaCl₂), 10 g/L FeSO₄·7H₂O, 10 g/L MnSO₄·H₂O, 1 g/L ZnSO₄·7H₂O, 0.2 g/L CuSO₄, 0.02 g/L NiCl₂·6H₂O, 0.2 g/L biotin (pH 7) and 0.03 g/L protocatechuic acid. The minimal medium for the shake flasks (MMsf) experiments contained 20 g/L (NH₄)2SO₄, 5 g/L urea, 1 g/L KH₂PO₄, 1 g/L K₂HPO₄, 0.25 g/L MgSO₄·7H₂O, 42 g/L MOPS, from 10 up to 30 g/L glucose or another carbon source including but not limited to fructose, maltose, sucrose, glycerol and maltotriose when specified in the examples and 1 ml/L trace element mix. Depending on the experiment lactose, LNB, LacNAc and/or LN3 could be added to the medium. The TY medium consisted of 1.6% tryptone (Difco, Erembodegem, Belgium), 1% yeast extract (Difco) and 0.5% sodium chloride (VWR. Leuven, Belgium). TY agar (TYA) plates consisted of the TY media, with 12 g/L agar (Difco, Erembodegem, Belgium) added. Complex medium, e.g., TY, was sterilized by autoclaving (121° C., 21 min) and minimal medium by filtration (0.22 μm Sartorius). When necessary, the medium was made selective by adding an antibiotic.

### Strains and Mutations

Corynebacterium glutamicum was used as available at the American Type Culture Collection (ATCC 13032). Integrative plasmid vectors were made using the Cre/loxP technique as described by Suzuki et al. (Appl. Microbiol. Biotechnol., 2005 April, 67 (2): 225-33) and temperature-sensitive shuttle vectors as described by Okibe et al. (Journal of Microbiological Methods 85, 2011, 155-163) are constructed for gene deletions, mutations and insertions. Suitable promoters for (heterologous) gene expression can be derived from Yim et al. (Biotechnol. Bioeng., 2013 November, 110(11):2959-69). Cloning can be performed using Gibson Assembly, Golden Gate assembly, Cliva assembly, LCR or restriction ligation.

In an example for LN3 production, the mutant strain was derived from C glutamicum and modified to comprise a transcriptional unit for a lactose permease like e.g., E. coli LacY (UniProt ID P02920) and a galactoside beta-1,3-N-acetylglucosaminyltransferase like e.g., LgtA from N. meningitidis (UniProt ID Q9JXQ6. In another example, the C. glutamicum strain engineered for LN3 production is further modified with a transcriptional unit for an N-acetylglucosamine beta-1,3-galactosyltransferase like e.g., WbgO (Uniprot ID D3QY14) from F. coli 055:H7 to produce LNT or with an N-acetylglucosamine beta-1,4-galactosyltransferase like e.g., LgtB (Uniprot ID Q51116, sequence version 02, 1 Dec. 2000) from N. meningitidis to produce LNnT.

In an example for sialic acid and CMP-sialic acid production, the mutant strain was derived from C. glutamicum and modified as described e.g., in WO2022129470 or WO22034067. To allow sialylated oligosaccharide production, the mutant (′. glutamicum strain producing CMP-sialic acid was further modified with one or more transcriptional unit(s) encoding one or more sialyltransferases. The strain could additionally be modified to comprise a transcriptional unit for a lactose permease like e.g., E. coli LacY (UniProt ID P02920).

In an example for fucosylated oligosaccharide production, the mutant strain is derived from (′. glutamicum and modified as described e.g., in WO22034069 to comprise one or more transcriptional unit(s) encoding one or more fucosyltransferases. Additionally, the mutant strain could be further modified with a transcriptional unit for a lactose permease like e.g., E. coli LacY (UniProt ID P02920) if necessary.

In an example to produce one or more fucosylated non-charged oligosaccharide(s), a (′. glutamicum strain is modified for production of GDP-fucose, LN3, LNT and/or LNnT as described herein and for expression of one or more compatible fucosyltransferase(s). In an example to produce one or more sialylated oligosaccharide(s) like e.g., LSTa and LSTb, a (′. glutamicum strain is modified for production of CMP-sialic acid, LN3 and LNT as described herein and for expression of one or more compatible sialyltransferase(s). In an example to produce one or more sialylated oligosaccharide(s) like e.g., LSTc and LSTd, a C. glutamicum strain is modified for production of CMP-sialic acid, LN3 and LNnT as described herein and for expression of one or more compatible sialyltransferase(s).

Optionally, the mutant strains as described herein can further be modified for expression of a saccharide importer having uptake activity for LN3 chosen from the list comprising SEQ ID NOs: 01, 02, 03, 04, 09, 12, 16, 19, 20 and 21.

### Cultivation Conditions

A preculture was started from a cryovial or a single colony from a TY plate, in 6 mL TY and was incubated overnight at 37° C. on an orbital shaker at 200 rpm. Subsequent 125 mL shake flask experiments were inoculated with 2% of this preculture, in 25 mL MMsf medium. These shake flasks were incubated at 37° C. with an orbital shaking of 200 rpm for 72 h, or shorter of longer. At the end of the cultivation experiment samples were taken to measure the supernatant concentration (extracellular sugar concentrations, after 5 min. spinning down the cells), or by boiling the culture broth for 60 min at 60° C. before spinning down the cells (=whole broth concentration, i.e., intra- and extracellular sugar concentrations).

E. Chlamydomonas reinhardtii

### Media

Chlamydomonas reinhardtii cells were cultured in Tris-acetate-phosphate (TAP) medium (pH 7). The TAP medium uses a 1000×stock Hutner's trace element mix. Hutner's trace element mix consisted of 50 g/L Na₂EDTA·H₂O (Titriplex III), 22 g/L ZnSO₄·7H₂O, 11.4 g/L H₃BO₃, 5 g/L MnCl₂·4H₂O, 5 g/L FeSO₄·7H₂O, 1.6 g/L CoCl₂·6H₂O, 1.6 g/L CuSO₄·5H₂O and 1.1 g/L (NH₄)₆MoO₃. The TAP medium contained 2.42 g/L Tris (tris(hydroxymethyl)aminomethane), 25 mg/L salt stock solution, 0.108 g/L K₂HPO₄, 0.054 g/L KH₂PO₄ and 1.0 mL/L glacial acetic acid. The salt stock solution consisted of 15 g/L NH₄CL, 4 g/L MgSO₄·7H₂O and 2 g/L CaCl₂·2H₂O. As precursor(s) and/or acceptor(s) for saccharide synthesis, compounds like e.g., galactose, glucose, fructose, fucose, lactose, LacNAc, LNB, and/or LN3 could be added. Medium was sterilized by autoclaving (121° C., 21 min). For stock cultures on agar slants TAP medium was used containing 1% agar (of purified high strength, 1000 g/cm²).

### Strains, Plasmids and Mutations

C. reinhardtii wild-type strains 21 gr (CC-1690, wild-type, mt+), 6145C (CC-1691, wild-type, mt-), CC-125 (137c, wild-type, mt+), CC-124 (137c, wild-type, mt-) as available from the Chlamydomonas Resource Center (chlamycollection.org) (University of Minnesota, U.S.A.) were used. Expression plasmids originated from pSI103, as available from the Chlamydomonas Resource Center. Cloning can be performed using Gibson Assembly, Golden Gate assembly, Cliva assembly, LCR or restriction ligation. Suitable promoters for (heterologous) gene expression can be derived from e.g., Scranton et al. (Algal Res. 2016, 15:135-142). Targeted gene modification (like gene knock-out or gene replacement) can be carried using the Crispr-Cas technology as described e.g., by Jiang et al. (Eukaryotic Cell 2014, 13 (11): 1465-1469). Transformation via electroporation was performed as described by Wang et al. (Biosci. Rep. 2019, 39: BSR2018210) and as described like e.g., in WO22034067 or in WO22034069.

In an example for UDP-galactose synthesis, the mutant strain was derived from C. reinhardtii and modified as described e.g., in WO22034067.

In an example for LN3 production, the mutant strain was derived from C. reinhardtii and modified to comprise a transcriptional unit for a galactoside beta-1,3-N-acetylglucosaminyltransferase like e.g., LgtA from N. meningitidis (UniProt ID Q9JXQ6). In another example, the (′. glutamicum strain engineered for UDP-galactose synthesis as described e.g., in WO22034067 is further modified to comprise a transcriptional unit for a galactoside beta-1,3-N-acetylglucosaminyltransferase like e.g., LgtA from N. meningitidis (UniProt ID Q9JXQ6) for LN3 production and i) with a transcriptional unit for an N-acetylglucosamine beta-1,3-galactosyltransferase like e.g., WbgO (Uniprot ID D3QY14) from E. coli 055:H7 to produce LNT or ii) with a transcriptional unit for an N-acetylglucosamine beta-1,4-galactosyltransferase like e.g., LgtB (Uniprot ID Q51116, sequence version 02, 1 Dec. 2000) from N. meningitidis to produce LNnT.

In an example for sialic acid and CMP-sialic acid production, the mutant strain was derived from C. reinhardtii and modified as described e.g., in WO22034067. In an example for production of sialylated oligosaccharides, C. reinhardtii cells are modified with a CMP-sialic acid transporter like e.g., CST from Mus musculus (UniProt ID Q61420), and a Golgi-localized sialyltransferase chosen from species like e.g., Homo sapiens, Mus musculus, Rattus norvegicus.

In an example for GDP-fucose synthesis, the mutant strain was derived from C. reinhardtii and modified as described e.g., in WO22034067. In an example for fucosylation, C. reinhardtii cells can be modified with an expression plasmid comprising one or more transcriptional unit(s) encoding one or more fucosyltransferases.

In an example for UDP-galactose synthesis, the mutant strain was derived from (′. reinhardtii and modified as described e.g., in WO22034067.

In an example to produce one or more fucosylated non-charged oligosaccharide(s), a (′. reinhardtii strain is modified for production of GDP-fucose, UDP-galactose, LN3, LNT and/or LNnT as described herein and for expression of one or more compatible fucosyltransferase(s). In an example to produce one or more sialylated oligosaccharide(s) like e.g., LSTa and LSTb, a C. reinhardtii strain is modified for production of CMP-sialic acid, UDP-galactose, LN3 and LNT as described herein and for expression of one or more compatible sialyltransferase(s). In an example to produce one or more sialylated oligosaccharide(s) like e.g., LSTc and LSTd, a C. reinhardtii strain is modified for production of CMP-sialic acid, UDP-galactose, LN3 and LNnT as described herein and for expression of one or more compatible sialyltransferase(s).

Optionally, the mutant strains as described herein can further be modified for expression of a saccharide importer having uptake activity for LN3 chosen from the list comprising SEQ ID NOs: 01, 02, 03, 04, 09, 12, 16, 19, 20 and 21.

### Cultivation Conditions

Cells of C. reinhardtii were cultured in selective TAP-agar plates at 23+/−0.5° C. under 14/10 h light/dark cycles with a light intensity of 8000 Lx. Cells were analyzed after 5 to 7 days of cultivation. For high-density cultures, cells could be cultivated in closed systems like e.g., vertical or horizontal tube photobioreactors, stirred tank photobioreactors or flat panel photobioreactors as described by Chen et al. (Bioresour. Technol. 2011, 102:71-81) and Johnson et al. (Biotechnol. Prog. 2018, 34:811-827).

### F. Animal Cells

Isolation of Mesenchymal Stem Cells from Adipose Tissue of Different Animals

Fresh adipose tissue is obtained from slaughterhouses (e.g., cattle, pigs, sheep, chicken, ducks, catfish, snake, frogs) or liposuction (e.g., in case of humans, after informed consent) and kept in phosphate buffer saline supplemented with antibiotics. Enzymatic digestion of the adipose tissue is performed followed by centrifugation to isolate mesenchymal stem cells. The isolated mesenchymal stem cells are transferred to cell culture flasks and grown under standard growth conditions, e.g., 37° C., 5% CO2. The initial culture medium includes DMEM-F12, RPMI, and Alpha-MEM medium (supplemented with 15% fetal bovine serum), and 1% antibiotics. The culture medium is subsequently replaced with 10% FBS (fetal bovine serum)-supplemented media after the first passage. For example, Ahmad and Shakoori (2013, Stem Cell Regen. Med. 9 (2): 29-36), which is incorporated herein by reference in its entirety for all purposes, describes certain variation(s) of the method(s) described herein in this example.

Isolation of Mesenchymal Stem Cells from Milk

This example illustrates isolation of mesenchymal stem cells from milk collected under aseptic conditions from human or any other mammal(s) such as described herein. An equal volume of phosphate buffer saline is added to diluted milk, followed by centrifugation for 20 min. The cell pellet is washed thrice with phosphate buffer saline and cells are seeded in cell culture flasks in DMEM-F12, RPMI, and Alpha-MEM medium supplemented with 10% fetal bovine serum and 1% antibiotics under standard culture conditions. For example, Hassiotou et al. (2012, Stem Cells. 30 (10): 2164-2174), which is incorporated herein by reference in its entirety for all purposes, describes certain variation(s) of the method(s) described herein in this example.

### Differentiation of Stem Cells Using 2D and 3D Culture Systems

The mesenchymal cells isolated from adipose tissue of different animals or from milk as described above can be differentiated into mammary-like epithelial and luminal cells in 2D and 3D culture systems. See, for example, Huynh et al. 1991. Exp Cell Res. 197 (2): 191-199; Gibson et al. 1991, In Vitro Cell Dev Biol Anim. 27 (7): 585-594; Blatchford et al. 1999; Animal Cell Technology′: Basic & Applied Aspects, Springer, Dordrecht. 141-145; Williams et al. 2009, Breast Cancer Res 11(3): 26-43; and Arevalo et al. 2015, Am J Physiol Cell Physiol. 310(5): C348-C356; each of which is incorporated herein by reference in their entireties for all purposes.

For 2D culture, the isolated cells were initially seeded in culture plates in growth media supplemented with 10 ng/mL epithelial growth factor and 5 μg/mL insulin. At confluence, cells were fed with growth medium supplemented with 2% fetal bovine serum, 1% penicillin-streptomycin (100 U/mL penicillin, 100 μg/mL streptomycin), and 5 μg/mL insulin for 48 h. To induce differentiation, the cells were fed with complete growth medium containing 5 μg/mL insulin, 1 μg/mL hydrocortisone, 0.65 ng/mL triiodothyronine, 100 nM dexamethasone, and 1 μg/mL prolactin. After 24 h, serum is removed from the complete induction medium.

For 3D culture, the isolated cells were trypsinized and cultured in Matrigel, hyaluronic acid, or ultra-low attachment surface culture plates for six days and induced to differentiate and lactate by adding growth media supplemented with 10 ng/mL epithelial growth factor and 5 μg/mL insulin. At confluence, cells were fed with growth medium supplemented with 2% fetal bovine serum, 1% penicillin-streptomycin (100 U/mL penicillin, 100 μg/mL streptomycin), and 5 μg/mL insulin for 48 h. To induce differentiation, the cells were fed with complete growth medium containing 5 μg/mL insulin, 1 μg/mL hydrocortisone, 0.65 ng/ml triiodothyronine, 100 nM dexamethasone, and 1 μg/mL prolactin. After 24 h, serum is removed from the complete induction medium.

### Method of Making Mammary-Like Cells

In a next step, the cells are brought to induced pluripotency by reprogramming with viral vectors encoding for Oct4, Sox2, Klf4, and c-Myc. The resultant reprogrammed cells are then cultured in Mammocult media (available from Stem Cell Technologies), or mammary cell enrichment media (DMEM, 3% FBS, estrogen, progesterone, heparin, hydrocortisone, insulin, EGF) to make them mammary-like, from which expression of select milk components can be induced. Alternatively, epigenetic remodeling is performed using remodeling systems such as CRISPR/Cas9, to activate select genes of interest, such as casein, a-lactalbumin to be constitutively on, to allow for the expression of their respective proteins, and/or to down-regulate and/or knock-out select endogenous genes as described e.g., in WO21067641, which is incorporated herein by reference in its entirety for all purposes. In an example for production of one or more oligosaccharide(s), isolated mesenchymal cells re-programmed into mammary-like cells are modified via CRISPR-CAS as described e.g., in WO2022157213, WO22034067, WO22034070 and WO22034075. Optionally, the mutant cells as described herein can further be modified for expression of a saccharide importer having uptake activity for LN3 chosen from the list comprising SEQ ID NOs: 01, 02, 03, 04, 09, 12, 16, 19, 20 and 21.

### Cultivation

Completed growth media includes high glucose DMEM/F12, 10% FBS, 1% NEAA, 1% pen/strep, 1% ITS-X, 1% F-Glu, 10 ng/mL EGF, and 5 μg/mL hydrocortisone. Completed lactation media includes high glucose DMEM/F12, 1% NEAA, 1% pen/strep, 1% ITS-X, 1% F-Glu, 10 ng/mL EGF, 5 μg/mL hydrocortisone, and 1 μg/mL prolactin (5 ug/mL in Hyunh 1991). Cells are seeded at a density of 20,000 cells/cm² onto collagen coated flasks in completed growth media and left to adhere and expand for 48 hours in completed growth media, after which the media is switched out for completed lactation media. Upon exposure to the lactation media, the cells start to differentiate and stop growing. Within about a week, the cells start secreting lactation product(s) such as milk lipids, lactose, LN3, casein and whey into the media. A desired concentration of the lactation media can be achieved by concentration or dilution by ultrafiltration. A desired salt balance of the lactation media can be achieved by dialysis, for example, to remove unwanted metabolic products from the media. Hormones and other growth factors used can be selectively extracted by resin purification, for example, the use of nickel resins to remove His-tagged growth factors, to further reduce the levels of contaminants in the lactated product.

### G. General

### Heterologous and Homologous Expression

Genes that needed to be expressed, be it from a plasmid or from the genome were synthetically synthetized with one of the following companies: IDT or Twist Bioscience. Proteins described in present disclosure are summarized in Table 1. Unless stated otherwise, the UniProt IDs of the proteins described correspond to their sequence version 01 as present in the UniProt Database version release 2021_03 of 9 Jun. 2021. Expression could be further facilitated by optimizing the codon usage to the codon usage of the expression host. Genes were optimized using the tools of the supplier.

TABLE 1
Overview of proteins with corresponding SEQ ID NOs or UniProt IDs (sequence version
01, UniProt Database 2021_03 of 9 Jun. 2021) as described in this disclosure

| | | | | Country of origin of |
|---|---|---|---|---|
| | | | | digital sequence |
| SEQ ID NO | Name | Organism | Origin | information |
| 01 | LN3 importer | Bacillus sp. BK245 | Synthetic | Unknown |
| 02 | LN3 importer | Bacillus sp. AFS096315 | Synthetic | USA |
| 03 | LN3 importer | N.A. | Synthetic | N.A. |
| 04 | LN3 importer | N.A. | Synthetic | N.A. |
| 05 | MFS transporter | Acholeplasmataceae bacterium | Synthetic | South Korea |
| 06 | MFS transporter | Tenericutes bacterium | Synthetic | USA |
| 07 | MFS transporter | Clostridium disporicum | Synthetic | United Kingdom |
| 08 | MFS transporter | Paenibacillus yonginensis | Synthetic | South Korea |
| 09 | LN3 importer | Oscillospiraceae bacterium | Synthetic | Japan |
| 10 | MFS transporter | Erwinia iniecta | Synthetic | USA |
| 11 | MFS transporter | Bacillus sp. AFS002410 | Synthetic | USA |
| 12 | LN3 importer | Paenibacillus sp. OV219 | Synthetic | Unknown |
| 13 | MFS transporter | Vibrio kanaloae | Synthetic | USA |
| 16 | LN3 importer | N.A. | Synthetic | N.A. |
| 17 | Conserved domain | Artificial sequence | Synthetic | N.A. |
| 18 | Conserved domain | Artificial sequence | Synthetic | N.A. |
| 19 | LN3 importer | Paenibacillus pectinilyticus | Synthetic | South Korea |
| 20 | LN3 importer | Paenibacillus sp. FSL A5-0031 | Synthetic | USA |
| 21 | LN3 importer | Candidatus Fimenecus | Synthetic | Unknown |
| | | excrementigallinarum | | |
| 22 | melB | Salmonella typhimurium | Synthetic | South Korea |
| | | strain LT2 | | |

| | | | | Country of origin of |
|---|---|---|---|---|
| | | | | digital sequence |
| UniProt ID | Name | Organism | Origin | information |
| A0A077DGH3 | a13- | Basilea psitaccipulmonis | Synthetic | Switzerland |
| | fucosyltransferase | | | |
| A7LVG6 | AGE | Bacteroides ovatus | Synthetic | USA |
| A0ZZH6 | BaSP | Bifidobacterium adolescentis | Synthetic | Germany |
| E0IXR1 | cscB | Escherichia coli W | Synthetic | USA |
| Q61420 | CST | Mus musculus | Synthetic | USA |
| Q03417 | Frk | Zymomonas mobilis | Synthetic | United Kingdom |
| P09147 | galE | Escherichia coli K-12 | Synthetic | USA |
| | | MG1655 | | |
| O49213 | GER1 | Arabidopsis thaliana | Synthetic | USA |
| Q06210 | GFPT1 | Homo sapiens | Synthetic | Unknown |
| Q13630 | GFUS | Homo sapiens | Synthetic | Unknown |
| P31120* | glmM | Escherichia coli K-12 | Synthetic | USA |
| | | MG1655 | | |
| P17169** | glmS | Escherichia coli K-12 | Synthetic | USA |
| | | MG1655 | | |
| Q8NND3*** | glmS | Corynebacterium glutamicum | Synthetic | USA |
| P0ACC7 | glmU | Escherichia coli K-12 | Synthetic | USA |
| | | MG1655 | | |
| Q96EK6 | GNA1 | Homo sapiens | Synthetic | Unknown |
| P43577 | GNA1 | Saccharomyces cerevisiae | Synthetic | USA |
| O30511 | HpFucT | Helicobacter pylori UA1234 | Synthetic | Canada |
| Q9X435 | HpFutC | Helicobacter pylori UA1234 | Synthetic | Canada |
| Q9SEE5 | KIN1 | Arabidopsis thaliana | Synthetic | USA |
| P07921 | LAC12 | Kluyveromyces lactis | Synthetic | USA |
| P02920 | LacY | Escherichia coli K-12 | Synthetic | USA |
| | | MG1655 | | |
| Q9JXQ6 | LgtA | Neisseria meningitidis MC58 | Synthetic | United Kingdom |
| Q51116**** | LgtB | Neisseria meningitidis MC58 | Synthetic | United Kingdom |
| A0A2T7ANQ9 | MdfA | Cronobacter muytjensii | Synthetic | USA |
| D4BC23 | MdfA | Citrobacter youngae | Synthetic | USA |
| P0AEY8 | MdfA | Escherichia coli K-12 | Synthetic | USA |
| | | MG1655 | | |
| Q99KK2 | NeuA | Mus musculus | Synthetic | USA |
| A0A849CI62 | NeuA | Pasteurella multocida | Synthetic | USA |
| E0NCD4 | NeuB | Neisseria meningitidis | Synthetic | United Kingdom |
| Q93MP8 | NeuC | Campylobacter jejuni | Synthetic | USA |
| O66375 | PdST6 | Photobacterium damselae | Synthetic | Japan |
| O95394 | PGM3 | Homo sapiens | Synthetic | Unknown |
| Q9CLP3 | PmultST3 | Pasteurella multocida | Synthetic | USA |
| Q11203 | ST3GAL3 | Homo sapiens | Synthetic | Unknown |
| P13721 | ST6 | Rattus norvegicus | Synthetic | USA |
| Q16222 | UAP1 | Homo sapiens | Synthetic | Unknown |
| Q9C5I1 | USP | Arabidopsis thaliana | Synthetic | USA |
| D3QY14 | wbgO | Escherichia coli O55:H7 | Synthetic | Germany |

*Sequence version 03 (23 Jan. 2007) as present in the UniProt Database 2021_03 of 9 Jun. 2021
**Sequence version 04 (23 Jan. 2007) as present in the UniProt Database 2021_03 of 9 Jun. 2021
***Sequence version 02 (23 Jan. 2007) as present in the UniProt Database 2021_03 of 9 Jun. 2021
****Sequence version 02 (1 Dec. 2000) as present in the UniProt Database 2021_03 of 9 Jun. 2021

### Analytical Analysis

Standards such as but not limited to sucrose, lactose, LN3, LNT, LNnT, pLNnH were purchased from Carbosynth (UK), Elicityl (France) and IsoSep (Sweden). Other compounds were analyzed with in-house made standards.

Neutral oligosaccharides were analyzed on a Waters Acquity H-class UPLC with Evaporative Light Scattering Detector (ELSD) or a Refractive Index (RI) detection. A volume of 0.7 μL sample was injected on a Waters Acquity UPLC BEH Amide column (2.1×100 mm; 130 Å;1.7 μm) column with an Acquity UPLC BEH Amide VanGuard column, 130 Å, 2.1×5 mm. The column temperature was 50° C. The mobile phase consisted of a ¼ water and ¾ acetonitrile solution to which 0.2% triethylamine was added. The method was isocratic with a flow of 0.130 mL/min. The ELS detector had a drift tube temperature of 50° C. and the N2 gas pressure was 50 psi, the gain 200 and the data rate 10 pps. The temperature of the RI detector was set at 35° C.

Sialylated oligosaccharides were analyzed on a Waters Acquity H-class UPLC with Refractive Index (RI) detection. A volume of 0.5 μL sample was injected on a Waters Acquity UPLC BEH Amide column (2.1×100 mm; 130 Å;1.7 μm). The column temperature was 50° C. The mobile phase consisted of a mixture of 70% acetonitrile, 26% ammonium acetate buffer (150 mM) and 4% methanol to which 0.05% pyrrolidine was added. The method was isocratic with a flow of 0.150 mL/min. The temperature of the RI detector was set at 35° C.

Both neutral and sialylated sugars were analyzed on a Waters Acquity H-class UPLC with Refractive Index (RI) detection. A volume of 0.5 μL sample was injected on a Waters Acquity UPLC BEH Amide column (2.1×100 mm; 130 Å;1.7 μm). The column temperature was 50° C. The mobile phase consisted of a mixture of 72% acetonitrile and 28% ammonium acetate buffer (100 mM) to which 0.1% triethylamine was added. The method was isocratic with a flow of 0.260 mL/min. The temperature of the RI detector was set at 35° C.

### Example 3. Production of LNT with a Modified E. coli Host

An E. coli K12 MG1655 strain was modified as described in Example 2 comprising genomic knock-outs of the E. coli genes lacZ, nagB, galT and ushA and genomic knock-ins of constitutive transcriptional units containing the sucrose transporter (CscB) from E. coli W (UniProt ID EOIXR1), the fructose kinase (Frk) from Z. mobilis (UniProt ID Q03417) and the sucrose phosphorylase (BaSP) from B. adolescentis (UniProt ID AOZZH6) and the N-acetylglucosamine beta-1,3-galactosyltransferase wbgO (Uniprot ID D3QY14) from E. coli O55:H7. In a next step, the mutant strain was transformed with an expression plasmid containing a constitutive transcriptional unit for a saccharide importer having uptake activity for LN3 with SEQ ID NO:01. The novel strain was evaluated in a growth experiment for production of LNT according to the culture conditions provided in Example 2, in which the strain was cultivated in minimal medium supplemented with 15 g/L sucrose and 20 g/L LN3. A reference strain was used with the same genetic make-up as the novel mutant strain but lacking a saccharide importer sequence having uptake activity for LN3. Important to note here is that both strains were not able to make LN3 due to the lack of lactose in the medium and the lack of expression of a galactoside beta-1,3-N-acetylglucosaminyltransferase. The strains were grown in eight biological replicates in a 96-well plate. After 72 h of incubation, the culture broth was harvested, and the sugars were analyzed as described in Example 2. For each strain, the measured LNT concentration was averaged over all biological replicates. Also, a dilution of the cultures was made to measure the optical density at 600 nm. The cell performance index or CPI was determined by dividing the LNT concentrations measured in the whole broth by the biomass (g LNT/g X). The biomass is empirically determined to be approximately ⅓rd of the optical density measured at 600 nm. The experiment demonstrated that the strain expressing a saccharide importer with SEQ ID NO:01 was able to produce LNT out of the LN3 that was internalized by the saccharide importer having uptake activity for LN3 with SEQ ID NO:01 with a CPI of 0.55±0.26 (g LNT/g X), whereas the reference strain was not able to produce LNT due to the lack of a saccharide importer with uptake activity for the LN3 that was present in the cultivation medium.

### Example 4. Production of LNnT with a Modified E. coli Host

An E. coli K12 MG1655 strain is modified as described in Example 2 comprising genomic knock-outs of the E. coli genes lacZ, nagB, galT and ushA and genomic knock-ins of constitutive transcriptional units containing the sucrose transporter (CscB) from E. coli W (UniProt ID EOIXR1), the fructose kinase (Frk) from Z. mobilis (UniProt ID Q03417), the sucrose phosphorylase (BaSP) from B. adolescentis (UniProt ID AOZZH6) and the N-acetylglucosamine beta-1,4-galactosyltransferase LgtB (Uniprot ID Q51116, sequence version 02, 1 Dec. 2000) from N. meningitidis. In a next step, the mutant strain is transformed with an expression plasmid containing a constitutive transcriptional unit for a saccharide importer having uptake activity for LN3 chosen from the list comprising SEQ ID NOs: 01, 02, 03, 04, 09 and 12. The novel strains are evaluated in a growth experiment for production of LNnT according to the culture conditions provided in Example 2, in which the strains are cultivated in minimal medium supplemented with 15 g/L sucrose and 20 g/L LN3.

### Example 5. Production of LNT with a Modified E. coli Host when Evaluated in a Fed-Batch Fermentation Process with Sucrose and Lactose

An E. coli K12 MG1655 strain is modified for production of LNT as described in Example 2, comprising genomic knock-outs of the E. coli genes lacZ, nagB, galT and ushA and genomic knock-ins of constitutive transcriptional units containing the sucrose transporter (CscB) from E. coli W (UniProt ID EOIXR1), the fructose kinase (Frk) from Z. mobilis (UniProt ID Q03417) and the sucrose phosphorylase (BaSP) from B. adolescentis (UniProt ID AOZZH6), the galactoside beta-1,3-N-acetylglucosaminyltransferase IgtA (UniProt ID Q9JXQ6) from N. meningitidis and the N-acetylglucosamine beta-1,3-galactosyltransferase wbgO (Uniprot ID D3QY14) from E. coli 055:H7. In a next step, the mutant strain is further modified with a genomic knock-in of a constitutive transcriptional unit containing a saccharide importer having uptake activity for LN3 with SEQ ID NOs: 01, 02, 03, 04, 09, or 12. The novel strains are evaluated in a fed-batch fermentation using a 5 L bioreactor according to the culture conditions provided in Example 2. A reference strain having the same genetic make-up as the novel mutant strains but lacking expression of a saccharide importer having uptake activity for LN3 is evaluated in a similar fed-batch fermentation process. Sucrose is added as a carbon source, and lactose is added in the batch medium. During fed-batch, sucrose is added via an additional feed. In contrast to the cultivation experiments that are described herein and wherein only end samples are taken at the end of cultivation (i.e., 72 hours as described herein), regular broth samples are taken at several time points during the fermentation process and the LNT produced is measured as described in Example 2.

### Example 6. Production of LNnT with a Modified E. coli Host when Evaluated in a Fed-Batch Fermentation Process with Sucrose and Lactose

An E. coli K12 MG1655 strain is modified for production of LNnT as described in Example 2, comprising genomic knock-outs of the E. coli genes lacZ, nagB, galT and ushA and genomic knock-ins of constitutive transcriptional units containing the sucrose transporter (CscB) from E. coli W (UniProt ID EOIXR1), the fructose kinase (Frk) from Z. mobilis (UniProt ID Q03417), the sucrose phosphorylase (BaSP) from B. adolescentis (UniProt ID AOZZH6), the galactoside beta-1,3-N-acetylglucosaminyltransferase lgtA (UniProt ID Q9JXQ6) from N. meningitidis and the N-acetylglucosamine beta-1,4-galactosyltransferase LgtB (Uniprot ID Q51116, sequence version 02, 1 Dec. 2000) from N. meningitidis. In a next step, the mutant strain is further modified with a genomic knock-in of a constitutive transcriptional unit containing a saccharide importer having uptake activity for LN3 with SEQ ID NOs: 01, 02, 03, 04, 09, or 12. The novel strains are evaluated in a fed-batch fermentation using a 5 L bioreactor according to the culture conditions provided in Example 2. A reference strain having the same genetic make-up as the novel mutant strains but lacking expression of a saccharide importer having uptake activity for LN3 is evaluated in a similar fed-batch fermentation process. Sucrose is added as a carbon source, and lactose is added in the batch medium. During fed-batch, sucrose is added via an additional feed. In contrast to the cultivation experiments that are described herein and wherein only end samples are taken at the end of cultivation (i.e., 72 hours as described herein), regular broth samples are taken at several time points during the fermentation process and the LNnT produced is measured as described in Example 2.

### Example 7. Production of LNnT and LNFP-III with a Modified E. coli Host

The mutant E. coli K12 MG1655 strains expressing a saccharide importer having uptake activity for LN3 chosen from the list comprising SEQ ID NOs: 01, 02, 03, 04, 09 and 12 as described in Example 4 are further modified for production of GDP-fucose as described in Example 2 and transformed with an expression plasmid containing a constitutive transcriptional unit for the alpha-1,3-fucosyltransferase HpFucT from H. pylori (UniProt ID 030511). The novel strains are evaluated in a growth experiment for production of LNnT and LNFP-III according to the culture conditions provided in Example 2, in which the strains are cultivated in minimal medium supplemented with 15 g/L sucrose and 20 g/L LN3. After 72 h of incubation, the culture broth is harvested, and the sugars are analyzed on UPLC.

### Example 8. Production of LNnT and LSTc with a Modified E. coli Host

The mutant E. coli K12 MG1655 strains expressing a saccharide importer having uptake activity for LN3 chosen from the list comprising SEQ ID NOs: 01, 02, 03, 04, 09 and 12 as described in Example 4 are further modified for production of CMP-sialic acid as described in Example 2 and transformed with an expression plasmid containing a constitutive transcriptional unit for the alpha-2,6-sialyltransferase PdST6 from Photobacterium damselae (UniProt ID O66375). The novel strains are evaluated in a growth experiment for production of LNnT and LSTc according to the culture conditions provided in Example 2, in which the strains are cultivated in minimal medium supplemented with 15 g/L sucrose and 20 g/L LN3. After 72 h of incubation, the culture broth is harvested, and the sugars are analyzed on UPLC.

### Example 9. Production of an Oligosaccharide Mixture Comprising Fucosylated and Sialylated Oligosaccharide Structures with a Modified E. coli Host

A mutant E. coli K12 MG1655 modified for production of GDP-fucose as described in Example 2 is further modified with genomic knock-outs of the E. coli nagA, nagB, nanA, nanE and nanK genes and genomic knock-ins of constitutive expression cassettes for the sucrose transporter (CscB) from E. coli W (UniProt ID EOIXR1), the fructose kinase (Frk) from Z. mobilis (UniProt ID Q03417), the sucrose phosphorylase (BaSP) from B. adolescentis (UniProt ID AOZZH6), glmS*54 from E. coli (differing from the wild-type E. coli glmS (UniProt ID P17169) by an A39T, an R250C and an G472S mutation), the UDP-N-acetylglucosamine 2-epimerase (neuC) from C. jejuni (UniProt ID Q93MP8), the N-acetylneuraminate (Neu5Ac) synthase (neuB) from N. meningitidis (UniProt ID EONCD4), the N-acylneuraminate cytidylyltransferase (NeuA) from P. multocida (UniProt ID A0A849CI62), the UDP-glucose-4-epimerase galE from E. coli (UniProt ID P09147) and the N-acetylglucosamine beta-1,3-galactosyltransferase wbgO ((Uniprot ID D3QY14) from E. coli 055:H7. In a next step, the novel strain is transformed with two compatible expression plasmids wherein a first plasmid comprises constitutive transcriptional units for the alpha-1,2-fucosyltransferase (HpFutC) from H. pylori (UniProt ID Q9X435), the alpha-1,3-fucosyltransferase HpFucT from H. pylori (UniProt ID O30511), the alpha-2,3-sialyltransferase PmultST3 from P. multocida (UniProt ID Q9CLP3) and the alpha-2,6-sialyltransferase PdST6 from Photobacterium damselae (UniProt ID 066375) and wherein the second plasmid comprises a constitutive transcriptional unit for a saccharide importer with uptake activity for LN3 chosen from the list comprising SEQ ID NOs: 01, 02, 03, 04, 09 and 12. The novel strains are evaluated for production of an oligosaccharide mixture comprising fucosylated and sialylated structures such as LNFP-I, LNFP-V, LSTa, LNT, 3'S-LN3 and 6'S-LN3 in a growth experiment according to the culture conditions provided in Example 2 using appropriate selective medium comprising sucrose and LN3. After 72 h of incubation, the culture broth is harvested, and the sugars are analyzed on UPLC.

### Example 10. Production of LNnT with a Modified S. cerevisiae Host

A S. cerevisiae strain is modified as described in Example 2 with a first yeast expression plasmid comprising constitutive transcriptional units for the UDP-glucose-4-epimerase galE from E. coli (UniProt ID P09147), and the N-acetylglucosamine beta-1,4-galactosyltransferase LgtB (Uniprot ID Q51116, sequence version 02, 1 Dec. 2000) from N. meningitidis and with a second yeast expression plasmid comprising a constitutive transcriptional unit for a saccharide importer with uptake activity for LN3 chosen from the list comprising SEQ ID NOs: 01, 02, 03, 04, 09 and 12. The novel strains are evaluated for production of LNnT in a growth experiment according to the culture conditions provided in Example 2 using appropriate selective medium comprising LN3. After 72 h of incubation, the culture broth is harvested, and the sugars are analyzed on UPLC.

### Example 11. Production of LNT with a Modified B. subtilis Host

A wild-type B. subtilis strain is first modified with genomic knockouts of the B. subtilis genes nagB and gamA together with genomic knock-ins of constitutive transcriptional units for the N-acetylglucosamine beta-1,3-galactosyltransferase wbgO ((Uniprot ID D3QY14) from E. coli 055:H7 and a saccharide importer with uptake activity for LN3 chosen from the list comprising SEQ ID NOs: 01, 02, 03, 04, 09 and 12. The novel strains are evaluated for production of LNT in a growth experiment according to the culture conditions provided in Example 2 using appropriate selective medium comprising LN3. After 72 h of incubation, the culture broth is harvested, and the sugars are analyzed on UPLC.

### Example 12. Production of LNT and LNFP-V with a Modified C. glutamicum Strain

A C. glutamicum strain is first modified as described in Example 2 by genomic knock-out of the ldh, cgl2645 and nagB genes and genomic knock-ins of constitutive transcriptional units comprising genes encoding the sucrose transporter (CscB) from E. coli W (UniProt ID EOIXR1), the fructose kinase (Frk) from Z. mobilis (UniProt ID Q03417) and the sucrose phosphorylase (BaSP) from B. adolescentis (UniProt ID AOZZH6). In a next step, the mutant strain is further modified with genomic knock-ins of constitutive transcriptional units comprising the N-acetylglucosamine beta-1,3-galactosyltransferase WbgO from E. coli 055:H7 (UniProt ID D3QY14) and the alpha-1,3-fucosyltransferase HpFucT from H. pylori (UniProt ID O30511). In a subsequent step, the mutant strain is transformed with an expression plasmid comprising constitutive transcriptional units for a saccharide importer with uptake activity for LN3 chosen from the list comprising SEQ ID NOs: 01, 02, 03, 04, 09 and 12. The novel strains are evaluated for the production of LNT and LNFP-V in a growth experiment on MMsf medium comprising sucrose as carbon source and LN3 as precursor according to the culture conditions provided in Example 2. After 72 h of incubation, the culture broth is harvested, and the sugars are analyzed on UPLC.

### Example 13. Production of an Oligosaccharide Mixture Comprising Fucosylated and Sialylated Oligosaccharide Structures with a Modified C. glutamicum Strain

A C. glutamicum strain is first modified as described in Example 2 by genomic knock-out of the ldh, cgl2645 and nagB genes and genomic knock-ins of constitutive transcriptional units comprising genes encoding the sucrose transporter (CscB) from E. coli W (UniProt ID EOIXR1), the fructose kinase (Frk) from Z. mobilis (UniProt ID Q03417) and the sucrose phosphorylase (BaSP) from B. adolescentis (UniProt ID AOZZH6) for growth on sucrose. In a next step, the mutant strain is further modified with genomic knock-ins of constitutive transcriptional units comprising the N-acetylglucosamine beta-1,4-galactosyltransferase LgtB (Uniprot ID Q51116, sequence version 02, 1 Dec. 2000) from N. meningitidis and the alpha-1,3-fucosyltransferase HpFucT from H. pylori (UniProt ID O30511). In a next step, the mutant strain is further modified with a genomic knock-in of a constitutive transcriptional unit comprising the native fructose-6-P-aminotransferase (UniProt ID Q8NND3), GNA1 from S. cerevisiae (UniProt ID P43577), AGE from B. ovatus (UniProt ID A7LVG6), and the N-acetylneuraminate synthase from N. meningitidis (UniProt ID E0NCD4) to produce sialic acid. In a next step, the novel strain is transformed with a first expression plasmid comprising constitutive transcriptional units for the NeuA enzyme from P. multocida ((UniProt ID A0A849CI62), the beta-galactoside alpha-2,3-sialyltransferase PmultST3 from P. multocida (UniProt ID Q9CLP3) and the beta-galactoside alpha-2,6-sialyltransferase PdST6 from P. damselae (UniProt ID 066375) and a second compatible plasmid comprising constitutive transcriptional units for a saccharide importer with uptake activity for LN3 chosen from the list comprising SEQ ID NOs: 01, 02, 03, 04, 09 and 12. The novel strains are evaluated for the production of an oligosaccharide mixture comprising fucosylated and sialylated structures such as LNnT, LNFP-III, LSTc, LSTd, 3'S-LN3 and 6'S-LN3 in a growth experiment on MMsf medium comprising sucrose as carbon source and LN3 as precursor according to the culture conditions provided in Example 2. After 72 h of incubation, the culture broth is harvested, and the sugars are analyzed on UPLC.

### Example 14. Production of LNnT and LNFP-III in Modified C. reinhardtii Cells

C. reinhardtii cells are engineered as described in Example 2 for production of UDP-Gal and GDP-Fuc with genomic knock-ins of constitutive transcriptional units comprising the galactokinase from A. thaliana (KIN, UniProt ID Q9SEE5), the UDP-sugar pyrophosphorylase (USP) from A. thaliana (UniProt ID Q9C511) and the GDP-fucose synthase from Arabidopsis thaliana (GER1, UniProt ID 049213). In a next step, the mutant cells are transformed with an expression plasmid comprising transcriptional units comprising the N-acetylglucosamine beta-1,4-galactosyltransferase lgtB from N. meningitidis (Uniprot ID Q51116), the alpha-1,3-fucosyltransferase HpFucT from H. pylori (UniProt ID O30511) and a saccharide importer with uptake activity for LN3 chosen from the list comprising SEQ ID NOs: 01, 02, 03, 04, 09 and 12. The novel strains are evaluated in a cultivation experiment on TAP-agar plates comprising fucose, galactose, glucose and LN3 according to the culture conditions provided in Example 48. After 5 days of incubation, the cells are harvested, and the production of LNnT and LNFP-III is analyzed on UPLC.

### Example 15. Production of an Oligosaccharide Mixture Comprising Fucosylated and Sialylated Oligosaccharides in a Non-Mammary Adult Stem Cell

Isolated mesenchymal cells and re-programmed into mammary-like cells as described in Example 2 are modified via CRISPR-CAS to over-express the GlcN6P synthase GFPT1 from Homo sapiens (UniProt ID Q06210), the glucosamine 6-phosphate N-acetyltransferase GNA1 from Homo sapiens (UniProt ID Q96EK6), the phosphoacetylglucosamine mutase PGM3 from H. sapiens (UniProt ID 095394), the UDP-N-acetylhexosamine pyrophosphorylase UAP1 (UniProt ID Q16222), the N-acetylglucosamine beta-1,4-galactosyltransferase lgtB from N. meningitidis (Uniprot ID Q51116), the GDP-fucose synthase GFUS from H. sapiens (UniProt ID Q13630), the alpha-1,2-fucosyltransferase (HpFutC) from H. pylori (UniProt ID Q9X435), the alpha-1,3-fucosyltransferase HpFucT from H. pylori (UniProt ID O30511), the N-acylneuraminate cytidylyltransferase NeuA from Mus musculus (UniProt ID Q99KK2), the CMP-N-acetylneuraminate-beta-1,4-galactoside alpha-2,3-sialyltransferase ST3GAL3 from H. sapiens (UniProt ID Q11203), the alpha-2,6-sialyltransferase (UniProt ID P13721) from Rattus norvegicus and a saccharide importer with uptake activity for LN3 chosen from the list comprising SEQ ID NOs: 01, 02, 03, 04, 09 and 12. Cells are seeded at a density of 20,000 cells/cm² onto collagen coated flasks in completed growth media and left to adhere and expand for 48 hours in completed growth media, after which the media is switched out for completed lactation media with LN3 for about 7 days. After cultivation as described in Example 2, cells are subjected to UPLC to analyze for production of an oligosaccharide mixture comprising LNFP-III, LSTc, LSTd, LNnT, 3'S-LN3 and 6'S-LN3.

### Example 16. Production of LNT with a Modified E. coli Host

An E. coli K12 MG1655 strain was modified as described in Example 2 comprising genomic knock-outs of the E. coli genes lacZ, nagB, galT and ushA and genomic knock-ins of constitutive transcriptional units containing the sucrose transporter (CscB) from E. coli W (UniProt ID EOIXR1), the fructose kinase (Frk) from Z. mobilis (UniProt ID Q03417) and the sucrose phosphorylase (BaSP) from B. adolescentis (UniProt ID AOZZH6) and the N-acetylglucosamine beta-1,3-galactosyltransferase wbgO (Uniprot ID D3QY14) from E. coli O55:H7. In a next step, the mutant strain was transformed with an expression plasmid containing a constitutive transcriptional unit for (a) a saccharide importer having uptake activity for LN3 with SEQ ID NOs: 01, 02, 03, 04, 12 or 16, or (b) a polypeptide sequence with SEQ ID NOs: 05, 06, 07, 08, 10, 11 or 13, wherein expression of all the sequences is controlled by the same promoter and 5′ untranslated region (UTR) sequence. The novel strains were evaluated in a growth experiment for production of LNT according to the culture conditions provided in Example 2, in which the strains were cultivated in minimal medium supplemented with 15 g/L sucrose and 20 g/L LN3. A reference strain was used with the same genetic make-up as the novel mutant strains but lacking a saccharide importer sequence having uptake activity for LN3. Important to note here is that the strains were not able to make LN3 due to the lack of lactose in the medium and the lack of expression of a galactoside beta-1,3-N-acetylglucosaminyltransferase. The strains were grown in two biological replicates in a 96-well plate. After 72 h of incubation, the culture broth was harvested, and the sugars were analyzed as described in Example 2. For each strain, the measured LNT concentration was averaged over both biological replicates. Also, a dilution of the cultures was made to measure the optical density at 600 nm. The cell performance index or CPI was determined by dividing the LNT concentrations measured in the whole broth by the biomass (g LNT/g X). The biomass is empirically determined to be approximately ⅓rd of the optical density measured at 600 nm. The experimental data is depicted in Table 2. The data demonstrates that the strains expressing a saccharide importer with SEQ ID NOs: 01, 02, 03, 04, 12 and 16 were able to produce LNT out of the LN3 that was internalized by the saccharide importer having uptake activity for LN3 with SEQ ID NOs: 01, 02, 03, 04, 12 and 16 whereas the reference strain was not able to produce LNT due to the lack of a saccharide importer with uptake activity for the LN3 that was present in the cultivation medium. Also, the strains expressing a polypeptide sequence with SEQ ID NOs: 05, 06, 07, 08, 10, 11 or 13 were not able to produce LNT due to the lack of LN3 uptake activity for the polypeptide sequences. The saccharide importers with SEQ ID NOs: 01, 02, 03, 04, 12 and 16 originate from the major facilitator superfamily (MFS) of transporters and comprise a polypeptide sequence comprising an IPR001927 domain as defined by InterPro 90.0 as released on 4 Aug. 2022, and comprise a polypeptide sequence comprising 12 transmembrane (TM) domains with a conserved domain [AGSV][HNQ][ACDEGNQSTV]XX[FWY]XXXXX(no L), wherein X can be any amino acid residue, as represented by with SEQ ID NO: 17 present in the first TM domain, wherein the second amino acid residue of SEQ ID NO: 17 is aligned to Lys18 of the polypeptide with SEQ ID NO:22. The polypeptide sequences with SEQ ID NOs: 05, 06, 07, 08, 10, 11 and 13 also originate from the major facilitator superfamily (MFS) of transporters and comprise a polypeptide sequence comprising an IPR001927 domain as defined by InterPro 90.0 as released on 4 Aug. 2022 and comprise a polypeptide sequence comprising 12 transmembrane (TM) domains, but do not comprise a conserved domain as represented by SEQ ID NO:17 in their first TM domain, demonstrating the presence of the conserved domain as represented by SEQ ID NO: 17 within the first TM domain as essential for uptake activity for LN3 of the polypeptide sequences.

TABLE 2
Relative CPI of LNT (%) of the reference strain REF without saccharide
importer and of modified E. coli strains B, C, D, E and L
expressing (a) a saccharide importer with SEQ ID NOs: 02, 03, 04,
12 or 16 or (b) a polypeptide with SEQ ID NOs: 05, 06, 07, 08,
10, 11 or 13, compared to a modified E. coli strain A expressing
a saccharide importer with SEQ ID NO: 01. Strains were evaluated
in a growth experiment according to the cultivation conditions
provided in Example 2, in which the cultivation medium contained
15 g/L sucrose and 20 g/L LN3.

| Strain | SEQ ID NO | % CPI (g LNT/g X) ± sd |
|---|---|---|
| REF | / | 0 |
| A | 01 | 100% ± 37% |
| B | 02 | 940% ± 137% |
| C | 03 | 1320% ± 383% |
| D | 04 | 1526% ± 260% |
| E | 12 | 2207% ± 292% |
| L | 16 | 21% ± 30% |
| M | 05 | 0 |
| N | 06 | 0 |
| O | 07 | 0 |
| P | 08 | 0 |
| Q | 10 | 0 |
| R | 11 | 0 |
| S | 13 | 0 |

### Example 17. Production of LNT with a Modified E. coli Host

An E. coli K12 MG1655 strain was modified as described in Example 2 comprising genomic knock-outs of the E. coli genes lacZ, nagB, galT and ushA and genomic knock-ins of constitutive transcriptional units containing the sucrose transporter (CscB) from E. coli W (UniProt ID EOIXR1), the fructose kinase (Frk) from Z. mobilis (UniProt ID Q03417) and the sucrose phosphorylase (BaSP) from B. adolescentis (UniProt ID AOZZH6) and the N-acetylglucosamine beta-1,3-galactosyltransferase wbgO (Uniprot ID D3QY14) from E. coli O55:H7. In a next step, the mutant strain thus obtained was further engineered to create five new strains (F, G, H, I and J) wherein each strain was transformed with an expression plasmid containing a different promotor (P) and 5′ untranslated region (UTR) sequence combined with a common terminator (T6) sequence (Table 3) leading to different constitutive transcriptional units for a saccharide importer with SEQ ID NO:04 having uptake activity for LN3. The novel strains were evaluated in a growth experiment for production of LNT according to the culture conditions provided in Example 2, in which the strains were cultivated in minimal medium supplemented with 15 g/L sucrose and 20 g/L LN3. A reference strain was used with the same genetic make-up as the novel mutant strain but lacking a saccharide importer sequence having uptake activity for LN3. Important to note here is that the strains were not able to make LN3 due to the lack of lactose in the medium and the lack of expression of a galactoside beta-1,3-N-acetylglucosaminyltransferase. The strains were grown in four biological replicates in a 96-well plate. After 72 h of incubation, the culture broth was harvested, and the sugars were analyzed as described in Example 2. For each strain, the measured LNT concentration was averaged over all biological replicates. Also, a dilution of the cultures was made to measure the optical density at 600 nm. The cell performance index or CPI was determined by dividing the LNT concentrations measured in the whole broth by the biomass (g LNT/g X). The biomass is empirically determined to be approximately ⅓rd of the optical density measured at 600 nm. The experimental data is depicted in Table 4. The data demonstrated that all strains expressing a saccharide importer with SEQ ID NO:04 were able to produce LNT out of the LN3 that was internalized by the saccharide importer, whereas the reference strain was not able to produce LNT due to the lack of a saccharide importer with uptake activity for the LN3 that was present in the cultivation medium. The data also indicated that varying the expression strength of the saccharide importer can improve its activity.

TABLE 3
Promoter (P), untranslated region (UTR) and terminator (T) sequences used to express
the saccharide importer with SEQ ID NO: 4 integrated in an expression plasmid in
the mutant E. coli strains F, G, H, I and J as given in Table 4.

| Promoter sequence | Reference |
|---|---|
| P5 = PROM0005 = Mutalik_P5 | Mutalik et al. (Nat. Methods 2013, 10, 354-360) |
| P22 = PROM0022 = Anderson_J23111 | parts.igem.org/Promoters/Catalog/Anderson |
| P25 = PROM0025 = Mutalik_apFAB104 | Mutalik et al. (Nat. Methods 2013, 10, 354-360) |
| P30 = PROM0030 = Mutalik_apFAB140 | Mutalik et al. (Nat. Methods 2013, 10, 354-360) |
| P46 = PROM0046 = Mutalik_apFAB66 | Ross et al. (J. Bacteriol, 1998, 180(20), 5375-83) |
| UTR sequence | Reference/SEQ ID NO |
| UTR6 = UTR0006_OmpC_BCD12 | Mutalik et al. (Nat. Methods 2013, 10, 354-360) |
| UTR9 = UTR0009_GalE_BCD2 | Mutalik et al. (Nat. Methods 2013, 10, 354-360) |
| UTR22 = UTR0022_ThrA_BCD5 | Mutalik et al. (Nat. Methods 2013, 10, 354-360) |
| UTR201 | SEQ ID NO: 14 |
| Terminator sequence | SEQ ID NO |
| T6 | SEQ ID NO: 15 |

TABLE 4
CPI of LNT (g LNT/g X) of the reference strain REF without saccharide
importer and of the modified E. coli strains F, G, H, I, J, each
expressing the saccharide importer with SEQ ID NO: 4 from a different
expression cassette integrated on an expression plasmid (see Table
3). Strains were evaluated in a growth experiment according to the
cultivation conditions provided in Example 2, in which the cultivation
medium contained 15 g/L sucrose and 20 g/L LN3.

| | Expression cassette for the saccharide | |
|---|---|---|
| STRAIN | importer with SEQ ID NO: 04 | CPI (g LNT/g X) ± sd |
| REF | / | 0 |
| F | P22 - UTR201 - SEQ ID NO: 04 - T6 | 0.298 ± 0.028 |
| G | P25 - UTR6 - SEQ ID NO: 04 - T6 | 0.060 ± 0.008 |
| H | P30 - UTR9 - SEQ ID NO: 04 - T6 | 0.411 ± 0.043 |
| I | P46 - UTR22 - SEQ ID NO: 04 - T6 | 0.473 ± 0.055 |
| J | P5 - UTR22 - SEQ ID NO: 04 - T6 | 0.589 ± 0.086 |

### Example 18. Production of LNT with a Modified E. coli Host when Evaluated in a Fed-Batch Fermentation Process with Sucrose and Lactose

An E. coli K12 MG1655 strain was modified for production of LNT as described in Example 2, comprising genomic knock-outs of the E. coli genes lacZ, nagB, galT and ushA and genomic knock-ins of constitutive transcriptional units containing the sucrose transporter (CscB) from E. coli W (UniProt ID EOIXR1), the fructose kinase (Frk) from Z. mobilis (UniProt ID Q03417) and the sucrose phosphorylase (BaSP) from B. adolescentis (UniProt ID AOZZH6), the galactoside beta-1,3-N-acetylglucosaminyltransferase lgtA (UniProt ID Q9JXQ6) from N. meningitidis and the N-acetylglucosamine beta-1,3-galactosyltransferase wbgO (Uniprot ID D3QY14) from E. coli 055:H7. In a next step, the mutant strain was further modified with a genomic knock-in of a constitutive transcriptional unit containing a saccharide importer having uptake activity for LN3 with SEQ ID NO:04. The novel strain was evaluated in a fed-batch fermentation using a 5 L bioreactor according to the culture conditions provided in Example 2. A reference strain having the same genetic make-up as the novel mutant strain but lacking expression of a saccharide importer having uptake activity for LN3 was evaluated in a similar fed-batch fermentation process. Sucrose was added as a carbon source, and lactose was added in the batch medium. During fed-batch, sucrose was added via an additional feed. In contrast to the cultivation experiments that are described herein and wherein only end samples are taken at the end of cultivation (i.e., 72 hours as described herein), regular broth samples were taken at several time points during the fermentation process and the LN3 and LNT produced was measured as described in Example 2. The cell performance index or CPI was determined by dividing the LN3 and LNT concentrations measured in the whole broth by the biomass. The biomass is empirically determined to be approximately ⅓rd of the optical density measured at 600 nm. Table 5 shows the CPI values for LNT and LN3 obtained in broth samples taken at the end of the fed-batch fermentation, as well as the purity of LNT obtained in the samples. The data demonstrated a higher CPI value for LNT and a lower CPI value for LN3 in the strain expressing a saccharide importer with SEQ ID NO:04 compared to the reference strain lacking the saccharide importer. Also, the purity of LNT obtained at the end of the fed-batch fermentation in the broth sample of strain K was higher than that obtained in the reference strain.

TABLE 5
CPI of LNT and LN3 measured in a broth sample obtained at
the end of a fermentation of the reference strain REF without
saccharide importer and of modified E. coli strain K expressing
a saccharide importer with SEQ ID NO: 04. Strains were evaluated
in a fermentation experiment according to the cultivation
conditions provided in Example 2, in which the cultivation
medium contained sucrose and lactose.

| | CPI for LNT | CPI for LN3 | LNT purity |
|---|---|---|---|
| STRAIN | (g LNT/g X) | (g LN3/g X) | (g LNT/(g LNT + g LN3)) |
| REF | 1.385 | 0.067 | 95.4% |
| K | 1.474 | 0.049 | 96.7% |

### Example 19. Evaluation of Uptake of LNT or LNnT with a Modified E. coli Host

An E. coli K12 MG1655 strain was modified as described in Example 2 comprising genomic knock-outs of the E. coli genes lacZ, nagB, galT and ushA and genomic knock-ins of constitutive transcriptional units containing the sucrose transporter (CscB) from E. coli W (UniProt ID EOIXR1), the fructose kinase (Frk) from Z. mobilis (UniProt ID Q03417), the sucrose phosphorylase (BaSP) from B. adolescentis (UniProt ID AOZZH6). In a next step, the mutant strain was modified for production of GDP-fucose as described in Example 2. Furthermore, the mutant strain was transformed with an expression plasmid containing a constitutive transcriptional unit for (a) a saccharide importer having uptake activity for LN3 with SEQ ID NO: 01 and for (b) the alpha-1,3-fucosyltransferase from B. psittacipulmonis (UniProt ID A0A077DGH3).

The novel strain was evaluated in a growth experiment for production of LNFP-V according to the culture conditions provided in Example 2, in which the strain was cultivated in minimal medium supplemented with 15 g/L sucrose and 20 g/L LNT. The data showed that the mutant strain was not able to uptake LNT and did not produce LNFP-V (Results not shown).

The novel strain was also evaluated in a growth experiment for production of LNFP-III according to the culture conditions provided in Example 2, in which the strain was cultivated in minimal medium supplemented with 15 g/L sucrose and 20 g/L LNnT. The data showed that the mutant strain was not able to uptake LNnT and did not produce LNFP-III (Results not shown).

### Example 20. Identification of Protein Sequences Useful in the Methods of the Disclosure

MFS transporters can be obtained from sequence databases like Uniprot (www.uniprot.org/), NCBI nr or nt databases (ncbi.nlm.nih.gov/) and others. This example describes how to extract IPR001927 transporters comprising a polypeptide sequence comprising a conserved domain [AGSV][HNQ][ACDEGNQSTV]XX[FWY]XXXXX(no L) with SEQ ID NO: 17, wherein X can be any amino acid residue, present in the first TM domain and wherein the second amino acid residue [HNQ] of SEQ ID NO:17 is aligned to Lys18 of the polypeptide with SEQ ID NO:22. Members of IPR001927 were extracted using the Uniprot sequence database. In total 21203 protein sequences were found (21 Jun. 2023). Sequences were aligned with the polypeptide with SEQ ID NO:22 using clustalomega (ebi.ac.uk/Tools/msa/clustalo) and were filtered for sequences comprising [AGSV][HNQ][ACDEGNQSTV]XX[FWY]XXXXX(no L) with SEQ ID NO: 17, wherein X can be any amino acid residue, wherein the second amino acid [HNQ] is aligned to Lys18. Amino acid sequences were clustered using CD-HIT (weizhongli-lab.org/cd-hit/) with a sequence identity threshold of 80% and further filtered on completeness and source. The representative sequences are the next 701 identifiers: A0A0M4NCQ8, A0A1V5TMR7, A0A949VI13, A0A097IHW1, A0A161YHX3, A0A1C5Q8V8, A0A1S8LKB4, A0A6A2STI5, A0A288Q7N6, A0A353WPN0, A0A349MYV7, A0A5X6ERA2, A0A659GUW8, A0A288Q779, A0A0A1GPY0, U2WC19, A0A1H9Q7N1, E6K2V4, A0A0R2L4K3, USMR15, B0NC69, D1PXQ7, A0A5A5UIC6, A0A1C6HZ72, C0WW86, D4L2L1, A0A2A5RXL5, A0A355G430, A0A370AT03, A0A946P6G5, A0A7L4WFL9, A0A6V6ZI71, A0A2N1RIC1, A0A2N1SNE9, A0A2N2CGC0, A0A920CVE1, A0A919XRI3, A0A1C1A620, A0A1C1A609, A0A6P1TIK1, F6CWF5, A0A947QGS0, A0A174QTR6, R7KAA3, A0A9D1MV45, A0A9D1NB54, A0A3D5TR77, A0A3D2TMX2, A0A355L5X4, A0A355KWA6, A0A970D9T4, A0A972ECN2, A0A972EE24, A0A6M0YL09, A0A7C6NXV0, A0A9D1NCS8, A0A971H402, A0A970RP56, A0A3C1LSB5, A0A6A0B8P8, A0A7X7Q369, A0A9D2C702, A0A9D1V4T0, A0A832KNJ0, A0A357R216, A0A3D4VPX6, A0A1H7ZD46, A0A1M5LHP4, A0A1H3BUC5, A0A1V5TLZ3, 18TWS5, J8AVR3, A0A1V6FPG8, A0A1V5 TMW2, A0A1V5S6E0, A0A928JQ48, A0A943PJ01, A0A9D5YCZ8, A0A943Q645, A0A9D6APP5, A0A316RBM9, A0A928PS37, A0A943K8B6, A0A7Y7ASR5, A0A2A7WQ92, A0A419ECE9, A0A9D5YAM1, A0A1F9ZSC8, A0A841RAJ2, A0A6A4UZY5, A0A2A8SME2, A0A1R0YCE6, A0A2V2DR69, A0A1R0ZVT2, A0A942KFU8, G8QSG5, G8QRP4, A0A087D6F9, R0J6Z3, A0A4S2B8J7, A0A928FYL2, A0A4R0U1H7, Q97L68, A4QGN8, K9IB44, A0A837IWL5, B2GBT3, A0A0H3B1S7, D5T091, A0A0J1FY73, 17IVF3, N1ZU84, A0A087DGU1, B7GNN4, A0A0R2A3L4, A0A0F3RYP2, A0A0R1GT62, A0A0R2E3U5, A0A0R1W961, A0A0R2CQX4, A0A0R1YJL0, A0A0R1LEV3, B8HCK1, A0A0C2Z3W6, A0A062X1H3, X0QQ49, A0A1A7P2F8, A0A0R1W0X1, A0A511DLS7, A0A143YLG7, A0A1V5S2T2, A0A0R1 L7 MI, A0A0R1 T563, A0A261FIE1, Q04GZ1, A0A0R2DH03, S0L2K9, A0A0R1 P5L6, A0A498RE57, A0A0B3VFV1, A0A0R1 RL35, A0A922PUF8, A0A0R1 GFH7, A0A0R1 W4C4, A0A143YFA8, A0A0R1 R2C5, A0A0E3UU18, A0A0R1 UVT1, A0A0R1 L1Y7, K6CQH1, K6D384, A0A0A2UU02, X0PNS7, A0A0R1 L8W6, A0A0K2 MG78, A0A081NRK1, A0A1V1I112, A0A0R1 VAD5, A0A0R2BGZ4, A0A0R1V755, A0A261G7A6, A0A0R1 TH64, A0A0R2DJJ2, A0A149PHS3, A0A0R1 KXG8, A0A0R2BMS3, A0A143YZ79, A0A0R2B1O0, A0A0J5P9A5, A0A0R1GRF1, A0A0R1 TS06, R9JTV1, R9IH22, G9WJA3, A0A0B3WRS8, R9M0M4, A0A0E3M467, A0A0BOD119, A0A0R1 XA29, G2T5P9, B812N3, C7REK1, R9K2Y1, R9JDA9, A0A162J2A9, Q03GR8, A0A0F3RRA2, A0A081KGX7, A0A0F3RS37, G9WEW2, V6J221, A0AOR2A492, A0A075KEU2, A0A0R1 QI74, A0A0R1 VBB4, S5SWY5, A0A0R1 YPP0, A0A0R1U042, A0A847HDD8, A0A4V3REA8, A0A211IZ22, A0A087BE03, A0A5N0A0L2, A0A351V794, A0A1W6NDN7, A0A4V3R6A0, A0A3D5VYH4, A0A7X7S2W4, A0A430FW05, A0A615MXY7, A0A087E0V4, A0A7M4BQ82, A0A087BE04, A0A7X7JUH2, A0A7Y0HVL9, A0A7Y0HU57, A0A7K0K1R2, A0A3Q8D1C1, A0A612T4G0, A0A2B7VXX5, A0A1R1IE54, A0A4S4F7J8, A0A7W3UJQ4, A0A852Y734, R4KCI8, R5FI43, A0A7W4UHR6, A0A7Y0HTP0, A0A928JYE1, A0A315S501, A0A387BLS6, A0A3E0VE10, A0A2T9XM72, A0A970UKW6, A0A1R1ESD0, A0A6N7EIR0, A0A1X2YV29, A0A927YDP0, A0A928PUV2, A0A1W6NC63, A0A425XRN5, A0A7X9N432, A0A242CYF9, A0A1B2IX47, A0A2S9D6K0, A0A9D1MHS0, A0A5M9Q3A6, A0A5M9ZSM0, A0A7Y0EPK8, K7RK22, A0A8I0HRB6, A0A4R1K265, A0A928PLZ9, A0A928PSR4, K7RKW1, A0A2N5JCN2, A0A7C6IDK4, A0A921JLZ2, A0A6N4HYH4, A0A7X1ZCT8, A0A4P6YUS7, A0A0Q9KZL3, A0A943KDB4, A0A6N7EMF0, A0A9D1L1W4, A0A0V8BVW4, A0A938WYS4, A0A1W6NDP8, R6Q979, R5N0J3, A0A9D1MVC6, A0A7T4EHC4, A0A9D1UGF5, A0A1Z518T6, A0A940JKZ7, A0A943VF81, A0A9D1IFA8, A0A943S2G8, A0A2V2D8M4, A0A1R1L6S6, A0A3D5YPS1, A0A431ISA9, A0A4P6EL23, A0A7W3R354, A0A970RQ55, A0A5P8JMF7, A0A291H255, A0A839NE64, A0A482PV20, A0A4S2FCC0, A0A847ELX9, A0A0L0R1A8, A0A9D1NBR0, A0A317ZLP4, A0A317ZYQ4, A0A7Y0EPV7, A0A7Y0F2R5, A0A854ZKS5, A0A1V8YV37, A0A291GSH9, A0A2T0VE54, A0A9D2 LB09, A0A9D5MM67, A0A927WV36, A0A1V8PSM4, A0A7W3TSD5, A0A5D4H5J1, A0A316R9K7, A0A511DZG9, A0A3Q8GWH2, A0A1X4JK84, A0A4R5NUC4, A0A0U4WW71, A0A2T5IHH7, A0A921KGI9, A0A970XKN2, A0A2N6TZC4, A0A2S5XIL6, A0A4V2KRA9, A0A7L5UJ94, A0A4Z0RW49, A0A4U0CDX9, A0A9D5NQ35, A0A973JH97, A0A4R3K8P2, A0A7MIQSN5, A0A930CLP8, A0A612T3B4, A0A971WCS4, A0A0J6ZS11, A0A8E1X3F7, A0A921F9C5, A0A5N0A0J0, A0A2V2D466, A0A971PUT1, A0A1U7NQR7, A0A5B8TG66, A0A857A9V8, A0A6G8B1H6, A0A371IXW9, A0A1Y4R0D3, A0A7J5LHF4, A0A848B0Y4, A0A7HIMMA7, A0A9D1SMN9, A0A3R8HU62, A0A6N4HJW4, A0A2N5ETT8, A0A0G3HBT8, A0A6N914Y2, A0A7S8W4N4, A0A2S9A6LI, A0A975SU96, A0A5M9DJF2, A0A4S2HM38, A0A2V5IZ57, A0A316MWI2, A0A4S3PSQ7, A0A5P2TWJ3, A0A5M9ZRY7, A0A6H1WWY1, A0A6N7E1H3, A0A7Z0KV28, A0A346MUT7, A0A1B3X2K1, A0A2T0PYC4, A0A7X0HTL4, A0A7Y0HSN0, A0A9D9E6G0, A0A970YXS5, A0A3E2DNK8, A0A7Y8S487, A0A9D5NPF1, A0A807D1H1, A0A9E1HSS1, A0A1Q6 FT65, A0A4R1J9F7, A0A2T4QJT2, A0A0Q5J530, A0A1F3GWF6, A0A938WS78, A0A921F1E6, A0A0K1FHY8, A0A943H2R0, A0A5N1GI99, A0A5M9DPD4, A0A2N6V048, A0A6N7W6Y5, A0A3N4Z6A6, A0A556UD18, A0A927WRS0, A0A351M1T4, A0A613Q6U9, A0A3G5HSG6, A0A417YC50, A0A4R5Y214, A0A6H1WX58, A0A6L8VDM2, A0A7X8ANQ9, A0A927TP51, A0A9D9H7Y9, A0A2N0W3H0, A0A385AG69, A0A1Y3XCV0, A0A9D5NUX9, A0A2K4BV99, A0A6N7WHY2, A0A7T4MWZ0, A0A4Q1B0X3, A0A4R1VFI6, A0A5B81461, A0A7X7BY38, A0A7X7XN22, A0A847K1Q8, A0A2C1Z3S1, A0A7X9N4W3, A0A9D8RZ55, A0A9D9ZDU9, A0A943JE26, A0A6G8JJZ4, A0A150JZE6, A0A0Q7VFU7, A0A0M2XRE6, A0A109N342, A0A9E1HRB2, A0A1R1C2J1, A0A1V9BEE9, A0A2T5INE8, A0A2W1ZDT7, A0A3S9H9G9, A0A498D104, A0A5J5HMM5, A0A7K0K513, A0A7X7V9VI, A0A927R1D6, A0A928EK38, A0A938WN38, A0A947VP36, A0A9D8WYK5, A0A9D9M413, A0A2A9DK13, A0A1L6RB82, A0A6N4HMZ3, R₄K7X4, A0A222WTM3, A0A0C1PUY6, A0A9D1GLL5, A0A927CX17, A0A1C1A6L9, A0A1Q6N8J3, A0A2C1YWL4, A0A1B2IVR0, A0A927HBQ2, A0A4R2J1B4, A0A355GW30, A0A3N2NI83, A0A6H1WWN1, A0A7X0HSS4, A0A855XWH9, A0A9D1RD66, C6D6D6, A0A7X0ZTQ4, A0A482PY26, A0A4S2EL08, A0A347T7K7, A0A5J5E1Z5, A0A941A6L3, A0A921EFN1, A0A1Y3TDF2, A0A9E1FVJ6, A0A2T4Q082, A0A0Q9X1T4, A0A558LSN9, A0A921KW76, A0A1C7I1M5, A0A5N7INB0, A0A3A0VVU9, A0A2N5HW07, A0A943IJ00, A0A354D2N3, A0A7X9RBT4, A0A4Y4F2H0, A0A9D1PR79, A0A9D2L7P5, A0A9D5RKZ3, R7N921, A0A7H5CXV6, A0A125QSD4, A0A855LDU9, A0A9E0GWA4, A0A0H4QGR7, A0A4Q7PMP7, A0A613LFT9, A0A9D1DQS2, A0A9D9N7N2, A0A252CCI7, A0A3D4CB94, A0A9D1TWZ3, A0A255SNW6, A0A3N1VYR9, A0A354D2H1, A0A252F318, A0A943BTY1, A0A971AYU2, A0A9D2H9E2, A0A9D2JRN4, A0A6I1MQP5, A0A6N7VU87, A0A2V3Y393, A0A177KLQ3, A0A2N5PGG7, A0A0V8D3X3, A0A5M9EJI6, A0A345YT19, A0A3B9G3F9, A0A7X2PBY5, A0A9D1R7X2, A0A4R2LZ65, R5T190, A0A7L5UK50, A0A1Y4J4K3, A0A224XBT5, A0A329U599, A0A837XWD8, A0A2P2BN63, A0A351XK17, A0A8B5W4G1, A0A858N3A3, A0A0Q9MWS9, A0A0U4WXZ4, A0A109RGQ5, A0A2J6NL75, A0A844DQX9, A0A9E1HNA8, A0A347STM8, A0A846RSK6, A0A7X2TDB2, A0A9D1IEP7, A0A9D1IF46, A0A9D1XEQ2, A0A231PUE0, A0A2B2CDW6, A0A3D8Q2W0, A0A9E1M6F8, A0A3P1TB73, A0A6A8HKA6, A0A847KNV2, A0A927NUJ4, A0A9D8S524, A0A9E2KCE8, A0A351VDB0, A0A0G3HIH9, A0A7X7DKK3, A0A928IZV5, A0A9D5YPS5, A0A2A9Y0R8, A0A9E1HNT9, A0A6F9XP93, A0A7C6PXH0, A0A9E1GNF5, R7HC86, A0A4S2F8U5, A0A7Z0HUG3, A0A211MR29, A0A7V8C8E3, A0A1R1EMB4, A0A7Y0HQ40, A0A971JFE8, A0A1B1EGU6, A0A841R987, A0A3D4XDP1, A0A2A7MBG7, A0A7W7H1J5, A0A840PAK4, A0A368VRB5, A0A615XG98, A0A3D8WZ48, A0A2T4SD22, A0A943BAN9, A0A972GUZ5, A0A9D5SWR1, A0A7X3MM69, A0A1C1A8C7, A0A1D9FS27, A0A3D8PR51, A0A5C8BSU1, A0A9D1QP37, A0A431IX87, A0A971JWJ9, A0A927YA85, A0A5B0VV11, A0A7Y4MCZ6, A0A2N6V0V5, A0A616IB44, A0A5C8B5B9, A0A3D9SJ21, A0A7X7ZHS7, A0A1L3KBK7, A0A928N2L6, A0A853BA14, A0A5B8T146, A0A4R2JM64, A0A3M1Y164, A0A940ZTW6, A0A8E2VFH5, A0A839K2Q7, A0A179CKK3, A0A350WZ19, A0A5B8T975, A0A43111F4, A0A841ABP3, A0A327Z072, A0A363E972, A0A6A8HL09, A0A5B8TDT6, A0A4V6NYX4, A0A1V0GDT7, A0A5M9EH01, A0A5M9DJT4, A0A6A8M9W7, A0A2Z5Y4F8, A0A1Z5I1Q2, A0A1Z5IR17, A0A229VXN6, A0A127A0F0, M5AFC9, A0A653W3A3, A0A127A518, A0A069D2T3, A0A6V8SHB5, A0A6V8SHR7, A0A6V8SBP2, A0A6V8SPQ4, A0A5Q2F9U3, A5UFL8, A0A37512Z6, A0A5Q2F779, A0A062X9L8, A0A062X829, A0A1M4RV76, A0A1G6GFK7, A0A4Y3 KM38, A0A1C4H4W0, A0A919K8Y6, A0A1G6H423, A0A919K908, A0A401FJ76, A0A133KY14, S2WN03, S2WXM7, A0A1F1W8X9, F3AC95, D2EHP1, A0A1F0PG23, C6PWS4, F7JLQ1, F7JIC3, A0A828RX65, A0A401FJ64, A0A919L107, A0A8J3AJQ6, A0A1G7A2E5, A0A1G9N786, A0A1X7HVB1, A0A1H9TNT8, A0A1H5EJ42, A0A1H4INR2, A0A1G9IBK0, A0A1IIJQC9, A0A116LZP3, A0A1H1KRX9, A0A1H9SJP4, A0A1H7BQJ6, A0A1H1MBT6, A0A285VM07, B0A7B3, A0A1G8DVX2, A0A1H9NZ12, A0A1I1JNL2, A0A1H3E7G2, A0A1H9U916, A0A1D3TR42, A0A1H1MBB5, A0A1M5J1K4, A0A1G5CZS1, A0A1G7H604, A0A1M6SX26, A0A1H8S6G7, A0A1G9WJI5, A0A1M4UC49, A0A1W1X812, A0A1G9YAW9, A0A1I1RRF6, A0A1G8P825, A0A1H0BYD7, A0A115D7Z2, A0A1M6K6R5, S6CPQ1, A0A7D0PIZ9, G2SYD4, A0A553SUQ6.

### Example 21. Identification of Protein Sequences Useful in the Methods of the Disclosure

MFS transporters can be obtained from sequence databases like Uniprot (uniprot.org/), NCBI nr or nt databases (ncbi.nlm.nih.gov/) and others. This example describes how to extract IPR001927 transporters comprising a polypeptide sequence comprising a conserved domain [AGS]Q[ACGNQSTV]XX[FWY] with SEQ ID NO: 18, wherein X can be any amino acid residue, present in the first TM domain and wherein the second amino acid residue Q of SEQ ID NO: 18 is aligned to Lys 18 of the polypeptide with SEQ ID NO:22. Members of IPR001927 were extracted using the Uniprot sequence database. In total 21203 protein sequences were found (21/06/2023). Sequences were aligned with the polypeptide with SEQ ID NO:22 using clustalomega (ebi.ac.uk/Tools/msa/clustalo) and were filtered for sequences comprising [AGS]Q[ACGNQSTV]XX[FWY] with SEQ ID NO: 18, wherein X can be any amino acid residue, wherein the second amino acid Q is aligned to Lys18. Amino acid sequences were clustered using CD-HIT (weizhongli-lab.org/cd-hit/) with a sequence identity threshold of 80% and further filtered on completeness and source. The representative sequences are the next 217 identifiers: R7KAA3, A0A0A1GPY0, A0A0M4NCQ8, A0A211IZ22, A0A4R0U1H7, A0A6A2STI5, A0A928JQ48, A0A612T3B4, A0A0R2F2A6, A0A971WCS4, A0A857A9V8, A0A6G8B1H6, A0A7J5LHF4, A0A1Y4R0D3, A0A174QTR6, A0A9D1SMN9, A0A7S8W4N4, A0A430FW05, A0A0H3BIS7, A0A316MWI2, A0A288Q7N6, A0A7Y7ASR5, A0A970YXS5, A0A9D5NPF1, A0A1F3GWF6, A0A938WS78, A0A1Q6 FT65, A0A7C6NXV0, A0A615MXY7, A0A9D1NCS8, A0A949VI13, A0A971H402, A0A351MIT4, A0A353WPN0, A0A9D5NUX9, A0A6N7WHY2, A0A1H9U916, A0A2NOW3H0, A0A7X7BY38, A0A087E0V4, A0A9D9ZDU9, A0A0M2XRE6, A0A0A2UU02, A0A1C1A620, A0A938WN38, A0A970RP56, A0A9D9M413, DIPXQ7, A0A0R1 XTX4, A0A0C1PUY6, A0A943PJ01, A0A355GW30, A0A3N2NI83, A0A1M5J1K4, A0A7X0ZTQ4, A0A347T7K7, A0A0R2DJJ2, 18TWS5, A0A7H5CXV6, A0A1F0PG23, A0A1V5TLZ3, A0A2A7WQ92, A0A255SNW6, A0A3C1LSB5, A0A7W0FMJ6, A0A419ECE9, A0A1G7H604, A0A349MYV7, A0A0B0D119, A0A1G9WJI5, A0A2N1R1C1, A0A1W6A0Q8, A0A5X6ERA2, A0A8B5W4G1, A0A9D1IEP7, A0A1C6HZ72, R9K2Y1, A0A087BE04, A0A9E1HNT9, A0A2A9Y0R8, A0A4S2F8U5, A0A971JFE8, F6CWF5, A0A0F3RRA2, A0A355G430, G8QSG5, A0A2A5RXL5, J8AVR3, A0A7L4WFL9, A0A659GUW8, A0A947QGS0, A0A9D5SWR1, A0A1C1A609, A0A1C5Q8V8, A0A7Y0HU57, A0A7X3MM69, A0A9D5YAM1, A0A1F9ZSC8, A0A6A0B8P8, F7J1C3, A0A1H7ZD46, A0A087BE03, A0A7X7Q369, A0A946P6G5, A0A0R2A492, A0A1M5LHP4, A0A841RAJ2, A0A919XRI3, A0A9D2C702, A0A1H0BYD7, A0A1V5TMW2, A0A6A4UZY5, A0A1H3BUC5, A0A2A8SME2, A0A920CVE1, A0A1R0YCE6, A0A612T4G0, A0A2V2DR69, A0A9D1V4T0, G8QRP4, A0A2N2CGC0, A0A3M1Y164, A0A832KNJ0, A0A115D7Z2, A0A087DGU1, A0A940ZTW6, A0A828RX65, A0A1R0ZVT2, A0A6P1TIK1, A0A839K2Q7, A0A357R216, A0A6V6ZI71, A0A841R8N9, A0A350WZ19, A0A1V5S6E0, A0A3D4VPX6, A0A942KFU8, A0A43111F4, A0A7U9SH75, A0A4S4F7J8, B7GNN4, A0A3D5TR77, R5FI43, A0A2N1SNE9, S6CPQ1, A0A3D2TMX2, A0A928JYE1, A0A9D5YCZ8, A0A315S501, A0A179B3D5, A0A355L5X4, A0A943Q645, A0A387BLS6, A0A9D6APP5, A0A970UKW6, A0A5N0A0L2, A0A3D8U880, A0A1R1ESD0, A0A316RBM9, A0A1W6NC63, A0A7X9N432, A0A1V5TMR7, A0A242CYF9, A0A928PUV2, A0A355KWA6, A0A0V8BVW4, A0A9D1 MV45, A0A7Y0EPK8, A0A970D9T4, A0A0R2CQX4, A0A133KY14, A0A928PLZ9, A0A928PSR4, A0A2N5JCN2, A0A921JLZ2, A0A6N4HYH4, A0A1V6FPG8, A0A4P6YUS7, A0A943KDB4, A0A972ECN2, A0A938WYS4, A0A972EE24, R6Q979, R5N0J3, A0A940JKZ7, A0A943VF81, A0A9D1UGF5, A0A9D1NB54, A0A9D1IFA8, A0A9D1MVC6, A0A0C2Z3W6, A0A943S2G8, E6K2V4, A0A4P6EL23, A0A970RQ55, A0A1A7P2F8, A0A062X1H3, A0A4Q7DLW6, A0A4V3R6A0, A0A482PV20, A0A928PS37, A0A1H1KRX9, A0A943K8B6, A0A9D1NBR0, A0A1Z5IR17, A0A927WV36, A0A1V8PSM4, A0A5D4H5J1, A0A1V5S2T2, A0A6M0YL09, A0A3Q8GWH2, A0A1X4JK84, A0A970XKN2, A0A316R9K7, A0A4Z0RW49, A0A9D5NQ35, A0A930CLP8.

1.-39. (canceled)

40. A cell for producing one or more bioproduct(s), wherein the cell is able to produce one or more precursor(s) used in producing at least one of the one or more bioproduct(s), optionally the cell is genetically engineered to produce one or more precursor(s) used in producing at least one of the one or more bioproduct(s) and wherein the cell comprises:
an exogenous polynucleotide encoding a saccharide importer that internalizes at least one of the one or more precursor(s) into the cell.

41. The cell of claim 40, wherein the saccharide importer has uptake activity for lacto-N-triose (LN3, GlcNAc-β1,3-Gal-β1,4-Glc).

42. The cell of claim 40, wherein:
one of the one or more precursor(s) is LN3, or
one of the one or more precursor(s) is LN3 and the saccharide importer has uptake activity for the LN3 being produced by the cell.

43. A cell for producing one or more bioproduct(s), wherein the exogenous polynucleotide has uptake activity for lacto-N-triose (LN3, GlcNAc-β1,3-Gal-β1,4-Glc).

44. The cell of claim 40, wherein the saccharide importer comprises a polypeptide:
that originates from the major facilitator superfamily (MFS) of transporters,
comprising an IPR domain selected from the group consisting of IPR001927, IPR002178, IPR016152, IPR018043, IPR020846, IPR036259 and IPR039672 as defined by InterPro 90.0 as released on 4 Aug. 2022,
comprising PF13347 domain and/or PF00359 domain as defined by PFAM 32.0 as released on September 2018,
comprising a PANTHER domain selected from the group consisting of PTHR11328, PTHR11328:SF24, PTHR11328:SF36 and PTHR11328:SF39 as defined by PANTHER 18.0 as released on 17 Sep. 2023,
comprising cd17332 domain and/or cd00211 domain as defined by the Conserved Domain Database CDD 3.20 as released on September 2022,
that is at least 25% identical over a stretch of at least 50 amino acid residues to any one of the polypeptides as represented by SEQ ID NOs: 4, 12, 2, 1, 3, 9, 16, 19, 20 or 21,
that is at least 25% identical to any one of the full-length polypeptides as represented by SEQ ID NOs: 4, 12, 2, 1, 3, 9, 16, 19, 20 or 21,
as represented by any one of SEQ ID NOs: 4, 12, 2, 1, 3, 9, 16, 19, 20 or 21,
that has a deletion, insertion and/or a mutation of one or more amino acid residue(s) N-terminally from the first transmembrane (TM) domain of any one of SEQ ID NOs: 12, 2, 1, 9, 19, 20 or 21, optionally the deletion, insertion and/or mutation is in or N-terminally of a non-TM helix, and/or
that has a deletion, insertion and/or a mutation of one or more amino acid residue(s) C-terminally from the last TM domain of any one of SEQ ID NOs: 12, 2, 1, 9, 19, 20 or 21, optionally the deletion, insertion and/or mutation is in or C-terminally of a non-TM helix.

45. The cell of claim 40, wherein the saccharide importer originates from the major facilitator superfamily (MFS) of transporters and comprises a polypeptide comprising:
an IPR001927 domain as defined by InterPro 90.0 as released on 4 Aug. 2022, and
a polypeptide comprising 12 transmembrane (TM) domains with a conserved domain [AGSV][HNQ][ACDEGNQSTV]XX[FWY]XXXXX(no L) as represented by SEQ ID NO: 17, wherein X can be any amino acid residue, present in the first TM domain, optionally a polypeptide comprising 12 TM domains with a conserved domain [AGS]Q[ACGNQSTV]XX[FWY] as represented by SEQ ID NO: 18, wherein X can be any amino acid residue, present in the first TM domain,
optionally, wherein the second amino acid residue of SEQ ID NO: 17 is aligned to Lys18 of the polypeptide with SEQ ID NO: 22, and
optionally, wherein the second amino acid residue of SEQ ID NO: 18, is aligned to Lys18 of the polypeptide with SEQ ID NO: 22.

46. The cell of claim 41, wherein the saccharide importer further has uptake activity for one or more other saccharide(s) different from LN3, wherein the one or more other saccharide(s) different from LN3 are selected from the group consisting of monosaccharide, disaccharide, oligosaccharide, and polysaccharide.

47. The cell of claim 41, wherein the saccharide importer has uptake activity for LN3 but not for a) LNT (lacto-N-tetraose, Galβ1-3GlcNAcβ1-3Galβ1-4Glc) and/or b) LNnT (lacto-N-neotetraose, Galβ1-4GlcNAcβ1-3Galβ1-4Glc).

48. The cell of claim 43, wherein the saccharide importer comprises a polypeptide:
that originates from the major facilitator superfamily (MFS) of transporters,
comprising an IPR domain selected from the group consisting of IPR001927, IPR002178, IPR016152, IPR018043, IPR020846, IPR036259 and IPR039672 as defined by InterPro 90.0 as released on 4 Aug. 2022,
comprising PF13347 domain and/or PF00359 domain as defined by PFAM 32.0 as released on September 2018,
comprising a PANTHER domain selected from the group consisting of PTHR11328, PTHR11328:SF24, PTHR11328:SF36 and PTHR11328:SF39 as defined by PANTHER 18.0 as released on 17 Sep. 2023,
comprising cd17332 domain and/or cd00211 domain as defined by the Conserved Domain Database CDD 3.20 as released on September 2022,
that is at least 25% identical over a stretch of at least 50 amino acid residues to any one of the polypeptides as represented by SEQ ID NOs: 4, 12, 2, 1, 3, 9, 16, 19, 20 or 21,
that is at least 25% identical to any one of the full-length polypeptides as represented by SEQ ID NOs: 4, 12, 2, 1, 3, 9, 16, 19, 20 or 21,
as represented by any one of SEQ ID NOs: 4, 12, 2, 1, 3, 9, 16, 19, 20 or 21,
that has a deletion, insertion and/or a mutation of one or more amino acid residue(s) N-terminally from the first transmembrane (TM) domain of any one of SEQ ID NOs: 12, 2, 1, 9, 19, 20 or 21, optionally the deletion, insertion and/or mutation is in or N-terminally of a non-TM helix, and/or
that has a deletion, insertion and/or a mutation of one or more amino acid residue(s) C-terminally from the last TM domain of any one of SEQ ID NOs: 12, 2, 1, 9, 19, 20 or 21, optionally the deletion, insertion and/or mutation is in or C-terminally of a non-TM helix.

49. The cell of claim 43, wherein the saccharide importer originates from the major facilitator superfamily (MFS) of transporters and comprises a polypeptide comprising:
an IPR001927 domain as defined by InterPro 90.0 as released on 4 Aug. 2022, and
a polypeptide comprising 12 transmembrane (TM) domains with a conserved domain [AGSV][HNQ][ACDEGNQSTV]XX[FWY]XXXXX(no L) as represented by SEQ ID NO: 17, wherein X can be any amino acid residue, present in the first TM domain, optionally a polypeptide comprising 12 TM domains with a conserved domain [AGS]Q[ACGNQSTV]XX[FWY] as represented by SEQ ID NO: 18, wherein X can be any amino acid residue, present in the first TM domain,
optionally, wherein the second amino acid residue of SEQ ID NO: 17 is aligned to Lys18 of the polypeptide with SEQ ID NO: 22,
optionally, wherein the second amino acid residue of SEQ ID NO: 18, is aligned to Lys18 of the polypeptide with SEQ ID NO: 22.

50. The cell of claim 43, wherein the saccharide importer further has uptake activity for one or more other saccharide(s) different from LN3, wherein the one or more other saccharide(s) different from LN3 are selected from the group consisting of monosaccharide, disaccharide, oligosaccharide, and polysaccharide.

51. The cell of claim 43, wherein the saccharide importer has uptake activity for LN3 but not for a) LNT (lacto-N-tetraose, Galβ1-3GlcNAcβ1-3Galβ1-4Glc) and/or b) LNnT (lacto-N-neotetraose, Galβ1-4GlcNAcβ1-3Galβ1-4Glc).

52. The cell of claim 40, wherein the one or more bioproduct(s) is selected from the group consisting of saccharide, monosaccharide, activated monosaccharide, phosphorylated monosaccharide, disaccharide, oligosaccharide, polysaccharide, milk saccharide, mammalian milk saccharide, mammalian milk oligosaccharide (MMO), human milk saccharide, human milk oligosaccharide (HMO), neutral (non-charged) saccharide, negatively charged saccharide, fucosylated saccharide, sialylated saccharide, neutral (non-charged) oligosaccharide, negatively charged oligosaccharide, fucosylated oligosaccharide, sialylated oligosaccharide, N-acetylglucosamine containing oligosaccharide, N-acetyllactosamine containing oligosaccharide, lacto-N-biose containing oligosaccharide, lactose containing oligosaccharide, non-fucosylated neutral (non-charged) oligosaccharide, O-antigen, enterobacterial common antigen (ECA), the oligosaccharide repeats present in capsular polysaccharides, peptidoglycan, amino-sugar, Lewis-type antigen oligosaccharide, antigen of the human ABO blood group system, animal oligosaccharide, animal N-glycans, animal O-glycans, plant oligosaccharide, plant N-glycans, plant O-glycans, LN3, lacto-N-tetraose (LNT), lacto-N-neotetraose (LNnT), oligosaccharide derived from LN3, 3′sialyllactose (3′SL), 6′sialyllactose (6′SL), sialyllacto-N-tetraose a (LSTa), sialyllacto-N-tetraose b (LSTb), sialyllacto-N-tetraose c (LSTc), sialyllacto-N-tetraose d (LSTd), lacto-N-fucopentaose I, lacto-N-neofucopentaose, lacto-N-fucopentaose II, lacto-N-fucopentaose III, lacto-N-fucopentaose V, lacto-N-neofucopentaose V, lacto-N-difucohexaose I, lacto-N-neodifucohexaose, lacto-N-difucohexaose II, monofucosyllacto-n-hexaose III, difucosyllacto-N-hexaose a, 6′-galactosyllactose, 3′-galactosyllactose, lacto-N-hexaose, lacto-N-neohexaose, 2′-fucosyllactose (2′FL), 3-fucosyllactose (3-FL), difucosyllactose (DiFL), chitosan, chitosan comprising oligosaccharide, heparosan, glycosaminoglycan oligosaccharide, heparin, heparan sulphate, chondroitin sulphate, dermatan sulphate, hyaluronan, hyaluronic acid keratan sulphate, and one or more LN3-derived oligosaccharide(s).

53. The cell of claim 43, wherein the one or more bioproduct(s) is selected from the group consisting of saccharide, monosaccharide, activated monosaccharide, phosphorylated monosaccharide, disaccharide, oligosaccharide, polysaccharide, milk saccharide, mammalian milk saccharide, mammalian milk oligosaccharide (MMO), human milk saccharide, human milk oligosaccharide (HMO), neutral (non-charged) saccharide, negatively charged saccharide, fucosylated saccharide, sialylated saccharide, neutral (non-charged) oligosaccharide, negatively charged oligosaccharide, fucosylated oligosaccharide, sialylated oligosaccharide, N-acetylglucosamine containing oligosaccharide, N-acetyllactosamine containing oligosaccharide, lacto-N-biose containing oligosaccharide, lactose containing oligosaccharide, non-fucosylated neutral (non-charged) oligosaccharide, O-antigen, enterobacterial common antigen (ECA), the oligosaccharide repeats present in capsular polysaccharides, peptidoglycan, amino-sugar, Lewis-type antigen oligosaccharide, antigen of the human ABO blood group system, animal oligosaccharide, animal N-glycans, animal O-glycans, plant oligosaccharide, plant N-glycans, plant O-glycans, LN3, lacto-N-tetraose (LNT), lacto-N-neotetraose (LNnT), oligosaccharide derived from LN3, 3′sialyllactose (3′SL), 6′sialyllactose (6′SL), sialyllacto-N-tetraose a (LSTa), sialyllacto-N-tetraose b (LSTb), sialyllacto-N-tetraose c (LSTc), sialyllacto-N-tetraose d (LSTd), lacto-N-fucopentaose I, lacto-N-neofucopentaose, lacto-N-fucopentaose II, lacto-N-fucopentaose III, lacto-N-fucopentaose V, lacto-N-neofucopentaose V, lacto-N-difucohexaose I, lacto-N-neodifucohexaose, lacto-N-difucohexaose II, monofucosyllacto-n-hexaose III, difucosyllacto-N-hexaose a, 6′-galactosyllactose, 3′-galactosyllactose, lacto-N-hexaose, lacto-N-neohexaose, 2′-fucosyllactose (2′FL), 3-fucosyllactose (3-FL), difucosyllactose (DiFL), chitosan, chitosan comprising oligosaccharide, heparosan, glycosaminoglycan oligosaccharide, heparin, heparan sulphate, chondroitin sulphate, dermatan sulphate, hyaluronan, hyaluronic acid keratan sulphate, and one or more LN3-derived oligosaccharide(s).

54. The cell of claim 40, wherein the cell is further genetically engineered to produce the one or more bioproduct(s).

55. The cell of claim 43, wherein the cell is further genetically engineered to produce the one or more bioproduct(s).

56. The cell of claim 43, wherein the cell is able to produce the one or more bioproduct(s) from one or more precursor(s).

57. The cell of claim 56, wherein the cell is able to produce at least one of the one or more precursor(s), optionally the cell is genetically engineered to produce at least one of the one or more precursor(s).

58. The cell of claim 56, wherein at least one of the one or more precursor(s) is internalized in the cell via the saccharide importer.

59. The cell of claim 40, wherein the one or more precursor(s) is selected from the group consisting of saccharide, monosaccharide, activated monosaccharide, phosphorylated monosaccharide, disaccharide, oligosaccharide, polysaccharide, milk saccharide, mammalian milk saccharide, mammalian milk oligosaccharide (MMO), human milk saccharide, human milk oligosaccharide (HMO), neutral (non-charged) saccharide, negatively charged saccharide, fucosylated saccharide, sialylated saccharide, neutral (non-charged) oligosaccharide, negatively charged oligosaccharide, fucosylated oligosaccharide, sialylated oligosaccharide, N-acetylglucosamine containing oligosaccharide, N-acetyllactosamine containing oligosaccharide, lacto-N-biose containing oligosaccharide, lactose containing oligosaccharide, non-fucosylated neutral (non-charged) oligosaccharide, O-antigen, enterobacterial common antigen (ECA), the oligosaccharide repeats present in capsular polysaccharides, peptidoglycan, amino-sugar, Lewis-type antigen oligosaccharide, antigen of the human ABO blood group system, animal oligosaccharide, animal N-glycans, animal O-glycans, plant oligosaccharide, plant N-glycans, plant O-glycans, LN3, lacto-N-tetraose (LNT), lacto-N-neotetraose (LNnT), oligosaccharide derived from LN3, 3′sialyllactose (3′SL), 6′sialyllactose (6′SL), sialyllacto-N-tetraose a (LSTa), sialyllacto-N-tetraose b (LSTb), sialyllacto-N-tetraose c (LSTc), sialyllacto-N-tetraose d (LSTd), lacto-N-fucopentaose I, lacto-N-neofucopentaose, lacto-N-fucopentaose II, lacto-N-fucopentaose III, lacto-N-fucopentaose V, lacto-N-neofucopentaose V, lacto-N-difucohexaose I, lacto-N-neodifucohexaose, lacto-N-difucohexaose II, monofucosyllacto-n-hexaose III, difucosyllacto-N-hexaose a, 6′-galactosyllactose, 3′-galactosyllactose, lacto-N-hexaose, lacto-N-neohexaose, 2′-fucosyllactose (2′FL), 3-fucosyllactose (3-FL), difucosyllactose (DiFL), chitosan, chitosan comprising oligosaccharide, heparosan, glycosaminoglycan oligosaccharide, heparin, heparan sulphate, chondroitin sulphate, dermatan sulphate, hyaluronan, hyaluronic acid, keratan sulphate, and one or more LN3-derived oligosaccharide(s).

60. The cell of claim 56, wherein the one or more precursor(s) is selected from the group consisting of saccharide, monosaccharide, activated monosaccharide, phosphorylated monosaccharide, disaccharide, oligosaccharide, polysaccharide, milk saccharide, mammalian milk saccharide, mammalian milk oligosaccharide (MMO), human milk saccharide, human milk oligosaccharide (HMO), neutral (non-charged) saccharide, negatively charged saccharide, fucosylated saccharide, sialylated saccharide, neutral (non-charged) oligosaccharide, negatively charged oligosaccharide, fucosylated oligosaccharide, sialylated oligosaccharide, N-acetylglucosamine containing oligosaccharide, N-acetyllactosamine containing oligosaccharide, lacto-N-biose containing oligosaccharide, lactose containing oligosaccharide, non-fucosylated neutral (non-charged) oligosaccharide, O-antigen, enterobacterial common antigen (ECA), the oligosaccharide repeats present in capsular polysaccharides, peptidoglycan, amino-sugar, Lewis-type antigen oligosaccharide, antigen of the human ABO blood group system, animal oligosaccharide, animal N-glycans, animal O-glycans, plant oligosaccharide, plant N-glycans, plant O-glycans, LN3, lacto-N-tetraose (LNT), lacto-N-neotetraose (LNnT), oligosaccharide derived from LN3, 3′sialyllactose (3′SL), 6′sialyllactose (6′SL), sialyllacto-N-tetraose a (LSTa), sialyllacto-N-tetraose b (LSTb), sialyllacto-N-tetraose c (LSTc), sialyllacto-N-tetraose d (LSTd), lacto-N-fucopentaose I, lacto-N-neofucopentaose, lacto-N-fucopentaose II, lacto-N-fucopentaose III, lacto-N-fucopentaose V, lacto-N-neofucopentaose V, lacto-N-difucohexaose I, lacto-N-neodifucohexaose, lacto-N-difucohexaose II, monofucosyllacto-n-hexaose III, difucosyllacto-N-hexaose a, 6′-galactosyllactose, 3′-galactosyllactose, lacto-N-hexaose, lacto-N-neohexaose, 2′-fucosyllactose (2′FL), 3-fucosyllactose (3-FL), difucosyllactose (DiFL), chitosan, chitosan comprising oligosaccharide, heparosan, glycosaminoglycan oligosaccharide, heparin, heparan sulphate, chondroitin sulphate, dermatan sulphate, hyaluronan, hyaluronic acid, keratan sulphate, and one or more LN3-derived oligosaccharide(s).

61. The cell of claim 40, wherein the cell:
i) possesses, optionally expresses or overexpresses, one or more glycosyltransferase(s) selected from the group consisting of fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-B-L-altrosamine transaminases, UDP-N-acetylglucosamine enolpyruvyl transferases and fucosaminyltransferases,
ii) is able to produce one or more nucleotide-activated sugars,
iii) comprises at least one pathway selected from the group consisting of fucosylation pathway, sialylation pathway, galactosylation pathway, N-acetylglucosaminylation pathway, N-acetylgalactosaminylation pathway, mannosylation pathway and N-acetylmannosaminylation pathway, and/or
iv) comprises a catabolic pathway for selected mono-, di- or oligosaccharides which is at least partially inactivated, the mono-, di-, or oligosaccharides being involved in and/or required for producing the one or more bioproduct(s).

62. The cell of claim 43, wherein the cell:
i) possesses, optionally expresses or overexpresses, one or more glycosyltransferase(s) selected from the group consisting of fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-B-L-altrosamine transaminases, UDP-N-acetylglucosamine enolpyruvyl transferases and fucosaminyltransferases,
ii) is able to produce one or more nucleotide-activated sugars,
iii) comprises at least one pathway selected from the group consisting of fucosylation pathway, sialylation pathway, galactosylation pathway, N-acetylglucosaminylation pathway, N-acetylgalactosaminylation pathway, mannosylation pathway and N-acetylmannosaminylation pathway, and/or
iv) comprises a catabolic pathway for selected mono-, di- or oligosaccharides which is at least partially inactivated, the mono-, di-, or oligosaccharides being involved in and/or required for producing the one or more bioproduct(s).

63. The cell of claim 40, wherein the cell is selected from the group consisting of prokaryotic cells, eukaryotic cells, yeast cells, bacterial cells, archaebacterial cells, algae cells, and fungal cells.

64. The cell of claim 43, wherein the cell is selected from the group consisting of prokaryotic cells, eukaryotic cells, yeast cells, bacterial cells, archaebacterial cells, algae cells, and fungal cells.

65. The cell of claim 40, wherein the cell is an E. coli or yeast with a lactose permease positive phenotype, optionally wherein the lactose permease is coded by the gene LacY or LAC12, respectively.

66. The cell of claim 43, wherein the cell is an E. coli or yeast with a lactose permease positive phenotype, optionally wherein the lactose permease is coded by the gene LacY or LAC12, respectively.

67. A method of producing one or more bioproduct(s), the method comprising the steps of:
a) cultivating and/or incubating the cell of claim 40 under conditions permissive to express the saccharide importer and to produce the one or more bioproduct(s), and
b) optionally separating the one or more bioproduct(s) from the cultivation or incubation.

68. A method of producing one or more bioproduct(s), the method comprising the steps of:
a) cultivating and/or incubating the cell of claim 43 under conditions permissive to express the saccharide importer and to produce the one or more bioproduct(s), and
b) optionally separating the one or more bioproduct(s) from the cultivation or incubation.

69. The method according to claim 67, wherein the one or more bioproduct(s) is selected from the group consisting of saccharide, monosaccharide, activated monosaccharide, phosphorylated monosaccharide, disaccharide, oligosaccharide, polysaccharide, milk saccharide, mammalian milk saccharide, mammalian milk oligosaccharide (MMO), human milk saccharide, human milk oligosaccharide (HMO), neutral (non-charged) saccharide, negatively charged saccharide, fucosylated saccharide, sialylated saccharide, neutral (non-charged) oligosaccharide, negatively charged oligosaccharide, fucosylated oligosaccharide, sialylated oligosaccharide, N-acetylglucosamine containing oligosaccharide, N-acetyllactosamine containing oligosaccharide, lacto-N-biose containing oligosaccharide, lactose containing oligosaccharide, non-fucosylated neutral (non-charged) oligosaccharide, O-antigen, enterobacterial common antigen (ECA), the oligosaccharide repeats present in capsular polysaccharides, peptidoglycan, amino-sugar, Lewis-type antigen oligosaccharide, antigen of the human ABO blood group system, animal oligosaccharide, animal N-glycans, animal O-glycans, plant oligosaccharide, plant N-glycans, plant O-glycans, LN3, lacto-N-tetraose (LNT), lacto-N-neotetraose (LNnT), oligosaccharide derived from LN3, 3′sialyllactose (3′SL), 6′sialyllactose (6′SL), sialyllacto-N-tetraose a (LSTa), sialyllacto-N-tetraose b (LSTb), sialyllacto-N-tetraose c (LSTc), sialyllacto-N-tetraose d (LSTd), lacto-N-fucopentaose I, lacto-N-neofucopentaose, lacto-N-fucopentaose II, lacto-N-fucopentaose III, lacto-N-fucopentaose V, lacto-N-neofucopentaose V, lacto-N-difucohexaose I, lacto-N-neodifucohexaose, lacto-N-difucohexaose II, monofucosyllacto-n-hexaose III, difucosyllacto-N-hexaose a, 6′-galactosyllactose, 3′-galactosyllactose, lacto-N-hexaose, lacto-N-neohexaose, 2′-fucosyllactose (2′FL), 3-fucosyllactose (3-FL), difucosyllactose (DiFL), chitosan, chitosan comprising oligosaccharide, heparosan, glycosaminoglycan oligosaccharide, heparin, heparan sulphate, chondroitin sulphate, dermatan sulphate, hyaluronan, hyaluronic acid, keratan sulphate and one or more LN3-derived oligosaccharide(s).

70. The method according to claim 68, wherein the one or more bioproduct(s) is selected from the group consisting of saccharide, monosaccharide, activated monosaccharide, phosphorylated monosaccharide, disaccharide, oligosaccharide, polysaccharide, milk saccharide, mammalian milk saccharide, mammalian milk oligosaccharide (MMO), human milk saccharide, human milk oligosaccharide (HMO), neutral (non-charged) saccharide, negatively charged saccharide, fucosylated saccharide, sialylated saccharide, neutral (non-charged) oligosaccharide, negatively charged oligosaccharide, fucosylated oligosaccharide, sialylated oligosaccharide, N-acetylglucosamine containing oligosaccharide, N-acetyllactosamine containing oligosaccharide, lacto-N-biose containing oligosaccharide, lactose containing oligosaccharide, non-fucosylated neutral (non-charged) oligosaccharide, O-antigen, enterobacterial common antigen (ECA), the oligosaccharide repeats present in capsular polysaccharides, peptidoglycan, amino-sugar, Lewis-type antigen oligosaccharide, antigen of the human ABO blood group system, animal oligosaccharide, animal N-glycans, animal O-glycans, plant oligosaccharide, plant N-glycans, plant O-glycans, LN3, lacto-N-tetraose (LNT), lacto-N-neotetraose (LNnT), oligosaccharide derived from LN3, 3′sialyllactose (3′SL), 6′sialyllactose (6′SL), sialyllacto-N-tetraose a (LSTa), sialyllacto-N-tetraose b (LSTb), sialyllacto-N-tetraose c (LSTc), sialyllacto-N-tetraose d (LSTd), lacto-N-fucopentaose I, lacto-N-neofucopentaose, lacto-N-fucopentaose II, lacto-N-fucopentaose III, lacto-N-fucopentaose V, lacto-N-neofucopentaose V, lacto-N-difucohexaose I, lacto-N-neodifucohexaose, lacto-N-difucohexaose II, monofucosyllacto-n-hexaose III, difucosyllacto-N-hexaose a, 6′-galactosyllactose, 3′-galactosyllactose, lacto-N-hexaose, lacto-N-neohexaose, 2′-fucosyllactose (2′FL), 3-fucosyllactose (3-FL), difucosyllactose (DiFL), chitosan, chitosan comprising oligosaccharide, heparosan, glycosaminoglycan oligosaccharide, heparin, heparan sulphate, chondroitin sulphate, dermatan sulphate, hyaluronan, hyaluronic acid, keratan sulphate and one or more LN3-derived oligosaccharide(s).

71. The method according to claim 67, wherein the cell produces the one or more bioproduct(s) with a higher yield and/or higher purity compared to a cell with the same genetic make-up but lacking expression of the saccharide importer.

72. The method according to claim 68, wherein the cell produces the one or more bioproduct(s) with a higher yield and/or higher purity compared to a cell with the same genetic make-up but lacking expression of the saccharide importer.

73. The method according to claim 67, wherein the one or more bioproduct(s) is one or more LN3-derived oligosaccharide(s) and wherein the cell produces the one or more LN3-derived oligosaccharide(s) with a higher yield and/or higher purity compared to a cell with the same genetic make-up but lacking expression of the saccharide importer.

74. The method according to claim 68, wherein the one or more bioproduct(s) is one or more LN3-derived oligosaccharide(s) and wherein the cell produces the one or more LN3-derived oligosaccharide(s) with a higher yield and/or higher purity compared to a cell with the same genetic make-up but lacking expression of the saccharide importer.

75. The method according to claim 67, wherein the:
cultivation medium contains at least one carbon source selected from the group consisting of glucose, fructose, sucrose and glycerol,
cultivation or incubation medium contains at least one compound selected from the group consisting of lactose, galactose, glucose, UDP-GlcNAc, GlcNAc, UDP-Gal, UDP-Glc and LN3, and/or
cultivation or incubation medium comprises one or more precursor(s) that is/are used for production of the one or more bioproduct(s).

76. The method according to claim 68, wherein the:
cultivation medium contains at least one carbon source selected from the group consisting of glucose, fructose, sucrose and glycerol,
cultivation or incubation medium contains at least one compound selected from the group consisting of lactose, galactose, glucose, UDP-GlcNAc, GlcNAc, UDP-Gal, UDP-Glc and LN3, and/or
cultivation or incubation medium comprises one or more precursor(s) that is/are used for production of the one or more bioproduct(s).

77. The method according to claim 75, wherein the one or more precursor(s) in the cultivation or incubation medium is/are produced by the cell and/or wherein the one or more precursor(s) is/are taken up by the cell via the saccharide importer.

78. The method according to claim 76, wherein the one or more precursor(s) in the cultivation or incubation medium is/are produced by the cell and/or wherein the one or more precursor(s) is/are taken up by the cell via the saccharide importer.

79. The method according to claim 67, wherein the one or more bioproduct(s) is recovered from the cultivation or incubation medium and/or the cell.

80. The method according to claim 68, wherein the one or more bioproduct(s) is recovered from the cultivation or incubation medium and/or the cell.

81. The method according to claim 67, wherein the method results in a production of at least 50 g/L of the one or more bioproduct(s) in the final volume of the cultivation or incubation.

82. The method according to claim 68, wherein the method results in a production of at least 50 g/L of the one or more bioproduct(s) in the final volume of the cultivation or incubation.

83. A method of using the cell of claim 40 to produce of one or more bioproduct(s), the method comprising: cultivating the cell.

84. A method of using the cell of claim 43 to produce of one or more bioproduct(s), the method comprising: cultivating the cell.

85. The method according to 83, wherein the bioproduct(s) comprise one or more LN3-derived oligosaccharide(s).

86. The method according to 84, wherein the bioproduct(s) comprise one or more LN3-derived oligosaccharide(s).

87. A saccharide importer having uptake activity for lacto-N-triose (LN3, GlcNAc-β1,3-Gal-β1,4-Glc) and comprising a polypeptide that has a deletion, insertion and/or a mutation of one or more amino acid residue(s):
N-terminally from the first transmembrane (TM) domain of any one of SEQ ID NOs: 12, 2, 1, 9, 19, 20 or 21, optionally, the deletion, insertion and/or mutation is in or N-terminally of a non-TM helix, and/or
C-terminally from the last TM domain of any one of SEQ ID NOs: 12, 2, 1, 9, 19, 20 or 21, optionally the deletion, insertion and/or mutation is in or C-terminally of a non-TM helix.

88. The saccharide importer of claim 87, wherein the saccharide importer further has uptake activity for one or more other saccharide(s) selected from the group consisting of monosaccharide, disaccharide, oligosaccharide, and polysaccharide.

89. The saccharide importer of claim 87, wherein the saccharide importer has uptake activity for LN3 but not for a) LNT and/or b) LNnT.

90. A method of producing one or more bioproduct(s), the method comprising:
utilizing a saccharide importer in the method, the saccharide importer having uptake activity for lacto-N-triose (LN3, GlcNAc-β1,3-Gal-β1,4-Glc) for producing one or more bioproduct(s), wherein the saccharide importer comprises a polypeptide:
that originates from the major facilitator superfamily (MFS) of transporters,
comprising an IPR domain selected from the group consisting of IPR001927, IPR002178, IPR016152, IPR018043, IPR020846, IPR036259 and IPR039672 as defined by InterPro 90.0 as released on 4 Aug. 2022,
comprising PF13347 domain and/or PF00359 domain as defined by PFAM 32.0 as released on September 2018,
comprising a PANTHER domain selected from the group consisting of PTHR11328, PTHR11328:SF24, PTHR11328:SF36 and PTHR11328:SF39 as defined by PANTHER 18.0 as released on 17 Sep. 2023,
comprising cd17332 domain and/or cd00211 domain as defined by the Conserved Domain Database CDD 3.20 as released on September 2022,
that is at least 25% identical over a stretch of at least 50 amino acid residues to any one of the polypeptides as represented by SEQ ID NOs: 4, 12, 2, 1, 3, 9, 16, 19, 20 or 21,
that is at least 25% identical to any one of the full-length polypeptides as represented by SEQ ID NOs: 4, 12, 2, 1, 3, 9, 16, 19, 20 or 21,
as represented by any one of SEQ ID NOs: 4, 12, 2, 1, 3, 9, 16, 19, 20 or 21,
that has a deletion, insertion and/or a mutation of one or more amino acid residue(s) N-terminally from the first transmembrane (TM) domain of any one of SEQ ID NOs: 12, 2, 1, 9, 19, 20 or 21, optionally, the deletion, insertion and/or mutation is in or N-terminally of a non-TM helix, and/or
that has a deletion, insertion and/or a mutation of one or more amino acid residue(s) C-terminally from the last TM domain of any one of SEQ ID NOs: 12, 2, 1, 9, 19, 20 or 21, optionally, the deletion, insertion and/or mutation is in or C-terminally of a non-TM helix,
and wherein the bioproduct(s) is/are selected from the group consisting of saccharide, monosaccharide, activated monosaccharide, phosphorylated monosaccharide, disaccharide, oligosaccharide, polysaccharide, milk saccharide, mammalian milk saccharide, mammalian milk oligosaccharide (MMO), human milk saccharide, human milk oligosaccharide (HMO), neutral (non-charged) saccharide, negatively charged saccharide, fucosylated saccharide, sialylated saccharide, neutral (non-charged) oligosaccharide, negatively charged oligosaccharide, fucosylated oligosaccharide, sialylated oligosaccharide, N-acetylglucosamine containing oligosaccharide, N-acetyllactosamine containing oligosaccharide, lacto-N-biose containing oligosaccharide, lactose containing oligosaccharide, non-fucosylated neutral (non-charged) oligosaccharide, O-antigen, enterobacterial common antigen (ECA), the oligosaccharide repeats present in capsular polysaccharides, peptidoglycan, amino-sugar, Lewis-type antigen oligosaccharide, antigen of the human ABO blood group system, animal oligosaccharide, animal N-glycans, animal O-glycans, plant oligosaccharide, plant N-glycans, plant O-glycans, LN3, lacto-N-tetraose (LNT), lacto-N-neotetraose (LNnT), oligosaccharide derived from LN3, 3′sialyllactose (3′SL), 6′sialyllactose (6′SL), sialyllacto-N-tetraose a (LSTa), sialyllacto-N-tetraose b (LSTb), sialyllacto-N-tetraose c (LSTc), sialyllacto-N-tetraose d (LSTd), lacto-N-fucopentaose I, lacto-N-neofucopentaose, lacto-N-fucopentaose II, lacto-N-fucopentaose III, lacto-N-fucopentaose V, lacto-N-neofucopentaose V, lacto-N-difucohexaose I, lacto-N-neodifucohexaose, lacto-N-difucohexaose II, monofucosyllacto-n-hexaose III, difucosyllacto-N-hexaose a, 6′-galactosyllactose, 3′-galactosyllactose, lacto-N-hexaose, lacto-N-neohexaose, 2′-fucosyllactose (2′FL), 3-fucosyllactose (3-FL), difucosyllactose (DiFL), chitosan, chitosan comprising oligosaccharide, heparosan, glycosaminoglycan oligosaccharide, heparin, heparan sulphate, chondroitin sulphate, dermatan sulphate, hyaluronan, hyaluronic acid, keratan sulphate and one or more LN3-derived oligosaccharide(s).

91. The method according to claim 90, wherein the saccharide importer originates from the major facilitator superfamily (MFS) of transporters and comprises a polypeptide comprising:
an IPR001927 domain as defined by InterPro 90.0 as released on 4 Aug. 2022, and
a polypeptide comprising 12 transmembrane (TM) domains with a conserved domain [AGSV][HNQ][ACDEGNQSTV]XX[FWY]XXXXX(no L) as represented by SEQ ID NO: 17, wherein X can be any amino acid residue, present in the first TM domain, optionally a polypeptide comprising 12 TM domains with a conserved domain [AGS]Q[ACGNQSTV]XX[FWY] as represented by SEQ ID NO: 18, wherein X can be any amino acid residue,
optionally, wherein the second amino acid residue of SEQ ID NO: 17 is aligned to Lys18 of the polypeptide with SEQ ID NO: 22,
optionally, wherein the second amino acid residue of SEQ ID NO: 18 is aligned to Lys18 of the polypeptide with SEQ ID NO: 22.

92. The method according to claim 90, wherein the saccharide importer further has uptake activity for one or more other saccharide(s) different from LN3 selected from the group consisting of monosaccharide, disaccharide, oligosaccharide and polysaccharide.

93. The method according to claim 90, wherein the saccharide importer has uptake activity for LN3 but not for a) LNT and/or b) LNnT.

94. A polynucleotide encoding a saccharide importer having uptake activity for lacto-N-triose (LN3, GlcNAc-β1,3-Gal-β1,4-Glc), wherein the saccharide importer comprises a polypeptide:
that originates from the major facilitator superfamily (MFS) of transporters,
comprising an IPR domain selected from the group consisting of IPR001927, IPR002178, IPR016152, IPR018043, IPR020846, IPR036259 and IPR039672 as defined by InterPro 90.0 as released on 4 Aug. 2022,
comprising PF13347 domain and/or PF00359 domain as defined by PFAM 32.0 as released on September 2018,
comprising a PANTHER domain selected from the group consisting of PTHR11328, PTHR11328:SF24, PTHR11328:SF36, and PTHR11328:SF39 as defined by PANTHER 18.0 as released on 17 Sep. 2023,
comprising cd17332 domain and/or cd00211 domain as defined by the Conserved Domain Database CDD 3.20 as released on September 2022,
that is at least 25% identical over a stretch of at least 50 amino acid residues to any one of the polypeptides as represented by SEQ ID NOs: 4, 12, 2, 1, 3, 9, 16, 19, 20 or 21,
that is at least 25% identical to any one of the full-length polypeptides as represented by SEQ ID NOs: 4, 12, 2, 1, 3, 9, 16, 19, 20 or 21,
as represented by any one of SEQ ID NOs: 4, 12, 2, 1, 3, 9, 16, 19, 20 or 21,
that has a deletion, insertion and/or a mutation of one or more amino acid residue(s) N-terminally from the first transmembrane (TM) domain of any one of SEQ ID NOs: 12, 2, 1, 9, 19, 20 or 21, optionally the deletion, insertion and/or mutation is in or N-terminally of a non-TM helix, and/or
that has a deletion, insertion and/or a mutation of one or more amino acid residue(s) C-terminally from the last TM domain of any one of SEQ ID NOs: 12, 2, 1, 9, 19, 20 or 21, optionally the deletion, insertion and/or mutation is in or C-terminally of a non-TM helix.

95. The polynucleotide of claim 94, wherein the saccharide importer originates from the major facilitator superfamily (MFS) of transporters and comprises a polypeptide comprising:
an IPR001927 domain as defined by InterPro 90.0 as released on 4 Aug. 2022, and
a polypeptide comprising 12 transmembrane (TM) domains with a conserved domain [AGSV][HNQ][ACDEGNQSTV]XX[FWY]XXXXX(no L) as represented by SEQ ID NO: 17, wherein X can be any amino acid residue, present in the first TM domain, optionally a polypeptide comprising 12 TM domains with a conserved domain [AGS]Q[ACGNQSTV]XX[FWY] as represented by SEQ ID NO: 18, wherein X can be any amino acid residue,
optionally, wherein the second amino acid residue of SEQ ID NO: 17 is aligned to Lys18 of the polypeptide with SEQ ID NO: 22,
optionally, wherein the second amino acid residue of SEQ ID NO: 18 is aligned to Lys18 of the polypeptide with SEQ ID NO: 22.

96. A vector comprising the polynucleotide of claim 94.

97. A method of producing one or more bioproduct(s) selected from the group consisting of saccharide, monosaccharide, activated monosaccharide, phosphorylated monosaccharide, disaccharide, oligosaccharide, polysaccharide, milk saccharide, mammalian milk saccharide, mammalian milk oligosaccharide (MMO), human milk saccharide, human milk oligosaccharide (HMO), neutral (non-charged) saccharide, negatively charged saccharide, fucosylated saccharide, sialylated saccharide, neutral (non-charged) oligosaccharide, negatively charged oligosaccharide, fucosylated oligosaccharide, sialylated oligosaccharide, N-acetylglucosamine containing oligosaccharide, N-acetyllactosamine containing oligosaccharide, lacto-N-biose containing oligosaccharide, lactose containing oligosaccharide, non-fucosylated neutral (non-charged) oligosaccharide, O-antigen, enterobacterial common antigen (ECA), the oligosaccharide repeats present in capsular polysaccharides, peptidoglycan, amino-sugar, Lewis-type antigen oligosaccharide, antigen of the human ABO blood group system, animal oligosaccharide, animal N-glycans, animal O-glycans, plant oligosaccharide, plant N-glycans, plant O-glycans, LN3, lacto-N-tetraose (LNT), lacto-N-neotetraose (LNnT), oligosaccharide derived from LN3, 3′sialyllactose (3′SL), 6′sialyllactose (6′SL), sialyllacto-N-tetraose a (LSTa), sialyllacto-N-tetraose b (LSTb), sialyllacto-N-tetraose c (LSTc), sialyllacto-N-tetraose d (LSTd), lacto-N-fucopentaose I, lacto-N-neofucopentaose, lacto-N-fucopentaose II, lacto-N-fucopentaose III, lacto-N-fucopentaose V, lacto-N-neofucopentaose V, lacto-N-difucohexaose I, lacto-N-neodifucohexaose, lacto-N-difucohexaose II, monofucosyllacto-n-hexaose III, difucosyllacto-N-hexaose a, 6′-galactosyllactose, 3′-galactosyllactose, lacto-N-hexaose, lacto-N-neohexaose, 2′-fucosyllactose (2′FL), 3-fucosyllactose (3-FL), difucosyllactose (DiFL), chitosan, chitosan comprising oligosaccharide, heparosan, glycosaminoglycan oligosaccharide, heparin, heparan sulphate, chondroitin sulphate, dermatan sulphate, hyaluronan, hyaluronic acid, keratan sulphate and one or more LN3 derived oligosaccharide(s), the method comprising using the polynucleotide of claim 94 to produce the bioproduct(s).

98. A method of producing one or more bioproduct(s) selected from the group consisting of saccharide, monosaccharide, activated monosaccharide, phosphorylated monosaccharide, disaccharide, oligosaccharide, polysaccharide, a milk saccharide, a mammalian milk saccharide, a mammalian milk oligosaccharide (MMO), a human milk saccharide, a human milk oligosaccharide (HMO), a neutral (non-charged) saccharide, a negatively charged saccharide, a fucosylated saccharide, a sialylated saccharide, a neutral (non-charged) oligosaccharide, a negatively charged oligosaccharide, a fucosylated oligosaccharide, a sialylated oligosaccharide, N-acetylglucosamine containing oligosaccharide, N-acetyllactosamine containing oligosaccharide, lacto-N-biose containing oligosaccharide, lactose containing oligosaccharide, non-fucosylated neutral (non-charged) oligosaccharide, O-antigen, enterobacterial common antigen (ECA), the oligosaccharide repeats present in capsular polysaccharides, peptidoglycan, an amino-sugar, a Lewis-type antigen oligosaccharide, an antigen of the human ABO blood group system, an animal oligosaccharide, animal N-glycans, animal O-glycans, a plant oligosaccharide, plant N-glycans, plant O-glycans, LN3, lacto-N-tetraose (LNT), lacto-N-neotetraose (LNnT), an oligosaccharide derived from LN3, 3′sialyllactose (3′SL), 6′sialyllactose (6′SL), sialyllacto-N-tetraose a (LSTa), sialyllacto-N-tetraose b (LSTb), sialyllacto-N-tetraose c (LSTc), sialyllacto-N-tetraose d (LSTd), lacto-N-fucopentaose I, lacto-N-neofucopentaose, lacto-N-fucopentaose II, lacto-N-fucopentaose III, lacto-N-fucopentaose V, lacto-N-neofucopentaose V, lacto-N-difucohexaose I, lacto-N-neodifucohexaose, lacto-N-difucohexaose II, monofucosyllacto-n-hexaose III, difucosyllacto-N-hexaose a, 6′-galactosyllactose, 3′-galactosyllactose, lacto-N-hexaose, lacto-N-neohexaose, 2′-fucosyllactose (2′FL), 3-fucosyllactose (3-FL), difucosyllactose (DiFL), chitosan, chitosan comprising oligosaccharide, heparosan, chondroitin sulphate, glycosaminoglycan oligosaccharide, heparin, heparan sulphate, chondroitin sulphate, dermatan sulphate, hyaluronan, hyaluronic acid, keratan sulphate, and one or more LN3-derived oligosaccharide(s), the method comprising:
utilizing the vector of claim 96 to produce the one or more bioproduct(s).
